SEQUENCE LISTING (63 sequences)

SEQ ID NO: 1 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 2 - DNA, 1530 bp, Artificial Sequence
  Description of Artificial Sequence Synthetic polynucleotide
atgagcatgggcgctcctagaagcctgctgctggccctggctgccggcctggccgtggctagacctccaaacatcgtgctgatcttcgccgacgacctgggctatggtgacctgggctgctacggccacccctcttctacaacacccaatctggaccagctggccgctggcggcctgagattcacagacttctacgtgccagtgtccctgtgcaccccttctagagccgctctcctgaccggcagactgcctgtgcggatgggcatgtaccccggagtgctggtgcccagcagtagaggaggactgcctctggaagaggtgaccgtggccgaggtgctggccgccagaggctacctgacaggaatggccggaaaatggcacctgggagtgggcccagaaggcgccttcctgccaccacaccagggctttcaccggttcctggggatcccttacagccacgaccaaggcccttgtcagaacctgacatgcttcccccccgccacaccttgcgacggcggctgtgaccagggccttgtgcctatccccctgctggccaacctgagcgtggaagcccagcctccatggctgcctggcctcgaggccagatacatggccttcgctcatgatctgatggccgatgcccagagacaggacagaccttttttcctgtattacgccagccaccacacccactaccctcagttcagcggacagagcttcgccgagcggagcggcagaggccccttcggcgacagcctgatggaactggacgccgctgttggaaccctgatgaccgccattggcgatctgggcctgctcgaggaaaccctggtgatcttcaccgccgataacggccctgagacaatgcggatgtctagaggcggctgcagcggcctgctgcggtgcggcaagggcaccacctacgagggcggcgtgcgggaacccgccctggctttttggcctggccacatcgcccctggcgttacccacgagctggcttctagcctggacctgctgcccaccctggccgcactggccggagctccactgcctaatgtgaccctggatggcttcgacctgtcccctctgctgctcggcaccggcaagagccctagacagagcctgttcttctacccctcctaccctgatgaggtgcggggcgtctttgccgtcaggaccggcaaatacaaggcccatttctttacacagggcagcgcccactctgataccacagccgaccctgcctgccacgccagctccagcctgaccgcccacgagcctcctctgctatacgacctgagcaaggaccctggcgagaactacaacctgctgggtggcgtggccggcgctacacctgaggtgctgcaggccctgaagcagctgcagctgcttaaggcccaactggacgccgctgtgaccttcggccctagccaggtggccagaggagaagatcccgccctgcaaatctgctgccaccctggatgtacccctcggcccgcttgttgtcactgccccgaccctcacgcctga

SEQ ID NO: 3 - DNA, 1530 bp, Artificial Sequence
  Description of Artificial Sequence Synthetic polynucleotide
atgtctatgggagcccctagatctctgctgctggctctggctgctggactggcagttgccagacctcctaacatcgtgctgatcttcgccgacgatctcggctatggcgatctgggctgttacggacaccccagcagcaccacacctaacctggatcaacttgccgctggcggcctgagattcaccgatttctacgtgcccgtgtctctgtgcaccccttctagagctgctctgctgacaggcagactccctgtgcggatgggaatgtatcctggcgtgctggtgcctagctctagaggcggactgcctctggaagaagtgacagttgccgaagtgctggccgccagaggatatctgactggcatggccggaaagtggcacctcggagttggacctgaaggcgcttttctgcctcctcaccagggcttccaccggtttctgggcatcccttactctcacgatcagggcccctgccagaacctgacctgttttcctcctgccacaccttgcgacggcggctgtgatcaaggactggtgccaattcctctgctggccaacctgagcgtggaagctcaacctccttggctgccaggactggaagcccggtatatggccttcgctcacgacctgatggccgacgctcagagacaggacagaccattcttcctgtactacgccagccaccacacacactaccctcagtttagcggccagagcttcgccgagagatctggcagaggacctttcggcgacagcctgatggaactggatgccgctgtgggcacactgatgacagccatcggagatctgggactgctggaagagacactggtcatcttcaccgccgacaacggccccgagacaatgagaatgagcagaggcggctgtagcggcctgctgagatgtggcaagggcaccacatatgaaggcggcgtcagagaacctgctctggccttttggcctggccatattgctccaggcgtgacacacgagctggcctcttctctggatctgctgcctacactggcagctcttgctggtgctcccctgcctaatgtgaccctggatggcttcgatctgagcccactgctgctcggcacaggcaagtctccaagacagagcctgttcttctaccctagctaccccgatgaagtgcggggagtgtttgccgtgcggaccggaaagtataaggcccacttcttcacccaaggcagcgcccactctgacaccacagctgatcctgcttgtcacgccagctctagcctgacagcccatgaacctccactgctgtacgacctgagcaaggaccccggcgagaactacaatctgcttggcggagttgccggcgctacacctgaagttctgcaggccctgaaacagctccagctgctgaaagcccagctggacgctgccgtgacatttggacctagtcaggtggccagaggcgaggatcctgctctgcagatctgttgtcaccctggctgcacacccagacctgcctgctgtcattgtcctgatcctcacgcctga

SEQ ID NO: 4 - DNA, 1530 bp, Homo sapiens
atgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctga

SEQ ID NO: 5 - protein, 507 aa, Homo sapiens
MGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 6 - protein, 507 aa, Artificial Sequence
  Description of Artificial Sequence Synthetic polypeptide
MGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYVAFAHDLMADAQRQDRPFFLYYASHHTHYPQFSGQSFAERSGRGPFGDSLMELDAAVGTLMTAIGDLGLLEETLVIFTADNGPELMRMSNGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 7 - DNA, 1530 bp, Artificial Sequence
  Description of Artificial Sequence Synthetic polynucleotide
atgagcatgggcgctcctagaagcctgctgctggccctggctgccggcctggccgtggctagacctccaaacatcgtgctgatcttcgccgacgacctgggctatggtgacctgggctgctacggccacccctcttctacaacacccaatctggaccagctggccgctggcggcctgagattcacagacttctacgtgccagtgtccctgtgcaccccttctagagccgctctcctgaccggcagactgcctgtgcggatgggcatgtaccccggagtgctggtgcccagcagtagaggaggactgcctctggaagaggtgaccgtggccgaggtgctggccgccagaggctacctgacaggaatggccggaaaatggcacctgggagtgggcccagaaggcgccttcctgccaccacaccagggctttcaccggttcctggggatcccttacagccacgaccaaggcccttgtcagaacctgacatgcttcccccccgccacaccttgcgacggcggctgtgaccagggccttgtgcctatccccctgctggccaacctgagcgtggaagcccagcctccatggctgcctggcctcgaggccagatacgtggccttcgctcatgatctgatggccgatgcccagagacaggacagaccttttttcctgtattacgccagccaccacacccactaccctcagttcagcggacagagcttcgccgagcggagcggcagaggccccttcggcgacagcctgatggaactggacgccgctgttggaaccctgatgaccgccattggcgatctgggcctgctcgaggaaaccctggtgatcttcaccgccgataacggccctgagctgatgcggatgtctaacggcggctgcagcggcctgctgcggtgcggcaagggcaccacctacgagggcggcgtgcgggaacccgccctggctttttggcctggccacatcgcccctggcgttacccacgagctggcttctagcctggacctgctgcccaccctggccgcactggccggagctccactgcctaatgtgaccctggatggcttcgacctgtcccctctgctgctcggcaccggcaagagccctagacagagcctgttcttctacccctcctaccctgatgaggtgcggggcgtctttgccgtcaggaccggcaaatacaaggcccatttctttacacagggcagcgcccactctgataccacagccgaccctgcctgccacgccagctccagcctgaccgcccacgagcctcctctgctatacgacctgagcaaggaccctggcgagaactacaacctgctgggtggcgtggccggcgctacacctgaggtgctgcaggccctgaagcagctgcagctgcttaaggcccaactggacgccgctgtgaccttcggccctagccaggtggccagaggagaagatcccgccctgcaaatctgctgccaccctggatgtacccctcggcccgcttgttgtcactgccccgaccctcacgcctga

SEQ ID NO: 8 - DNA, 1530 bp, Artificial Sequence
  Description of Artificial Sequence Synthetic polynucleotide
atgtctatgggagcccctagatctctgctgctggctctggctgctggactggcagttgccagacctcctaacatcgtgctgatcttcgccgacgatctcggctatggcgatctgggctgttacggacaccccagcagcaccacacctaacctggatcaacttgccgctggcggcctgagattcaccgatttctacgtgcccgtgtctctgtgcaccccttctagagctgctctgctgacaggcagactccctgtgcggatgggaatgtatcctggcgtgctggtgcctagctctagaggcggactgcctctggaagaagtgacagttgccgaagtgctggccgccagaggatatctgactggcatggccggaaagtggcacctcggagttggacctgaaggcgcttttctgcctcctcaccagggcttccaccggtttctgggcatcccttactctcacgatcagggcccctgccagaacctgacctgttttcctcctgccacaccttgcgacggcggctgtgatcaaggactggtgccaattcctctgctggccaacctgagcgtggaagctcaacctccttggctgccaggactggaagcccggtatgtggccttcgctcacgacctgatggccgacgctcagagacaggacagaccattcttcctgtactacgccagccaccacacacactaccctcagtttagcggccagagcttcgccgagagatctggcagaggacctttcggcgacagcctgatggaactggatgccgctgtgggcacactgatgacagccatcggagatctgggactgctggaagagacactggtcatcttcaccgccgacaacggccccgagctgatgagaatgagcaacggcggctgtagcggcctgctgagatgtggcaagggcaccacatatgaaggcggcgtcagagaacctgctctggccttttggcctggccatattgctccaggcgtgacacacgagctggcctcttctctggatctgctgcctacactggcagctcttgctggtgctcccctgcctaatgtgaccctggatggcttcgatctgagcccactgctgctcggcacaggcaagtctccaagacagagcctgttcttctaccctagctaccccgatgaagtgcggggagtgtttgccgtgcggaccggaaagtataaggcccacttcttcacccaaggcagcgcccactctgacaccacagctgatcctgcttgtcacgccagctctagcctgacagcccatgaacctccactgctgtacgacctgagcaaggaccccggcgagaactacaatctgcttggcggagttgccggcgctacacctgaagttctgcaggccctgaaacagctccagctgctgaaagcccagctggacgctgccgtgacatttggacctagtcaggtggccagaggcgaggatcctgctctgcagatctgttgtcaccctggctgcacacccagacctgcctgctgtcattgtcctgatcctcacgcctga

SEQ ID NO: 9 - DNA, 334 bp, Artificial Sequence
  Description of Artificial Sequence Synthetic polynucleotide
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagct

SEQ ID NO: 10 - DNA, 1212 bp, Artificial Sequence
  Description of Artificial Sequence Synthetic polynucleotide
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggccaagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 11 - DNA, 1212 bp, Artificial Sequence
  Description of Artificial Sequence Synthetic polynucleotide
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagcccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggccaagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 12 - DNA, 584 bp, Artificial Sequence
  Description of Artificial Sequence Synthetic polynucleotide
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 13 - DNA, 204 bp, Artificial Sequence
  Description of Artificial Sequence Synthetic polynucleotide
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagct

SEQ ID NO: 14 - DNA, 602 bp, Artificial Sequence
  Description of Artificial Sequence Synthetic polynucleotide
gcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcaga

SEQ ID NO: 15 - DNA, 589 bp, Artificial Sequence
  Description of Artificial Sequence Synthetic polynucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 16 - DNA, 124 bp, Artificial Sequence
  Description of Artificial Sequence Synthetic polynucleotide
atttgtgaaatttgtgatgctattgctttatttgtaaccattataagctgcaataaacaagttaacaacaacaattgcattcattttatgtttcaggttcagggggaggtgtgggaggtttttt

SEQ ID NO: 17 - DNA, 91 bp, Artificial Sequence
  Description of Artificial Sequence Synthetic oligonucleotide
gcgcgctcgctcgctcactgaggccgcccgggcaaagcccgggcgtcgggcgacctttggtcgcccggcctcagtgagcgagcgagcgcgc

SEQ ID NO: 18 - DNA, 145 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 19 - DNA, 3766 bp, Artificial Sequence
  Description of Artificial Sequence Synthetic polynucleotide
gcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggccaagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacagatctatgagcatgggcgctcctagaagcctgctgctggccctggctgccggcctggccgtggctagacctccaaacatcgtgctgatcttcgccgacgacctgggctatggtgacctgggctgctacggccacccctcttctacaacacccaatctggaccagctggccgctggcggcctgagattcacagacttctacgtgccagtgtccctgtgcaccccttctagagccgctctcctgaccggcagactgcctgtgcggatgggcatgtaccccggagtgctggtgcccagcagtagaggaggactgcctctggaagaggtgaccgtggccgaggtgctggccgccagaggctacctgacaggaatggccggaaaatggcacctgggagtgggcccagaaggcgccttcctgccaccacaccagggctttcaccggttcctggggatcccttacagccacgaccaaggcccttgtcagaacctgacatgcttcccccccgccacaccttgcgacggcggctgtgaccagggccttgtgcctatccccctgctggccaacctgagcgtggaagcccagcctccatggctgcctggcctcgaggccagatacatggccttcgctcatgatctgatggccgatgcccagagacaggacagaccttttttcctgtattacgccagccaccacacccactaccctcagttcagcggacagagcttcgccgagcggagcggcagaggccccttcggcgacagcctgatggaactggacgccgctgttggaaccctgatgaccgccattggcgatctgggcctgctcgaggaaaccctggtgatcttcaccgccgataacggccctgagacaatgcggatgtctagaggcggctgcagcggcctgctgcggtgcggcaagggcaccacctacgagggcggcgtgcgggaacccgccctggctttttggcctggccacatcgcccctggcgttacccacgagctggcttctagcctggacctgctgcccaccctggccgcactggccggagctccactgcctaatgtgaccctggatggcttcgacctgtcccctctgctgctcggcaccggcaagagccctagacagagcctgttcttctacccctcctaccctgatgaggtgcggggcgtctttgccgtcaggaccggcaaatacaaggcccatttctttacacagggcagcgcccactctgataccacagccgaccctgcctgccacgccagctccagcctgaccgcccacgagcctcctctgctatacgacctgagcaaggaccctggcgagaactacaacctgctgggtggcgtggccggcgctacacctgaggtgctgcaggccctgaagcagctgcagctgcttaaggcccaactggacgccgctgtgaccttcggccctagccaggtggccagaggagaagatcccgccctgcaaatctgctgccaccctggatgtacccctcggcccgcttgttgtcactgccccgaccctcacgcctgaggtaccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgagctcatttgtgaaatttgtgatgctattgctttatttgtaaccattataagctgcaataaacaagttaacaacaacaattgcattcattttatgtttcaggttcagggggaggtgtgggaggttttttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 20 - DNA, 3766 bp, Artificial Sequence
  Description of Artificial Sequence Synthetic polynucleotide
gcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggccaagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacagatctatgagcatgggcgctcctagaagcctgctgctggccctggctgccggcctggccgtggctagacctccaaacatcgtgctgatcttcgccgacgacctgggctatggtgacctgggctgctacggccacccctcttctacaacacccaatctggaccagctggccgctggcggcctgagattcacagacttctacgtgccagtgtccctgtgcaccccttctagagccgctctcctgaccggcagactgcctgtgcggatgggcatgtaccccggagtgctggtgcccagcagtagaggaggactgcctctggaagaggtgaccgtggccgaggtgctggccgccagaggctacctgacaggaatggccggaaaatggcacctgggagtgggcccagaaggcgccttcctgccaccacaccagggctttcaccggttcctggggatcccttacagccacgaccaaggcccttgtcagaacctgacatgcttcccccccgccacaccttgcgacggcggctgtgaccagggccttgtgcctatccccctgctggccaacctgagcgtggaagcccagcctccatggctgcctggcctcgaggccagatacgtggccttcgctcatgatctgatggccgatgcccagagacaggacagaccttttttcctgtattacgccagccaccacacccactaccctcagttcagcggacagagcttcgccgagcggagcggcagaggccccttcggcgacagcctgatggaactggacgccgctgttggaaccctgatgaccgccattggcgatctgggcctgctcgaggaaaccctggtgatcttcaccgccgataacggccctgagctgatgcggatgtctaacggcggctgcagcggcctgctgcggtgcggcaagggcaccacctacgagggcggcgtgcgggaacccgccctggctttttggcctggccacatcgcccctggcgttacccacgagctggcttctagcctggacctgctgcccaccctggccgcactggccggagctccactgcctaatgtgaccctggatggcttcgacctgtcccctctgctgctcggcaccggcaagagccctagacagagcctgttcttctacccctcctaccctgatgaggtgcggggcgtctttgccgtcaggaccggcaaatacaaggcccatttctttacacagggcagcgcccactctgataccacagccgaccctgcctgccacgccagctccagcctgaccgcccacgagcctcctctgctatacgacctgagcaaggaccctggcgagaactacaacctgctgggtggcgtggccggcgctacacctgaggtgctgcaggccctgaagcagctgcagctgcttaaggcccaactggacgccgctgtgaccttcggccctagccaggtggccagaggagaagatcccgccctgcaaatctgctgccaccctggatgtacccctcggcccgcttgttgtcactgccccgaccctcacgcctgaggtaccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgagctcatttgtgaaatttgtgatgctattgctttatttgtaaccattataagctgcaataaacaagttaacaacaacaattgcattcattttatgtttcaggttcagggggaggtgtgggaggttttttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 21 - DNA, 3617 bp, Artificial Sequence
  Description of Artificial Sequence Synthetic polynucleotide
gcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacctctaggaagaccttcaatattggccattagccatattattcattggttatatagcataaatcaatattggctattggccattgcatacgttgtatctatatcataatatgtacatttatattggctcatgtccaatatgaccgccatgttgcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagacactttgtggcggtagtttatcacagttaaattgctaacgcagtcagtgcttctgacacaacagtctcgaacttaagctgcagaagttggtcgtgaggcactgggcaggtaagtatcaaggttacaagacaggtttaaggcgaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggagatctatgagcatgggcgctcctagaagcctgctgctggccctggctgccggcctggccgtggctagacctccaaacatcgtgctgatcttcgccgacgacctgggctatggtgacctgggctgctacggccacccctcttctacaacacccaatctggaccagctggccgctggcggcctgagattcacagacttctacgtgccagtgtccctgtgcaccccttctagagccgctctcctgaccggcagactgcctgtgcggatgggcatgtaccccggagtgctggtgcccagcagtagaggaggactgcctctggaagaggtgaccgtggccgaggtgctggccgccagaggctacctgacaggaatggccggaaaatggcacctgggagtgggcccagaaggcgccttcctgccaccacaccagggctttcaccggttcctggggatcccttacagccacgaccaaggcccttgtcagaacctgacatgcttcccccccgccacaccttgcgacggcggctgtgaccagggccttgtgcctatccccctgctggccaacctgagcgtggaagcccagcctccatggctgcctggcctcgaggccagatacatggccttcgctcatgatctgatggccgatgcccagagacaggacagaccttttttcctgtattacgccagccaccacacccactaccctcagttcagcggacagagcttcgccgagcggagcggcagaggccccttcggcgacagcctgatggaactggacgccgctgttggaaccctgatgaccgccattggcgatctgggcctgctcgaggaaaccctggtgatcttcaccgccgataacggccctgagacaatgcggatgtctagaggcggctgcagcggcctgctgcggtgcggcaagggcaccacctacgagggcggcgtgcgggaacccgccctggctttttggcctggccacatcgcccctggcgttacccacgagctggcttctagcctggacctgctgcccaccctggccgcactggccggagctccactgcctaatgtgaccctggatggcttcgacctgtcccctctgctgctcggcaccggcaagagccctagacagagcctgttcttctacccctcctaccctgatgaggtgcggggcgtctttgccgtcaggaccggcaaatacaaggcccatttctttacacagggcagcgcccactctgataccacagccgaccctgcctgccacgccagctccagcctgaccgcccacgagcctcctctgctatacgacctgagcaaggaccctggcgagaactacaacctgctgggtggcgtggccggcgctacacctgaggtgctgcaggccctgaagcagctgcagctgcttaaggcccaactggacgccgctgtgaccttcggccctagccaggtggccagaggagaagatcccgccctgcaaatctgctgccaccctggatgtacccctcggcccgcttgttgtcactgccccgaccctcacgcctgaggtaccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgagctcatttgtgaaatttgtgatgctattgctttatttgtaaccattataagctgcaataaacaagttaacaacaacaattgcattcattttatgtttcaggttcagggggaggtgtgggaggttttttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 22 - DNA, 3617 bp, Artificial Sequence
  Description of Artificial Sequence Synthetic polynucleotide
gcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacctctaggaagaccttcaatattggccattagccatattattcattggttatatagcataaatcaatattggctattggccattgcatacgttgtatctatatcataatatgtacatttatattggctcatgtccaatatgaccgccatgttgcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagacactttgtggcggtagtttatcacagttaaattgctaacgcagtcagtgcttctgacacaacagtctcgaacttaagctgcagaagttggtcgtgaggcactgggcaggtaagtatcaaggttacaagacaggtttaaggcgaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggagatctatgagcatgggcgctcctagaagcctgctgctggccctggctgccggcctggccgtggctagacctccaaacatcgtgctgatcttcgccgacgacctgggctatggtgacctgggctgctacggccacccctcttctacaacacccaatctggaccagctggccgctggcggcctgagattcacagacttctacgtgccagtgtccctgtgcaccccttctagagccgctctcctgaccggcagactgcctgtgcggatgggcatgtaccccggagtgctggtgcccagcagtagaggaggactgcctctggaagaggtgaccgtggccgaggtgctggccgccagaggctacctgacaggaatggccggaaaatggcacctgggagtgggcccagaaggcgccttcctgccaccacaccagggctttcaccggttcctggggatcccttacagccacgaccaaggcccttgtcagaacctgacatgcttcccccccgccacaccttgcgacggcggctgtgaccagggccttgtgcctatccccctgctggccaacctgagcgtggaagcccagcctccatggctgcctggcctcgaggccagatacgtggccttcgctcatgatctgatggccgatgcccagagacaggacagaccttttttcctgtattacgccagccaccacacccactaccctcagttcagcggacagagcttcgccgagcggagcggcagaggccccttcggcgacagcctgatggaactggacgccgctgttggaaccctgatgaccgccattggcgatctgggcctgctcgaggaaaccctggtgatcttcaccgccgataacggccctgagctgatgcggatgtctaacggcggctgcagcggcctgctgcggtgcggcaagggcaccacctacgagggcggcgtgcgggaacccgccctggctttttggcctggccacatcgcccctggcgttacccacgagctggcttctagcctggacctgctgcccaccctggccgcactggccggagctccactgcctaatgtgaccctggatggcttcgacctgtcccctctgctgctcggcaccggcaagagccctagacagagcctgttcttctacccctcctaccctgatgaggtgcggggcgtctttgccgtcaggaccggcaaatacaaggcccatttctttacacagggcagcgcccactctgataccacagccgaccctgcctgccacgccagctccagcctgaccgcccacgagcctcctctgctatacgacctgagcaaggaccctggcgagaactacaacctgctgggtggcgtggccggcgctacacctgaggtgctgcaggccctgaagcagctgcagctgcttaaggcccaactggacgccgctgtgaccttcggccctagccaggtggccagaggagaagatcccgccctgcaaatctgctgccaccctggatgtacccctcggcccgcttgttgtcactgccccgaccctcacgcctgaggtaccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgagctcatttgtgaaatttgtgatgctattgctttatttgtaaccattataagctgcaataaacaagttaacaacaacaattgcattcattttatgtttcaggttcagggggaggtgtgggaggttttttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 23 - DNA, 2190 bp, Artificial Sequence
  Description of Artificial Sequence Synthetic polynucleotide
atgtatcgtatgcaactcctttcttgcatcgccctctctctggctctcgtgaccaattccgaggtgcagctggtggagagcggcggcggcctggtgcagcctggcggcagcctgaggctgagctgcgccgcctccggcttcaacatcaaggacacctacatccactgggtccggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaactccctgagggccgaggacaccgccgtgtactactgcagcagatggggcggcgacggcttatacgccatggactactggggccagggcaccctggtgaccacctccagcgccagcaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctatacagcaagctcaccgtggacaagagcaggtggcagcaggggaacgtcttctcatgctccgtgatgcatgaggctctgcacaaccactacacgcagaagagcctctccctgtctccgggtaaacgtgccaagcgtggctctggggctacaaactttagcctcctgaagcaggctggcgacgtggaggaaaatcccggcccaatgtataggatgcagttgctgtcctgtatcgcactcagtcttgcactcgttacaaacagtgacatccagatgacccagagcccctccagcctgtccgccagcgtgggcgacagggtgaccatcacctgccgggcctcccaggacgtgaacaccgccgtggcctggtaccagcagaagcccggcaaggcccccaagctgctgatctacagcgccagcttcctgtacagcggcgtgcccagcaggttctccggcagcaggagcggcaccgacttcaccctgaccatcagcagcctgcagcccgaggacttcgccacctactactgccagcagcactacaccaccccccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 24 - DNA, 3360 bp, Artificial Sequence
  Description of Artificial Sequence Synthetic polynucleotide
gcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagacactttgtggcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctgccgccaccatgtatcgtatgcaactcctttcttgcatcgccctctctctggctctcgtgaccaattccgaggtgcagctggtggagagcggcggcggcctggtgcagcctggcggcagcctgaggctgagctgcgccgcctccggcttcaacatcaaggacacctacatccactgggtccggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaactccctgagggccgaggacaccgccgtgtactactgcagcagatggggcggcgacggcttatacgccatggactactggggccagggcaccctggtgaccacctccagcgccagcaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctatacagcaagctcaccgtggacaagagcaggtggcagcaggggaacgtcttctcatgctccgtgatgcatgaggctctgcacaaccactacacgcagaagagcctctccctgtctccgggtaaacgtgccaagcgtggctctggggctacaaactttagcctcctgaagcaggctggcgacgtggaggaaaatcccggcccaatgtataggatgcagttgctgtcctgtatcgcactcagtcttgcactcgttacaaacagtgacatccagatgacccagagcccctccagcctgtccgccagcgtgggcgacagggtgaccatcacctgccgggcctcccaggacgtgaacaccgccgtggcctggtaccagcagaagcccggcaaggcccccaagctgctgatctacagcgccagcttcctgtacagcggcgtgcccagcaggttctccggcagcaggagcggcaccgacttcaccctgaccatcagcagcctgcagcccgaggacttcgccacctactactgccagcagcactacaccaccccccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaa

SEQ ID NO: 25 - DNA, 3962 bp, Artificial Sequence
  Description of Artificial Sequence Synthetic polynucleotide
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagcccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggccaagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagaccactttgtggcggtagtttatcacagttaaattgctaacgcagtcagtgcttctgacacaacagtctcgaacttaagctgcagaagttggtcgtgaggcactgggcaggtaagtatcaaggttacaagacaggtttaaggcgaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctgccgccaccatgtatcgtatgcaactcctttcttgcatcgccctctctctggctctcgtgaccaattccgaggtgcagctggtggagagcggcggcggcctggtgcagcctggcggcagcctgaggctgagctgcgccgcctccggcttcaacatcaaggacacctacatccactgggtccggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaactccctgagggccgaggacaccgccgtgtactactgcagcagatggggcggcgacggcttatacgccatggactactggggccagggcaccctggtgaccacctccagcgccagcaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctatacagcaagctcaccgtggacaagagcaggtggcagcaggggaacgtcttctcatgctccgtgatgcatgaggctctgcacaaccactacacgcagaagagcctctccctgtctccgggtaaacgtgccaagcgtggctctggggctacaaactttagcctcctgaagcaggctggcgacgtggaggaaaatcccggcccaatgtataggatgcagttgctgtcctgtatcgcactcagtcttgcactcgttacaaacagtgacatccagatgacccagagcccctccagcctgtccgccagcgtgggcgacagggtgaccatcacctgccgggcctcccaggacgtgaacaccgccgtggcctggtaccagcagaagcccggcaaggcccccaagctgctgatctacagcgccagcttcctgtacagcggcgtgcccagcaggttctccggcagcaggagcggcaccgacttcaccctgaccatcagcagcctgcagcccgaggacttcgccacctactactgccagcagcactacaccaccccccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaa

SEQ ID NO: 26 - DNA, 3360 bp, Artificial Sequence
  Description of Artificial Sequence Synthetic polynucleotide
gcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagacactttgtggcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctgccgccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgcgtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcatcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccagaaagcggagagcccccgtgaagcagaccctgaacttcgacctgctgaagctggccggcgacgtggaaagcaaccctggccctatgtacagaatgcagctgctgctgctgatcgccctgagcctggccctggtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtatacgcctgcgaggtgacccaccagggactgagcagcccagtgaccaagagcttcaaccgcggagagtgctgataaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaa

SEQ ID NO: 27 - DNA, 3360 bp, Artificial Sequence
  Description of Artificial Sequence Synthetic polynucleotide
gcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagacactttgtggcggtagtttatcacagttaaattgctaacgcagtcagtgcttctgacacaacagtctcgaacttaagctgcagaagttggtcgtgaggcactgggcaggtaagtatcaaggttacaagacaggtttaaggcgaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctgccgccaccatgtatcgtatgcaactcctttcttgcatcgccctctctctggctctcgtgaccaattccgaagtccaactggtggagagcggaggcgggctggtgcaaccaggcggaagccttcggctgtcatgtgccgcttctggcttcaacatcaaggatacctacatccactgggtaagacaggctccagggaagggactggaatgggtagcccgtatttatcccacaaatggttacacccgttacgccgatagcgtgaaggggaggttcacaatctccgccgatacaagtaagaacaccgcttacttgcagatgaacagtcttcgtgctgaagataccgctgtttactattgtagccgttggggaggggacgggttctatgctatggactactggggtcagggcacacttgtgaccgtgtcctccgcatccaccaagggacccagcgtgttccccttggcaccttcctctaaatcaacatctggtggaactgctgccctcggctgtttggtcaaggactactttcctgagccagttaccgtatcttggaactctggagccctgaccagcggagttcacacgttccccgctgttctccagtcttcaggactctacagcctgtccagcgtcgtgaccgtgccgtcctcttccctcggcacccaaacttatatctgcaatgtgaaccataaaccctccaacactaaggtggacaagaaagtagagcccaagagttgcgacaaaacccatacctgtccaccctgtcctgcccctgaactgctcggaggcccttctgtgttcctctttccgccaaagcccaaggatactcttatgatttcacgcacccctgaggtgacatgtgttgtggtagatgtgtcacacgaagaccctgaggtgaagttcaactggtatgtggacggcgtagaagtccacaatgctaaaaccaaaccccgcgaggagcagtataatagcacctaccgtgtcgtgagcgttctgaccgtgctgcatcaggactggctgaacggaaaggaatacaagtgtaaggtaagcaataaggctctccctgcccccattgagaagaccatttccaaggcaaaggggcagccccgcgaacctcaggtttacaccctcccgcccagccgcgatgaattgactaaaaatcaggtgagccttacatgtctggtgaagggcttttatccttccgacatcgctgtggaatgggagagcaacggacaacctgagaataactataagaccacacccccagtgctggacagcgacggctcctttttcctgtattccaaactgacagtggacaagtcccgctggcaacagggcaacgttttctcttgtagcgtcatgcacgaggctctgcacaaccattacacccagaaatccttgtctctgtcccctggcaagcgtgccaagcgtggctctggggctacaaactttagcctcctgaagcaggctggcgacgtggaggaaaatcccggcccaatgtataggatgcagttgctgtcctgtatcgcactcagtcttgcactcgttacaaacagtgacatccagatgacacagagcccttccagcctgtcagcgtcagtcggcgaccgcgtgaccatcacttgcagagcctcacaggatgtgaatactgctgtggcgtggtatcaacagaagcccggcaaagcccccaaactgctcatctactccgccagtttcctctacagcggcgtcccatcacggttctctggctctcgtagcggcacggatttcacccttactatctctagtcttcagcctgaggattttgccacttactattgccaacagcactatactacaccacctacatttgggcagggcactaaggtagaaatcaaacgcaccgtggctgccccttcagttttcatcttcccacccagcgacgagcaactgaagtcaggaactgccagcgtggtctgcctgctcaataacttctacccccgcgaggctaaagttcagtggaaagtggacaacgctctccaaagtggcaattcccaagaaagcgtgaccgagcaggacagtaaggatagcacatacagcctgtcttcaacacttaccctttccaaagccgactacgaaaaacataaggtttatgcctgcgaagttacccatcagggtctgtcctcacctgttaccaagtctttcaaccgcggcgaatgttaatagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaa

SEQ ID NO: 28 - DNA, 3360 bp, Artificial Sequence
  Description of Artificial Sequence Synthetic polynucleotide
gcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagacactttgtggcggtagtttatcacagttaaattgctaacgcagtcagtgcttctgacacaacagtctcgaacttaagctgcagaagttggtcgtgaggcactgggcaggtaagtatcaaggttacaagacaggtttaaggcgaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctgccgccaccatgtatcgtatgcaactcctttcttgcatcgccctctctctggctctcgtgaccaattccgacatccagatgacacagagcccttccagcctgtcagcgtcagtcggcgaccgcgtgaccatcacttgcagagcctcacaggatgtgaatactgctgtggcgtggtatcaacagaagcccggcaaagcccccaaactgctcatctactccgccagtttcctctacagcggcgtcccatcacggttctctggctctcgtagcggcacggatttcacccttactatctctagtcttcagcctgaggattttgccacttactattgccaacagcactatactacaccacctacatttgggcagggcactaaggtagaaatcaaacgcaccgtggctgccccttcagttttcatcttcccacccagcgacgagcaactgaagtcaggaactgccagcgtggtctgcctgctcaataacttctacccccgcgaggctaaagttcagtggaaagtggacaacgctctccaaagtggcaattcccaagaaagcgtgaccgagcaggacagtaaggatagcacatacagcctgtcttcaacacttaccctttccaaagccgactacgaaaaacataaggtttatgcctgcgaagttacccatcagggtctgtcctcacctgttaccaagtctttcaaccgcggcgaatgtcgtgccaagcgtggctctggggctacaaactttagcctcctgaagcaggctggcgacgtggaggaaaatcccggcccaatgtataggatgcagttgctgtcctgtatcgcactcagtcttgcactcgttacaaacagtgaagtccaactggtggagagcggaggcgggctggtgcaaccaggcggaagccttcggctgtcatgtgccgcttctggcttcaacatcaaggatacctacatccactgggtaagacaggctccagggaagggactggaatgggtagcccgtatttatcccacaaatggttacacccgttacgccgatagcgtgaaggggaggttcacaatctccgccgatacaagtaagaacaccgcttacttgcagatgaacagtcttcgtgctgaagataccgctgtttactattgtagccgttggggaggggacgggttctatgctatggactactggggtcagggcacacttgtgaccgtgtcctccgcatccaccaagggacccagcgtgttccccttggcaccttcctctaaatcaacatctggtggaactgctgccctcggctgtttggtcaaggactactttcctgagccagttaccgtatcttggaactctggagccctgaccagcggagttcacacgttccccgctgttctccagtcttcaggactctacagcctgtccagcgtcgtgaccgtgccgtcctcttccctcggcacccaaacttatatctgcaatgtgaaccataaaccctccaacactaaggtggacaagaaagtagagcccaagagttgcgacaaaacccatacctgtccaccctgtcctgcccctgaactgctcggaggcccttctgtgttcctctttccgccaaagcccaaggatactcttatgatttcacgcacccctgaggtgacatgtgttgtggtagatgtgtcacacgaagaccctgaggtgaagttcaactggtatgtggacggcgtagaagtccacaatgctaaaaccaaaccccgcgaggagcagtataatagcacctaccgtgtcgtgagcgttctgaccgtgctgcatcaggactggctgaacggaaaggaatacaagtgtaaggtaagcaataaggctctccctgcccccattgagaagaccatttccaaggcaaaggggcagccccgcgaacctcaggtttacaccctcccgcccagccgcgatgaattgactaaaaatcaggtgagccttacatgtctggtgaagggcttttatccttccgacatcgctgtggaatgggagagcaacggacaacctgagaataactataagaccacacccccagtgctggacagcgacggctcctttttcctgtattccaaactgacagtggacaagtcccgctggcaacagggcaacgttttctcttgtagcgtcatgcacgaggctctgcacaaccattacacccagaaatccttgtctctgtcccctggcaagtaatagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaa

SEQ ID NO: 29 - DNA, 1350 bp, Artificial Sequence
  Description of Artificial Sequence Synthetic polynucleotide
gaagtccaactggtggagagcggaggcgggctggtgcaaccaggcggaagccttcggctgtcatgtgccgcttctggcttcaacatcaaggatacctacatccactgggtaagacaggctccagggaagggactggaatgggtagcccgtatttatcccacaaatggttacacccgttacgccgatagcgtgaaggggaggttcacaatctccgccgatacaagtaagaacaccgcttacttgcagatgaacagtcttcgtgctgaagataccgctgtttactattgtagccgttggggaggggacgggttctatgctatggactactggggtcagggcacacttgtgaccgtgtcctccgcatccaccaagggacccagcgtgttccccttggcaccttcctctaaatcaacatctggtggaactgctgccctcggctgtttggtcaaggactactttcctgagccagttaccgtatcttggaactctggagccctgaccagcggagttcacacgttccccgctgttctccagtcttcaggactctacagcctgtccagcgtcgtgaccgtgccgtcctcttccctcggcacccaaacttatatctgcaatgtgaaccataaaccctccaacactaaggtggacaagaaagtagagcccaagagttgcgacaaaacccatacctgtccaccctgtcctgcccctgaactgctcggaggcccttctgtgttcctctttccgccaaagcccaaggatactcttatgatttcacgcacccctgaggtgacatgtgttgtggtagatgtgtcacacgaagaccctgaggtgaagttcaactggtatgtggacggcgtagaagtccacaatgctaaaaccaaaccccgcgaggagcagtataatagcacctaccgtgtcgtgagcgttctgaccgtgctgcatcaggactggctgaacggaaaggaatacaagtgtaaggtaagcaataaggctctccctgcccccattgagaagaccatttccaaggcaaaggggcagccccgcgaacctcaggtttacaccctcccgcccagccgcgatgaattgactaaaaatcaggtgagccttacatgtctggtgaagggcttttatccttccgacatcgctgtggaatgggagagcaacggacaacctgagaataactataagaccacacccccagtgctggacagcgacggctcctttttcctgtattccaaactgacagtggacaagtcccgctggcaacagggcaacgttttctcttgtagcgtcatgcacgaggctctgcacaaccattacacccagaaatccttgtctctgtcccctggcaag

SEQ ID NO: 30 - DNA, 642 bp, Artificial Sequence
  Description of Artificial Sequence Synthetic polynucleotide
gacatccagatgacacagagcccttccagcctgtcagcgtcagtcggcgaccgcgtgaccatcacttgcagagcctcacaggatgtgaatactgctgtggcgtggtatcaacagaagcccggcaaagcccccaaactgctcatctactccgccagtttcctctacagcggcgtcccatcacggttctctggctctcgtagcggcacggatttcacccttactatctctagtcttcagcctgaggattttgccacttactattgccaacagcactatactacaccacctacatttgggcagggcactaaggtagaaatcaaacgcaccgtggctgccccttcagttttcatcttcccacccagcgacgagcaactgaagtcaggaactgccagcgtggtctgcctgctcaataacttctacccccgcgaggctaaagttcagtggaaagtggacaacgctctccaaagtggcaattcccaagaaagcgtgaccgagcaggacagtaaggatagcacatacagcctgtcttcaacacttaccctttccaaagccgactacgaaaaacataaggtttatgcctgcgaagttacccatcagggtctgtcctcacctgttaccaagtctttcaaccgcggcgaatgt

SEQ ID NO: 31 - DNA, 1353 bp, Artificial Sequence
  Description of Artificial Sequence Synthetic polynucleotide
gaagtgcagctggtggaaagcggcggaggactggtgcagcctggcggatctctgagactgagctgtgccgccagcggcttcaacatcaaggacacctacatccactgcgtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcagcatcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccagaaag

SEQ ID NO: 32 - DNA, 642 bp, Artificial Sequence
  Description of Artificial Sequence Synthetic polynucleotide
gatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtatacgcctgcgaggtgacccaccagggactgagcagcccagtgaccaagagcttcaaccgcggagagtgc

SEQ ID NO: 33 - DNA, 1350 bp, Artificial Sequence
  Description of Artificial Sequence Synthetic polynucleotide
gaggtgcagctggtggagagcggcggcggcctggtgcagcctggcggcagcctgaggctgagctgcgccgcctccggcttcaacatcaaggacacctacatccactgggtccggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaactccctgagggccgaggacaccgccgtgtactactgcagcagatggggcggcgacggcttatacgccatggactactggggccagggcaccctggtgaccacctccagcgccagcaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctatacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - DNA, 642 bp, Artificial Sequence
  Description of Artificial Sequence Synthetic polynucleotide
gacatccagatgacccagagcccctccagcctgtccgccagcgtgggcgacagggtgaccatcacctgccgggcctcccaggacgtgaacaccgccgtggcctggtaccagcagaagcccggcaaggcccccaagctgctgatctacagcgccagcttcctgtacagcggcgtgcccagcaggttctccggcagcaggagcggcaccgacttcaccctgaccatcagcagcctgcagcccgaggacttcgccacctactactgccagcagcactacaccaccccccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 35 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 78 bp, Artificial Sequence
  Description of Artificial Sequence Synthetic oligonucleotide
cgtgccaagcgtggctctggggctacaaactttagcctcctgaagcaggctggcgacgtggaggaaaatcccggccca

SEQ ID NO: 38 - DNA, 60 bp, Artificial Sequence
  Description of Artificial Sequence Synthetic oligonucleotide
atgtatcgtatgcaactcctttcttgcatcgccctctctctggctctcgtgaccaattcc

SEQ ID NO: 39 - DNA, 3360 bp, Artificial Sequence
  Description of Artificial Sequence Synthetic polynucleotide
gcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagacactttgtggcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctgccgccaccatgtatcgtatgcaactcctttcttgcatcgccctctctctggctctcgtgaccaattccgaggtgcagctggtggagagcggcggcggcctggtgcagcctggcggcagcctgaggctgagctgcgccgcctccggcttcaacatcaaggacacctacatccactgggtccggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaactccctgagggccgaggacaccgccgtgtactactgcagcagatggggcggcgacggcttatacgccatggactactggggccagggcaccctggtgaccacctccagcgccagcaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctatacagcaagctcaccgtggacaagagcaggtggcagcaggggaacgtcttctcatgctccgtgatgcatgaggctctgcacaaccactacacgcagaagagcctctccctgtctccgggtaaacgtgccaagcgtggctctggggctacaaactttagcctcctgaagcaggctggcgacgtggaggaaaatcccggcccaatgtataggatgcagttgctgtcctgtatcgcactcagtcttgcactcgttacaaacagtgacatccagatgacccagagcccctccagcctgtccgccagcgtgggcgacagggtgaccatcacctgccgggcctcccaggacgtgaacaccgccgtggcctggtaccagcagaagcccggcaaggcccccaagctgctgatctacagcgccagcttcctgtacagcggcgtgcccagcaggttctccggcagcaggagcggcaccgacttcaccctgaccatcagcagcctgcagcccgaggacttcgccacctactactgccagcagcactacaccaccccccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaa

SEQ ID NO: 40 - DNA, 1350 bp, Artificial Sequence
  Description of Artificial Sequence Synthetic polynucleotide
gaggtgcagctggtggagagcggcggcggcctggtgcagcctggcggcagcctgaggctgagctgcgccgcctccggcttcaacatcaaggacacctacatccactgggtccggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaactccctgagggccgaggacaccgccgtgtactactgcagcagatggggcggcgacggcttatacgccatggactactggggccagggcaccctggtgaccacctccagcgccagcaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctatacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 41 - DNA, 563 bp, Artificial Sequence
  Description of Artificial Sequence Synthetic polynucleotide
aggacgtgaacaccgccgtggcctggtaccagcagaagcccggcaaggcccccaagctgctgatctacagcgccagcttcctgtacagcggcgtgcccagcaggttctccggcagcaggagcggcaccgacttcaccctgaccatcagcagcctgcagcccgaggacttcgccacctactactgccagcagcactacaccaccccccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 42 - DNA, 360 bp, Artificial Sequence
  Description of Artificial Sequence Synthetic polynucleotide
gaagtccaactggtggagagcggaggcgggctggtgcaaccaggcggaagccttcggctgtcatgtgccgcttctggcttcaacatcaaggatacctacatccactgggtaagacaggctccagggaagggactggaatgggtagcccgtatttatcccacaaatggttacacccgttacgccgatagcgtgaaggggaggttcacaatctccgccgatacaagtaagaacaccgcttacttgcagatgaacagtcttcgtgctgaagataccgctgtttactattgtagccgttggggaggggacgggttctatgctatggactactggggtcagggcacacttgtgaccgtgtcctcc

SEQ ID NO: 43 - DNA, 321 bp, Artificial Sequence
  Description of Artificial Sequence Synthetic polynucleotide
gacatccagatgacacagagcccttccagcctgtcagcgtcagtcggcgaccgcgtgaccatcacttgcagagcctcacaggatgtgaatactgctgtggcgtggtatcaacagaagcccggcaaagcccccaaactgctcatctactccgccagtttcctctacagcggcgtcccatcacggttctctggctctcgtagcggcacggatttcacccttactatctctagtcttcagcctgaggattttgccacttactattgccaacagcactatactacaccacctacatttgggcagggcactaaggtagaaatcaaa

SEQ ID NO: 44 - DNA, 363 bp, Artificial Sequence
  Description of Artificial Sequence Synthetic polynucleotide
gaagtgcagctggtggaaagcggcggaggactggtgcagcctggcggatctctgagactgagctgtgccgccagcggcttcaacatcaaggacacctacatccactgcgtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgca

SEQ ID NO: 45 - DNA, 321 bp, Artificial Sequence
  Description of Artificial Sequence Synthetic polynucleotide
gatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaag

SEQ ID NO: 46 - DNA, 360 bp, Artificial Sequence
  Description of Artificial Sequence Synthetic polynucleotide
gaggtgcagctggtggagagcggcggcggcctggtgcagcctggcggcagcctgaggctgagctgcgccgcctccggcttcaacatcaaggacacctacatccactgggtccggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaactccctgagggccgaggacaccgccgtgtactactgcagcagatggggcggcgacggcttatacgccatggactactggggccagggcaccctggtgaccacctccagc

SEQ ID NO: 47 - DNA, 321 bp, Artificial Sequence
  Description of Artificial Sequence Synthetic polynucleotide
gacatccagatgacccagagcccctccagcctgtccgccagcgtgggcgacagggtgaccatcacctgccgggcctcccaggacgtgaacaccgccgtggcctggtaccagcagaagcccggcaaggcccccaagctgctgatctacagcgccagcttcctgtacagcggcgtgcccagcaggttctccggcagcaggagcggcaccgacttcaccctgaccatcagcagcctgcagcccgaggacttcgccacctactactgccagcagcactacaccaccccccccaccttcggccagggcaccaaggtggagatcaag

SEQ ID NO: 48 - DNA, 33 bp, Artificial Sequence
  Description of Artificial Sequence Synthetic oligonucleotide
tggggcggcgacggcttatacgccatggactac

SEQ ID NO: 49 - protein, 11 aa, Artificial Sequence
  Description of Artificial Sequence Synthetic peptide
WGGDGLYAMDY

SEQ ID NO: 50 - DNA, 33 bp, Artificial Sequence
  Description of Artificial Sequence Synthetic oligonucleotide
tggggaggcgacggcttctacgccatggactat

SEQ ID NO: 51 - protein, 11 aa, Artificial Sequence
  Description of Artificial Sequence Synthetic peptide
WGGDGFYAMDY

SEQ ID NO: 52 - DNA, 17 bp, Artificial Sequence
  Description of Artificial Sequence Synthetic primer
gcttctggcgtgtgacc

SEQ ID NO: 53 - DNA, 23 bp, Artificial Sequence
  Description of Artificial Sequence Synthetic primer
tgatgagacagcacaataaccag

SEQ ID NO: 54 - DNA, 14 bp, Artificial Sequence
  Description of Artificial Sequence Synthetic probe
gctcctgggcaacg

SEQ ID NO: 55 - DNA, 17 bp, Artificial Sequence
  Description of Artificial Sequence Synthetic primer
gaacctgaaacttcaca

SEQ ID NO: 56 - DNA, 19 bp, Artificial Sequence
  Description of Artificial Sequence Synthetic primer
ccatttaaggagtggttat

SEQ ID NO: 57 - DNA, 15 bp, Artificial Sequence
  Description of Artificial Sequence Synthetic probe
cgcactaccacttac

SEQ ID NO: 58 - DNA, 17 bp, Artificial Sequence
  Description of Artificial Sequence Synthetic primer
cacagctcctgggcaac

SEQ ID NO: 59 - DNA, 17 bp, Artificial Sequence
  Description of Artificial Sequence Synthetic primer
agctcgaccaggatggg

SEQ ID NO: 60 - DNA, 11 bp, Artificial Sequence
  Description of Artificial Sequence Synthetic probe
aagggcgagga

SEQ ID NO: 61 - DNA, 18 bp, Artificial Sequence
  Description of Artificial Sequence Synthetic primer
gcgggttctgacctgaag

SEQ ID NO: 62 - DNA, 23 bp, Artificial Sequence
  Description of Artificial Sequence Synthetic primer
tccctgtacaatcggtaaagttg

SEQ ID NO: 63 - DNA, 16 bp, Artificial Sequence
  Description of Artificial Sequence Synthetic probe
ttggctattcagttgt

(19) United States
(12) Patent Application Publication
(10) Pub. No.: US 2023/0034817 A1
(43) Pub. Date: Feb. 2, 2023

(54) RECOMBINANT AAV FOR TREATMENT OF NEURAL DISEASE

(71) Applicant: AFFINIA THERAPEUTICS INC., Waltham, MA (US)

(72) Inventors: Laura K. RICHMAN, Damascus, MD (US); Roberto CALCEDO DEL HOYO, Concord, MA (US); Douglas N. SANDERS, Hudson, NH (US); Lisa M. STANEK, Natick, MA (US); Samantha SMITH, Watertown, MA (US); Richard LU, Somerville, MA (US); Christopher TIPPER, Cambridge, MA (US); Robert Steven Johnson, Oundle (GB)

(73) Assignee: AFFINIA THERAPEUTICS INC., Waltham, MA (US)

(21) Appl. No.: 17/717,858

(22) Filed: Apr. 11, 2022

Related U.S. Application Data
(60) Provisional application No. 63/173,992, filed on Apr. 12, 2021.
(60) Provisional application No. 63/186,655, filed on May 10, 2021.
(60) Provisional application No. 63/217,449, filed on Jul. 1, 2021.
(60) Provisional application No. 63/290,544, filed on Dec. 16, 2021.
(60) Provisional application No. 63/290,543, filed on Dec. 16, 2021.
(60) Provisional application No. 63/306,735, filed on Feb. 4, 2022.

(30) Foreign Application Priority Data
Dec. 16, 2021 (US) PCT/US2021/063882
Dec. 16, 2021 (US) PCT/US2021/063889

(51) Int. Cl. C12N 15/861; C12N 15/74; A61P 25/28

(52) U.S. Cl. CPC C12N 15/861; C12N 15/74; A61P 25/28

(57) ABSTRACT

The disclosure pertains to a recombinant adeno-associated virus (rAAV) comprising an Anc80L65 capsid for delivering a polynucleotide (e.g., a transgene) into the central nervous system (CNS). Further provided includes methods for treating CNS diseases using the rAAV and pharmaceutical compositions comprising the rAAV.

### 1. CROSS-REFERENCE TO RELATED APPLICATIONS

This application claims the priority benefit of U.S. provisional application nos. 63/173,992, filed Apr. 12, 2021, 63/186,655, filed May 10, 2021, 63/217,449, filed Jul. 1, 2021, 63/290,543, filed Dec. 16, 2021, 63/290,544, filed Dec. 16, 2021, and 63/306,735, filed Feb. 4, 2022, and PCT international application nos. PCT/US2021/063882, filed Dec. 16, 2021, and PCT/US2021/063889, filed Dec. 16, 2021, the contents of each of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Apr. 11, 2022 is named AFF-002B-US_SL.txt and is 112,021 bytes in size.

### 3. BACKGROUND OF THE INVENTION

Adeno-associated virus (AAV) has become the vector system of choice for in vivo gene therapy. A growing variety of recombinant AAVs (rAAVs) engineered to deliver therapeutic nucleic acids have been developed and tested in nonhuman primates and humans, and the FDA has recently approved two rAAV gene therapy products for commercialization.

Although AAV vectors are safer and less inflammatory than other viruses, toxicities have occurred following administration of high doses of rAAVs for gene therapy. Thus, local administration of rAAVs to a target tissue or organ has been used to improve targeting and reduce systemic toxicity. Further, various natural and synthetic AAV variants have been tested to develop an AAV vector with desired tropism and specificity.

In general, the capsid is thought to be the primary determinant of infectivity and host-vector related properties such as adaptive immune responses, tropism, specificity, potency, and bio-distribution. Indeed, several of these properties are known to vary between natural serotypes and engineered AAV variants.

Treatment of diseases of the central nervous system (CNS) e.g. remains an intractable problem. Examples of CNS diseases include inherited genetic diseases such as the lysosomal storage diseases such as metachromatic leukodystrophy (MLD), brain cancer such as brain metastasis of breast cancer (BMBC) and Alzheimer's disease. MLD is most commonly caused by a deficiency of the enzyme arylsulfatase A (ARSA). ARSA deficiency leads to a buildup of sulfatides in myelin-producing cells in the nervous system, causing progressive destruction of white matter throughout the nervous system. Collectively, the incidence of lysosomal storage diseases (LSD) is 1 in 10,000 births worldwide, and in 65% of cases, there is significant central nervous system (CNS) involvement. BMBC is observed in about 10-15% of women with stage IV breast cancer. The risk of the brain metastasis is usually highest for women with more aggressive subtypes of breast cancer, such as HER2-positive or triple-negative breast cancer. Currently, therapeutics for these CNS diseases are limited because many of them, when delivered intravenously, do not cross the blood-brain barrier, or, when delivered directly to the brain, are not widely distributed. Thus, therapies for the CNS diseases need to be developed.

To date, however, there is little understanding as to how changes on the AAV capsid alter their biological properties and AAV vectors with a desired tropism and specificity to therapeutic targets, such as the central nervous system (CNS), have not yet been available. Species-specific differences in AAV tropism, for example between mice and nonhuman primates (NHP), has made it difficult to develop AAV vectors that have a desired tropism in humans.

### 4. SUMMARY OF THE INVENTION

Applicant has demonstrated that a single injection of Anc80L65, a rationally designed synthetic vector (described in WO2015/054653, which is incorporated by reference in its entirety herein), into the CSF of adult cynomolgus monkeys leads to more efficient transduction of broad regions of the CNS and strikingly outperforms the capabilities of AAV9 to target the cortex and deep brain nuclei. A single CSF injection of Anc80L65 distributes more broadly throughout the cortex and into deep brain nuclei compared to AAV9 delivered with either ICM or LP injection. Anc80L65 distribution by LP injection throughout the cortex was on par with ICM delivery, while AAV9 showed little to no transduction in the cortex following the LP route of delivery. ICM and LP delivery of both Anc80L65 and AAV9 led to robust transduction of the spinal cord and ventral horn motor neurons. The ability of Anc80L65 to mediate efficient expression in neurons and astrocytes across large regions of the NHP brain following a single LP injection has broad implications for treatment of a wide range of neurologic disorders. Availability of a relatively noninvasive method of delivery makes Anc80L65 a superior therapeutic modality to other available AAVs, including AAV9.

Applicant further developed and tested Anc80L65 for delivery of coding sequences of ARSA and functional variants thereof for treatment of MLD. AAV constructs with a coding sequence of ARSA or a functional variant thereof operably linked to a different promoter (i.e., UbC promoter, CMV promoter, or CAG promoter) were tested for their capability to deliver and express the transgene in the CNS. The studies demonstrated that Anc80L65 rAAV vectors can successfully deliver a polynucleotide encoding ARSA or a functional variant thereof to the CNS of ARSA knock-out (KO) mice, resulting in ARSA protein expression and reduction in sulfatide levels in the CNS. The studies further demonstrated that AAV constructs containing ARSA and ARSA functional variants under the control of a UbC promoter were particularly effective in inducing CNS expression of ARSA and ARSA functional variants and reducing lysosulfatide and sulfatide levels compared to other constructs.

Applicant further developed and tested Anc80L65 for delivery of various coding sequences of anti-HER2 antigen binding protein (ABP) for treatment of BMBC. AAV genomic constructs with a coding sequence of anti-HER2 antigen (i.e., trastuzumab) operably linked to a different promoter (i.e., CMV promoter or UbC promoter) were tested for their capability to deliver and express the transgene in wide brain targets. Additionally, AAV genomic vectors with the heavy chain coding sequences and the light chain coding sequences of trastuzumab in different orders (5′-HC-LC-3′ or 5′-LC-HC-3′) were tested. The study demonstrated that a construct including UbC promoter operably linked to the heavy chain and the light chain coding sequences in the 5′ to 3′ order induces significantly better transduction and expression of trastuzumab compared to other constructs, when delivered to the mouse brain.

The Anc80L65 selected from these studies is expected to induce high level expression of a therapeutic protein (e.g., ARSA and functional variants thereof, trastuzumab, etc.) across broad CNS regions (e.g., broad brain regions), thereby effectively treating various neurologic disorders, such as MLD and BMBC.

Accordingly, the present disclosure provides a method of transferring a polynucleotide to the central nervous system (CNS) of a subject, the method comprising: administering to the subject an effective dose of: a recombinant adeno-associated virus (rAAV) comprising: a capsid comprising: a capsid protein having the amino acid sequence of SEQ ID NO: 1 or a variant thereof, and the polynucleotide encapsulated by the capsid; thereby transferring the polynucleotide to the CNS.

In some embodiments, the present disclosure provides a method of transferring a polynucleotide to the central nervous system (CNS) of a subject, the method comprising: administering to the subject an effective dose of: a recombinant adeno-associated virus (rAAV) comprising: a capsid comprising: a capsid protein having the amino acid sequence of SEQ ID NO: 1, and the polynucleotide encapsulated by the capsid; thereby transferring the polynucleotide to the CNS.

In some embodiments, the polynucleotide comprises a coding sequence of a therapeutic protein. In some embodiments, the subject has a CNS disease. In some embodiments, the CNS disease is a lysosomal storage disease (LSD). In some embodiments, the CNS disease is a leukodystrophy.

In some embodiments, the CNS disease is metachromatic leukodystrophy (MLD). In some embodiments, the polynucleotide comprises a coding sequence encoding Arylsulfatase A (ARSA) or a functional variant thereof. In some embodiments, the polynucleotide comprises a coding sequence selected from SEQ ID NO: 2-4. In other embodiments, the polynucleotide comprises a coding sequence selected from SEQ ID NO: 7-8.

In some embodiments, the polynucleotide comprises a coding sequence encoding ARSA or a functional variant thereof operably linked to a UbC promoter, CAG promoter, or CMV promoter.

In some embodiments, the polynucleotide comprises, in the 5′ to 3′ direction, (i) a 5′ inverted terminal repeat (ITR), (ii) a UbC promoter, a CAG promoter, or a CMV promoter, (iii) a polynucleotide encoding ARSA or a functional variant thereof, and (iv) a 3′ ITR.

The ARSA can be, for example, a native (wild-type) human ARSA protein, e.g., whose amino acid sequence is set forth in SEQ ID NO: 5, or an ARSA functional variant having one or more amino acid substitutions relative to a native human ARSA, e.g., a ARSA functional variant having at least 95% sequence identity with SEQ ID NO: 5. An exemplary ARSA functional variant is the “Hyper-ARSA” protein (SEQ ID NO: 6), which has M202V, T286L, and R291N substitutions.

In some embodiments, the coding sequence of the ARSA or functional variant is codon-optimized. Alternatively the coding sequence can comprise a non-optimized coding sequence, e.g., a native or wild-type coding sequence. Exemplary ARSA and ARSA functional variant coding sequences are set forth in SEQ ID NOs: 2-4 (encoding a native ARSA protein) and SEQ ID NOs: 7-8 (encoding Hyper-ARSA).

In some embodiments, the CNS disease is Krabbe's leukodystrophy. In some embodiments, the polynucleotide comprises a coding sequence of galactocerebroside beta-galactosidase or a functional variant thereof.

In some embodiments, the CNS disease is GM1 gangliosidosis. In some embodiments, the polynucleotide comprises a coding sequence of galactosidase beta 1 (GLB-1) or a functional variant thereof.

In some embodiments, the CNS disease is a cancer. In some embodiments, the CNS disease is metastatic breast cancer. In some embodiments, the therapeutic protein is an antigen binding protein against human epidermal growth factor receptor 2 (HER2). In some embodiments, the polynucleotide comprises a sequence of SEQ ID NO: 23.

In some embodiments, the polynucleotide comprises a coding sequence of an antigen. In some embodiments, the antigen is a viral or bacterial antigen. In some embodiments, the effective dose is sufficient to immunize the subject. In some embodiments, the effective dose is sufficient to induce an immune response to the antigen.

In some embodiments, the polynucleotide further comprises a regulatory sequence operably linked to the coding sequence. In some embodiments, the regulatory sequence comprises a CMV promoter, a UbC promoter, or a CAG promoter. In some embodiments, the regulatory sequence comprises a CMV promoter or a UbC promoter. In some embodiments, the regulatory sequence comprises a UbC promoter comprising a nucleotide sequence having at least 90%, at least 95%, at least 96%, at least 97%, or at least 98%, at least 99%, or 100% sequence identity to SEQ ID NO: 9, SEQ ID NO: 10 or SEQ ID NO: 11. In some embodiments, the regulatory sequence comprises a UbC promoter having the sequence of SEQ ID NO: 9, SEQ ID NO: 10 or SEQ ID NO: 11.

In some embodiments, the administration induces protein expression from the polynucleotide in the Substantia nigra of the subject. In some embodiments, the administration induces protein expression from the polynucleotide in the caudate nuclei of the subject. In some embodiments, the administration induces protein expression from the polynucleotide in the ependyma of the subject. In some embodiments, the administration induces protein expression from the polynucleotide in the cortex of the subject.

In some embodiments, the administration is to the cerebrospinal fluid (CSF) of the subject. In some embodiments, the administration is selected from intrathecal administration, intracranial administration, intracerebroventricular (ICV) administration and administration to the lateral ventricles of the brain of the subject. In some embodiments, the intrathecal administration is by lumbar puncture (LP) and/or intra cisterna magna (ICM) injection. In some embodiments, the step of administering is performed by ICM injection. In some embodiments, the step of administering is performed by lumbar puncture (LP).

In some embodiments wherein the administration is to the cerebrospinal fluid (CSF) of the subject, the effective dose is between 1E10 to 1E16 genome copy numbers (GC) of the AAV. In some embodiments, the effective dose is 1E9 GC to 1E14 GC per gram brain mass. In some embodiments, the effective dose is administered at a concentration of 1E12 GC/ml to 1E17 GC/ml.

In some embodiments, the effective dose is administered systemically. In some embodiments, the step of administration is performed intravenously. In some embodiments, the effective dose is between 1E10-1E16 genome copy numbers (GC) of the AAV. In some embodiments, the effective dose is between 1E9-1E15 genome copy numbers (GC) of the AAV per kg body weight.

In some embodiments, the effective dose is an amount sufficient to induce detectable expression of the therapeutic protein in the CNS. In some embodiments, the effective dose is an amount sufficient to induce detectable expression of the therapeutic protein in the Substantia nigra. In some embodiments, the effective dose is an amount sufficient to induce detectable expression of the therapeutic protein in the caudate nuclei. In some embodiments, the effective dose is an amount sufficient to induce detectable expression of the therapeutic protein in the ependyma. In some embodiments, the effective dose is an amount sufficient to induce detectable expression of the therapeutic protein in the cortex.

In another aspect, the present disclosure provides a method of treating a disease of the central nervous system (CNS), the method comprising: administering to the CNS of a subject an effective dose of: a recombinant adeno-associated virus (rAAV), the rAAV comprising: a capsid polypeptide having the amino acid sequence of SEQ ID NO: 1 or a variant thereof, and a polynucleotide encoding a therapeutic protein.

In yet another aspect, the present disclosure provides a method of vaccination with a transgene, the method comprising: administering to the central nervous system (CNS) of a subject an effective dose of: a recombinant adeno-associated virus (rAAV), the rAAV comprising: a capsid polypeptide having the amino acid sequence of SEQ ID NO: 1 or a variant thereof, and a polynucleotide encoding an antigen.

In one aspect, the present disclosure provides a recombinant adeno-associated virus (rAAV) comprising: a capsid comprising: a capsid protein having the amino acid sequence of SEQ ID NO: 1, and a polynucleotide encapsulated by the capsid, wherein the polynucleotide encodes a therapeutic protein associated with a CNS disease.

In some embodiments, the CNS disease is metachromatic leukodystrophy (MLD). In some embodiments, the therapeutic protein is Arylsulfatase A (ARSA) or a functional variant thereof, and the polynucleotide comprises a coding sequence selected from SEQ ID NOs: 2-4. In some embodiments, the therapeutic protein is Hyper ARSA, and the polynucleotide comprises a coding sequence selected from SEQ ID NOs: 7-8.

In some embodiments, the CNS disease is Krabbe's leukodystrophy. In some embodiments, the polynucleotide comprises a coding sequence of galactocerebrosidase or a functional variant thereof.

In some embodiments, the CNS disease is GM1 gangliosidosis. In some embodiments, the therapeutic protein is galactosidase beta 1 (GLB-1) or a functional variant thereof.

In some embodiments, the CNS disease is cancer. In some embodiments, the CNS disease is metastatic breast cancer. In some embodiments, the therapeutic protein is an antigen binding protein against human epidermal growth factor receptor 2 (HER2).

In some embodiments, the ABP against HER2 is trastuzumab. In some embodiments, the coding sequence comprises from 5′ to 3′, a coding sequence of a heavy chain of the ABP against HER2 and a coding sequence of a light chain of the ABP against HER2. In some embodiments, the coding sequence comprises from 5′ to 3′, a coding sequence of a light chain of the ABP against HER2 and a coding sequence of a heavy chain of the ABP against HER2.

In some embodiments, the coding sequence of a heavy chain comprises a sequence of SEQ ID NO: 29, 31 or 33. In some embodiments, the coding sequence of a light chain comprises a sequence of SEQ ID NO: 30, 32 or 34. In some embodiments, the coding sequence comprises: a heavy chain coding sequence of SEQ ID NO: 29 and a light chain coding sequence of SEQ ID NO: 30; a heavy chain coding sequence of SEQ ID NO: 31 and a light chain coding sequence of SEQ ID NO: 32; or a heavy chain coding sequence of SEQ ID NO: 33 and a light chain coding sequence of SEQ ID NO: 34.

In some embodiments, the coding sequence further comprises a self-cleaving peptide between the coding sequence of the heavy chain and the coding sequence of the light chain. In some embodiments, the self-cleaving peptide is selected from the group consisting of F2A, P2A, T2A and E2A. In some embodiments, the self-cleaving peptide has the sequence of SEQ ID NO: 37.

In some embodiments, the coding sequence further comprising one or more coding sequence of interleukin 2 signal sequence (IL2SS). In some embodiments, one coding sequence of IL2SS is located at 5′ end of the heavy chain coding sequence. In some embodiments, one coding sequence of IL2 SS is located at 5′ end of the light chain coding sequence. In some embodiments, a first coding sequence of IL2 SS is located at 5′ end of the heavy chain coding sequence and a second coding sequence of IL2 SS is located at 5′ end of the light chain coding sequence.

In some embodiments, the polynucleotide comprises a coding sequence of SEQ ID NO: 23. In some embodiments, the polynucleotide comprises a coding sequence having at least 80%, 90%, 95%, 96%, 97%, 98%, or 99% sequence identity to SEQ ID NO: 23.

In some embodiments, the polynucleotide comprises the sequence of SEQ ID NO: 24-34, or a fragment thereof.

In some embodiments, the polynucleotide comprises the sequence of SEQ ID NO: 24. In some embodiments, the polynucleotide comprises the sequence of SEQ ID NO: 25.

In some embodiments, the polynucleotide further comprises a regulatory sequence operably linked to the coding sequence. In some embodiments, the regulatory sequence comprises a CMV promoter or a UbC promoter. In some embodiments, the regulatory sequence comprises a UbC promoter having the sequence of SEQ ID NO: 11.

In another aspect, the present disclosure provides a pharmaceutical composition comprising any of the rAAV described herein. In yet another aspect, the present disclosure provides a unit dose comprising the pharmaceutical composition described herein.

In another aspect, the present disclosure provides a method of transferring a polynucleotide to the central nervous system (CNS) of a subject, the method comprising: administering to the subject an effective dose of: any of the rAAV described herein, any of the pharmaceutical compositions described herein, or any of the unit doses described herein.

In another aspect, the present disclosure provides a method of transferring a polynucleotide to the central nervous system (CNS) of a subject, the method comprising: administering to the CNS an effective dose of: a recombinant adeno-associated virus (rAAV) comprising: a capsid having the amino acid sequence of SEQ ID NO: 1 or a variant thereof (e.g., a variant as defined in Section 6.2.1), and a polynucleotide having the nucleic acid sequence of SEQ ID NO: 19 or SEQ ID NO: 20, wherein the polynucleotide is encapsulated by the capsid, wherein the subject has MLD.

In another aspect, the present disclosure provides a recombinant adeno-associated virus (rAAV) comprising: a capsid having the amino acid sequence of SEQ ID NO: 1, and a polynucleotide encapsulated by the capsid having the nucleic acid sequence of SEQ ID NO: 19 or SEQ ID NO: 20.

In another aspect, the present disclosure provides a method of transferring a polynucleotide to the central nervous system (CNS) of a subject, the method comprising: administering to the CNS an effective dose of: a recombinant adeno-associated virus (rAAV) comprising: a capsid having the amino acid sequence of SEQ ID NO: 1 or a variant thereof, and a polynucleotide having the nucleic acid sequence of SEQ ID NO: 24 or 25, wherein the polynucleotide is encapsulated by the capsid, wherein the subject has metastatic breast cancer.

In another aspect, the present disclosure provides a recombinant adeno-associated virus (rAAV) comprising: a capsid having the amino acid sequence of SEQ ID NO: 1, and a polynucleotide encapsulated by the capsid having the nucleic acid sequence of SEQ ID NO: 24 or 25.

### 5. BRIEF DESCRIPTION OF THE SEVERAL VIEWS OF THE DRAWINGS

These and other features, aspects, and advantages of the present invention will become better understood with regard to the following description, and accompanying drawings.

FIG. 1 summarizes the NHP study design described in Example 1.

FIGS. 2A-2D are immunohistochemistry (IHC) images of brain sections, obtained from NHPs administered with (i) Anc80L65-CAG-GFP or (ii) AAV9-CAG-GFP by intracisternal magna injection (ICM) or lumbar-puncture (LP). Brown stain=GFP expression (arrows). Inset in Anc80L65-LP (FIG. 2B) shows mostly neuronal staining. FIG. 2A shows GFP expression after administration of Anc80L65 via ICM injection. FIG. 2B shows GFP expression after administration of Anc80L65 via LP. FIG. 2C shows GFP expression after administration of AAV9 via ICM injection. FIG. 2D shows GFP expression after administration of AAV via LP.

FIGS. 3A-3C are IHC images of brain sections including cortex, obtained from a NHP administered with vehicle (FIG. 3A), Anc80L65-CAG-GFP (FIG. 3B), or AAV9-CAG-GFP (FIG. 3C). Brown stain=GFP expression.

FIGS. 4A-4B are IHC images of a brain section including ependyma and caudate nucleus, obtained from a NHP administered Anc80L65-CAG-GFP by ICM injection. FIG. 4B is an enlarged image of a portion of FIG. 4A. Brown stain=GFP expression.

FIGS. 5A-5B are IHC images of a brain section including caudate nucleus, obtained from a NHP administered with Anc80L65-CAG-GFP by ICM injection. FIG. 5B is an enlarged image of a portion of FIG. 5A. Brown stain=GFP expression.

FIG. 6 is an IHC image of a brain section including Substantia nigra, obtained from a NHP administered with Anc80L65-CAG-GFP by ICM injection. Brown stain=GFP expression.

FIGS. 7A and 7B are IHC images of a brain section including perivascular cells, obtained from a NHP administered with Anc80L65-CAG-GFP by ICM injection. FIG. 7B is an enlarged image of a portion of FIG. 7A. Brown stain=GFP expression.

FIGS. 8A and 8B are IHC images of a brain section including cortex, obtained from a NHP administered with Anc80L65-CAG-GFP by ICM injection. FIG. 8B is an enlarged image of a portion of FIG. 8A. Brown stain=GFP expression.

FIG. 9 is an IHC image of a brain section including cortex, obtained from a NHP administered with Anc80L65-CAG-GFP by lumbar puncture (LP). Brown stain=GFP expression.

FIGS. 10A-10C provide one-way analysis of transgene expression determined by measurement of mRNA transcript of eGFP calculated according to the equation: % eGFP expression=(eGFP cp/uL=RPP30 cp/uL)×100, in various brain regions in animals administered with AAV9-CAG-GFP by ICM injection or with Anc80L65-CAG-GFP by LP. FIG. 10A provides data for the frontal cortex; FIG. 10B provides data for the motor cortex; and FIG. 10C provides data for the parietal lobe of the cortex.

FIGS. 11A-11B provide one-way analysis of transgene expression determined by measurement of mRNA transcript of eGFP calculated according to the equation: % eGFP expression=(eGFP cp/uL=RPP30 cp/uL)×100, in various brain regions administered with AAV9-CAG-GFP by ICM injection or with Anc80L65-CAG-GFP by LP. FIG. 11A provides data for the caudate nucleus; and FIG. 11B provides data for the Globus pallidus.

FIGS. 12A-12B provide one-way analysis of transgene expression determined by measurement of mRNA transcript of eGFP calculated according to the equation: % eGFP expression=(eGFP cp/uL=RPP30 cp/uL)×100, in various brain regions administered with AAV9-CAG-GFP by ICM injection or with Anc80L65-CAG-GFP by LP. FIG. 12A provides data for the putamen; and FIG. 12B provides data for the Substantia nigra.

FIGS. 13A-17 provide one-way analysis of viral genome (DNA) copy per diploid genome (VGC/DG) determined by measurement of the genome copy numbers using ddPCR and calculation of (VGC/DG) values using the equation: VGC/DG=(eGFP cp/uL=RPP30 cp/uL)×2. Each figure provides data for a different brain region or liver, including cerebellar cortex (FIG. 13A), dorsal root ganglia, cervical (FIG. 13B), dorsal root ganglia, lumbar (FIG. 14A), frontal cortex (FIG. 14B), liver (FIG. 15A), motor cortex (FIG. 15B), spinal cord, cervical (FIG. 16A), spinal cord, lumbar (FIG. 16B), and sciatic nerve (FIG. 17).

FIGS. 18A, 18B, 19A, 19B, 20A, 20B and 21 provide one-way analysis of transgene expression determined by measurement of mRNA transcript of eGFP calculated according to the equation: % eGFP expression=(eGFP cp/uL=RPP30 cp/uL)×100. Each figure provides data for a different brain region, including caudate nucleus (FIG. 18A), frontal cortex (FIG. 18B), Globus pallidus (FIG. 19A), motor cortex (FIG. 19B), parietal cortex (FIG. 20A), putamen (FIG. 20B), and Substantia nigra (FIG. 21).

FIGS. 22A-22D are immunohistochemistry (IHC) images of brain sections, obtained from NHPs administered with Anc80L65-CAG-GFP or AAV9-CAG-GFP by intracisternal magna injection. Brown stain=GFP expression. FIG. 22A shows GFP expression in the cortex after administration of Anc80L65-CAG-GFP. FIG. 22B shows GFP expression in the caudate nucleus after administration of Anc80L65-CAG-GFP. FIG. 22C shows GFP expression in the cortex after administration of AAV9-CAG-GFP. FIG. 22D shows GFP expression in the caudate nucleus after administration of AAV9-CAG-GFP.

FIGS. 23 and 24 illustrate the GFP mRNA expression measured by ddPCR in the NHP brain and spinal cord 2 weeks after ICM or LP delivery of AAV9-CAG-GFP or Anc80L65-CAG-GFP. FIG. 23 provides % GFP expression in the frontal cortex, motor cortex, and parietal cortex. FIG. 24 provides % GFP expression in the caudate nucleus, Globus palidus, putamen, and Substantia nigra.

FIG. 25 illustrates the vector genome copy analysis via qPCR. VGCs per cell (presented as mean vector genome copies per diploid genome VGC/DG) in NHPs injected with Anc80L65-CAG-GFP and AAV9-CAG-GFP by LP or ICM injection are provided.

FIGS. 26A-26F are double immunofluorescence (IF) staining images of brain sections administered with Anc80L65-CAG-GFP (FIGS. 26A, 26B and 26C) or AAV9-CAG-GFP (FIGS. 26D, 26E and 26F). The transgene expression from the AAVs was detected by staining against GFP and cell types were detected by staining against cell-type specific markers, including NeuN for neurons (FIG. 26A and FIG. 26D), GFAP for astrocytes (FIG. 26B and FIG. 26E), and Iba1 for microglial cells (FIG. 26C and FIG. 26F). Examples were imaged from the motor cortex. In all cases, GFP+ cells are shown in red, the cell specific marker is shown in green, and the merged images are shown with double-labeled cells in yellow/orange (arrows for double-labeled cells).

FIGS. 27A-27F are double immunofluorescence (IF) staining images of brain sections from NHP administered with Anc80L65-CAG-GFP via LP (FIGS. 27A, 27B and 27C) or via ICM (FIGS. 27D, 27E and 27F). Examples were imaged from the motor cortex. The transgene expression from Anc80L65 was detected by staining against GFP and oligodendrocyte cells were detected by staining against oligodendrocyte specific marker OLIG2, shown in green (FIG. 27A and FIG. 27D). GFP+ cells are shown in red (FIG. 27B and FIG. 27E). The merged images are shown with double-labeled cells in yellow/orange (arrows for double-labeled cells) (FIG. 27C and FIG. 27F).

FIG. 28 provides a schematic of the experimental design for testing rAAV constructs encoding an antigen binding protein against human epidermal growth factor receptor 2 (HER2), as described in Example 2.

FIG. 29 illustrates brain samples obtained for testing transgene transfer and expression by rAAVs, as described in Example 2.

FIGS. 30A-30B provide one-way ANOVA analysis of viral genome (DNA) copy per diploid genome (VGC/DG) determined by measurement of the genome copy numbers using ddPCR and calculation of (VGC/DG) values using the equation: VGC/DG=(Trastuzumab cp/uL RPP30 cp/uL)×2 for each of the five treatment groups described in Example 2 on day 13 (FIG. 30A) and day 30 (FIG. 30B).

FIGS. 31A-31B provide one-way ANOVA analysis of transgene expression determined by measurement of mRNA transcript of Trastuzumab calculated according to the equation: Trastuzumab expression=(Trastuzumab cp/uL=RPP30 cp/uL)×100 for each of the five treatment groups described in Example 2 on day 13 (FIG. 31A) and day 30 (FIG. 31B).

FIGS. 32A-32B provide one-way ANOVA analysis of transgene protein expression determined by measuring Trastuzumab protein expression in brain tissue using a HER2-binding ELISA and presented as absorbance normalized to total protein loaded. HER2-binding ELISAs were performed for the five treatment groups described in Example 2 on day 13 (FIG. 32A) and day 30 (FIG. 32B).

FIG. 33 provides a schematic of a polynucleotide encoding Trastuzumab (Her2 Heavy Chain and Her2 Light Chain) according to one embodiment. ITR—inverted terminal repeat. CMV—human cytomegalovirus (CMV) immediate-early enhancer and promoter. (−)35 Signal. IL-2 SS—interleukin 2 signal sequence. Furin P2A—porcine teschovirus-1 2A self-cleaving peptide. SV40\polyA\signal—simian vacuolating virus 40 poly A signal.

FIG. 34 provides a schematic of a polynucleotide encoding Trastuzumab (Her2 Heavy Chain and Her2 Light Chain) according one embodiment. ITR—inverted terminal repeat. UbC— promoter of the human polyubiquitin C gene (UBC). (−)35 Signal. IL-2 SS—interleukin 2 signal sequence. Furin P2A—porcine teschovirus-1 2A self-cleaving peptide. SV40\polyA\signal—simian vacuolating virus 40 poly A signal.

FIG. 35 a schematic of the experimental procedure for testing and selecting candidate rAAV constructs, as described in Example 2 and Example 3.

FIG. 36 illustrates brain samples obtained for testing transgene transfer and expression, including the sagittal dissection and slab processing for forebrain, midbrain, and cerebellum.

FIG. 37A provides one-way ANOVA analysis of vector genome detection determined by measurement of AAV vector genomic DNA in forebrain tissue and presented as vector genome copies per diploid genome (VGC/DG) for each of the four treatment groups on day 28 as described in Example 3.

FIG. 37B provides one-way ANOVA analysis of transgene expression determined by measurement of mRNA transcript of Trastuzumab in forebrain tissue calculated according to the equation: % Trastuzumab expression=(Trastuzumab cp/uL=RPP30 cp/uL)×100 for each of the four treatment groups on day 28 as described in Example 3.

FIG. 37C provides one-way ANOVA analysis of transgene protein expression determined by measuring Trastuzumab protein expression in forebrain tissue using a HER2-binding ELISA and presented as absorbance normalized to total protein loaded. HER2-binding ELISAs were performed on day 28 for the five treatment groups described in Example 3.

FIG. 38A provides one-way ANOVA analysis of vector genome detection determined by measurement of AAV vector genomic DNA in midbrain tissue and presented as vector genome copies per diploid genome (VGC/DG) for each of the four treatment groups on day 28 as described in Example 3.

FIG. 38B provides one-way ANOVA analysis of transgene expression determined by measurement of mRNA transcript of Trastuzumab in midbrain tissue calculated according to the equation: % Trastuzumab expression=(Trastuzumab cp/uL=RPP30 cp/uL)×100 for each of the four treatment groups on day 28 as described in Example 3.

FIG. 38C provides one-way ANOVA analysis of transgene protein expression determined by measuring Trastuzumab protein expression in midbrain tissue using a HER2-binding ELISA and presented as absorbance normalized to total protein loaded. HER2-binding ELISAs were performed on day 28 for the five treatment groups described in Example 3.

FIG. 39A provides one-way ANOVA analysis of vector genome detection determined by measurement of AAV vector genomic DNA in cerebellum tissue and presented as vector genome copies per diploid genome (VGC/DG) for each of the four treatment groups on day 28 as described in Example 3.

FIG. 39B provides one-way ANOVA analysis of transgene expression determined by measurement of mRNA transcript of Trastuzumab in cerebellum tissue calculated according to the equation: % Trastuzumab expression=(Trastuzumab cp/uL=RPP30 cp/uL)×100 for each of the four treatment groups on day 28 as described in Example 3.

FIG. 39C provides one-way ANOVA analysis of transgene protein expression determined by measuring Trastuzumab protein expression in cerebellum tissue using a HER2-binding ELISA and presented as absorbance normalized to total protein loaded. HER2-binding ELISAs were performed on day 28 for the five treatment groups described in Example 3.

FIGS. 40A-40B are immunohistochemistry (IHC) images of brain sections, obtained from mice in each of the treatment groups described in Example 3 (FIG. 40A=Groups 1 and 2; FIG. 40B=Groups 3 and 4). Brown stain=IgG Fc expression (proxy for Trastuzumab protein). * indicates representative image for 3/10 animas. ** indicates representative image for 7/10 animals. *** indicates representative image for 2/10 animals. White arrows indicate cerebral cortex. Black arrows indicate choroid plexus. Double black arrows indicate hippocampus.

FIGS. 41A-41E show brain lysosulfatide (FIG. 41A), C16:0 sulfatide (FIG. 41B), C18:0 sulfatide (FIG. 41C), C24:0 sulfatide (FIG. 41D) and C24:1 sulfatide (FIG. 41E) levels in ARSA−/− and ARSA+/− mice (Example 7). Circles=ARSA−/−; squares=ARSA+/−.

FIGS. 42A-42E show spinal cord lysosulfatide (FIG. 42A), C16:0 sulfatide (FIG. 42B), C18:0 sulfatide (FIG. 42C), C24:0 sulfatide (FIG. 42D) and C24:1 sulfatide (FIG. 42E) levels in ARSA−/− and ARSA+/− mice (Example 7). Circles=ARSA−/−; squares=ARSA+/−.

FIG. 43 is a schematic illustrating brain slabs collected for analysis following administration of rAAVs (Example 8).

FIGS. 44A-44D show Lysosulfatide (FIG. 44A), C16 sulfatide (FIG. 44B), C18 sulfatide (FIG. 44C), and C24 sulfatide (FIG. 44D) levels in brain slab 1 of animals treated with ARSA rAAVs (Example 8).

FIGS. 45A-45D show Lysosulfatide (FIG. 45A), C16 sulfatide (FIG. 45B), C18 sulfatide (FIG. 45C) and C24 sulfatide (FIG. 45D) levels in brain slab 1 of animals treated with ARSA rAAVs and showing high levels of ARSA expression (UbC constructs) (Example 8).

FIGS. 46A-46D show Lysosulfatide (FIG. 46A), C16 sulfatide (FIG. 46B), C18 sulfatide (FIG. 46C) and C24 sulfatide (FIG. 46D) levels in thoracic spinal cord of animals treated with ARSA rAAVs (Example 8).

FIGS. 47A-47D show Lysosulfatide (FIG. 47A), C16 sulfatide (FIG. 47B), C18 sulfatide (FIG. 47C) and C24 sulfatide (FIG. 47D) levels in thoracic spinal cord of animals treated with ARSA rAAVs and showing high levels of ARSA expression (UbC constructs) (Example 8).

FIG. 48 shows genomic integrity of rAAVs having UbC and CAG promoters as analyzed by the Agilent TapeStation system (Example 9). 1: UbC-ARSA; 2: UbC-COGS; 3: UbC-COGA; 4: CAG-COGS; 5: CAG-COGA; 6: CAG-COGA-mutant-V1; 7: CAG-COGA-mutant-V2.

FIG. 49 shows genomic integrity of UbC-COGS, UbC-COS-Hyper, and CMV-COGS-Hyper rAAVs as analyzed by the Agilent TapeStation system (Example 9).

FIGS. 50A-50B show harvest yield of UbC-COGS, UbC-COS-Hyper, and CMV-COGS-Hyper rAAVs (Example 9). FIG. 50A: vector genomes/mL at harvest; FIG. 50B: relative fold change for three harvests.

FIG. 51 shows capsid purity of UbC-COGS, UbC-COS-Hyper, and CMV-COGS-Hyper rAAVs as analyzed by SDS-PAGE (Example 9).

FIGS. 52A-52B show rotarod results for ARSA knockout (KO) and ARSA+/− (Het) mice at eight months of age, prior to treatment with vehicle or a low or high dose of an ARSA-encoding rAAV (FIG. 52A), and at 12 months of age, three months after treatment (FIG. 52B) (Example 10).

FIGS. 53A-53B show hindlimb clasping (splay) behavior results for ARSA knockout (KO) and ARSA+/− (Het) mice at eight months of age, prior to treatment with vehicle or a low or high dose of an ARSA-encoding rAAV (FIG. 53A), and at 12 months of age, three months after treatment (FIG. 53B) (Example 10).

FIGS. 54A-54B show pole test results for ARSA knockout (KO) and ARSA+/− (Het) mice at 12 months of age, three months after treatment with vehicle or a low or high dose of an ARSA-encoding rAAV (Example 10). FIG. 54A shows total time for individual runs; FIG. 54B shows total time for all trials.

FIG. 55 shows success rate on the pole test for ARSA knockout (KO) and ARSA+1-(Het) mice at 12 months of age, three months after treatment with vehicle or a low or high dose of an ARSA-encoding rAAV (Example 10).

FIGS. 56A-56B show success rate on the pole test for female (FIG. 56A) and male (FIG. 56B) ARSA knockout (KO) and ARSA+/− (Het) mice at 12 months of age, three months after treatment with vehicle or a low or high dose of an ARSA-encoding rAAV (Example 10).

FIGS. 57A-57B show body weight (FIG. 57A) and brain weight (FIG. 57B) for ARSA knockout (KO) and ARSA+/− (Het) mice at 12 months of age, three months after treatment with vehicle or a low or high dose of an ARSA-encoding rAAV (Example 10).

FIGS. 58A-58F show a schematic of brain slabs obtained from ARSA knockout (KO) and ARSA+/− (Het) mice at 12 months of age, three months after treatment with vehicle or a low or high dose of an ARSA-encoding rAAV (FIG. 58A) and levels of lysosulfatide (FIG. 58B), C16 sulfatide (FIG. 58C), C18 sulfatide (FIG. 58D), C24 sulfatide (FIG. 58E), and C24:1 sulfatide (FIG. 58F) in slab 1 (Example 10).

FIGS. 59A-59E show levels of lysosulfatide (FIG. 59A), C16 sulfatide (FIG. 59B), C18 sulfatide (FIG. 59C), C24 sulfatide (FIG. 59D), and C24:1 sulfatide (FIG. 59E) in thoracic spinal cord of ARSA knockout (KO) and ARSA+/− (Het) mice at 12 months of age, three months after treatment with vehicle or a low or high dose of an ARSA-encoding rAAV (Example 10).

FIGS. 60A-60B show brain slabs (FIG. 60A) used for analysis of vector genome biodistribution in ARSA knockout mice at 12 months of age, three months after treatment with a low or high dose of an ARSA-encoding rAAV, and vector genome biodistribution in slabs 2-6 (Example 10).

FIG. 61 shows ARSA enzyme activity in combined brain slabs from WT mice and from ARSA knockout (KO) and ARSA+/−(Het) mice at 12 months of age, three months after treatment with vehicle or a low or high dose of an ARSA-encoding rAAV (Example 10).

### 6. DETAILED DESCRIPTION OF THE INVENTION

### 6.1. Definitions

The term “antigen binding protein” or “ABP” as used herein includes an antibody, or functional fragment thereof. The ABP can exist in a variety of form including, for example, a polyclonal antibody, monoclonal antibody, camelized single domain antibody, intracellular antibody (“intrabodies”), recombinant antibody, multispecific antibody, antibody fragment, such as, Fv, Fab, F(ab)2, F(ab)3, Fab′, Fab′-SH, F(ab′)2, single chain variable fragment antibody (scFv), tandem/bis-scFv, Fc, pFc′, scFvFc (or scFv-Fc), disulfide Fv (dsfv), bispecific antibody (bc-scFv) such as BITE antibody; camelid antibody, resurfaced antibody, humanized antibody, fully human antibody, single-domain antibody (sdAb, also known as NANOBODY®), chimeric antibody, chimeric antibody comprising at least one human constant region, and the like. “Antibody fragment” refers to at least a portion of the variable region of the immunoglobulin that binds to its target, e.g., the tumor cell.

As used herein, the term “CDR” or “complementarity determining region” refers to the noncontiguous antigen combining sites found within the variable region of heavy chain and light chain polypeptides that are involved in antigen binding.

### 6.2. Recombinant Adeno-Associated Virus

One aspect of the present disclosure provides an rAAV comprising a capsid comprising: a capsid protein comprising the amino acid sequence of SEQ ID NO: 1 or a variant thereof, and the polynucleotide encapsulated by the capsid. The polynucleotide can encode a therapeutic protein. In a particular embodiment, the polynucleotide includes a coding sequence of ARSA or a functional variant thereof. In some embodiments, the ARSA or functional variant has an amino acid sequence comprising the amino acid sequence of SEQ ID NO: 5 or SEQ ID NO: 6. In another particular embodiment, the polynucleotide includes a coding sequence of Trastuzumab, including a heavy chain (SEQ ID NO: 35) and a light chain (SEQ ID NO: 36).

### 6.2.1. Capsid

The rAAV used in various embodiments of the present disclosure comprises a capsid formed with VP1, VP2 and VP3 capsid proteins. In a particular embodiment, the capsid is formed with VP1, VP2 and VP3 capsid proteins of Anc80L65. In some embodiments, VP1 protein has the amino acid sequence of SEQ ID NO: 1. In some embodiments, the VP1 protein comprises a sequence having at least 80%, 90%, 95%, 96%, 97%, 98%, or 99% sequence identity to SEQ ID NO: 1. In some embodiments, VP2 and VP3 proteins have a portion of the amino acid sequence of SEQ ID NO: 1. In some embodiments, VP2 protein has a sequence corresponding to amino acids 138 to 736 of SEQ ID NO: 1 and VP3 protein can have a sequence corresponding to amino acids 203 to 736 of SEQ ID NO: 1. In some embodiments, VP2 protein has a sequence corresponding to a sequence having at least 80%, 90%, 95%, 96%, 97%, 98%, or 99% sequence identity to amino acids 138 to 736 of SEQ ID NO: 1 and/or VP3 protein can have a sequence corresponding to a sequence having at least 80%, 90%, 95%, 96%, 97%, 98%, or 99% sequence identity to amino acids 203 to 736 of SEQ ID NO: 1.

### 6.2.2. Polynucleotide

The rAAV disclosed herein comprises a polynucleotide encapsulated by the capsid. The polynucleotide comprises a sequence encoding a protein, peptide or RNA for treatment of a CNS disease. In some embodiments, the polynucleotide comprises a coding sequence of a protein associated with a CNS disease.

In some embodiments, the polynucleotide comprises a coding sequence of a therapeutic protein (e.g., genetically deficient protein in a subject with a CNS disease, antigen binding protein), RNAs (e.g., inhibitory RNAs or catalytic RNAs), or target antigens (e.g., oncogenic antigens, autoimmune antigens). In some embodiments, the rAAV comprises a polynucleotide encoding a tRNA, miRNA, gene editing guide RNA, or RNA-editing guide RNA.

In some embodiments, the polynucleotide comprises a coding sequence of a secretory protein. A secretory protein is a protein, whether it be endocrine or exocrine, which is secreted by a cell. Secretory proteins include but are not limited to hormones, enzymes, toxins, and antimicrobial peptides. In some embodiments, secretory proteins are synthesized in the endoplasmic reticulum. In some embodiments, the polynucleotide comprises a coding sequence of a secretory protein associated with a CNS disease.

In some embodiments of the present disclosure, the rAAV comprises one or more transgene. A transgene may be, for example, a reporter gene (e.g., beta-lactamase, beta-galactosidase (LacZ), alkaline phosphatase, thymidine kinase, green fluorescent polypeptide (GFP), chloramphenicol acetyltransferase (CAT), or luciferase, or fusion polypeptides that include an antigen tag domain such as hemagglutinin or Myc), or a therapeutic gene (e.g., genes encoding hormones or receptors thereof, growth factors or receptors thereof, differentiation factors or receptors thereof, immune system regulators (e.g., cytokines and interleukins) or receptors thereof, enzymes, RNAs (e.g., inhibitory RNAs or catalytic RNAs), or target antigens (e.g., oncogenic antigens, autoimmune antigens)). In some embodiments, the rAAV comprises an expressible polynucleotide encoding a therapeutic tRNA, miRNA, gene editing guide RNA, or RNA-editing guide RNA.

In some embodiments, the polynucleotide comprises a coding sequence of a protein deficient in a subject (e.g., a human) having a CNS disease. In some embodiments, the coding sequence encodes one or more of a protein known to be associated with a disease selected from: Adrenoleukodystrophy, Alexander Disease, Alzheimer disease, Amyotrophic lateral sclerosis, Angelman syndrome, Ataxia telangiectasia, Canavan disease, Charcot-Marie-Tooth syndrome, Cockayne syndrome, Chronic inflammatory demyelinating polyneuropathy (CIDP), Deafness, Duchenne muscular dystrophy, Epilepsy, Essential tremor, Fragile X syndrome, Friedreich's ataxia, Gaucher disease, GM1 gangliosidosis, GM2 gangliosidoses, Huntington disease, Frontotemporal Degeneration (FTD), Lesch-Nyhan syndrome, Maple syrup urine disease, Menkes syndrome, Metachromatic leukodystrophy (MLD), Myotonic dystrophy, Multiple sclerosis, Narcolepsy, Neurofibromatosis, Niemann-Pick disease, Parkinson's disease, Phenylketonuria, Prader-Willi syndrome, Refsum disease, Rett syndrome, Spinal muscular atrophy, Spinocerebellar ataxia, Tangier disease, Tay-Sachs disease, Tuberous sclerosis, Von Hippel-Lindau syndrome, Williams syndrome, Wilson's disease, or Zellweger syndrome.

In some embodiments, the coding sequence encodes a protein known to be associated with a lysosomal storage disease, as known in the art and as described herein.

In some embodiments, the coding sequence encodes a protein known to be associated with a demyelinating or white matter disease, as known in the art and as described herein.

In some embodiments, the polynucleotide comprises a coding sequence of an antigen that can induce an immune response in a subject when administered. In some embodiments, the polynucleotide comprises a coding sequence of viral or bacterial antigen. In some embodiments, the antigen is useful for immunizing a subject (e.g., a human, an animal (e.g., a companion animal, a farm animal, an endangered animal). For example, antigen can be obtained from an organism (e.g., a pathogenic organism) or an immunogenic portion or component thereof (e.g., a toxin polypeptide or a by-product thereof). By way of example, pathogenic organisms from which immunogenic polypeptides can be obtained include viruses (e.g., picornavirus, enteroviruses, orthomyxovirus, reovirus, retrovirus), prokaryotes (e.g., Pneumococci, Staphylococci, Listeria, Pseudomonas), and eukaryotes (e.g., amebiasis, malaria, leishmaniasis, nematodes). It would be understood that the methods described herein and compositions produced by such methods are not to be limited by any particular transgene. In some embodiments, the polynucleotide comprises a coding sequence which has been codon optimized.

In some embodiments, the polynucleotide comprises a coding sequence of hASPA (aminoacylase 2) for treatment of Canavan disease. In some embodiments, the polynucleotide comprises a coding sequence of hAADC for treatment of AADC deficiency. In some embodiments, the polynucleotide comprises a coding sequence of one or more of NTN, hGDNF, and hAADC for treatment of Parkinson's disease. In some embodiments, the polynucleotide comprises a coding sequence of one or more of hNGF and hAPOE2 for treatment of Alzheimer's disease. In some embodiments, the polynucleotide comprises a coding sequence of SMN for treatment of SMA1. In some embodiments, the polynucleotide comprises a coding sequence of Glial fibrillary acidic protein (GFAP) for treatment of Alexander Disease. In some embodiments, the polynucleotide comprises a coding sequence of one or more selected from: allograft inflammatory factor 1 (AIF-1), lymphatic hyaluronan receptor (LYVE-1/XLKD1), FYN binding protein (FYB), P2RY1 (purinergic receptor P2Y, G-protein-coupled, 1), and MLLT3 (myeloid/lymphoid or mixed-lineage leukemia translocated to, 3), for treatment of chronic inflammatory demyelinating polyneuropathy (CIDP). In some embodiments, the polynucleotide comprises a coding sequence of one or more of a gene described in D'Netto M J, et al. “Risk alleles for multiple sclerosis in multiplex families.” Neurology. 2009 Jun. 9; 72(23):1984-8 (incorporated herein by reference), for treatment of multiple sclerosis. In some embodiments, the polynucleotide comprises a coding sequence of one or more of a gene selected from IL2RA, IL7R, EVI5, KIAA0350, and CD58, for treatment of multiple sclerosis.

In some embodiments, the polynucleotide further comprises a regulatory sequence regulating expression from the coding sequence. In some embodiments, the polynucleotide comprises a regulatory sequence directing expression of the gene product in a target cell. In some embodiments, when the polynucleotide comprises a regulatory sequence directing expression of the gene product in a target cell, the regulatory sequence and the gene are considered operably linked. In some embodiments, the regulatory sequence is a promoter sequence. In some embodiments, the regulatory sequence is a combination of one or more promoter sequences and one or more enhancer sequences. In some embodiments, the regulatory sequence comprises a UbC promoter, CMV promoter, or CAG promoter. In some embodiments, the regulatory sequence comprises a CMV or UbC promoter. In some embodiments, the regulatory sequence comprises a UbC promoter. In some embodiments, the regulatory sequence comprises a CMV promoter. In some embodiments, the regulatory sequence comprises a CAG promoter. In some embodiments, the regulatory sequence is selected from SEQ ID NOs: 9-14. In some embodiments, the regulatory sequence has at least 80%, 85%, 90%, 95%, 96%, 97%, 98%, 99% or 100% sequence identity to SEQ ID NO: 9, 10, 11, 12, 13, or 14. In some embodiments, the regulatory sequence has 80%, 85%, 90%, 95%, 96%, 97%, 98%, or greater sequence identity to SEQ ID NO: 9, 10, 11, 12, 13, or 14. In some embodiments, the regulatory sequence is selected from SEQ ID NO: 11 or 14. In some embodiments, the regulatory sequence has at least 80%, 85%, 90%, 95%, 96%, 97%, 98%, 99% or 100% sequence identity to SEQ ID NO: 11 or 14. In some embodiments, the regulatory sequence has 80%, 85%, 90%, 95%, 96%, 97%, 98%, or greater sequence identity to SEQ ID NO: 11 or 14.

In some embodiments, the polynucleotide further comprises non-coding sequences at 3′ to the coding sequence. Non-limiting examples of non-coding sequences at 3′ to the coding sequence include a poly(A) signal and a woodchuck hepatitis virus post-transcriptional regulatory element (WPRE). An exemplary WPRE sequence is set forth in SEQ ID NO: 15. In some embodiments, the nucleotide sequence of the WPRE comprises a nucleotide sequence having at least 90%, at least 95%, at least 96%, at least 97%, or at least 98%, at least 99%, or 100% sequence identity to SEQ ID NO: 15. An exemplary poly(A) signal is the SV40 late polyadenylation signal. An exemplary SV40 late polyadenylation signal nucleotide sequence is set forth in SEQ ID NO: 16.

In some embodiments, the polynucleotide further comprises a target sequence to one or more miRNA. In some embodiments, the miRNA is expressed or active only in a specific cell, tissue or organ. In some embodiments, the miRNA is expressed or active only in dorsal root ganglia (DRG). In some embodiments, the polynucleotide comprises a target sequence to miR-183, miR-182, or miR-96. In some embodiments, the polynucleotide comprises more than one target sequences, wherein each target sequence is specific to miR-183, miR-182, or miR-96. In some embodiments, the polynucleotide comprises at least two tandem repeats of the target sequences which comprise at least a first miRNA target sequence and at least a second miRNA target sequence which may be the same or different, as described in WO2020132455A1, the contents of which are incorporated by reference. In some embodiments, the target sequences to one or more miRNA are located at the 3′ end of the polynucleotide. In certain embodiments, the polynucleotide comprises at least two tandem repeats of the miRNA target sequences that are located at 3′ UTR. In certain embodiments, the polynucleotide comprises three tandem repeats of miRNA target sequences. In certain embodiments, the at least two DRG-specific miRNA target sequences are located at both the 5′ UTR and the 3′ UTR. In some embodiments, the two or more consecutive miRNA target sequences are continuous and not separated by a spacer.

In some embodiments, the polynucleotide comprises more than one coding sequence. In some embodiments, the multiple coding sequences are separated by one or more self-cleaving peptides. The self-cleaving peptides can be 2A self-cleaving peptides. Non-limiting examples of self-cleaving peptides include 2A peptides (18-22 amino acids), including a peptide from foot-and-mouth disease virus (F2A), porcine teschovirus-1 (P2A), Thoseaasigna virus (T2A), or equine rhinitis A virus (E2A). In some embodiments, the polypeptide comprises Furin P2A. In some embodiments, the Furin P2A has the sequence of SEQ ID NO: 37. In some embodiments, the multiple coding sequences are separated by one or more internal ribosome entry site (IRES).

In some embodiments, the polynucleotide further comprises AAV's inverted terminal repeats (ITRs). Exemplary 5′ ITR and 3′ ITR nucleotide sequences are set forth in SEQ ID NOs: 17-18, respectively.

In some embodiments, the polynucleotide further comprises a signal sequence encoding a signal peptide. In some embodiments, a signal peptide enhances secretion of a polypeptide (e.g., any of the antigen-binding proteins (ABP)) encoded by the coding sequences described herein) from the cell in which the polynucleotide is transferred. A non-limiting example of a signal sequence includes an interleukin-2 (IL-2) signal sequence. In some embodiments, the signal sequence has the sequence of SEQ ID NO: 38. One of skill in the art would appreciate that other signal sequences could be used along with the methods described herein to enhance secretion.

### 6.2.2.1 Polynucleotide for Treatment of Lysosomal Storage Disease

In some embodiments, the rAAV provided herein is used to transfer a polynucleotide to a subject having a lysosomal storage disease, e.g., a lack or deficiency in a lysosomal storage enzyme. In some embodiments, the polynucleotide comprises a coding sequence of ZFN for safe insertion of hIDUA for treatment of MPS1. In some embodiments, the polynucleotide comprises a coding sequence of ZFN for safe insertion of hIDS for treatment of MPSII. In some embodiments, the polynucleotide comprises a coding sequence of hSGSH for treatment of MPS IIIA. In some embodiments, the polynucleotide comprises a coding sequence of hNAGLU for treatment of MPSIIIB. In some embodiments, the polynucleotide comprises a coding sequence of hCLN2, hCLN3, or hCNL6 for treatment of LINCL (Batten disease). In some embodiments, the polynucleotide comprises a coding sequence of human arylsulfatase A (hARSA) for treatment of MLD.

In some embodiments, the rAAV comprises a polynucleotide comprising a coding sequence of a gene associated with the lysosomal storage disease as provided in TABLE 1.

TABLE 1
Genes known to be involved in lysosomal storage diseases

| Lysosomal storage disease | Gene associated with lysosomal storage disease |
|---|---|
| mucopolysaccharidosis type I e.g., | alpha-L-iduronidase |
| Hurler syndrome and the variants Scheie | |
| syndrome and Hurler-Scheie syndrome | |
| Hunter syndrome | iduronate-2-sulfatase |
| mucopolysaccharidosis type III, e.g., | heparan sulfate sulfatase, N-acetyl-alpha-D- |
| Sanfilippo syndrome | glucosaminidase, acetyl CoA:alpha- |
| | glucosaminide N-acetyl transferase or N- |
| | acetylglucosamine-6-sulfate sulfatase |
| mucopolysaccharidosis type IV, e.g., | galactosamine-6-sulfate sulfatase or beta- |
| Morquio syndrome | galactosidase |
| mucopolysaccharidosis type VI, e.g., | arylsulfatase B |
| Maroteaux-Lamy syndrome | |
| mucopolysaccharidosis type II; | heparan sulfate sulfatase, N-acetyl-alpha-D- |
| mucopolysaccharidosis type III | glucosaminidase, acetyl CoA:alpha- |
| | glucosaminide N-acetyl transferase or N- |
| | acetylglucosamine-6-sulfate sulfatase |
| mucopolysaccharidosis type IV | galactosamine-6-sulfatase and beta- |
| | galactosidase |
| mucopolysaccharidosis type VI | arylsulfatase B |
| mucopolysaccharidosis type VII | beta-glucuronidase |
| mucopolysaccharidosis type VIII | glucosamine-6-sulfate sulfatase |
| mucopolysaccharidosis type IX | hyaluronidase |
| Tay-Sachs disease | beta-hexosaminidase |
| Sandhoff disease | alpha and beta subunit of beta- |
| | hexosaminidase |
| GM1 gangliosidosis | beta-galactosidase-1 (GLB-1) |
| Fabry disease | alpha galactosidase |
| Krabbe's leukodystrophy | Galactocerebrosidase |
| metachromatic leukodystrophy | aryl sulfatase A (ARSA) or prosaposin (PSAP) |
| Pompe disease | acid maltase |
| Fucosidosis deficiency | Fucosidosis |
| alpha-mannosidosis deficiency | alpha-mannosidosis |
| beta-mannosidosis deficiency | beta-mannosidosis |
| Gaucher disease | Glucocerebrosidase |
| Infantile Batten Disease | CNL1 |
| Classic Late Infantile Batten Disease | CNL2 |
| Juvenile Batten Disease | CNL3 |
| Batten, other forms | CNL4-CNL8 |
| Niemann-Pick disease | Sphingomyelinase |
| Niemann-Pick disease without | npc1 gene encoding a cholesterol |
| sphingomyelinase deficiency | metabolizing enzyme |
| Wolman disease | cholesterol ester hydrolase |
| Vanishing White Matter Disease (VWM) | EIF2B1, EIF2B2, EIF2B3, EIF2B4, or EIF2B5 |

In some embodiments, the rAAV comprises a polynucleotide containing a coding sequence of ARSA, or functional variant thereof, for treatment of arylsulfatase A deficiency or metachromatic leukodystrophy (MLD). In some embodiments, the coding sequence has been codon optimized. In some embodiments, the coding sequence encodes a functional variant of ARSA, having improved enzyme or other protein activity, and/or longer half-life compared to a naturally occurring ARSA protein. In some embodiments, the coding sequence of ARSA described in US 2019/0352624 (Univ Bonn Rheinische Friedrich Wilhems) is used, the patent publication is incorporated by reference in its entirety herein. In some embodiments, the coding sequence is selected from SEQ ID Nos: 2-4 and 7-8. In some embodiments, the coding sequence has at least 80%, 85%, 90%, 95%, 96%, 97%, 98%, 99% or 100% sequence identity to SEQ ID No: 2, 3, 4, 7, or 8. In some embodiments, the coding sequence has 80%, 85%, 90%, 95%, 96%, 97%, 98%, or greater sequence identity to SEQ ID No: 2, 3, 4, 7, or 8.

The coding sequence can encode for a full length ARSA or functional variant (e.g., having the amino acid sequence of SEQ ID NO: 5 or SEQ ID NO: 6) or a fragment thereof having ARSA activity. In some embodiments, the coding sequence encodes a protein whose amino acid sequence is at least 95%, at least 96%, at least 97%, at least 98%, at least 99%, or 100% identical to SEQ ID NO: 5 or SEQ ID NO: 6.

In some embodiments, coding sequence encodes an ARSA functional variant having one or more amino acid substitutions relative to SEQ ID NO: 5. For example, the ARSA functional variant can have M202V and/or T286L and/or R291N substitutions. In some embodiments, the ARSA functional variant is Hyper-ARSA (SEQ ID NO: 6), which has 202V, T286L, and R291N substitutions. Hyper-ARSA has been reported to have substantially increased activity compared to native human ARSA (see, Simonis et al., 2019, Human Molecular Genetics 28(11):1810-1821 and WO 2018/141958, the contents of each of which are incorporated herein by reference in their entireties).

The nucleotide sequence encoding ARSA or a functional variant thereof can be codon-optimized for expression in human cells. Codon-optimization tools are commercially available and include, for example, the Genscript GenSmartTM codon optimization tool (available at www.genscript.com/gensmart-free-gene-codon-optimization.html), the GeneArt codon optimization tool (available at www.thermofisher.com/us/en/home/life-science/cloning/gene-synthesis/geneart-gene-synthesis/geneoptimizer.html), the IDT codon-optimization tool (available at www.idtdna.com/pages/tools/codon-optimization-tool), and the VectorBuilder codon optimization tool (available at en.vectorbuilder.com/tool/codon-optimization.html). Exemplary codon optimized coding sequences are set forth in SEQ ID NOs: 2-3 (native ARSA) and SEQ ID NOs: 7-8 (Hyper-ARSA). In some embodiments, the coding sequence has at least 80%, at least 85%, at least 90%, at least 95%, at least 96%, at least 97%, or at least 98%, at least 99%, or 100% sequence identity to SEQ ID NO: 2, SEQ ID NO: 3, SEQ ID NO: 7, or SEQ ID NO: 8 and encodes a polypeptide whose amino acid sequence is at least at least 95%, at least 96%, at least 97%, at least 98%, at least 99%, or 100% sequence identity with SEQ ID NO: 5 or SEQ ID NO: 6.

In some embodiments, the rAAV comprises a polynucleotide containing a coding sequence of beta-galactosidase-1 (GLB-1), or functional variant thereof, for treatment of GM1 gangliosidosis. In some embodiments, the coding sequence has been codon optimized. In some embodiments, the coding sequence encodes a functional variant of GLB-1, having improved enzyme or other protein activity, and/or longer half-life, compared to naturally occurring GLB-1.

In some embodiments, the rAAV comprises a polynucleotide containing a coding sequence of galactocerebroside, or a functional variant thereof, for treatment of Krabbe's leukodystrophy. In some embodiments, the coding sequence has been codon optimized. In some embodiments, the coding sequence encodes a functional variant of galactocerebroside, having improved enzyme or other protein function and/or longer half-life, compared to naturally occurring galactocerebroside.

### 6.2.2.2 Polynucleotide for Treatment of Brain Cancer

In some embodiments, the rAAV provided herein is used for treating a subject having brain cancer. In some embodiments, rAAV comprises a polynucleotide comprising a coding sequence of a gene associated with treating cancer.

In some embodiments, the polynucleotide encapsulated by the capsid is a polynucleotide encoding an antigen binding protein (ABP). In some embodiments, the polynucleotide comprises a coding sequence of an ABP specific to a tumor cell. In some embodiments, the polynucleotide comprises a coding sequence of an ABP specific to a brain tumor antigen.

In some embodiments, the ABP is a monoclonal antibody. In some embodiments, the ABP is selected from a human antibody, a humanized antibody, or a chimeric antibody. In some embodiments, the antibody is a single chain variable fragment (scFv).

In some embodiments, the polynucleotide comprises a coding sequence of an immunoglobulin constant region. In some embodiments, the polynucleotide comprises a coding sequence of a Fab, Fab′, F(ab′)₂, Fv, scFv, (scFv)₂, single chain antibody molecule, dual variable domain antibody, single variable domain antibody, linear antibody, V domain antibody, or bispecific tandem bivalent scFvs.

In some embodiments, the polynucleotide comprises a coding sequence of a heavy chain constant region of a class selected from IgG, IgA, IgD, IgE, and IgM. In some embodiments, the polynucleotide comprises a coding sequence of a heavy chain constant region of the class IgG and a subclass selected from IgG1, IgG2, IgG3, and IgG4. In some embodiments, the polynucleotide comprises a coding sequence of a heavy chain constant region of IgG.

In some embodiments, the polynucleotide comprises a coding sequence of a heavy chain of an ABP. In some embodiments, the polynucleotide comprises a coding sequence of a light chain of an ABP. In some embodiments, the polynucleotide comprises coding sequences of a heavy chain and a light chain. In some embodiments, the polynucleotide comprises from 5′ to 3′ coding sequences of a heavy chain of an ABP and a light chain of an ABP. In some embodiments, the polynucleotide comprises from 5′ to 3′ coding sequences of a light chain of an ABP and a heavy chain of an ABP. In some embodiments, the polynucleotide comprises a self-cleaving peptide between the heavy chain coding sequence and the light chain coding sequence. In some embodiments, the heavy chain coding sequence is linked to interleukin 2 signal sequence. In some embodiments, the light chain coding sequence is linked to interleukin 2 signal sequence.

In some embodiments, the ABP encoded by the polynucleotide is an ABP specific to human epidermal growth factor receptor 2 (HER2). In some embodiments, the coding sequence encodes an antibody, (e.g., trastuzumab), or a modification thereof. In some embodiments, the coding sequence encodes an ABP comprising the CDRs of trastuzumab or variants thereof. In some embodiments, the coding sequence encodes trastuzumab having the sequence of a heavy chain of SEQ ID NO: 35 and a light chain of SEQ ID NO: 36. In some embodiments, the coding sequence has been codon optimized. In some embodiments, the anti-HER2 ABP is encoded by a coding sequence of trastuzumab described in US2013/0273650 (Wu), incorporated by reference in its entirety herein. In some embodiments, the anti-HER2 ABP is encoded by a coding sequence of trastuzumab described in U.S. Pat. No. 10,780,182 (Wilson), incorporated by reference in its entirety herein.

In some embodiments, the polynucleotide comprises a coding sequence of a heavy chain of trastuzumab or a coding sequence of a light chain of trastuzumab.

In some embodiments, the heavy chain coding sequence has the sequence of SEQ ID NO: 29, 31, or 33. In some embodiments, the heavy chain coding sequence has a sequence having at least 90%, 95%, 97%, 98%, or 99% identity to SEQ ID NO: 29, 31 or 33. In some embodiments, the heavy chain coding sequence is linked to interleukin 2 signal sequence.

In some embodiments, the light chain coding sequence has the sequence of SEQ ID NO: 30, 32, or 34. In some embodiments, the light chain coding sequence has a sequence having at least 90%, 95%, 97%, 98%, or 99% identity to SEQ ID NO: 30, 32, or 34. In some embodiments, the light chain coding sequence is linked to interleukin 2 signal sequence.

In some embodiments, the polynucleotide comprises both a coding sequence of a heavy chain of Trastuzumab and/or a coding sequence of a light chain of Trastuzumab. In some embodiments, the polynucleotide comprises a self-cleaving peptide between the coding sequence of a heavy chain and the coding sequence of a light chain. In some embodiments, the self-cleaving peptide is a 2A peptide (18-22 amino acids). In some embodiments, the 2A peptide is F2A, P2A, T2A, or E2A. In some embodiments, the self-cleaving peptide has the sequence of SEQ ID NO: 37.

In some embodiments, the heavy chain coding sequence comprises a heavy chain variable domain (VH) comprising a sequence having at least 90%, 95%, 97%, 98%, or 99% identity to SEQ ID NOs: 42, 44, or 46. In some embodiments, the light chain coding sequence comprises a light chain variable domain (VL) comprising a sequence having at least 90%, 95%, 97%, 98%, or 99% identity to SEQ ID NOs: 43, 45, or 47. In some embodiments, the polynucleotide comprises both a coding sequence of a heavy chain variable domain comprising a sequence having at least 90%, 95%, 97%, 98%, or 99% identity to SEQ ID NOs: 42, 44, or 46 and the light chain variable domain comprising a sequence having at least 90%, 95%, 97%, 98%, or 99% identity to SEQ ID NOs: 43, 45, or 47.

In some embodiments, the coding sequence encodes an anti-Her2 ABP comprising the CDRs of trastuzumab or variants thereof. In some embodiments, the heavy chain coding sequence comprises a sequence of SEQ ID NO: 48. In some embodiments, the coding sequence encodes trastuzumab comprising a CDR3 having a sequence of SEQ ID NO: 49.

In some embodiments, the polynucleotide comprises a coding sequence having the sequence of SEQ ID NO: 23. In some embodiments, the coding sequence has at least 80%, 85%, 90%, 95%, 96%, 97%, 98%, 99% or 100% sequence identity to SEQ ID NO: 23. In some embodiments, the coding sequence has 80%, 85%, 90%, 95%, 96%, 97%, 98%, 99% or greater sequence identity to SEQ ID NO: 23.

In some embodiments, the coding sequence of anti-Her2 ABP comprises from 5′ to 3′: a heavy chain coding sequence followed by a light chain coding sequence. In some embodiments, the coding sequence of anti-Her2 ABP comprises from 5′ to 3′: a light chain coding sequence followed by a heavy chain coding sequence.

In some embodiments, the polypeptide comprises from 5′ to 3′, coding sequences of interleukin 2 signal peptide, a heavy chain of anti-Her2 ABP, a self-cleaving peptide, interleukin 2 signal peptide, and a light chain of anti-Her2 ABP. In some embodiments, the polypeptide comprises from 5′ to 3′, coding sequences of interleukin 2 signal peptide, a light chain of anti-Her2 ABP, a self-cleaving peptide, interleukin 2 signal peptide, and a heavy chain of anti-Her2 ABP.

In some embodiments, the polynucleotide (e.g., a polynucleotide encoding an ABP specific to human epidermal growth factor receptor 2 (HER2)) is or comprises a sequence selected from SEQ ID NOs: 24-28. In some embodiments, the polynucleotide comprises a sequence having at least 80%, 85%, 90%, 95%, 96%, 97%, 98%, 99% or 100% sequence identity to SEQ ID NOs: 24-28. In some embodiments, the polynucleotide has 80%, 85%, 90%, 95%, 96%, 97%, 98%, 99% or greater sequence identity to SEQ ID NOs: 24-28.

In some embodiments, the polynucleotide (e.g., a polynucleotide encoding an ABP specific to HER2) is or comprises a sequence of SEQ ID NO: 24. In some embodiments, the polynucleotide is or comprises a sequence of SEQ ID NO: 25.

In some embodiments, the polynucleotide (e.g., a polynucleotide encoding an ABP specific to HER2) comprises one or more mutations in the heavy chain and/or light chain coding sequences that result in an amino acid substitution.

In some embodiments, the one or more mutations enhance antibody-dependent cellular cytotoxicity (ADCC). In some embodiments, the polynucleotide includes a coding sequence comprising one or more mutations that lead to amino acid substitutions at amino acid residues 239, 332, and/or 330. In some embodiments, the amino acid substitutions include S239D, 1332E, and/or A330L.

In some embodiments, one or more mutations enhance antibody effector function. In some embodiments, the polynucleotide includes a coding sequence comprising one or more mutations that lead to amino acid substitutions at amino acid residues 356 and/or 358 in the heavy chain amino acid sequence. In some embodiments, the amino acid substitutions include D356E and/or L358M. In some embodiments, the polynucleotide has the sequence of SEQ ID NOs: 23, 24, or 25.

In some embodiments, the ABP encoded by the polynucleotide is a recombinant humanized monoclonal antibody that targets the extracellular dimerization domain (Subdomain II) of the human epidermal growth factor receptor 2 protein (HER2). For example, pertuzumab can be used. The amino acid sequences of its heavy chain and light chain are provided, e.g., in drugbank.ca/drugs/DB06366 (synonyms include 2C4, MOAB 2C4, monoclonal antibody 2C4, and rhuMAb-2C4) on this database at accession number DB06366.

In some embodiments, the ABP encoded by the polynucleotide is MM-121/SAR256212, a fully human monoclonal antibody that targets the HER3 receptor [Merrimack's Network Biology] and which has been reported to be useful in the treatment of non-small cell lung cancer (NSCLC), breast cancer and ovarian cancer.

In some embodiments, the ABP encoded by the polynucleotide is SAR256212, a fully human monoclonal antibody that targets the HER3 (ErbB3) receptor [Sanofi Oncology].

In some embodiments, the ABP encoded by the polynucleotide is anti-Her3/EGFR antibody, RG7597 [Genentech], described as being useful in head and neck cancers.

In some embodiments, the ABP encoded by the polynucleotide is margetuximab (or MGAH22), a next-generation, Fc-optimized monoclonal antibody (mAb) that targets HER [MacroGenics].

In some embodiments, other human epithelial cell surface markers and/or other tumor receptors or antigens are targeted by a protein (e.g., ABP or enzyme) encoded by the polynucleotide encapsulated by the rAAV. Examples of other cell surface marker targets include: 5T4, CA-125, CEA (e.g., targeted by labetuzumab), CD3, CD19, CD20 (e.g., targeted by rituximab), CD22 (e.g., targeted by epratuzumab or veltuzumab), CD30, CD33, CD40, CD44, CD51 (also integin αvβ3), CD133 (e.g., glioblastoma cells), CTLA-4 (e.g., Ipilimumab used in treatment of neuroblastoma), Chemokine (C—X—C Motif) Receptor 2 (CXCR2) (expressed in different regions in brain; e.g., Anti-CXCR2 (extracellular) antibody #ACR-012 (Alomene Labs)); EpCAM, fibroblast activation protein (FAP) [see, e.g., WO 2012020006 A2, brain cancers], folate receptor alpha (e.g., pediatric ependymal brain tumors, head and neck cancers), fibroblast growth factor receptor 1 (FGFR1) (see, e.g., WO2012125124A1 for discussion treatment of cancers with anti-FGFR1 antibodies), FGFR2 (see, e.g., antibodies described in WO2013076186A and WO2011143318A2), FGFR3 (see, e.g., antibodies described in U.S. Pat. No. 8,187,601 and WO2010111367A1), FGFR4 (see, e.g., anti-FGFR4 antibodies described in WO2012138975A1), hepatocyte growth factor (HGF) (see, e.g., antibodies in WO2010119991A3), integrin α5β1, IGF-1 receptor, gangioloside GD2 (see, e.g., antibodies described in WO2011160119A2), ganglioside GD3, transmembrane glycoprotein NMB (GPNMB) (associated with gliomas, among others and target of the antibody glembatumumab (CR011), mucin, MUC1, phosphatidylserine (e.g., targeted by bavituximab, Peregrine Pharmaceuticals, Inc], prostatic carcinoma cells, PD-L1 (e.g., nivolumab (BMS-936558, MDX-1106, ONO-4538), a fully human gG4, e.g., metastatic melanoma], platelet-derived growth factor receptor, alpha (PDGFR α) or CD140, tumor associated glycoprotein 72 (TAG-72), tenascin C, tumor necrosis factor (TNF) receptor (TRAIL-R2), vascular endothelial growth factor (VEGF)-A (e.g., targeted by bevacizumab) and VEGFR2 (e.g., targeted by ramucirumab). Other antibodies and their targets include, e.g., APN301 (hu14.19-IL2), a monoclonal antibody [malignant melanoma and neuroblastoma in children, Apeiron Biologics, Vienna, Austria]. See, also, e.g., monoclonal antibody, 8H9, which has been described as being useful for the treatment of solid tumors, including metastatic brain cancer. The monoclonal antibody 8H9 is a mouse IgG1 antibody with specificity for the B7H3 antigen [United Therapeutics Corporation]. This mouse antibody can be humanized. Still other immunoglobulin constructs targeting the B7-H3 and/or the B7-H4 antigen may be used in various embodiments of the present disclosure. Another ABP is S58 (anti-GD2, neuroblastoma). CotaraTM [Perregrince Pharmaceuticals] is a monoclonal antibody described for treatment of recurrent glioblastoma. Other ABPs may include, e.g., avastin, ficlatuzumab, medi-575, and olaratumab. Still other immunoglobulin constructs or monoclonal antibodies may be selected for use in various embodiments of the present disclosure. See, e.g., Medicines in Development Biologics, 2013 Report, pp. 1-87, a publication of PhRMA's Communications & Public Affairs Department. (202) 835-3460, which is incorporated by reference herein.

In some embodiments, the polynucleotide is operably linked to a regulatory sequence. In some embodiments, the regulatory sequence comprises a promoter sequence. In some embodiments, the regulatory sequence comprises a CMV or UbC promoter. In some embodiments, the regulatory sequence is selected from SEQ ID NO: 11 or 14.

In some embodiments, the polynucleotide further comprises a poly(A) signal.

### 6.3. Methods of Treatment

In one aspect, the present disclosure provides a method of transferring a polynucleotide to the central nervous system (CNS) of a subject, the method comprising: administering to the subject an effective dose of a recombinant adeno-associated virus (rAAV) described herein. The rAAV comprises a capsid comprising a capsid protein having the amino acid sequence of SEQ ID NO: 1 or a variant thereof, and the polynucleotide encapsulated by the capsid.

In some embodiments, the present disclosure provides a method of treating a disease of the central nervous system (CNS), the method comprising: administering to the CNS of a subject a therapeutically effective dose of: a rAAV, the rAAV comprising: a capsid protein having the amino acid sequence of SEQ ID NO: 1 or a variant thereof, and a polynucleotide encoding a therapeutic protein.

In some embodiments, the present disclosure provides a method of vaccination with a transgene, the method comprising: administering to the central nervous system (CNS) of a subject an effective dose of: a rAAV, the rAAV comprising: a capsid protein having the amino acid sequence of SEQ ID NO: 1 or a variant thereof, and a polynucleotide encoding an antigen.

A rAAV as described herein can be used in research and/or therapeutic applications. In some embodiments, a rAAV is for genetically modifying a cell in vitro or in vivo. In some embodiments, a rAAV is used for gene therapy or for vaccination in a human or animal. More specifically, a rAAV can be used for gene addition, gene augmentation, genetic delivery of a polypeptide therapeutic, genetic vaccination, gene silencing, genome editing, gene therapy, RNAi delivery, cDNA delivery, mRNA delivery, miRNA delivery, miRNA sponging, genetic immunization, optogenetic gene therapy, transgenesis, DNA vaccination, or DNA immunization of brain cells or non-brain cells.

### 6.3.1. Subject

The present disclosure provides a method of transferring a polynucleotide to the central nervous system (CNS) of a subject, e.g., a mammal. In some embodiments, the subject is a human. In some embodiments, the subject has a CNS disease. In some embodiments, the subject has a genetic defect associated with CNS disease or disorder.

In some embodiments, the CNS disease or disorder is selected from Adrenoleukodystrophy, Alexander Disease, Alzheimer disease, Amyotrophic lateral sclerosis, Angelman syndrome, Ataxia telangiectasia, Canavan disease, Charcot-Marie-Tooth syndrome, Cockayne syndrome, Chronic inflammatory demyelinating polyneuropathy (CIDP), Deafness, Duchenne muscular dystrophy, Epilepsy, Essential tremor, Fragile X syndrome, Friedreich's ataxia, Gaucher disease, GM1 gangliosidosis, GM2 gangliosidoses, Huntington disease, Frontotemporal Degeneration (FTD), Lesch-Nyhan syndrome, Maple syrup urine disease, Menkes syndrome, Metachromatic leukodystrophy (MLD), Myotonic dystrophy, Multiple sclerosis, Narcolepsy, Neurofibromatosis, Niemann-Pick disease, Parkinson's disease, Phenylketonuria, Prader-Willi syndrome, Refsum disease, Rett syndrome, Spinal muscular atrophy, Spinocerebellar ataxia, Tangier disease, Tay-Sachs disease, Tuberous sclerosis, Von Hippel-Lindau syndrome, Williams syndrome, Wilson's disease, and Zellweger syndrome.

In some embodiments, the CNS disease or disorder is a demyelinating or white matter disease. In some embodiments, the subject has a monogenic defect. In some embodiments, the subject has a genetic defect in a protein expressed in the CNS. In some embodiments, the subject has a monogenetic defect in a protein expressed in the CNS.

In some embodiments, the subject has a lysosomal storage disease (LDS). In some embodiments, the subject has a disease selected from: mucopolysaccharidosis type I e.g., Hurler syndrome and the variants Scheie syndrome and Hurler-Scheie syndrome; Hunter syndrome; mucopolysaccharidosis type III, e.g., Sanfilippo syndrome; mucopolysaccharidosis type IV, e.g., Morquio syndrome; mucopolysaccharidosis type VI, e.g., Maroteaux-Lamy syndrome; mucopolysaccharidosis type II; mucopolysaccharidosis type III; mucopolysaccharidosis type IV; mucopolysaccharidosis type VI; mucopolysaccharidosis type VII; mucopolysaccharidosis type VIII; mucopolysaccharidosis type IX; Tay-Sachs disease; Sandhoff disease; GM1 gangliosidosis; Fabry disease; Krabbe's disease; leukodystrophy; metachromatic leukodystrophy; Pompe disease; Fucosidosis deficiency; alpha-mannosidosis deficiency; beta-mannosidosis deficiency; Gaucher disease; Infantile Batten Disease; Classic Late Infantile Batten Disease; Juvenile Batten Disease; Batten, other forms Niemann-Pick disease; Niemann-Pick disease without sphingomyelinase deficiency; and Wolman disease.

In some embodiments, the subject has a mutation in an ARSA gene(s). In some embodiments, the subject has an ARSA protein deficiency. In some embodiments, the subject has MLD.

In some embodiments, the subject has a brain cancer. In some embodiments, the subject has brain metastases of a cancer. In some embodiments, the subject has brain metastases of breast cancer. In some embodiments, the subject has brain metastases of HER2 positive breast cancer.

### 6.3.2. Route of Administration

The present disclosure provides a method of administering an rAAV to transfer a polynucleotide to the CNS. In some embodiments, the rAAV is administered locally or systematically.

In certain embodiments, the rAAV is administered locally to the CNS. In some embodiments, rAAV is administered to the cerebral spinal fluid (CSF) of said subject. In some embodiments, the rAAV is administered to the cisternae magna, intraventricular space, brain ventricle, subarachnoid space, intrathecal space and/or ependyma of the subject.

In some embodiments, rAAV is administered by intrathecal administration, intracranial administration, intracerebroventricular (ICV), or intraparenchymal administration or administration to the lateral ventricles of the brain.

In some embodiments, rAAV is administered by lumbar injection (e.g., into the lumbar cistern) and/or injection into the intra cisterna magna (ICM).

In some embodiments, rAAV is administered to the ventricular system. In some embodiments, rAAV is administered to the rostral lateral ventricle; and/or administered to the caudal lateral ventricle; and/or administered to the right lateral ventricle; and/or administered to the left lateral ventricle; and/or administered to the right rostral lateral ventricle; and/or administered to the left rostral lateral ventricle; and/or administered to the right caudal lateral ventricle; and/or administered to the left caudal lateral ventricle.

In some embodiments, rAAV is administered such that the rAAV contacts ependymal cells of said subject. Such ependymal cells express the encoded polypeptide and optionally the polypeptide is expressed by the cells.

In some embodiments, the polypeptide is expressed and/or is distributed in the lateral ventricle, CSF, and/or brain (e.g., striatum, thalamus, medulla, cerebellum, occipital cortex, and/or prefrontal cortex).

In some embodiments, rAAV is administered intravenously or systemically.

To deliver the rAAV specifically to a particular region of the CNS, especially to a particular region of the brain, it may be administered by stereotaxic microinjection. For example, on the day of surgery, patients can have the stereotaxic frame base fixed in place (screwed into the skull). The brain with stereotaxic frame base (MRI-compatible with fiduciary markings) can be imaged using high resolution MRI. The MRI images can then be transferred to a computer that runs stereotaxic software. A series of coronal, sagittal and axial images can be used to determine the target site of vector injection, and trajectory. The software directly translates the trajectory into 3-dimensional coordinates appropriate for the stereotaxic frame. Burr holes can be drilled above the entry site and the stereotaxic apparatus localized with the needle implanted at the given depth. The vector in a pharmaceutically acceptable carrier can then be injected. The AAV vector can be then administrated by direct injection to the primary target site and retrogradely transported to distal target sites via axons. Additional routes of administration can be used, e.g., superficial cortical application under direct visualization, or other non-stereotaxic application.

In some embodiments, rAAV is delivered by a pump. The pump may be implantable. Another convenient way to administer the rAAV is to use a cannula or a catheter.

In some embodiments, rAAV is administered by Convection-enhanced delivery (CED) (Nguyen et al., (2003) J. Neurosurg. 98:584-590), which has been used clinically in gene therapy (AAV2-hAADC) for Parkinson's disease (Fiandaca et al., (2008) Exp. Neurol. 209:51-57). The underlying principle of CED involves pumping infusate into brain parenchyma under sufficient pressure to overcome the hydrostatic pressure of interstitial fluid, thereby forcing the infused particles into close contact with the dense perivasculature of the brain. Pulsation of these vessels acts as a pump, distributing the particles over large distances throughout the parenchyma (Hadaczek et al., (2006) Hum. Gene Ther. 17:291-302). To increase the safety and efficacy of CED, a reflux-resistant cannula (Krauze et al., (2009) Methods Enzymol. 465:349-362) can be employed along with monitored delivery with real-time MRI. Monitored delivery allows for the quantification and control of aberrant events, such as cannula reflux and leakage of infusate into ventricles (Eberling et al., (2008) Neurology 70:1980-1983; Fiandaca et al., (2009) Neuroimage 47 Suppl. 2:T27-35; Saito et al., (2011) Journal of Neurosurgery Pediatrics 7:522-526). US20190111157A1 provides improved procedures to achieve widespread expression of AAV vectors in the cortex and/or striatum.

In some embodiments, the rAAV is administered to the striatum. In some embodiments, the rAAV is administered to at least the putamen and the caudate nucleus of the striatum. In some embodiments, the rAAV is administered to at least the putamen and the caudate nucleus of each hemisphere of the striatum. In some embodiments, the rAAV is administered to at least one site in the caudate nucleus and two sites in the putamen.

In some embodiments, rAAV is delivered by intraparenchymal administration to a specific area of the brain. In some embodiments, rAAV is delivered by intraparenchymal administration to putamen, striatum, basal forebrain region, Substantia nigra and/or ventral tegmental area.

In some embodiments of the above aspects and embodiments, the rAAV is delivered by stereotactic delivery. In some embodiments, the rAAV is delivered by convection enhanced delivery (CED). In some embodiments, the rAAV is delivered using a CED delivery system. In some embodiments, the CED system comprises a cannula. In some embodiments, the cannula is a reflux-resistant cannula or a stepped cannula. In some embodiments, the CED system comprises a pump. In some embodiments, the pump is a manual pump. In some embodiments, the pump is an osmotic pump. In some embodiments, the pump is an infusion pump.

### 6.4. Pharmaceutical Compositions

In another aspect, the present invention provides a pharmaceutical composition comprising the rAAV described above [See Section 6.2], and a pharmaceutically acceptable excipient.

In some embodiments, the pharmaceutical composition is formulated for local administration to the CNS or for systemic administration. In some embodiments, the pharmaceutical composition comprises a CSF, e.g., ultrafiltrate of plasma or synthetic cerebrospinal fluid. An rAAV of the present disclosure can be administered to a subject (e.g., a human or non-human mammal) in a suitable carrier. Suitable carriers include saline, which may be formulated with a variety of buffering solutions (e.g., phosphate buffered saline), lactose, sucrose, calcium phosphate, gelatin, dextran, agar, pectin, and water. An rAAV typically is administered in sufficient amounts to transduce or infect the desired cells and to provide sufficient levels of gene transfer and expression to provide a therapeutic benefit without undue adverse effects.

In some embodiments, the pharmaceutical composition can be used to deliver the polynucleotide to a target within a mammalian subject. When the pharmaceutical composition is administered, the rAAV of the present disclosure can achieve a higher infection of target cells following administration to a mammalian subject as compared to an rAAV comprising a AAV9 capsid protein administered by the same route of administration and in the same dose. In some embodiments, the rAAV of the present disclosure achieves higher expression in target cells of the polynucleotide encapsulated by the rAAV following administration to a subject as compared to the polynucleotide encapsulated by a rAAV comprising an AAV9 capsid protein administered by the same route of administration and in the same dose.

Targeting of rAAVs can be tested in an experimental animal by measuring rAAV infection or expression of a polynucleotide. In some embodiments, targeting is measured in a non-human primate (NHP), mice, rats, birds, rabbits, guinea pigs, hamsters, farm animals (including pigs and sheep), dogs, or cats.

Targeting of rAAVs can be measured after systemic or local administration of rAAVs. In some embodiments, targeting of rAAVs is measured after intravenous infusion of rAAVs or local administration to CNS. In certain embodiments, targeting is measured after administration to the CNS by lumbar puncture (LP) via injection into the lumbar cistern (e.g., approximately L3-L4) or intra cisterna magna (ICM) administration.

In some embodiments, targeting of rAAVs is measured by measuring the ratio between the copy numbers of the transgene transcripts and a housekeeping gene (e.g., RPP30, actin, GAPDH or ubiquitin) transcripts. In a particular embodiment, the transcripts are measured by RT-ddPCR. In some embodiments, the ratio is measured after a first administration into a mammal such as a primate, e.g., monkey (such as cynomolgus or rhesus macaque) or a mouse.

In some embodiments, rAAV of the present disclosure provides the ratio of infection (i.e., expression) in a brain (or target region of the brain) or other tissue (or non-target region of the brain) of at least 10, at least 20, at least 30, at least 40, at least 50, at least 60, at least 70, at least 80, at least 90, at least 100, at least 150, at least 200, at least 500, at least 1000 fold, compared to AAV9.

In some embodiments, a brain: comparative tissue infection ratio is measured by comparing the ratios between the copy numbers of the transgene transcripts and house keeping gene (e.g., RPP30) transcripts in the same organs (e.g., brain) or in the same tissues (e.g., caudate nucleus, frontal cortex, Globus pallidum, motor cortex, parietal cortex, putamen, Substantia nigra) in two individual or two groups of animals, each administered with a test rAAVtest (e.g., Anc80L65) or AAV9.

$$rAAV test : AAV 9 infection ratio = \frac{( \frac{tran sgene transcripts}{h o u s e k e eping transctips} ) in rAAV test}{( \frac{t r a n sgene transcripts}{h o u s e k e eping transctips} ) in AAV 9}$$

In some embodiments, the rAAVₜₑₛₜ achieves infection ratio of at least 2, at least 3, at least 4, at least 5, at least 6, at least 7, at least 8, at least, at least 10, at least 20, at least 30, at least 40, or at least 50 compared to AAV9 in the brain. In some embodiments, the rAAVₜₑₛₜ achieves infection ratio of at least 2, at least 3, at least 4, at least 5, at least 6, at least 7, at least 8, at least, at least 10, at least 20, at least 30, at least 40, or at least 50 compared to AAV9 at one of the target tissues, caudate nucleus, frontal cortex, Globus pallidum, motor cortex, parietal cortex, putamen, and Substantia nigra.

6.4.1. Effective Dose

The dose of a rAAV administered to a subject will depend primarily on factors such as the condition being treated, and the age, weight, and health of the subject. For example, a therapeutically effective dosage of the rAAV to be administered to a human subject generally is in the range of from about 0.1 ml to about 10 ml of a solution containing concentrations of from about 1E12 to 1E17 genome copies (GCs) of rAAV per ml. For systemic administration, a therapeutically effective dosage of the rAAV to be administered to a human subject generally is in the range of from about 0.1 ml to about 10 ml or a larger volume of a solution containing rAAV.

In some embodiments, the effective dose is between 1E10 to 1E16 genome copy numbers (GC) of the rAAV per subject. In some embodiments, the effective dose for a human patient corresponds to a monkey dose of 1E12 to 1E15 GC of the rAAV. In some embodiments, the effective dose for a human patient corresponds to a monkey dose of 1E13 to 1E14 GC of the rAAV. In some embodiments, the effective dose for a human patient corresponds to a monkey dose of about 4E13 GC of the rAAV.

In some embodiments, the effective dose is 1E11 to 1E15 GC of the rAAV per a gram brain mass. In some embodiments, the effective dose is 1E11 to 1E13 GC of the rAAV per a gram brain mass. In some embodiments, the effective dose is 1E11 to 1E12 GC of the rAAV per a gram brain mass. In some embodiments, the effective dose is 1E12 to 1E14 GC of the rAAV per a gram brain mass. In some embodiments, the effective dose is about 5E11 GC of the rAAV per a gram brain mass. In some embodiments, the effective dose is about 2.5E11 GC of the rAAV per a gram brain mass. In some embodiments, the effective dose is about 5E10 GC of the rAAV per a gram brain mass. In some embodiments, the effective dose is about 2.5E10 GC of the rAAV per a gram brain mass.

In some embodiments, the effective dose is between 1E10-1E16 genome copy numbers (GC) of the rAAV per kg body weight. In some embodiments, the effective dose is between 1E11-1E15 genome copy numbers (GC) of the rAAV per kg body weight. In some embodiments, the effective dose is between 1E12-5E14 genome copy numbers (GC) of the rAAV per kg body weight. In some embodiments, the effective dose is between 0.5E13-2E14 genome copy numbers (GC) of the rAAV per kg body weight.

In some embodiments, when the rAAV contains a polynucleotide having a coding sequence of ARSA or a functional variant thereof, the effective dose is an amount sufficient to induce detectable expression of ARSA or the functional variant in the CNS. In some embodiments, the effective dose is an amount sufficient to induce detectable expression of ARSA or the functional variant in the Substantia nigra. In some embodiments, the effective dose is an amount sufficient to induce detectable expression of ARSA or the functional variant in the caudate nuclei. In some embodiments, the effective dose is an amount sufficient to induce detectable expression of ARSA or the functional variant in the ependyma. In some embodiments, the effective dose is an amount sufficient to induce detectable expression of ARSA or the functional variant in the cortex. In some embodiments, an effective dose of rAAV is an amount effective to induce detectable levels of ARSA or a functional variant thereof in a subject's brain and/or spinal cord. In some embodiments, an effective amount of rAAV is an amount effective to reduce the amount a sulfatide (e.g., C16 sulfatide) and/or lysosulfatide in a subject's brain and/or spinal cord.

Transduction and/or expression of a transgene can be monitored at various time points following administration by DNA, RNA, or protein assays. In some instances, the levels of expression of the transgene can be monitored to determine the frequency and/or amount of dosage. Dosage regimens similar to those described for therapeutic purposes also may be utilized for immunization.

In one aspect, the present invention provides a unit dose of rAAV provided herein. The unit dose comprises about 0.1 ml to about 10 ml of a solution containing concentrations of from about 1E9 to 1E17 genome copies (GCs) per ml of rAAV described herein. In some embodiments, the unit dose contains about 1E10 to 1E16 genome copies (GCs) per ml of rAAV described herein. In some embodiments, the unit dose contains about 1E11 to 1E15 genome copies (GCs) per ml of rAAV described herein. In some embodiments, the unit dose contains about 1E12 to 1E14 genome copies (GCs) per ml of rAAV described herein. In some embodiments, the unit dose contains about 2E13 genome copies (GCs) per ml of rAAV described herein.

In some embodiments, the unit dose contains about 1E10 to 1E16 genome copies (GCs) of rAAV described herein. In some embodiments, the unit dose contains about 1E11 to 1E15 genome copies (GCs) of rAAV described herein. In some embodiments, the unit dose contains about 1E12 to 1E15 genome copies (GCs) of rAAV described herein. In some embodiments, the unit dose contains about 1E13 to E15 genome copies (GCs) of rAAV described herein.

The unit dose further comprises a pharmaceutically acceptable excipient.

### 6.5. Summary of Experimental Observations

Applicant evaluated distribution of AAV9 and Anc80L65 vectors (SEQ ID No: 1) encoding the EGFP reporter 14 days following injection by either lumbar puncture (LP) injection into the lumbar cistern (approximately L3-L4) or intra cisterna magna (ICM) injection (4E¹³gc/animal; 2E¹³ vg/ml) in adult cynomolgus macaques. Applicant demonstrated that a single injection of Anc80L65 into the CSF of adult cynomolgus monkeys led to the efficient transduction of broad regions of the CNS.

Following ICM injection, Anc80L65 distributes more broadly throughout the cortex and into deep brain nuclei compared to AAV9. Following LP injection, Anc80L65 distribution throughout the cortex was on par with ICM delivery and superior to that seen with AAV9 via ICM delivery. AAV9 showed limited transduction in the cortex following LP delivery. AAV9 and Anc80L65 efficiently transduced spinal cord ventral horn motor neurons with both routes of administration.

Specifically, Anc80L65 transducing both neurons and astrocytes. Rare oligodendrocyte transduction was also observed in cortical regions with Anc80L65, however no microglial cells were found to be transduced using the microglial marker Ibal. AAV9 showed a similar tropism in the nonhuman primate CNS to Anc80L65, transducing largely neurons and astrocytes. Similar to Anc80L65 no microglial double labeling was observed. Oligodendrocyte transduction was not observed with AAV9, however there was less transduction overall in the CNS compared to Anc80L65 making it a difficult comparison.

Applicant further tested delivery and expression of a therapeutic gene (a coding sequence of anti-Her2 antibody, trastuzumab) in an AAV genomic construct encapsulated by Anc80L65 capsid in RAG knockout mice after ICV injection. The tested AAV constructs contained codon optimized coding sequences of the heavy chain and light chain of trastuzumab, in the order of the heavy chain and the light chain coding sequences, or the light chain and the heavy chain coding sequences, from 5′ to 3′ direction. In the experiment, constructs containing a heavy chain coding sequence followed by a light chain coding sequence from 5′ to 3′ provided the highest levels of trastuzumab mRNA and protein expression.

Expression of trastuzumab was further tested using AAV constructs containing different regulatory sequences, either a CMV promoter or a UbC promoter. In the experiment, constructs having a UbC promoter provided significantly better mRNA and protein expression in various brain regions compared to similar AAV constructs containing a CMV promoter.

Applicant has further demonstrated that Anc80L65 rAAV vectors can successfully deliver polynucleotides encoding ARSA and ARSA functional variants to the CNS of ARSA knock-out (KO) mice, resulting in ARSA and ARSA functional variant protein expression and reduction in sulfatide levels in the CNS after ICV injection.

This work demonstrated the ability of Anc80L65 to target widespread regions of the CNS following CSF routes of delivery and outperforms the distribution of AAV9 in targeting cortical and deep brain regions. The ability of Anc80L65 to mediate efficient gene transfer and expression in neurons and astrocytes throughout the brain and spinal cord of NHPs supports use of Anc80L65 vector for treatment of a wide range of neurologic disorders. In particular, Anc80L65 was demonstrated to be effective in delivering and expressing ARSA and ARSA functional variants in the CNS and in delivering and expressing trastuzumab. Additionally, constructs under the control of the UbC promoter were found to be particularly effective: AAV constructs containing ARSA and ARSA functional variants under the control of a UbC promoter were particularly effective in inducing CNS expression of ARSA and ARSA functional variants and reducing lysosulfatide and sulfatide levels, and an AAV construct containing the trastuzumab heavy chain coding sequence followed by trastuzumab light chain coding sequence under the control of a UbC promoter was particularly effective in inducing high level expression of trastuzumab in various brain regions.

### 7. EXAMPLES

### 7.1. Experimental Procedures: Examples 1-3

### 7.1.1. Lumbar Puncture (LP) Injection

The animal was injected with anesthesia and were placed in lateral recumbency. A 22-gauge Gerti Marx spinal needle was percutaneously inserted into the lumbar cistern (approximately L3-L4). Fluoroscopy was used for guidance if necessary. Once the needle was placed, the stylet was removed, and positive cerebral spinal fluid (CSF) flow confirmed, and predose CSF was collected. The test article syringe was then attached to the needle and the test article slowly infused by hand as a slow bolus over approximately 120±5 seconds. After completion of the injection, the needle was removed, and brief pressure was applied by hand over the injection site. Animal was then be placed in the Trendelenburg position (30°, head down) for a minimum of approximately 10 minutes. The animal was then allowed to recover naturally from anesthesia. Lumbar puncture is an intrathecal injection.

### 7.1.2. Intracisternal Magna (ICM) Injection

The animal was injected with anesthesia and placed in lateral recumbency. A 22-gauge spinal needle was advanced percutaneously into the cisterna magna, correct needle placement was verified by the presence of positive cerebral spinal fluid (CSF) flow, and predose CSF was collected. An appropriate Test Article syringe was then be connected to the spinal needle and the Test Article was administered by hand via a slow bolus injection (120±5 seconds). After completion of the injection, the syringe was removed, and pressure was applied briefly by hand. Animal was then placed in the Trendelenburg position (30°, head down) for a minimum of approximately 10 minutes. The animal was then be allowed to recover naturally from anesthesia.

### 7.1.3. Immunohistochemistry (IHC)

Two weeks after injection, tissue samples were collected and preserved in 10% neutral buffered formalin (NBF) for 48-72 hours, then transferred to 70% ethanol. The brain was placed into a pre-chilled brain matrix and sliced into 4 mm sections, then hemisected. Even-numbered hemisected slabs were preserved in 10% NBF and used for immunohistochemistry (IHC). Odd-numbered hemisected brain slabs were frozen on dry ice and stored at −60 to −90° C. until used for ddPCR analysis.

For detection of GFP expression, slides were incubated with antibodies against GFP (GeneTex, GTX20290) diluted 1:1,000 in Monet Blue Diluent (Biocare Medical, PD901). The slides were washed with Valent Wash Buffer (Biocare Medical, VLT8013MX) and incubated with anti-rabbit antibody conjugated with Farma HRP for 30 minutes (Biocare Medical, BRR4009). The slides were washed and then reacted with Betazoid DAB for 5 minutes (Biocare Medical, BDB2004) and counterstained with Mayer's Hematoxylin for 5 minutes (StatLab, HXMMHPT). After the reactions with Betazoid DAB or Mayer's Hematoxylin, the slides were washed with Aqua Rinse (Biocare Medical, VLT8012MX).

GFP staining by 3,3′-diaminobenzidine (DAB): Sections (3 per each 6-mm block: separation of 2 mm) were washed 3 times in PBST followed by treatment with 1% H2O2. Sections were stained with the primary anti-GFP antibody diluted 1:1000 in Da Vinci Green Diluent as previously described (Lluis Samaranch, Ernesto A. Salegio, Waldy San Sebastian, Adrian P. Kells, John R. Bringas, John Forsayeth, and Krystof S. Bankiewicz Human Gene Therapy. Volume: 24 Issue 5: Mar. 20, 2013, incorporated herein by reference).

For detection of Trastuzumab expression, slides were incubated with antibodies against IgG (Fc). IgG (Fc) can serve as a proxy for Trastuzumab expression.

### 7.1.4. Double-Immunofluorescence

Fluorescence immunostaining of different cellular markers (NeuN, GFAP, Iba1, Olig2+) with GFP as previously described (San Sebastian et al., 2013).

Sample Collection:

Tissue samples were collected and preserved in 10% neutral buffered formalin (NBF) for 48-72 hours, then transferred to 70% ethanol. The brain was placed into a pre-chilled brain matrix and sliced into 4 mm sections, then hemisected. Even-numbered hemisected slabs were preserved in 10% NBF and used for immunohistochemistry (IHC). Odd-numbered hemisected brain slabs were frozen on dry ice and stored at −60 to −90° C. until used for ddPCR analysis.

Immunohistochemistry Protocol for GFP Expression:
a Bake slides for 15 minutes at 55-65 Celsius to remove paraffin
Load slides onto Valent Staining Platform (Biocare Medical)
Val DePar 8 minutes (Biocare Medical, VLT8001MM)
Lo pH AR at 98 Celsius for 60 minutes (Biocare Medical, VLT8004rMM)
Peroxidazed 1 for 5 minutes (Biocare Medical, PX968)
Background Punisher for 5 minutes (Biocare Medical, BP974)
GFP (GeneTex, GTX20290) 1:1,000 in Monet Blue Diluent (Biocare Medical, PD901)
Rabbit on Farma HRP for 30 minutes (Biocare Medical, BRR4009)
Betazoid DAB for 5 minutes (Biocare Medical, BDB2004)
Counterstain with Mayer's Hematoxylin for 5 minutes (StatLab, HXMMHPT)

Valent Wash Buffer (Biocare Medical, VLT8013MX) was used after all steps expect for Betazoid DAB and Mayer's Hematoxylin. Aqua Rinse (Biocare Medical, VLT8012MX) was used after these reagents.

Dual Staining Methods for the IBA1, NeuN and GFAP with the GFP:

Reagents:
GFP (GeneTex, GTX20290) 1:1,000, GFAP (Cell Signaling, 3670) 1:500 in Monet Blue Diluent (Biocare Medical, PD901)
GFP (GeneTex, GTX20290) 1:1,000, IBA1 (Millipore, MABN92) 1:250 in Monet Blue Diluent (Biocare Medical, PD901)
GFP (GeneTex, GTX20290) 1:1,000, NeuN (Abcam, ab104224) 1:250 in Monet Blue Diluent (Biocare Medical, PD901)

Protocol:
Bake slides for 15 minutes at 55-65 Celsius to help remove paraffin
Load slides onto Valent Staining Platform (Biocare Medical)
Val DePar 8 minutes (Biocare Medical, VLT8001MM)
Lo pH AR at 98 Celsius for 60 minutes (Biocare Medical, VLT8004MM)
Peroxidazed 1 for 5 minutes (Biocare Medical, PX968)
Background Punisher for 10 minutes (Biocare Medical, BP974)
Primary Antibody Cocktail: Rabbit 594 nm (Invitrogen, A32740) 1:500, Mouse 488 nm (Invitrogen, A-21202) 1:500, cocktailed together in Da Vinci Green for 60 minutes (Biocare Medical, PD900)
Coverslip with Prolong Diamond Antifade Reagent with DAPI
Valent Wash Buffer (Biocare Medical, VLT8013MX) was used after all steps.

### 7.1.5. ddPCR

After euthanasia and exsanguination, brains were placed into a pre-chilled brain matrix and sliced into 4 mm sections, then hemisected. Odd numbered hemisected slabs were frozen over dry ice, then stored at −60° C. to −90° C. until analyzed. Brain regions were isolated using 2 mm or 3 mm diameter tissue punches (Miltex, Cat. No.: 95039-098 and 98PUN6-4) prior to nucleic acid isolation.

Tissues were homogenized in a Qiagen Tissuelyser II (20 rps for 2 min) in lysis buffer from the Qiagen Dneasy Blood and Tissue Kit or the Qiagen RNeasy Lipid Tissue Mini Kit following the standard Qiagen protocol. Samples were eluted in 50 uL of buffer. Prior to analysis, DNA and RNA concentration and quality were determined using a NanoDrop One, using the nucleic acid (DNA or RNA) program. DNA samples were analyzed for biodistribution of vector genomes using a duplexed ddPCR method targeting the transgene (eGFP) and a reference gene (RPP30). RNA samples were analyzed for expression of the eGFP transgene using a duplexed, one-step RT-ddPCR method) and a reference gene (RPP30).

Dual Staining Method for Olig2 and GFP (Performed at StageBio):

Reagents:
GFP (GeneTex, GTX20290) 1:1,000, Olig2 (Millipore, MABN50) 1:250 in Monet Blue Diluent (Biocare Medical, PD901)

Protocol:
Bake slides for 15 minutes at 55-65 Celsius to help remove paraffin
Load slides onto Valent Staining Platform (Biocare Medical)
Val DePar 8 minutes (Biocare Medical, VLT8001MM)
Lo pH AR at 98 Celsius for 60 minutes (Biocare Medical, VLT8004MM)
Peroxidazed for 5 minutes (Biocare Medical, PX968)
Background Punisher for 10 minutes (Biocare Medical, BP974)
Primary Antibody Cocktail: Biotinylated Mouse (Vector Laboratories, BA-9200) 1:500 in Da Vinci Green Diluent
Rabbit 594 nm (Invitrogen, A32740) 1:500, Streptavidin 488 nm (Invitrogen, S11223) 1:500, cocktailed together in Da Vinci Green for 60 minutes (Biocare Medical, PD900)
Coverslip with Prolong Diamond Antifade Reagent with DAPIValent Wash Buffer (Biocare Medical, VLT8013MX) was used after all steps.

DNA Analysis:

For isolation of DNA, tissues were homogenized in a Qiagen Tissuelyser II (20 rps for 2 min) in lysis buffer from the Qiagen DNeasy Blood and Tissue Kit (Part No. 69506), following the standard Qiagen protocol. Samples were eluted in 50 uL of AE buffer. Prior to analysis, DNA concentration and quality were determined using a NanoDrop One, using the nucleic acid (DNA) program.

DNA samples were analyzed for biodistribution of vector genomes using a duplexed ddPCR method targeting the transgene (eGFP or Trastuzumab) and a reference gene (RPP30). Specific primer probe sequences are listed in the table below.

| Name | Target | Sequence |
|---|---|---|
| pCAG.eGFP_DNA | eGFP | GCTTCTGGCGTGTGACC (SEQ ID NO: |
| FWD Set 4 | | 52) |
| pCAG.eGFP_DNA | eGFP | TGATGAGACAGCACAATAACCAG |
| REV Set 4 | | (SEQ ID NO: 53) |
| pCAG.eGFP_DNA | eGFP | FAM/TTTCCTACA/ZEN/GCTCCTGGGCA |
| PRB Set 4 | | ACG/3IABkFQ (SEQ ID NO: 54) |
| RPP30_NHP_DNA | RPP30 | GAACCTGAAACTTCACA (SEQ ID NO: |
| FWD Set 3 | | 55) |
| RPP30_NHP_DNA | RPP30 | CCATTTAAGGAGTGGTTAT (SEQ ID |
| REV Set 3 | | NO: 56) |
| RPP30_NHP_DNA | RPP30 | HEX/TAAAGTCTA/ZEN/CGCACTACCAC |
| PRB Set 3 | | TTAC/3IABkFQ (SEQ ID NO: 57) |

The samples were analyzed following the standard Bio-Rad ddPCR protocol for probe-based analysis of DNA biodistribution. Briefly, reaction mixes containing the 2 primer probe sets, DNA samples and Bio-Rad ddPCR Supermix for Probes (no dUTP) (Part No. 186-3024) were prepared according to the recipe in the table below.

| Reagent | Vol/reaction |
|---|---|
| 2X ddPCR Supermix | 10 |
| 20X RPP30 PnP | 1 |
| 20X eGFP PnP or 20X | 1 |
| Trastuzumab PnP | |
| Water | 3 |
| Sample* | 3 |

*DNA samples were pre-diluted to 2 ng/μL (liver), 10 ng/μL (DRG, no dilution for the samples with the concentration <10 ng/μL) and 20 ng/μL (other samples) using nuclease-free water.

After droplet generation, reactions were amplified using the thermal cycling program indicated below.

| Parameter | Time | Temp ° C. | Cycles |
|---|---|---|---|
| Enzyme Activation | 10 min | 95° C. | 1 |
| Denaturation | 30 sec | 94° C. | 40 |
| Annealing | 30 sec | 54° C. | |
| Extension | 60 sec | 74° C. | |
| Enzyme deactivation | 10 min | 98° C. | 1 |
| Hold | ∞ (Press Cancel Run | 4° C. | 1 |
| | to end program) | | |

| | |
|---|---|
| Ramp Rate | 2° C./sec |
| Volume | 40 μL |
| Lid Temperature | 105° C. |

Data is reported in vector genomes copied per diploid genome (VGC/DG). The formula for calculating the output is VGC/DG=(eGFP cp/μL=RPP30 cp/μL)×2 for eGFP or VGC/DG=(Trastuzumab cp/μL=RPP30 cp/μL)×2 for Trastuzumab.

RNA Analysis:

For isolation of mRNA, tissues were homogenized in a Qiagen Tissuelyser II (20 rps for 1 min) in 1 ml of Qiazol from the Qiagen RNeasy Lipid Tissue Mini Kit (Part No. 74804), following the standard Qiagen protocol. Samples were eluted in 50 pL of Nuclease-free water. Prior to analysis, RNA concentration and quality were determined using a NanoDrop One, using the nucleic acid (RNA) program.

DNA samples were analyzed for expression of the eGFP transgene or the Trastuzumab transgene using a duplexed, one-step RT-ddPCR method targeting the transgene (eGFP or Trastuzumab) and a reference gene (RPP30). Specific primer probe sequences are listed in the table below.

| Name | Target | Sequence |
|---|---|---|
| pCAG.eGFP RNA | eGFP | CACAGCTCCTGGGCAAC (SEQ ID NO: |
| FWD Set 5 | | 58) |
| pCAG.eGFP RNA | eGFP | AGCTCGACCAGGATGGG (SEQ ID NO: |
| REV Set 5 | | 59) |
| pCAG.eGFP RNA | eGFP | FAM/ATGGTGAGC/ZEN/AAGGGCGAGG |
| PRB Set 5 | | A/3IABkFQ (SEQ ID NO: 60) |
| RPP30_NHP_RNA | RPP30 | GCGGGTTCTGACCTGAAG (SEQ ID NO: |
| FWD Set 3 | | 61) |
| RPP30_NHP_RNA | RPP30 | TCCCTGTACAATCGGTAAAGTTG (SEQ |
| REV Set 3 | | ID NO: 62) |
| RPP30_NHP_RNA | RPP30 | HEX/CGGCTCACC/ZEN/TTGGCTATTCAG |
| PRB Set 3 | | TTGT/3IABkFQ (SEQ ID NO: 63) |

The samples were analyzed following the standard Bio-Rad RT-ddPCR protocol for probe-based analysis of RNA expression. Briefly, reaction mixes containing the 2 primer probe sets, RNA samples and Bio-Rad One-Step RT-ddPCR Advanced Kit for Probes (Part No. 186-4021) were prepared according to the recipe in the table below.

| | Vol |
|---|---|
| Reagent | (uL)/reaction |
| Supermix | 5 |
| 300 mM DTT | 1 |
| Reverse Transcriptase | 2 |
| 20X RPP30 PnP | 1 |
| 20X eGFP PnP or 20x Trastuzumab | 1 |
| Nuclease-free water | 5 |
| RNA Sample* | 5 |

*RNA samples were pre-diluted to 20 ng/μL using nuclease-free water.

After droplet generation, reactions were amplified using the thermal cycling program indicated below.

| Parameter | Time | Temp ° C. | Cycles |
|---|---|---|---|
| Reverse Transcription | 60 min | 48° C. | 1 |
| Enzyme Activation | 10 min | 95° C. | 1 |
| Denaturation | 30 sec | 94° C. | |
| Annealing | 30 sec | 57 | 40 |
| Extension | 60 sec | 74° C. | |
| Enzyme deactivation | 10 min | 98° C. | 1 |
| Hold | ∞ (Press Cancel Run | 4° C. | 1 |
| | to end program) | | |

| | |
|---|---|
| Ramp Rate | 2° C./sec |
| Volume | 40 μL |
| Lid Temperature | 105° C. |

Data is reported as % eGFP expression, which is calculated according to the formula, % eGFP expression=(eGFP cp/μL=RPP30 cp/μL)×100 or % Trastuzumab expression=(Trastuzumab cp/μL=RPP30 cp/μL)×100.

### 7.1.6. Her2-Binding ELISA

For Her2-Binding ELISA, Eagle Biosciences Humanized Anti-Her-2 (Herceptin/Trastuzumab) ELISA Assay Kit (Cat. No. AHR31-K01) was used according to the manufacturer's instructions with variations as described herein. This anti-Her2 ELISA is a method used to quantify the binding of functional Trastuzumab using a sandwich method where a microwell titer plate is coated with recombinant HER2 protein.

Briefly, microwells from a microwell titer plate were coated with recombinant HER2 protein. Assay calibrators, controls, and test samples (brain tissue lysates) were added into the designated microwells. Immediately, 100 μL of 1× assay buffer was added and plate was sealed and incubated for 1 hour on a small orbit radius shaker at 400 to 450 rpm. Each microwell was washed with working wash solution (i.e., mild buffer), and a secondary antibody specific to Human IgG antibody was added to each well. The secondary antibody was conjugated to a Horseradish Peroxidase enzyme, which provided the mechanism for colorimetric quantification of Trastuzumab. A second wash step was performed to remove unbound secondary antibody. A substrate solution, which reacts with the Horseradish Peroxidase enzyme to create a colored product, was added to each well and incubated for 30 minutes. The color density at the end of the incubation period was proportional to the amount of Trastuzumab bound to the plate in the first step. A standard curve of known concentrations was used to calibrate the measurement of Trastuzumab in test samples. The reaction was stopped by the addition of a high pH buffer. The amount of colored product generated in each well was measured by a plate reader, which passed light through the liquid in the well and measured the absorbance of the colored liquid. The absorbance of the standard curve was plotted and the absorbance of the test samples was compared to the standard curve plot to determine the amount of Trastuzumab in the test sample. Data is presented as absorbance normalized to total protein loaded.

### 7.2. Example 1: Broad CNS Penetration and Wide Distribution of Anc80L65 Compared to AAV9

The objective of this study is to determine the biodistribution and initial feasibility of Anc80L65 vector compared to AAV9 vector, when administered by a single lumbar puncture or intra-cisterna magna administration. The results confirm broad penetration and wide distribution of Anc80L65 compared to AAV9.

Two AAV constructs were used in the experiment: (i) Anc80L65-CAG-GFP, and (ii) AAV9-CAG-GFP, each including an AAV genome construct containing a coding sequence of GFP. GFP was used to detect distribution of AAVs and expression of the transgene. Cynomolgus monkeys were used as the subject animals.

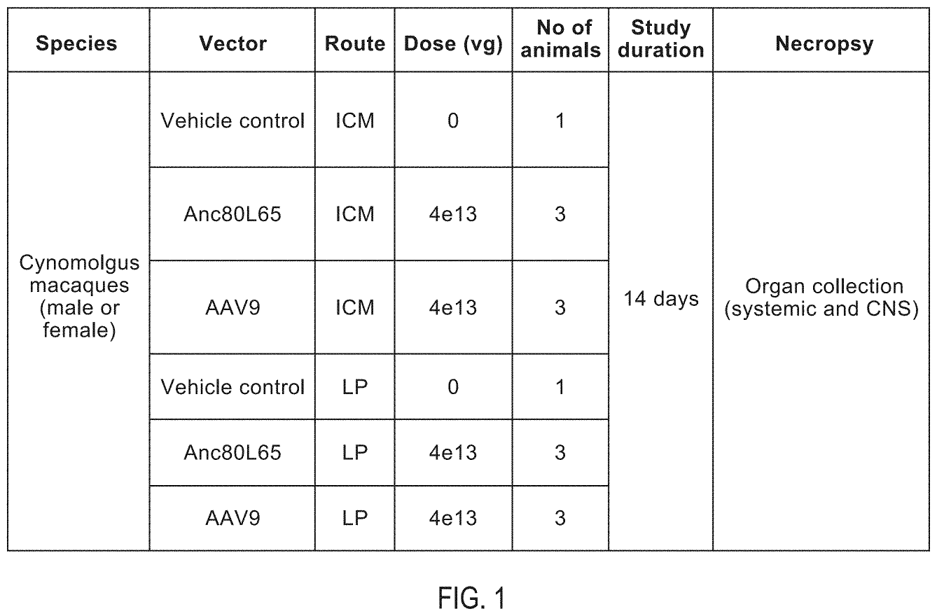

Total 14 animals were divided into 6 groups as summarized in the FIG. 1 and TABLE 2. Animals in Group 1 and 4 are control animals administered with vehicle. Animals in Group 2 and 5 were administered with 4E13vg (viral genome or GC) of Anc80L65, and animals in Group 3 and 6 were administered with 4E13vg of AAV9. Two routes of administration were tested—animals in Group 1-3 were administered by ICM, and animals in Group 4-6 were administered by LP. Animals were sacrificed on day 14 or 15 after the vehicle or AAV administration and their organ samples were collected for analysis.

TABLE 2
NHP Experiment Design

| | | | Dose | | Dose | |
|---|---|---|---|---|---|---|
| | Test | Dose | Level | Dose | Concen- | # of |
| GroupNo. | Material | Route | vg/animal | Volume(mL) | tration(vg/mL) | animals |
| 1 | Vehicle | ICM | 0 | 2 | 0 | 1 |
| 2 | Anc80L65 | ICM | 4E13 | 2 | 2E13 | 3 |
| 3 | AAV9 | ICM | 4E13 | 2 | 2E13 | 3 |
| 4 | Vehicle | LP | 0 | 2 | 0 | 1 |
| 5 | Anc80L65 | LP | 4E13 | 2 | 2E13 | 3 |
| 6 | AAV9 | LP | 4E13 | 2 | 2E13 | 3 |

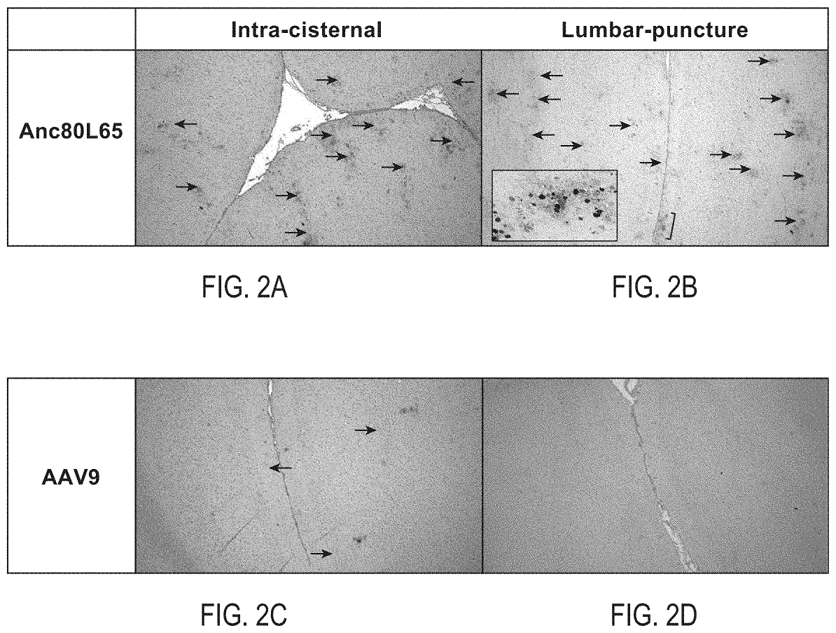

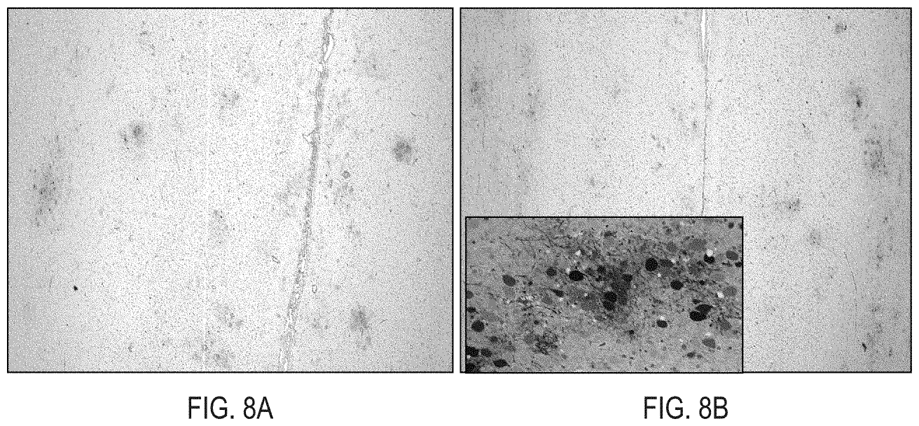

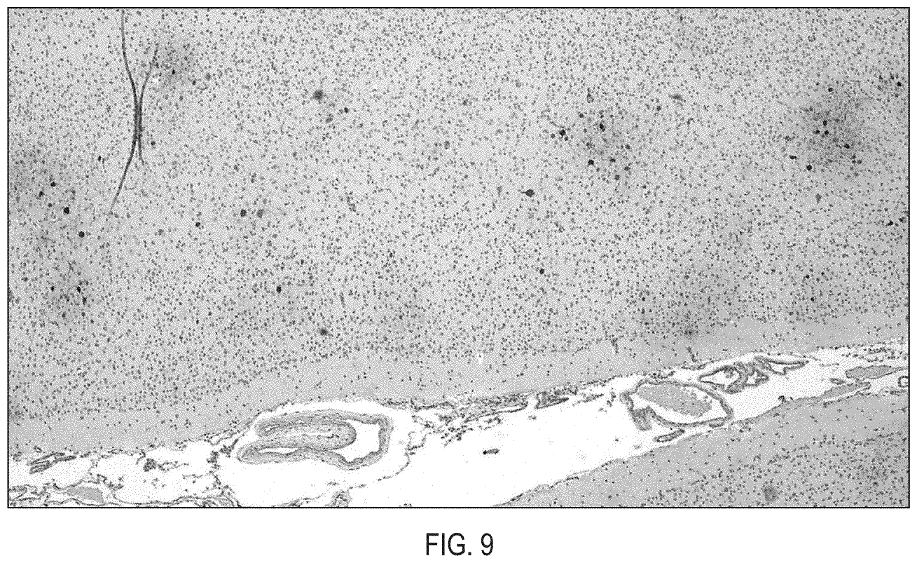

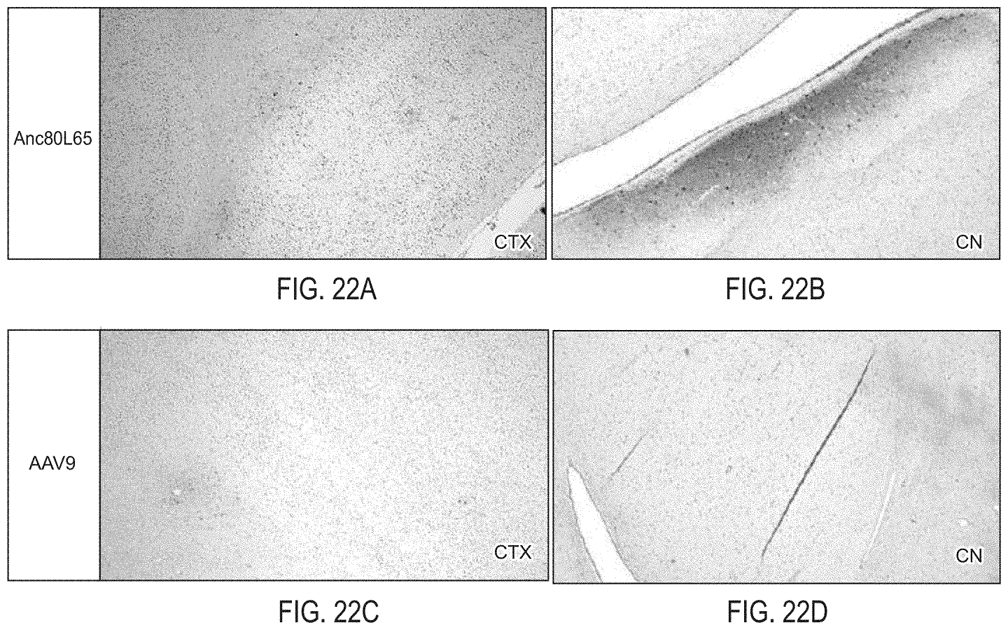

Collected samples were processed for IHC and stained with an antibody against GFP. Images of the IHC staining are provided in FIGS. 2A-9 and 22A-22D. FIGS. 2A-2D provide immunohistochemistry (IHC) images of cortical tissue from the brain sections obtained from NHPs administered with Anc80L65 or AAV9 by intracisternal magna injection or lumbar-puncture. FIGS. 22A-22D provide IHC images of brain sections of cortex and caudate nucleus obtained from NHPs administered with Anc80L65 or AAV9 by intracisternal magna injection.

These results show transgene (GFP) expression capabilities of Anc80L65 are superior compared to AAV9 both by ICM and LP administrations. More cells were stained for GFP expression in the cortex and caudate nucleus after administration of Anc80L65 compared to AAV9. FIGS. 2A-2D further show that ICM administration provides better results than LP administration with both vectors (i.e., Anc80L65 and AAV9) in terms of breadth of distribution within the brain.

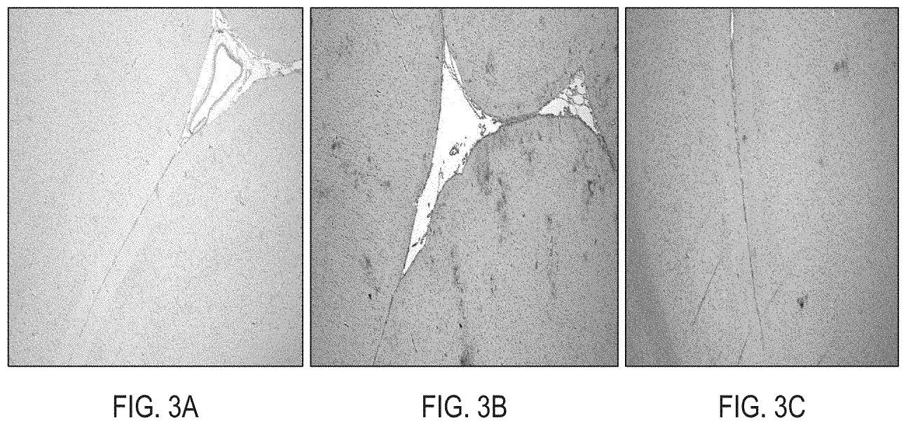

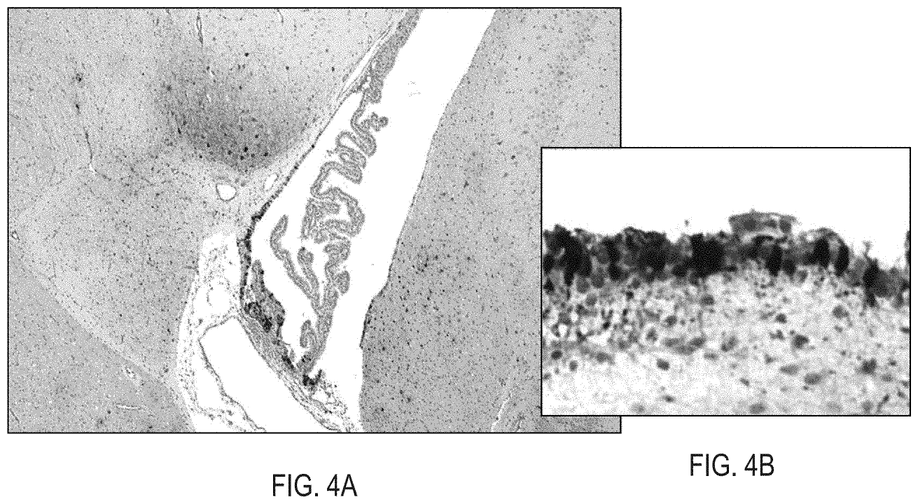

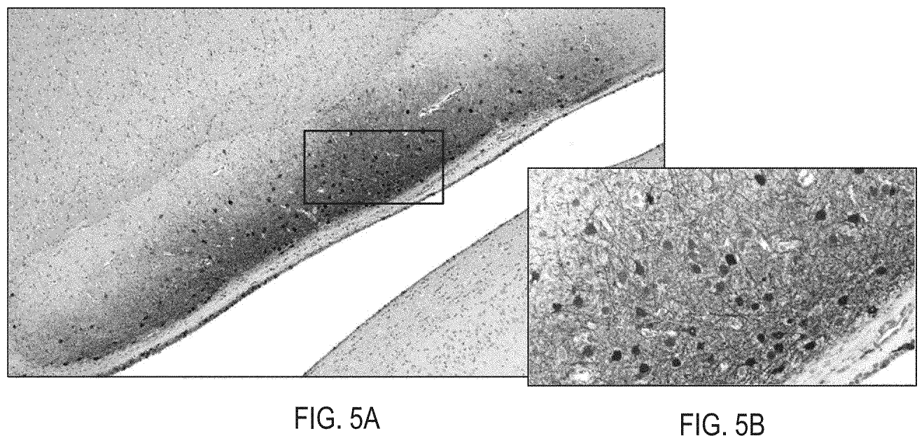

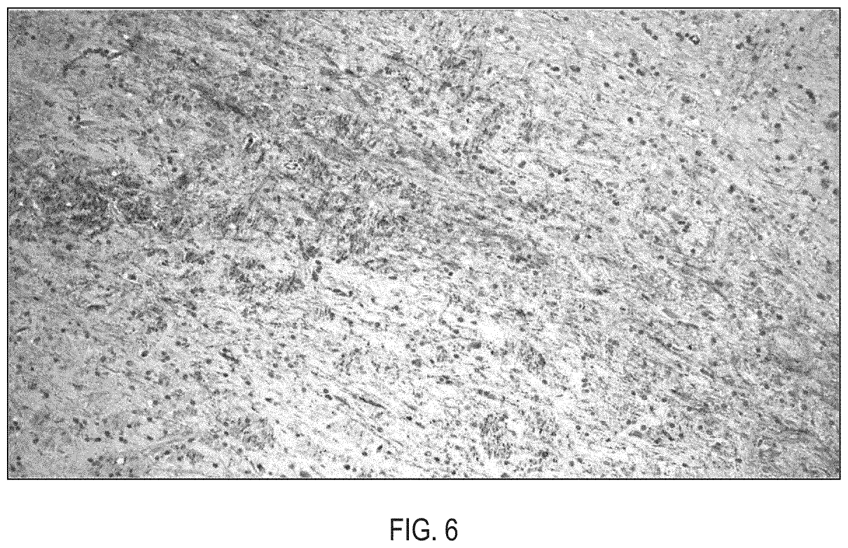

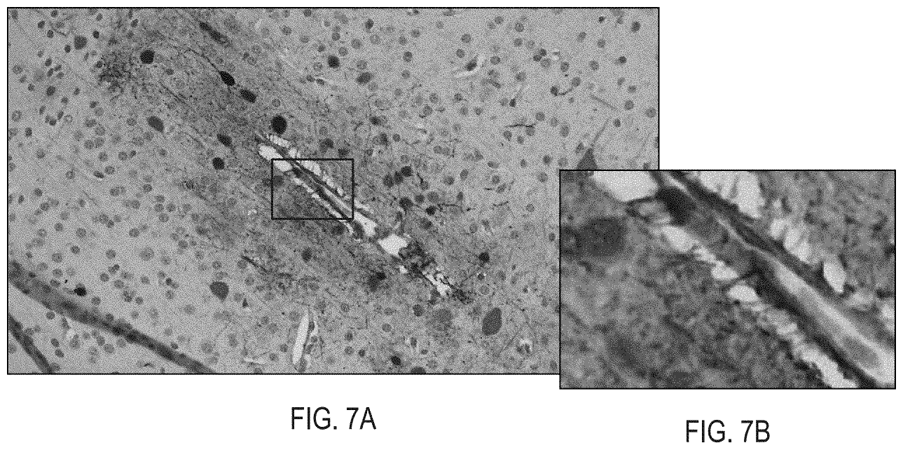

IHC results in other parts of the brain are also provided—specifically, in the cortex (FIGS. 3A-3C, 8A-8B and 9), ependyma and caudate nucleus (FIGS. 4A-4B), caudate nucleus (FIGS. 5A-5B), Substantia nigra (FIG. 6), and perivascular cells (FIG. 7A-7B). The results show broad penetration and wide distribution of Anc80L65 compared to AAV9.

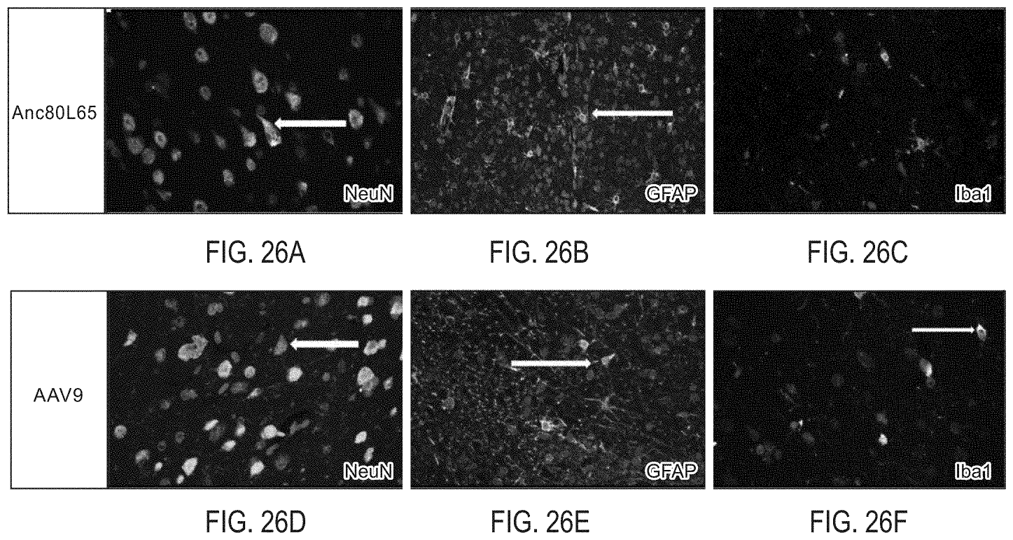

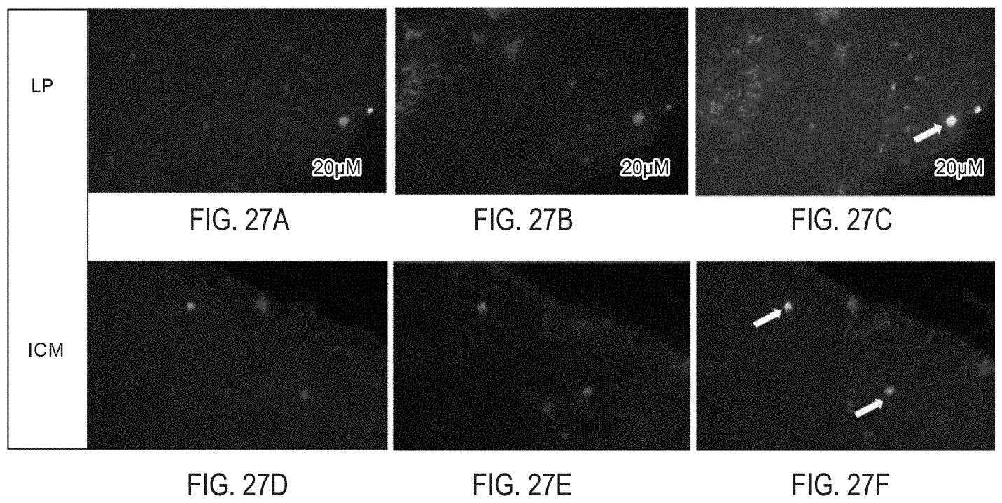

To characterize cell types expressing GFP after Anc80L65 or AAV9 administration, the NHP brain sections were double stained for GFP and a cell-type specific marker. FIGS. 26A-26F and FIGS. 27A-27F provide the images of the double staining—against GFP and a marker for neurons (NeuN) (FIGS. 26A and 26D), against GFP and a marker for astrocytes (FIGS. 26B and 26E), against GFP and a marker for microglial cells (ibal), against GFP and a marker for oligodendrocyte (FIGS. 27A, 27B and 27C) in the motor cortex transfected with Anc80L65 or AAV9. In all cases, GFP+ cells are shown in red, the cell specific marker is shown in green, and the merged images are shown with double-labeled cells in yellow/orange (arrows). The staining results show that Anc80L65 can mediate efficient transgene expression in neurons, astrocytes and oligodendrocytes across large regions of the NHP brain following a single LP or ICM injection. This suggests that Anc80L65 can be used for clinical applications to treat a wide range of neurologic disorders, particularly using a relatively noninvasive route of administration such as LP.

Transgene transfer and expression capabilities of Anc80L65 and AAV9 administered by ICM or LP to NHPs were also tested with ddPCR, by measuring amounts of DNA and mRNA of the transgene (eGFP) in the NHP brain and spinal cord 2 weeks after ICM or LP delivery. DNA genome copies and mRNA transcript copies of the transgene (eGFP) were quantified in comparison to the amounts of DNA genome copies or mRNA transcript copies of a house keeping gene (RPP30), respectively. Specifically, DNA genome copies are reported as vector genomes copies per diploid genome (VGC/DG). The formula for calculating the output is VGC/DG=(eGFP cp/μL=RPP30 cp/μL)×2. RNA transcript copies are reported as % eGFP expression, which is calculated according to the formula, % eGFP expression=(eGFP cp/μL RPP30 cp/μL)×100.

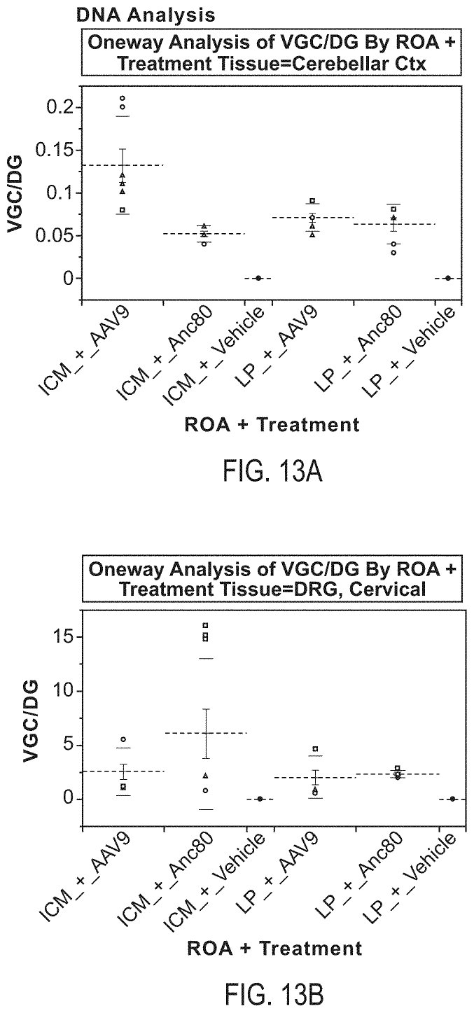

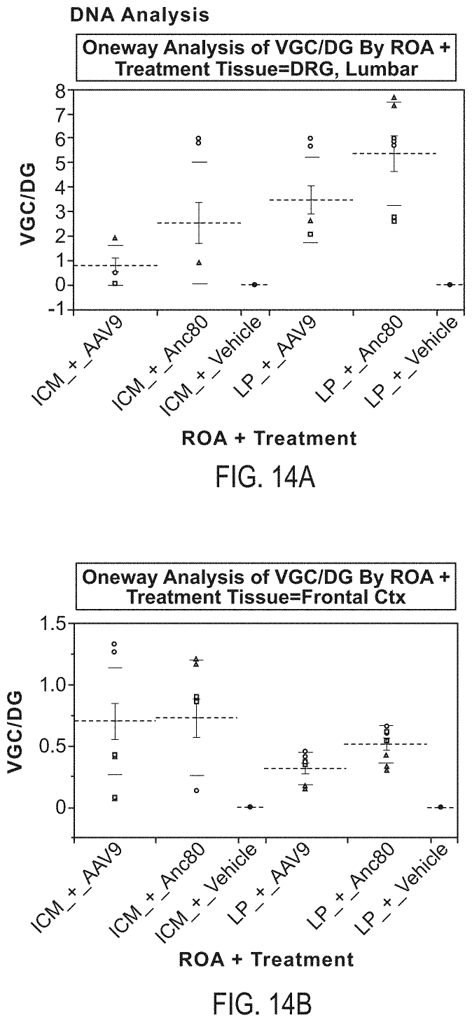

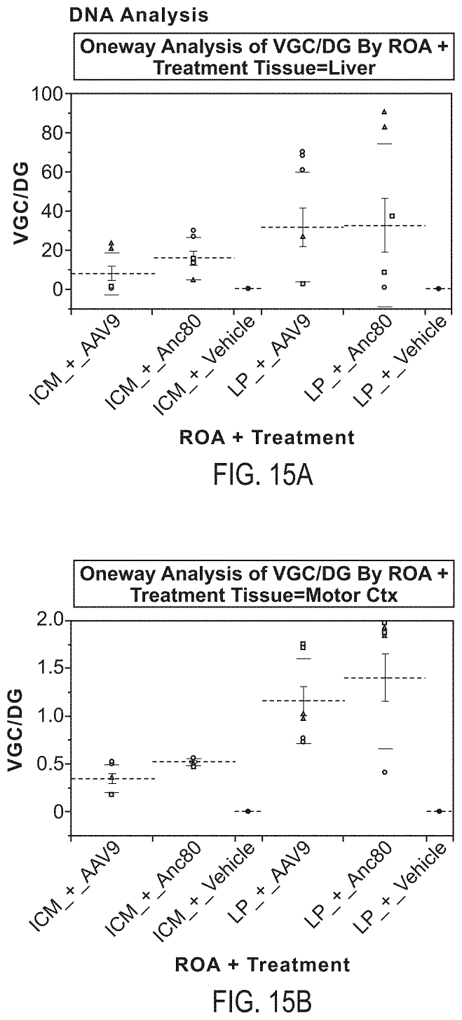

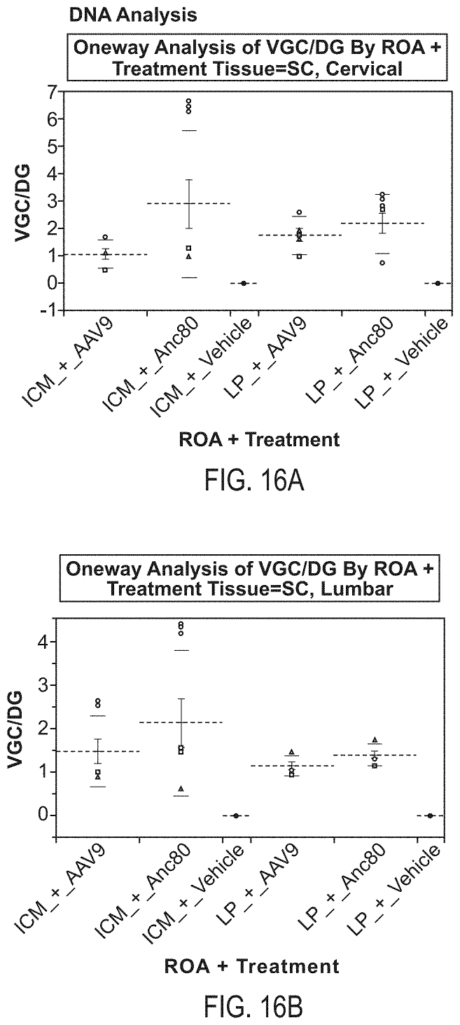

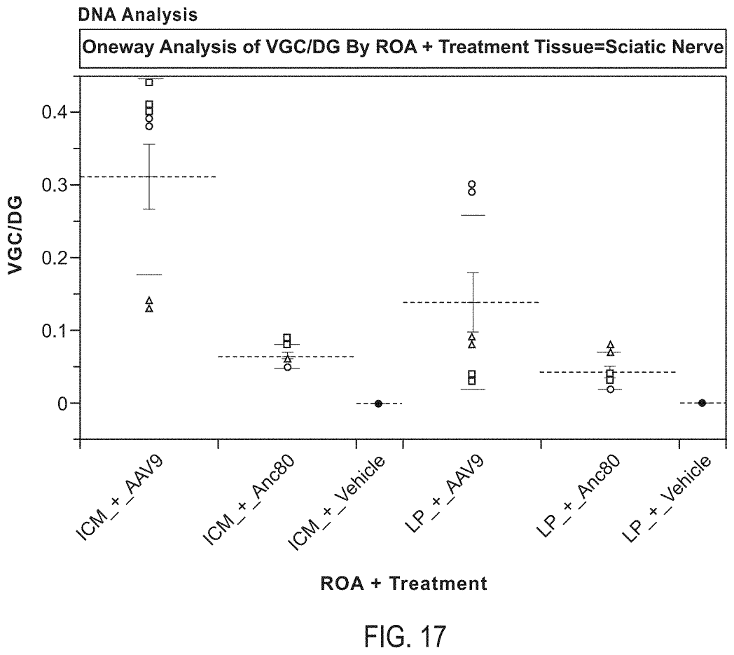

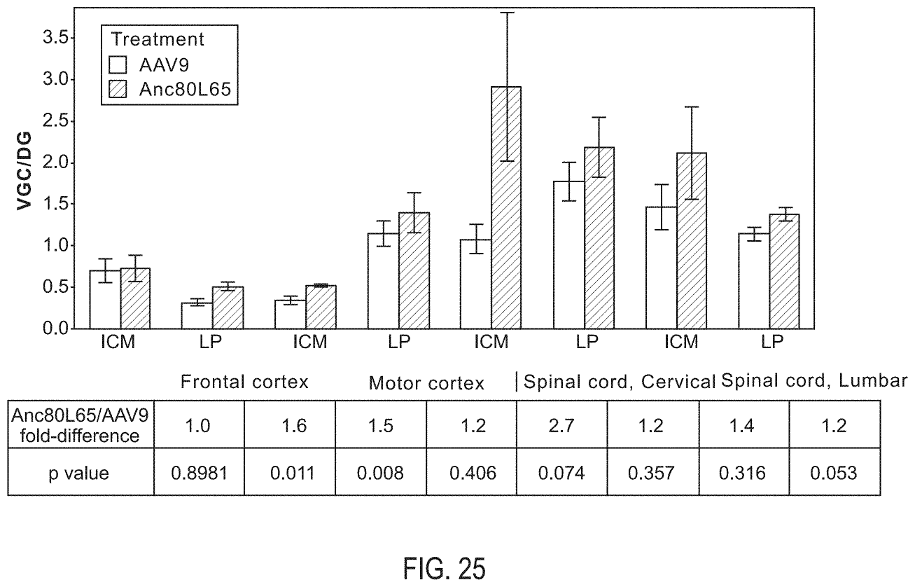

Viral DNA genome copies (VGCs) per diploid genome (i.e., VGCs per cell) measured in the experiment are provided in FIGS. 13A-17. Each figure provides data corresponding to different brain regions or liver, including cerebellar cortex (FIG. 13A), dorsal root ganglia, cervical (FIG. 13B), dorsal root ganglia, lumbar (FIG. 14A), frontal cortex (FIG. 14B), liver (FIG. 15A), motor cortex (FIG. 15B), spinal cord, cervical (FIG. 16A), spinal cord, lumbar (FIG. 16B), and sciatic nerve (FIG. 17). The VGCs data are further analyzed and summarized in FIG. 25.

The data show Anc80L65 led to more vector genome copies per cell in frontal cortex, motor cortex and spinal cord (cervical and lumbar) compared with AAV9, irrespective of injection route as shown in FIG. 25.

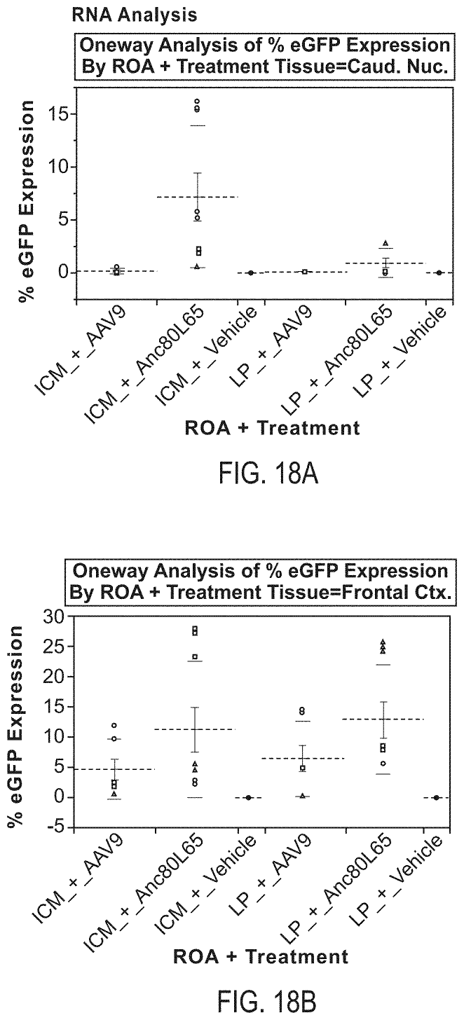

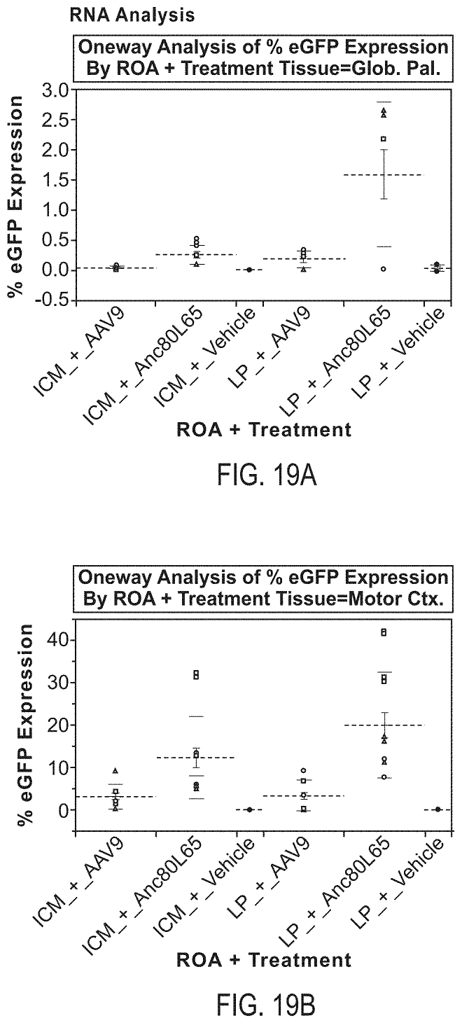

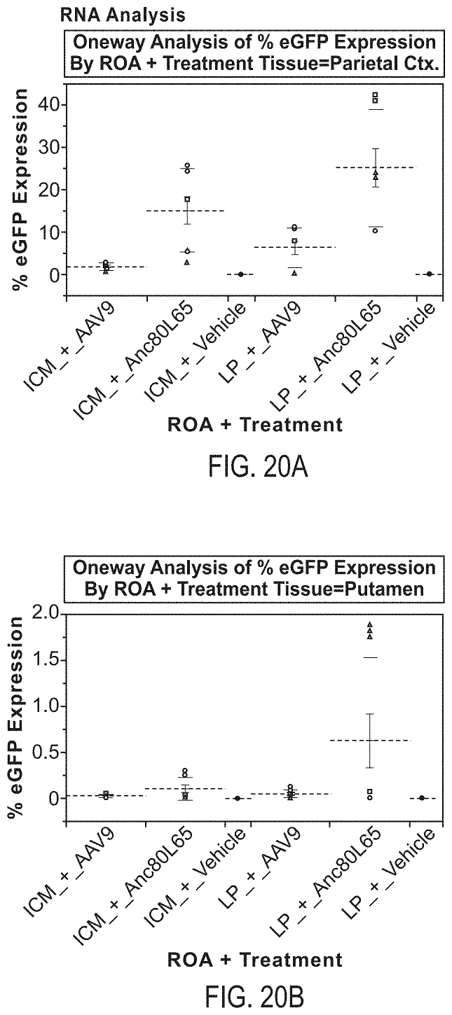

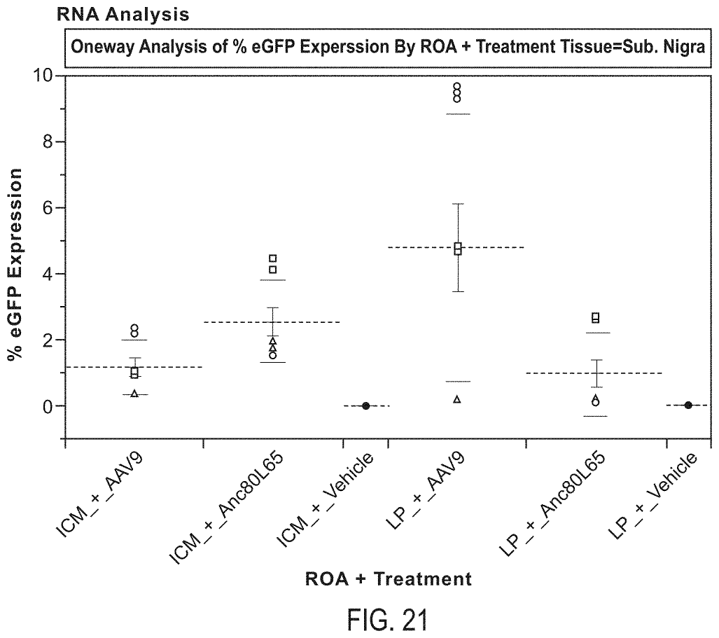

RNA transcripts measured from the experiment are provide in FIGS. 18A, 18B, 19A, 19B, 20A, 20B and 21. Each figure provides data corresponding to different brain regions, including caudate nucleus (FIG. 18A), frontal cortex (FIG. 18B), Globus pallidus (FIG. 19A), motor cortex (FIG. 19B), parietal cortex (FIG. 20A), putamen (FIG. 20B), and Substantia nigra (FIG. 21). Administration of Anc80L65 induced higher levels of GFP expression in several brain regions, including caudate nucleus after ICM administration, Globus pallidus after LP administration, motor cortex after both ICM and LP administration, parietal cortex after both ICM and LP administration, and putamen after LP administration.

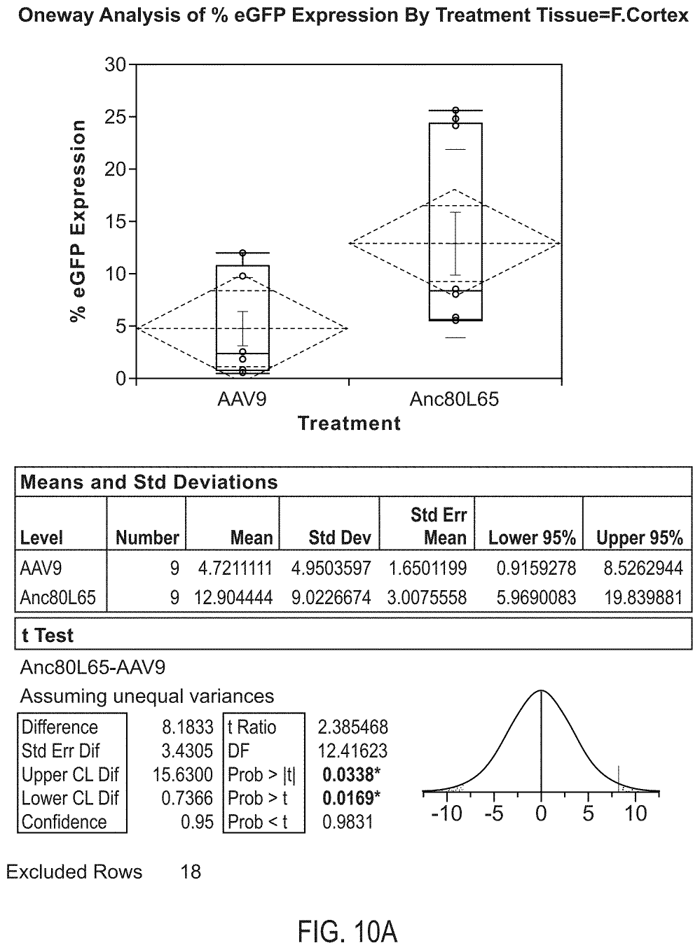

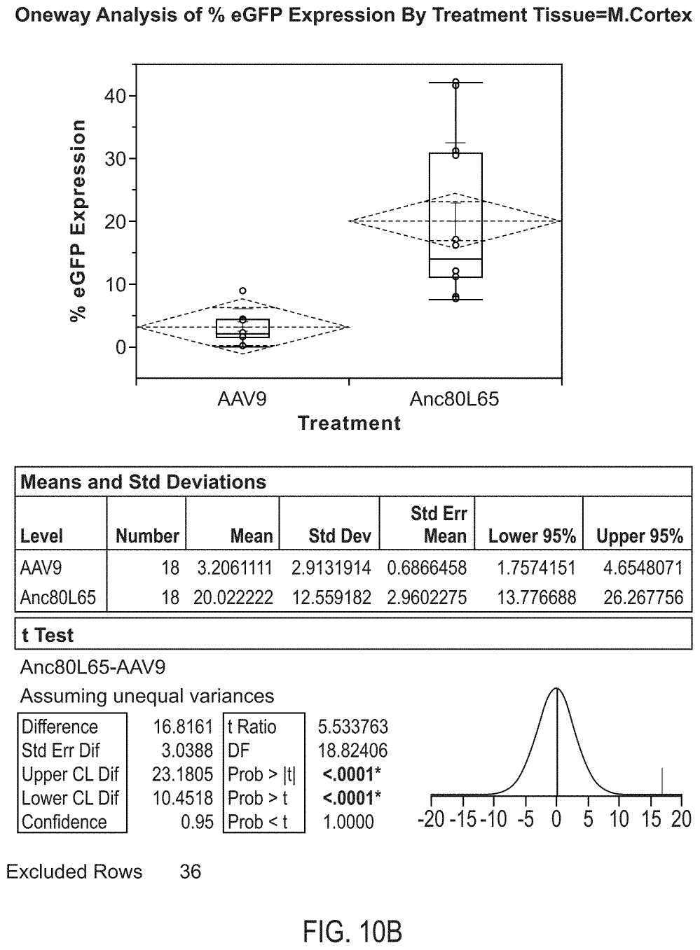

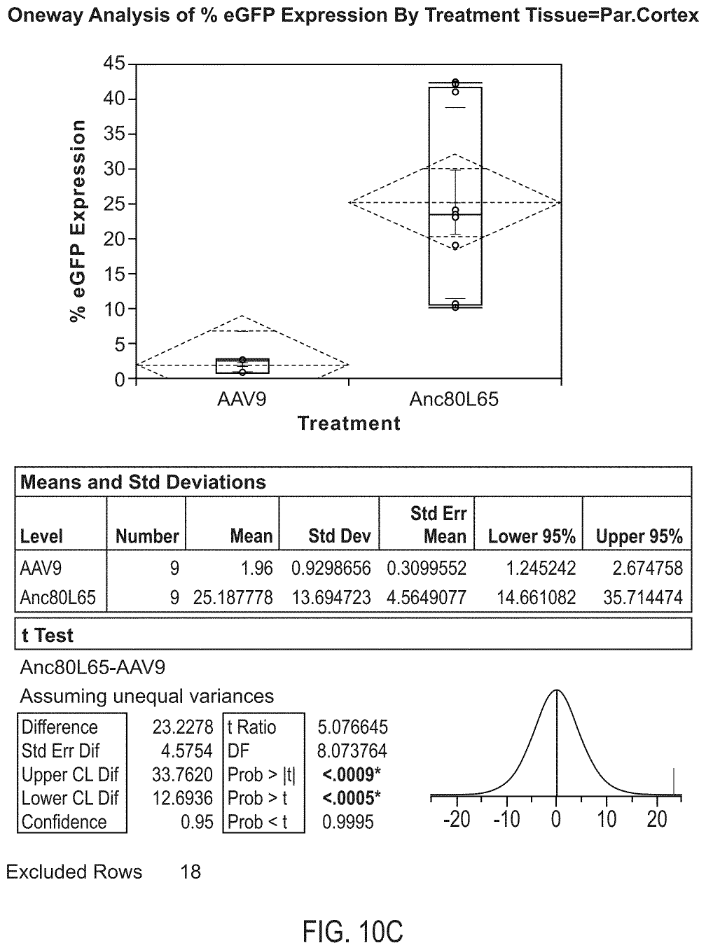

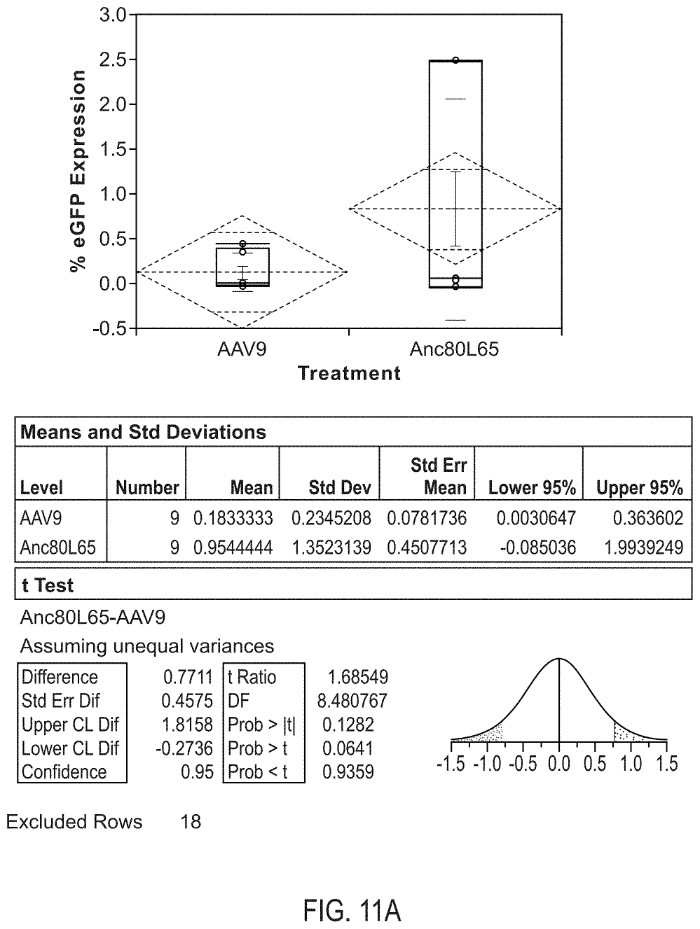

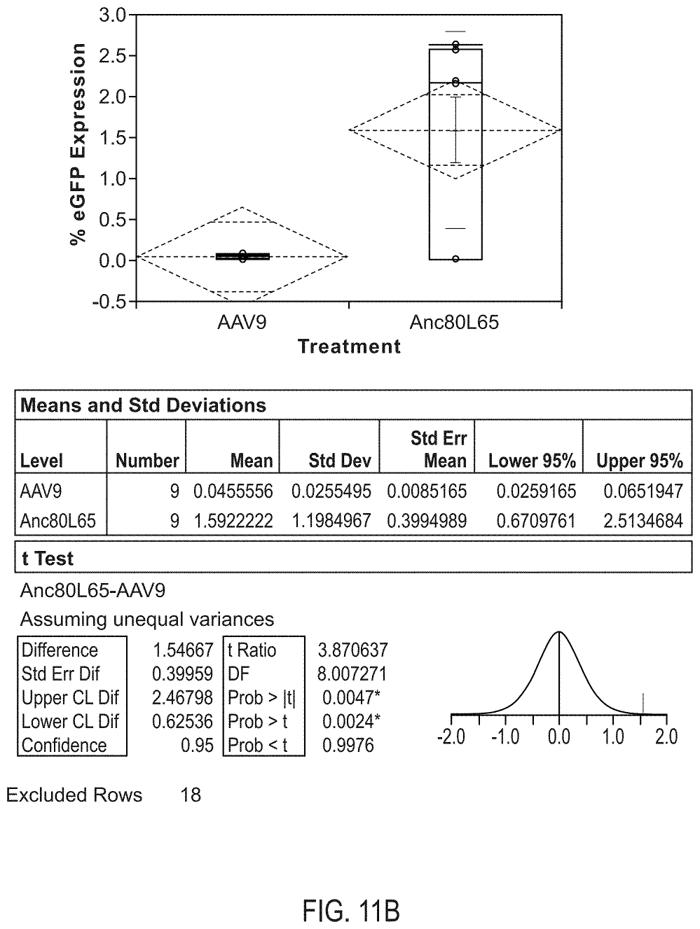

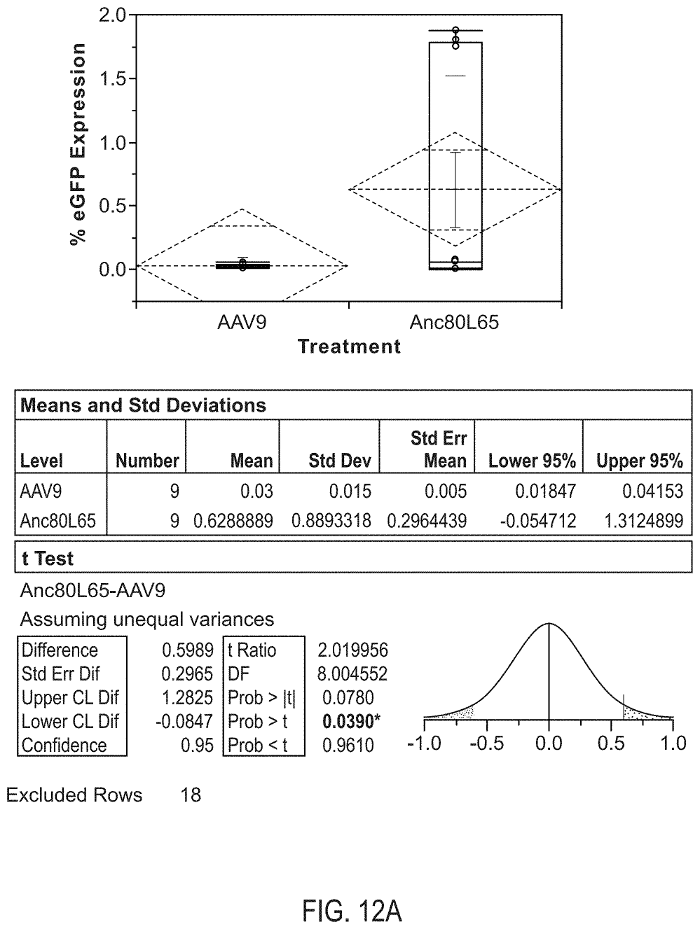

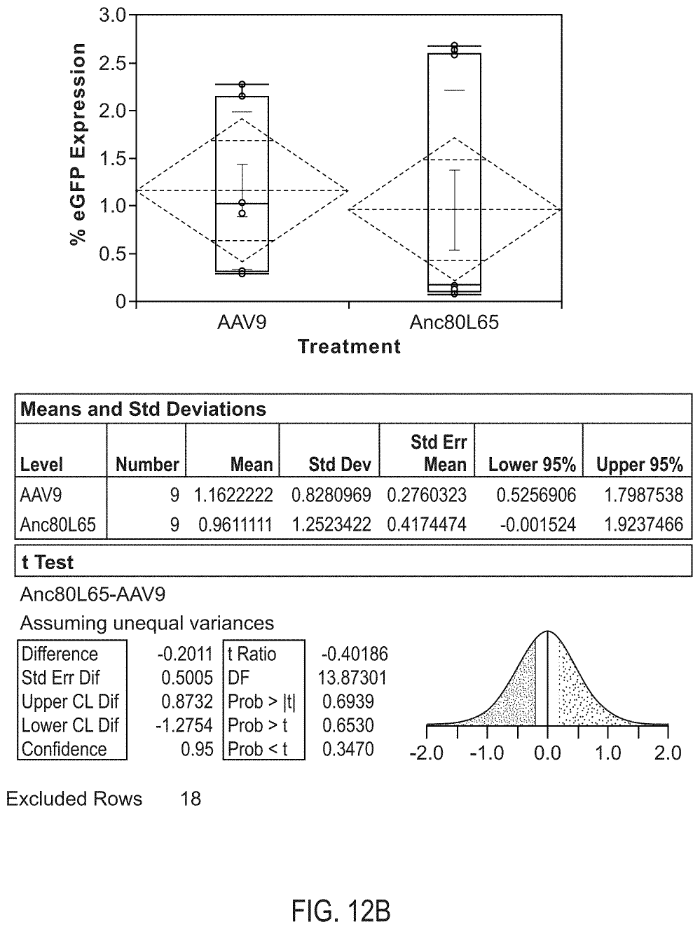

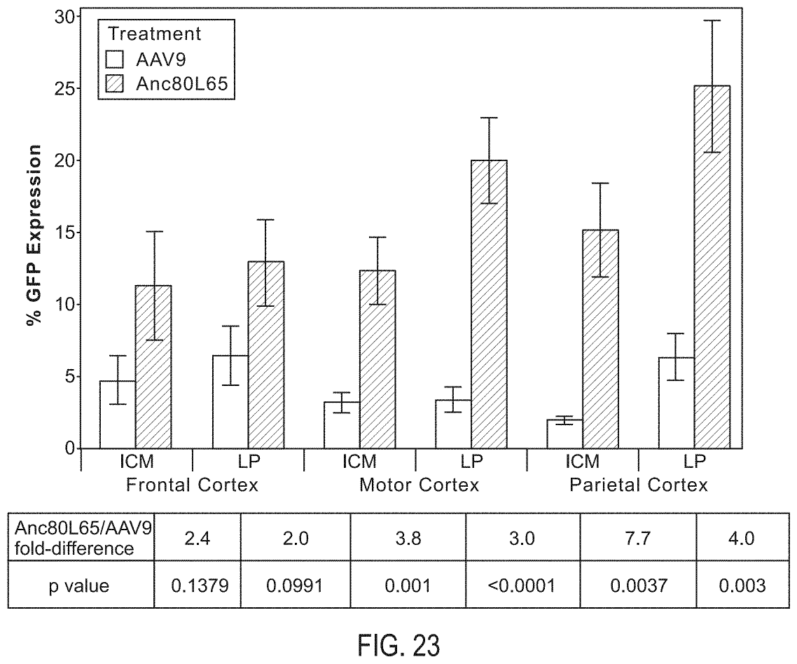

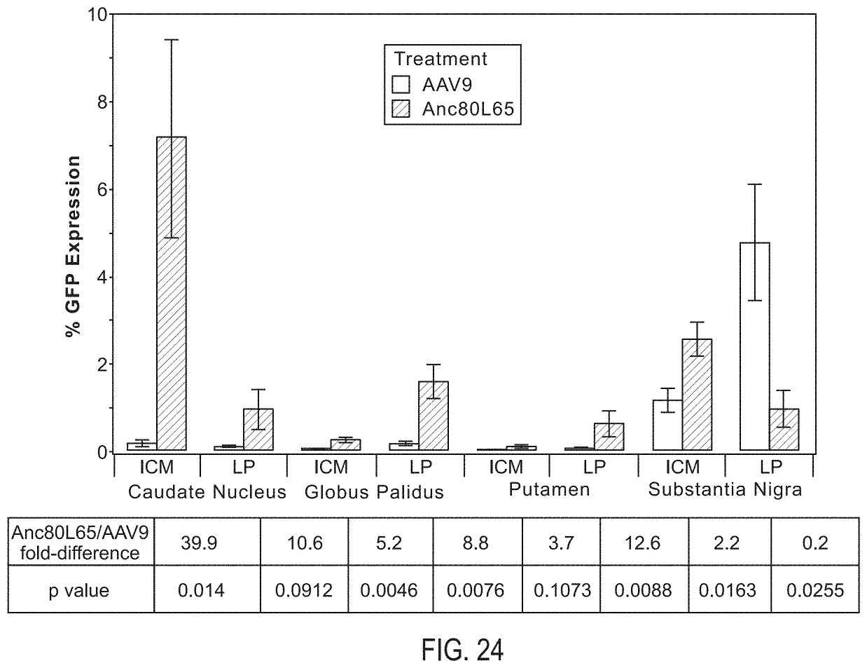

One-way statistical analysis of the expression data is provided in FIGS. 10A-FIG. 12B. The analysis results are also tabulated in FIG. 23 and FIG. 24. FIGS. 10A-10C and 23 provide analysis of the data from the frontal cortex (FIG. 10A, FIG. 23), motor cortex (FIG. 10B, FIG. 23); and parietal lobe of the cortex (FIG. 10C, FIG. 23). The data show significantly higher expression of GFP in the cortex of the animals injected with Anc80L65 by ICM or LP compared to AAV9 by ICM or LP. FIGS. 11A-11B, FIGS. 12A-12B and FIG. 24 show similar analysis in caudate nucleus (FIG. 11A, FIG. 24), Globus pallidus (FIG. 11B, FIG. 24), putamen (FIG. 12A, FIG. 24) and Substantia nigra (FIG. 12B, FIG. 24). These figures also show significantly higher GFP expression in the most brain areas of animals injected with Anc80L65 by ICM or LP compared to AAV9 by ICM or LP. These results suggest that both ICM and LP injections of Anc80L65 can be effective ways of delivering and expressing a transgene, superior to ICM administration of AAV9.

The statistical analysis of the ddPCR data is also provided below in TABLE 3. The table provides fold differences and p-value results from the Tukey-Kramer HSD test showing comparisons of GFP transcript (RNA) expression in various tissues between Anc80L65 (ICM) vs. AAV9 (ICM), Anc80L65 (LP) vs. AAV9 (ICM), and Anc80L65 (LP) vs. AAV9 (LP). Positive differences indicate the magnitude of expression advantage attributed to Anc80L65. Statistically significant p-Values are indicated in red (asterisk). The analysis shows that superiority of Anc80L65 is statistically significant compared to AAV9 in various brain regions.

TABLE 3

| | | | Anc80L65 Expression | |
|---|---|---|---|---|
| Tissue | Treatment 1 | Treatment 2 | Advantage | p-Value |
| Caud. Nuc. | ICM_+_Anc80L65 | ICM_+_AAV9 | +7.0 | 0.00* |
| Caud. Nuc. | LP_+_Anc80L65 | ICM_+_AAV9 | +0.8 | 0.96 |
| Caud. Nuc. | LP_+_Anc80L65 | LP_+_AAV9 | +0.9 | 0.95 |
| Frontal Ctx. | ICM_+_Anc80L65 | ICM_+_AAV9 | +6.6 | 0.35 |
| Frontal Ctx. | LP_+_Anc80L65 | ICM_+_AAV9 | +8.2 | 0.17 |
| Frontal Ctx. | LP_+_Anc80L65 | LP_+_AAV9 | +6.4 | 0.36 |
| Glob. Pal. | ICM_+_Anc80L65 | ICM_+_AAV9 | +0.2 | 0.88 |
| Glob. Pal. | LP_+_Anc80L65 | ICM_+_AAV9 | +1.5 | <.0001* |
| Glob. Pal. | LP_+_Anc80L65 | LP_+_AAV9 | +1.4 | 0.0001* |
| Motor Ctx. | ICM_+_Anc80L65 | ICM_+_AAV9 | +9.1 | 0.01* |
| Motor Ctx. | LP_+_Anc80L65 | ICM_+_AAV9 | +16.8 | <.0001* |
| Motor Ctx. | LP_+_Anc80L65 | LP_+_AAV9 | +16.6 | <.0001* |
| Parietal Ctx. | ICM_+_Anc80L65 | ICM_+_AAV9 | +13.2 | 0.02* |
| Parietal Ctx. | LP_+_Anc80L65 | ICM_+_AAV9 | +23.2 | <.0001* |
| Parietal Ctx. | LP_+_Anc80L65 | LP_+_AAV9 | +18.8 | 0.0004* |
| Putamen | ICM_+_Anc80L65 | ICM_+_AAV9 | +0.1 | 0.98 |
| Putamen | LP_+_Anc80L65 | ICM_+_AAV9 | +0.6 | 0.04* |
| Putamen | LP_+_Anc80L65 | LP_+_AAV9 | +0.6 | 0.05* |
| Sub. Nigra | ICM_+_Anc80L65 | ICM_+_AAV9 | +1.4 | 0.56 |
| Sub. Nigra | LP_+_Anc80L65 | ICM_+_AAV9 | −0.2 | 1.00 |
| Sub. Nigra | LP_+_Anc80L65 | LP_+_AAV9 | −3.8 | 0.01* |

### 7.3. Example 2: Selection of Candidate Nucleic Acid Sequences Encoding an Antigen Binding Protein Against HER2

This experiment was designed to select candidate nucleic acid sequences encoding an antigen binding protein specific to human epidermal growth factor receptor 2 (HER2) (e.g., an anti-Her2 antigen-binding protein (e.g., Trastuzumab)) for use in the methods described herein. In particular, the experiment was designed to evaluate Heavy Chain (HC) and Light Chain (LC) orientation within the coding sequence and optimized coding sequences for Trastuzumab. The coding sequence for each candidate was encapsulated by an AAV comprising an Anc80L65 capsid and administered to RAG knockout mice.

### 7.3.1. Experimental Design

RAG Knockout (RAG KO) (JAX Strain 002216) mice to be treated were divided into five treatment groups (1-5). Group 1 received an ICV saline injection and served as a vehicle control. Group 2 received Anc80L65.CMV.ATX.HCLC by ICV injection. Group 4 received Anc80L65.CMV.W2.HCLC by ICV injection. Group 5 received Anc80L65.CMV.ATX.LCHC by ICV injection. Group 6 was administered the same dose of AAV9.CMV.W1.HCLC by ICV injection as a control.

Anc80L65.CMV.ATX.HCLC comprises a construct comprising from 5′ to 3′, CMV promoter and a codon-optimized coding sequence (ATX) of a heavy chain (SEQ ID NO: 29) followed by a light chain (SEQ ID NO: 30), and Anc80L65 capsid encapsulating the construct. Anc80L65.CMV.W2.HCLC comprises a construct comprising from 5′ to 3′, CMV promoter and a codon-optimized coding sequence (W2) of a heavy chain (SEQ ID NO: 33) followed by a light chain (SEQ ID NO: 34), and Anc80L65 capsid encapsulating the construct. Anc80L65.CMV.ATX.LCHC comprises a construct comprising from 5′ to 3′, CMV promoter and a codon-optimized coding sequence (ATX) of a light chain (SEQ ID NO: 30) followed by a heavy chain (SEQ ID NO: 29), and Anc80L65 capsid encapsulating the construct. Anc80L65.CMV.W1.HCLC comprises a construct comprising from 5′ to 3′, CMV promoter and a codon-optimized coding sequence (W1) of a heavy chain (SEQ ID NO: 31) followed by a light chain (SEQ ID NO: 32), and Anc80L65 capsid encapsulating the construct.

Coding sequences for W1 heavy chain and W2 heavy chain have 88.5% sequence identity and encode for proteins having 98.9% sequence identity. W2 heavy chain includes a complementarity determining region 3 (CDR3) comprising a coding sequence of TGGGGCGGCGACGGCTTATACGCCATGGACTAC (SEQ ID NO: 48), encoding the amino acid sequence of WGGDGLYAMDY (SEQ ID NO: 49). W1 heavy chain includes a CDR3 comprising a coding sequence of TGGGGAGGCGACGGCTTCTACGCCATGGACTAT (SEQ ID NO: 50), encoding the amino acid sequence of WGGDGFYAMDY (SEQ ID NO: 51). Light chain coding sequences for W1 and W2 have 88.9% sequence identity, and each encode the amino acid sequence of SEQ ID NO: 36.

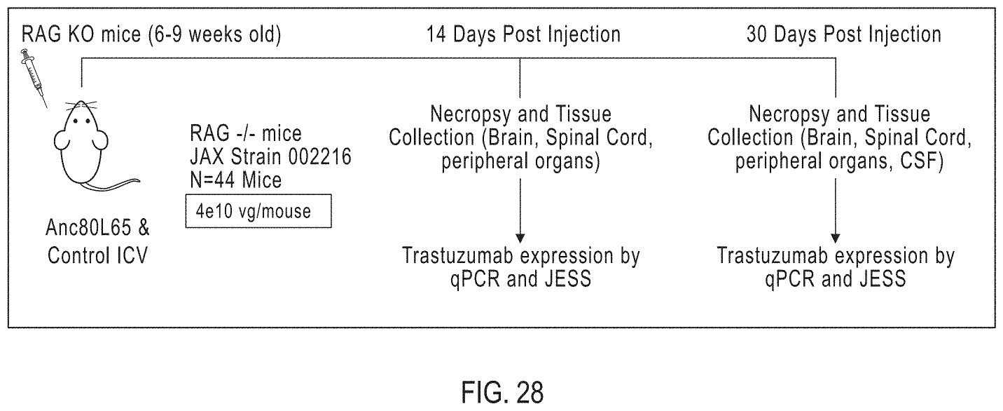

Table 4 provides a summary of the experimental design including experimental conditions for Groups 1-5 (see also FIG. 28).

TABLE 4
Experimental Design

| | | | | Dose (vg/g | N- |
|---|---|---|---|---|---|
| Group | Vector | Construct | ROA | Brain weight) | value |
| 1 | Vehicle | PBS control | ICV | NA | 2 + 2 |
| 2 | ATV-0038 | Anc80L65.CMV.ATX.HCLC | ICV | 3.85E+10 | 4 + 4 |
| | (SEQ ID NO: 27) | | | | |
| 4 | ATV-0040 | Anc80L65.CMV.W2.HCLC | ICV | 3.85E+10 | 4 + 4 |
| | (SEQ ID NO: 39) | | | | |
| 5 | ATV-0041 | Anc80L65.CMV.ATX.LCHC | ICV | 3.85E+10 | 4 + 4 |
| | (SEQ ID NO: 28) | | | | |
| 6 | AAV9-W1 | AAV9.CMV.W1.HCLC | ICV | 3.85E+10 | 4 + 4 |
| | (SEQ ID NO: 26) | | | | |

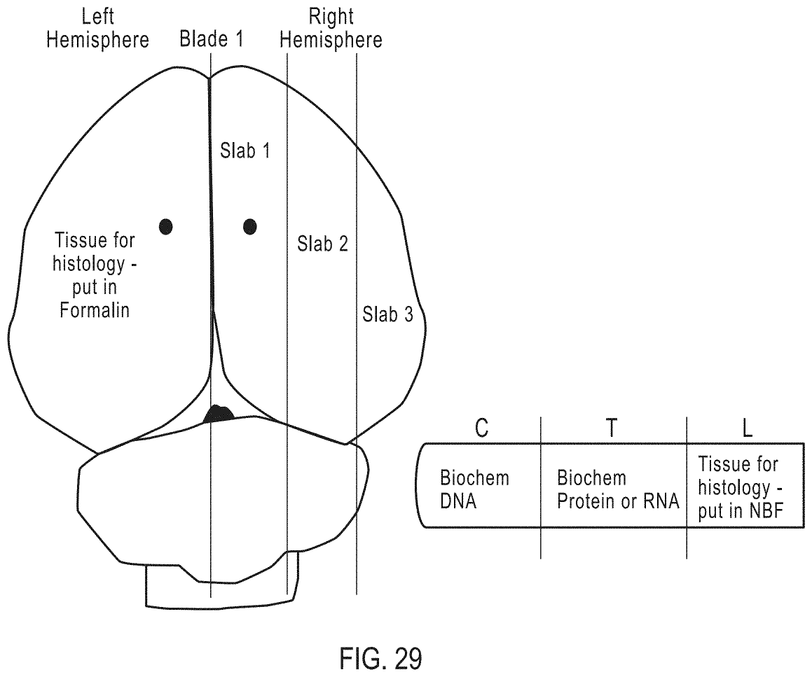

Tissues were collected at day 14 and day 30 post injection to assess vector biodistribution (AAV genomic DNA), Trastuzumab mRNA transcript expression, and Trastuzumab protein expression (Her2-binding detected by ELISA). Upon harvesting, brains were removed and placed in a Stainless Steel Sagittal Brain Matrix. Brains were cut in half in sagittal plans using Blade 1 and then slabs were collected as shown in FIG. 29. Slabs were placed into a tube and flash frozen or placed into fixative containing 10% NBF for 24 hours at room temperature for histology. Table 5 provides a summary of the tissue usage upon harvesting.

TABLE 5
Tissue Usage

| Tissue | Readout |
|---|---|
| Left Hemisphere | H&E and IHC |
| Slab 1 | Trastuzumab Protein ELISA or JESS |
| Slab 2 | Trastuzumab RNA ddPCR |
| Slab 3 | DNA |

### 7.3.2. Vector Genome Detection

Vector genome (i.e., AAV vector genomic DNA) was measured by ddPCR and presented as vector genome copies per diploid genome (VGC/DG). Tissues were harvested 13 and 30 days after injection and DNA was isolated. DNA was analyzed using the Bio-Rad ddPCR Supermix for Probes (no dUTP) (Bio-Rad 1863024) in combination with primers and probes specific for DNA encoding the Trastuzumab transgene and DNA encoding the non-human primate RPP30 reference. Primers and probes were designed to include intronic sequences to prevent contaminating RNA from interfering with accurate quantitation of vector genomes. After thermal cycling, samples were analyzed on the Bio-Rad QX200 Droplet Reader instrument using the Absolute Quantitation program. See Section 7.1.5 for additional experimental details.

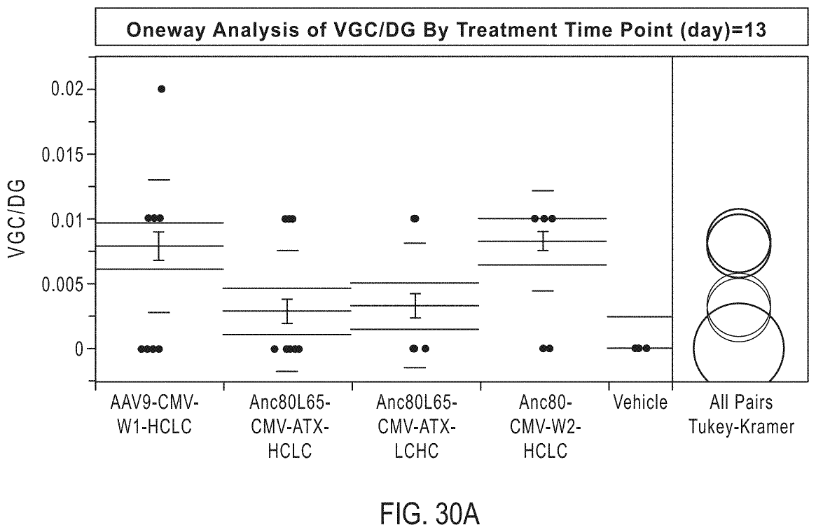

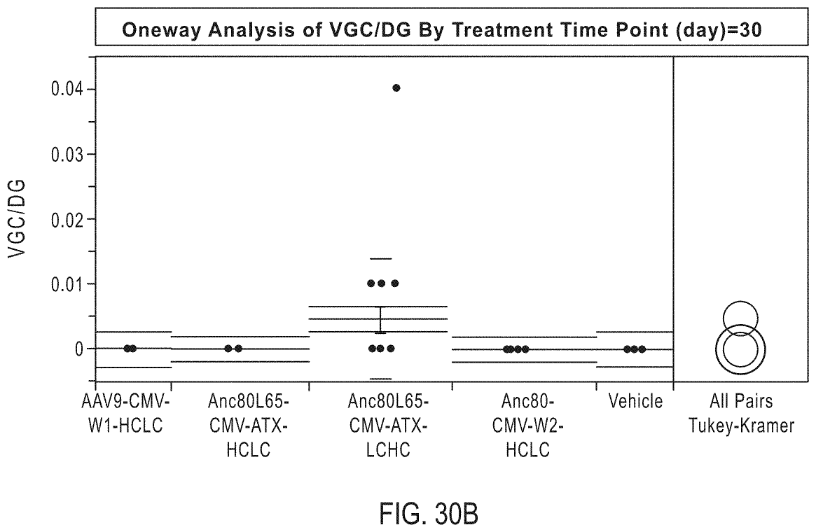

The results of the vector genome measurements are provided in FIG. 30A (day 13) and FIG. 30B (day 30). Using the All Pairs, Tukey HSD test (also called Tukey-Kramer), which shows the significance tests of all combinations of pairs, revealed Group 4 (Anc80L65.CMV.W2.HCLC) and Group 6 (AAV9.CMV.W1.HCLC) had the highest levels of AAV DNA at day 13 (FIG. 30A) and Anc80L65.CMV.ATX.LCHC had the highest level of AAV DNA at day 30 (FIG. 30B).

### 7.3.3. Gene Expression

Trastuzumab mRNA expression was measured by RT-ddPCR and presented as percentage of reference gene expression (Trastuzumab transcripts/RPP30 transcripts×100). Tissues were harvested at day 13 and 30 after injection and RNA was isolated. RNA from samples were analyzed using the Bio-Rad One-Step RT-ddPCR Advanced Kit for Probes (Bio-Rad 1864022) in combination with primers and probes specific for the Trastuzumab transgene and the non-human primate RPP30 reference gene. The reverse primer for both targets acted as the reverse transcription primer for the reverse transcription step. Where possible, primers and probes were designed across exon-exon junctions to prevent cross-reactivity with contaminating DNA. After thermal cycling, samples were analyzed on the QX200 Droplet Reader instrument using the Absolute Quantitation program. See Section 7.1.5 for additional experimental details.

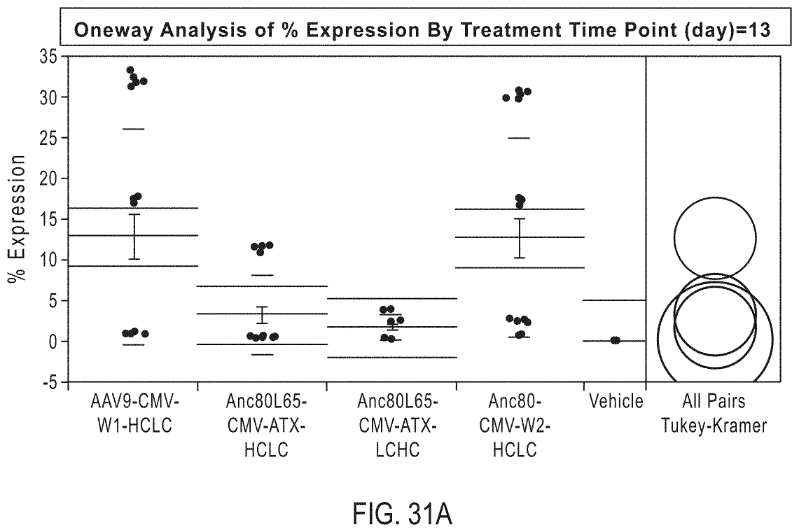

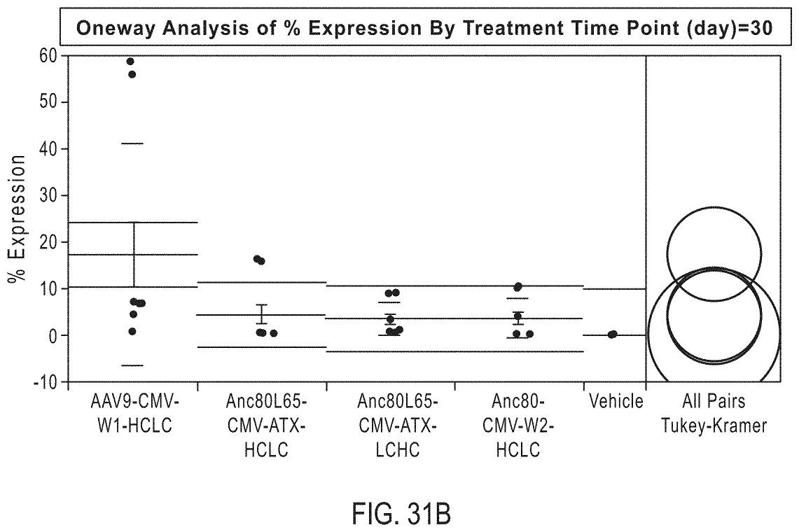

The results for Trastuzumab RNA expression measurements are provided in FIG. 31A (day 13) and FIG. 31B (day 30). Tukey-Kramer analysis revealed Group 4 (Anc80L65.CMV.W2.HCLC) and Group 6 (AAV9.CMV.W1.HCLC) had the highest levels of Trastuzumab RNA expression at day 13 (FIG. 31A) and Group 6 had the highest levels of Trastuzumab RNA expression at day 30 (FIG. 31B).

### 7.3.4. Protein Expression

Trastuzumab protein levels in brain tissue was measured by a HER2-binding ELISA and presented as absorbance normalized to total protein loaded. See Section 7.1.6 for additional experimental details.

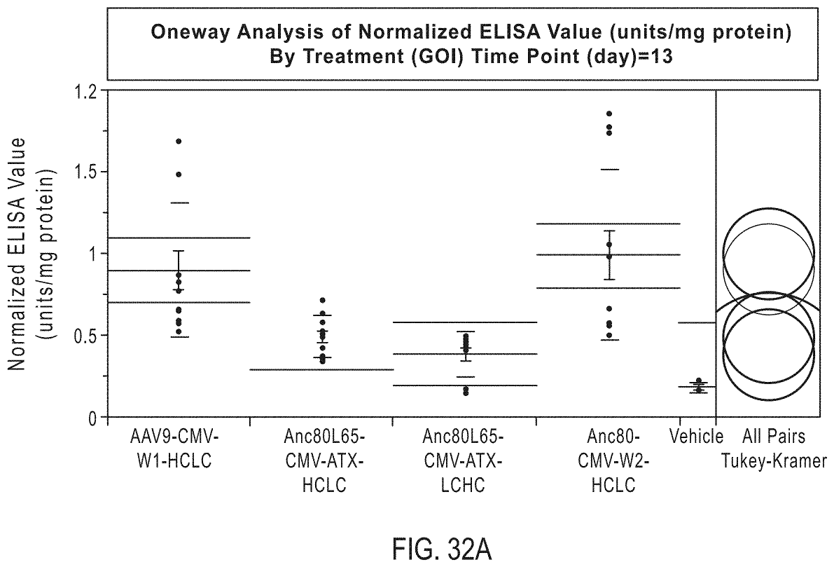

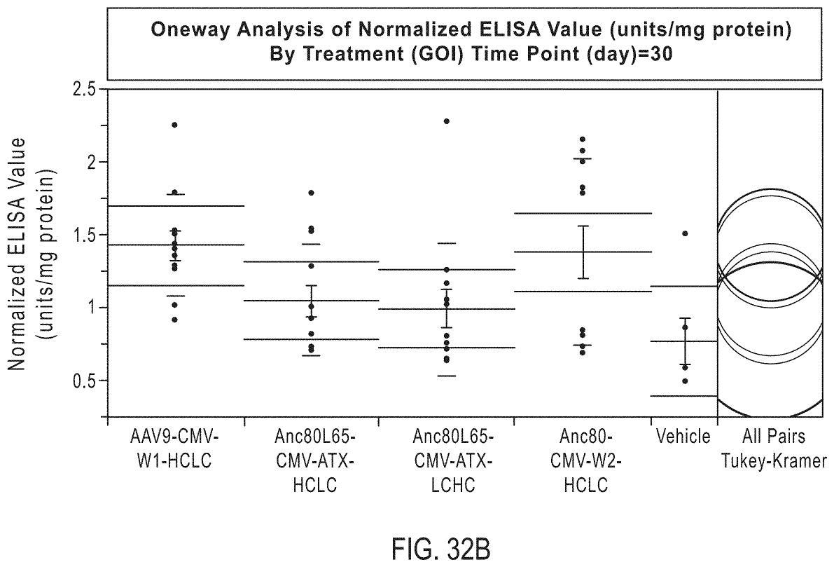

The results for Trastuzumab protein level measurements are provided in FIG. 32A (day 13) and FIG. 32B (day 30). Tukey-Kramer analysis revealed Group 4 (Anc80L65.CMV.W2.HCLC) had the highest levels of Trastuzumab protein at day 13 (FIG. 32A) followed by Group 6 (AAV9.CMV.W1.HCLC). At day 30, Group 4 and 6 had similar levels of Trastuzumab protein (FIG. 32B).

### 7.3.5. Conclusion

This experiment was designed to select candidate nucleic acid sequences encoding an anti-Her2 antigen-binding protein for use in the methods described herein.

Vector genomes and Trastuzumab RNA and protein were observed in brains for each treatment group. Additionally, vector genomes and Trastuzumab RNA were observed in the spinal cord for each treatment group (protein measurements were not performed for spinal cord tissue). Table 6 provides a summary for each treatment group.

TABLE 6
Summary of Candidate Selection

| | | | | RNA | Protein |
|---|---|---|---|---|---|
| | | | DNA | Expression | Expression |
| | | | Biodistribution | (% of | (Normalized |
| Group | Construct | Tissue | (VGC/DG) | reference) | Peak Area) |
| 6 | AAV9.CMV.W1.HCLC | Brain | 0.004 | 14.9 | 1.2 |
| 2 | Anc80L65.CMV.ATX.HCLC | Brain | 0.0015 | 3.7 | 0.8 |
| 5 | Anc80L65.CMV.ATX.LCHC | Brain | 0.0039 | 2.5 | 0.7 |
| 4 | Anc80L65.CMV.W2.HCLC | Brain | 0.0042 | 8.1 | 1.2 |
| 6 | AAV9.CMV.W1.HCLC | Spinal | 0.373 | 1 | ND |
| | | Cord | | | |
| 2 | Anc80L65.CMV.ATX.HCLC | Spinal | 0.0145 | 0.3 | ND |
| | | Cord | | | |
| 5 | Anc80L65.CMV.ATX.LCHC | Spinal | 0.0208 | 1.2 | ND |
| | | Cord | | | |
| 4 | Anc80L65.CMV.W2.HCLC | Spinal | 0.0182 | 0.3 | ND |
| | | Cord | | | |

Overall, administration of Group 4 Anc80L65.CMV.W2.HCLC produced the highest RNA and protein expression for Trastuzumab amongst the various AAVs tested comprising an Anc80L65 capsid.

### 7.4. Example 3: Promoter Selection Study

This experiment was performed to select candidate promoter sequences for further experimentation. In particular, the experiment was designed to evaluate Trastuzumab expression using either a CMV promoter (SEQ ID NO: 14) or a UbC promoter (SEQ ID NO: 11). The construct included a coding sequence of Trastuzumab (HER.W2.DELM) having the sequence of SEQ ID NO: 23. The HER.W2.DELM has a sequence identical to W2 tested in the experiments described in 6.3, except that it includes D356E and L358M mutations in the CH2.CH3 fragment.

Each Trastuzumab polynucleotide construct including either a CMV promoter or a UbC promoter was encapsulated by an Anc80L65 capsid and administered to RAG knockout mice.

### 7.4.1. Experimental Design

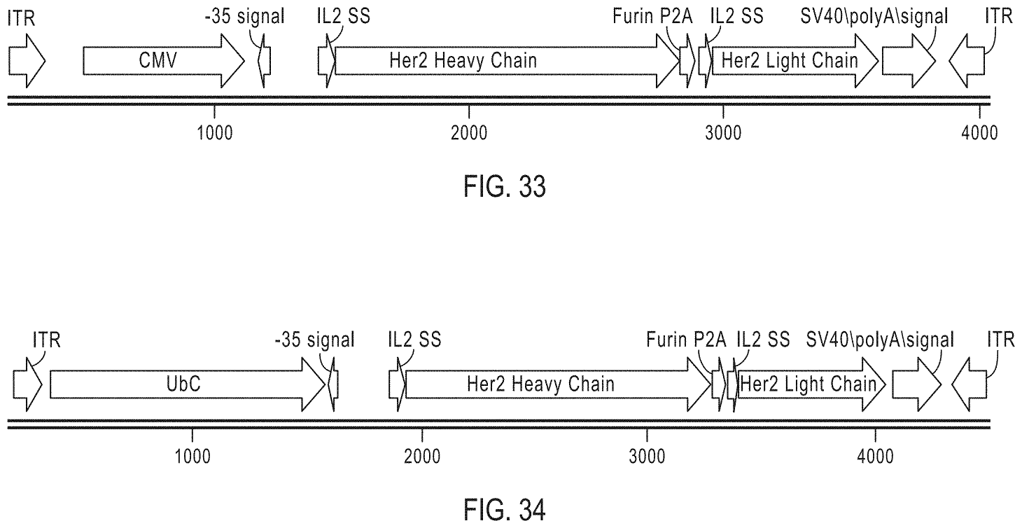

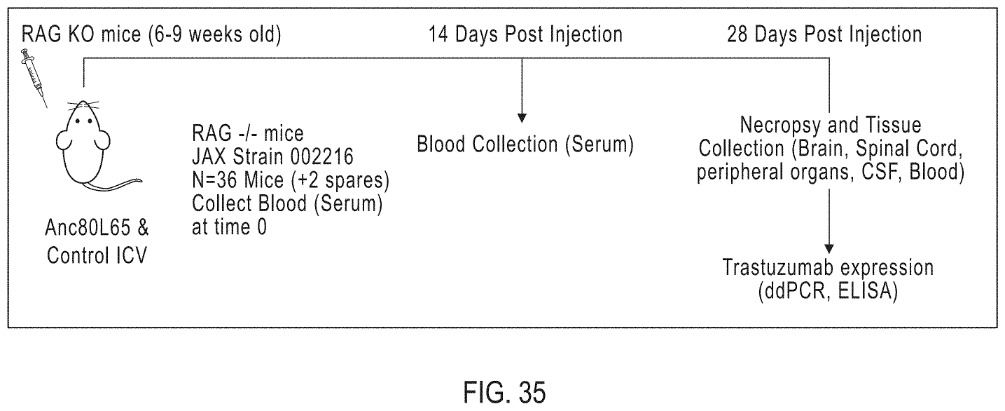

RAG KO mice to be treated were divided into four treatment groups (Group 1-4). Group 1 received an ICV injection of formulation buffer and served as a vehicle control. Group 2 received Anc80L65.CMV.HER.W2.DELM by ICV injection. CMV.HER.W2.DELM of the Anc80L65.CMV.HER.W2.DELM corresponds to SEQ ID NO: 24 (see FIG. 33 for a schematic of the construct). Group 3 received Anc80L65.UBC.HER.W2.DELM by ICV injection. UBC.Her2W2.DELM of the Anc80L65.UBC.HER.W2.DELM AAV corresponds to SEQ ID NO: 25 (see FIG. 34 for a schematic of the construct). Group 4 received Anc80L65.CMV-W1 by ICV injection. CMV-W1 of the Anc80L65.CMV-W1 corresponds to SEQ ID NO: 26. Table 7 provides a summary of the experimental design for experimental Groups 1˜4 (see also FIG. 35).

TABLE 7
Experimental Design

| | | | Dose (vg/g | | |
|---|---|---|---|---|---|
| | | | Brain | Dose | |
| Group | Vector | ROA | weight) | (vg/mouse) | N-value |
| 1 | Formulation Buffer | ICV | NA | NA | 6 |
| 2 | Anc80L65.CMV.HER.W2.DELM | ICV | 2.00E+11 | 8.00E+10 | 10 |
| | (SEQ ID NO: 24) | | | | |
| 3 | Anc80L65.UBC.HER.W2.DELM | ICV | 2.00E+11 | 8.00E+10 | 10 |
| | (SEQ ID NO: 25) | | | | |
| 4 | Anc80L65.CMV-W1 | ICV | 2.00E+11 | 8.00E+10 | 10 |
| | (SEQ ID NO: 26) | | | | |

Table 8 provides a summary of the sedimentation velocity (SV-AUC) for rAAV prepared for Groups 2, 3, and 4. Table 8 shows that about 20% of each virus prep included partial capsids.

TABLE 8
Summary Sedimentation velocity (SV-AUC)

| Group | Vector | vg/mL | EU/mL | pH | Osmolality |
|---|---|---|---|---|---|
| 2 | Anc80L65.CMV HER.W2 DELM | 1.72E+13 | <0.25 | 7.05 | 369 |
| 3 | Anc80L65.UBC HER.W2 DELM | 5.45E+13 | <0.25 | 7.11 | 373 |
| 4 | Anc80L65.CMV.W1 | 1.80E+13 | <0.25 | 7.01 | 367 |

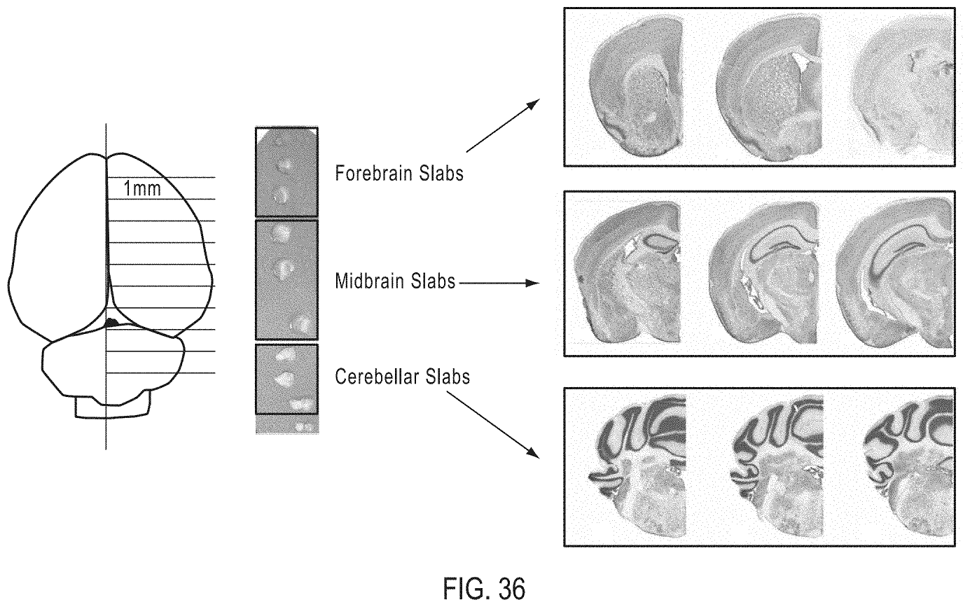

Tissues were collected at day 14 and day 28 post injection to assess vector biodistribution (AAV genomic DNA), Trastuzumab RNA expression, Trastuzumab protein expression (Her2-binding detected by ELISA), and cellular biodistribution using IHC (anti-IgG Fc). Upon harvesting, brains were removed and placed in a Stainless Steel Sagittal Brain Matrix. Brains were cut in half sagittal plans and then slabs were collected as shown in FIGS. 36A-D. Slabs were placed into a tube and flash frozen or placed into fixative for histology containing 10% neutral buffered formalin for 24 hours at room temperature.

### 7.4.2. Vector Genome Detection

Vector genome (i.e., AAV vector genomic DNA) was measured by ddPCR and presented as vector genome copies per diploid genome (VGC/DG). Tissues were processed as described in Section 7.3.2 except tissues were harvested at a single time point (day 28). See also Section 7.1.5 for additional experimental details.

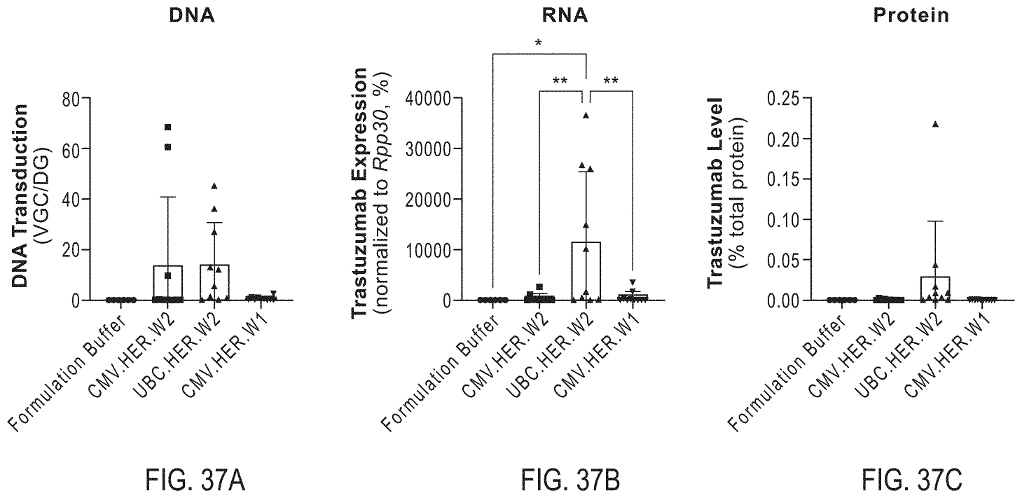

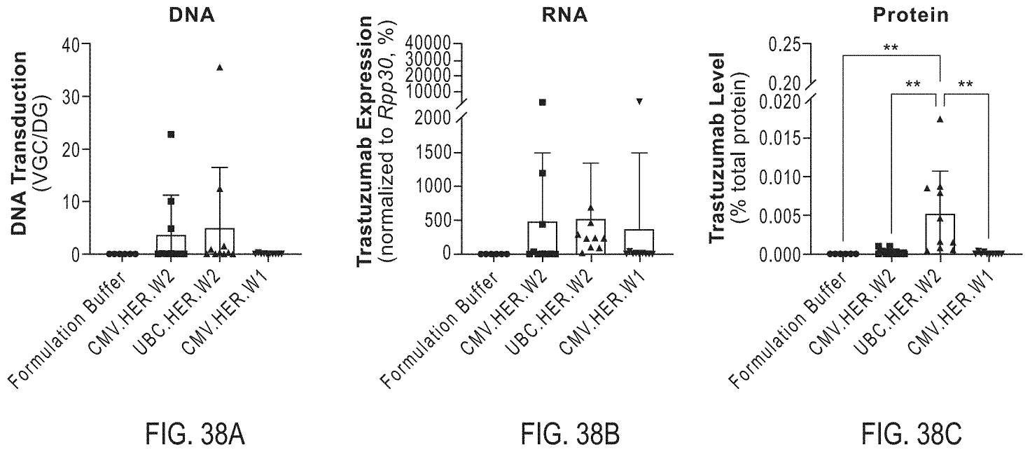

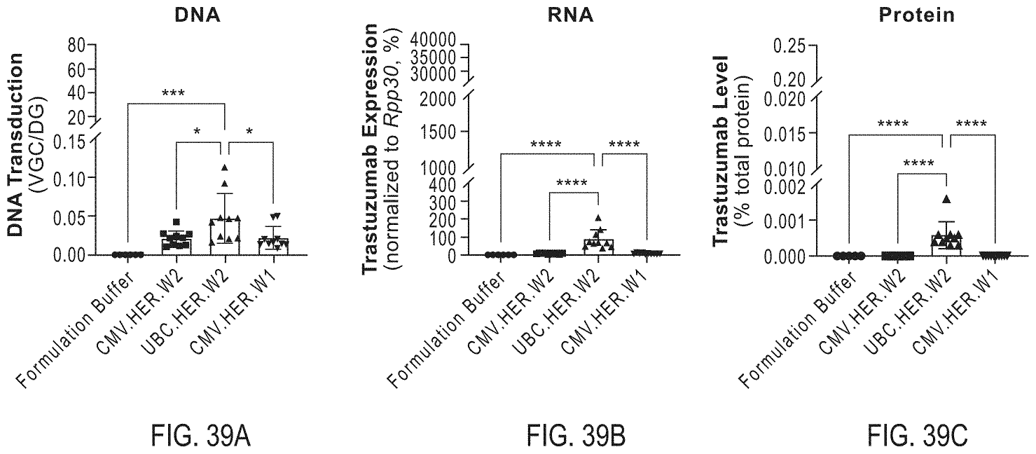

The results of the vector genome measurements are provided in FIG. 37A (forebrain), FIG. 38A (midbrain), and FIG. 39A (cerebellum). FIG. 37A and FIG. 38A show that transduction with Group 3 (Anc80L65.UBC.HER.W2.DELM) did not produce statistically significant difference in vector genome copies per diploid cell as compared to either Groups 2 or 4. FIG. 39A show that Group 3, which include the UbC promoter, produced statistically significant differences in vector genome copies per diploid genome in cerebellum (FIG. 37A) as compared to Group 2 and/or Group 4, both of which include a polynucleotide comprising the CMV promoter. Statistically significant differences between the means were determined by an ANOVA one-way test followed by Dunnett multiple comparison tests with P-values indicated with asterisks. *P<0.05, **p<0.005; ***p<0.001; ****p<0.0001, ns=not significant.

7.4.3. Gene Expression

Trastuzumab mRNA expression was measured by RT-ddPCR and presented as percentage of reference gene expression (Trastuzumab transcripts/RPP30 transcripts×100). Tissues were processed as described in Section 7.3.3 except tissues were harvested at a single time point (day 28). See also Section 7.1.5 for additional experimental details.

The results for Trastuzumab RNA expression measurements are provided in FIG. 37B (forebrain), FIG. 38B (midbrain), and FIG. 39B (cerebellum). FIG. 37B and FIG. 39B show that Group 3 (Anc80L65.UBC.HER.W2.DELM) produced statistically significant differences in Trastuzumab RNA expression in forebrain (FIG. 37B; P=0.0029) and cerebellum (FIG. 39B; P<0.0001) as compared to Group 2 and/or Group 4, both of which include a polynucleotide comprising the CMV promoter. Statistically significant differences between the means were determined by an ANOVA one-way test followed by Dunnett multiple comparison tests with P-values indicated with asterisks. *P<0.05, **p<0.005; ***p<0.001; ****p<0.0001, ns=not significant.

### 7.4.4. Protein Expression

Trastuzumab protein expression in brain tissue was measured by a HER2-binding ELISA and presented as absorbance normalized to total protein loaded. Tissues were processed as described in Section 7.3.4 except tissues were harvested on day 28.

The results of Trastuzumab protein expression measurements are provided in FIG. 37C (forebrain), FIG. 38C (midbrain), and FIG. 39C (cerebellum). FIG. 38C and FIG. 39C show that Group 3 (Anc80L65.UBC.HER.W2.DELM) produced statistically significant differences in Trastuzumab protein expression in midbrain (FIG. 38C; P=0.0007) and cerebellum (FIG. 39C; P<0.0001) as compared to Group 2 and/or Group 4, both of which include a polynucleotide comprising the CMV promoter. Statistically significant differences between the means were determined by an ANOVA one-way test followed by Dunnett multiple comparison tests with P-values indicated with asterisks. *P<0.05, **p<0.005; ***p<0.001; ****p<0.0001, ns=not significant.

For IHC analysis, Tissue samples were collected at day 28 post vector administration and immediately placed into 10% neutral buffered formalin for approximately 48 hours and then transferred to 70% ethanol. Samples in ethanol were shipped at ambient temperature to Histosery (Germantown, Md.). See Section 7.1.3 for additional experimental details.

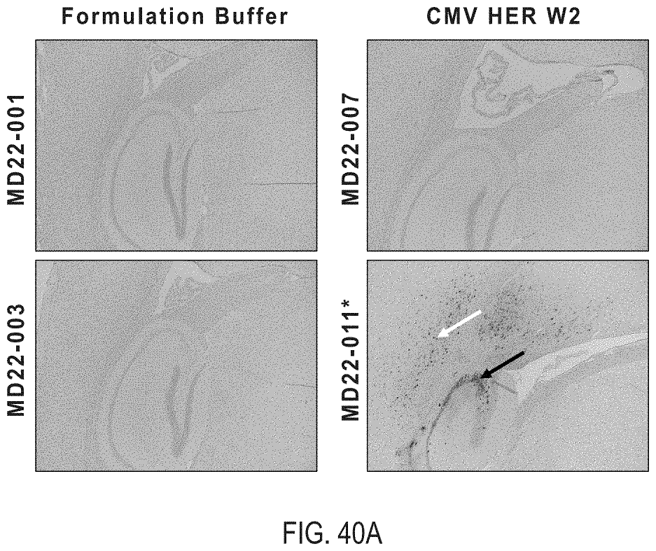

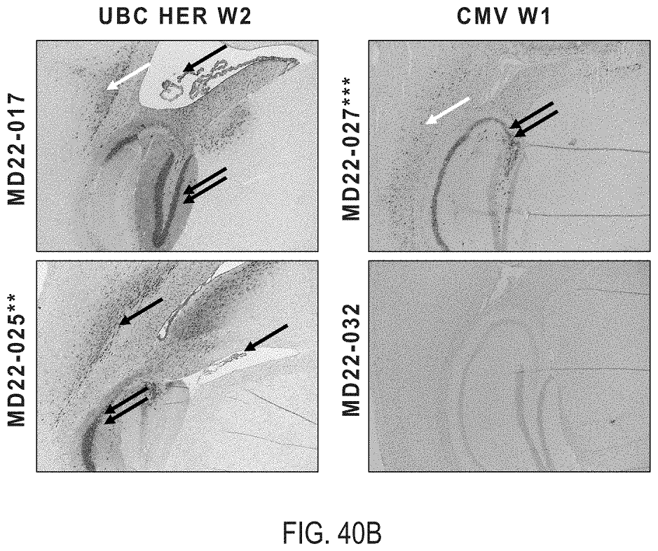

FIGS. 40A-40B show representative images of brain-cross sections obtained after staining with human IgG Fc (used as a proxy for Trastuzumab expression). Trastuzumab expression was higher and had greater biodistribution in Group 3 (Anc80L65.UBC.HER.W2.DELM) than Group 2 (Anc80L65.CM. HER.W2.DELM) and/or Group 4 (Anc80L65.CMV-W1).

### 7.4.5. Conclusion

This experiment was designed to evaluate Trastuzumab expression using either a CMV promoter or a UbC promoter. Each promoter-Trastuzumab polynucleotide construct was encapsulated by a rAAV comprising an Anc80L65 capsid and administered to RAG knockout mice.

Group 3 (RAG KO mice administered a rAAV comprising an Anc80L65 capsid and a polynucleotide including a UBC promoter driving Trastuzumab expression) resulted in statistically significant increases in Trastuzumab RNA expression and Trastuzumab protein levels as compared to mice in Group 2 or Group 4. In forebrain tissue, Group 3 had Trastuzumab RNA expression statistically significantly greater than Group 2 or Group 4 (FIG. 37B). For example, forebrains from Group 3 had 27× greater Trastuzumab RNA expression than to Group 2 and 20× greater Trastuzumab RNA expression than Group 4. In the midbrain, Group 3 had Trastuzumab protein expression statistically significantly greater than Group 2 or Group 4 (FIG. 38C). For example, midbrains from Group 3 had 21× greater Trastuzumab protein than Group 2 and 74× greater Trastuzumab protein than Group 4. In the cerebellum, Group 3 showed vector genome levels statically significantly greater than Group 2 or Group 4 (FIG. 39A). Additionally, in the cerebellum, Group 3 had expression of both Trastuzumab RNA (FIG. 39B) and Trastuzumab protein (FIG. 39C) at statistically significantly greater levels compared to Groups 2 or 4. In particular, cerebellum from Group 3 had 12× greater Trastuzumab RNA expression compared to Group 2 and 11× greater Trastuzumab RNA expression compared to Group 4. In both the forebrain and midbrain where there was no significant difference between vector genome copy number among Groups 2, 3, and 4, Group 3 produced statistically significant increases in Trastuzumab RNA in the forebrain (FIG. 37B) and Trastuzumab protein in the midbrain (FIG. 38C).

Table 9 provides a summary of vector genome detection, Trastuzumab RNA expression, and Trastuzumab protein expression from this experiment.

TABLE 9
Summary of Promoter Selection Study

| | Forebrain slab | | | Midbrain slab | |
|---|---|---|---|---|---|
| Treatment Group (Grp) | DNA | RNA | Protein | DNA | RNA |
| Formulation Buffer (Grp 1) | 0.00015 ± 0.00013 | <0.01 | 0 | <0.0001 | <0.01 |
| Anc80L65.CMV.HER.W2 (2) | 13.98 ± 26.92 | 421.2 ± 846.9 | 0.00048 ± 0.00092 | 3.791 ± 7.420 | 481.6 ± 1006.0 |
| Anc80L65.UBC.HER.W2 (3) | 14.23 ± 16.46 | 11706.0 ± 13754.0 | 0.03062 ± 0.06730 | 5.109 ± 11.350 | 515.3 ± 825.2 |
| Anc80L65.CMV.HER.W1 (4) | 0.65 ± 0.77 | 575.9 ± 1151 | 0.00018 ± 0.00031 | 0.589 ± 0.082 | 365.6 ± 1123 |

| | Midbrain slab | Cerebellum slab | | |
|---|---|---|---|---|
| Treatment Group (Grp) | Protein | DNA | RNA | Protein |
| Formulation Buffer (Grp 1) | 0 | 0 | <0.01 | 0 |
| Anc80L65.CMV.HER.W2 (2) | 0.00024 ± 0.00042 | 0.02121 ± 0.010 | 7.419 ± 2.609 | 0 |
| Anc80L65.UBC.HER.W2 (3) | 0.00519 ± 0.00549 | 0.0471 ± 0.032 | 86.850 ± 53.430 | 0.00058 ± 0.00038 |
| Anc80L65.CMV.HER.W1 (4) | 0.00007 ± 0.00015 | 0.022 ± 0.015 | 7.249 ± 3.365 | 0 |

Overall, administration of Anc80L65.UBC.HER.W2 to RAG KO mice (Group 3) resulted in statistically significant increases in Trastuzumab RNA and protein expression levels as compared to mice treated with either Anc80L65.CMV.HER.W2 (Group 2) or Anc80L65.CMV.HER.W1 (Group 4). Additionally, IHC using IgG Fc (a proxy for Trastuzumab protein expression) showed stronger expression and greater biodistribution of Trastuzumab protein in Group 3 compared to Group 2 or 4. This data supported selection of the UbC promoter.

### 7.5. Example 4: Design of Anc80L65 rAAVs for Treating MLD

Anc80L65 (SEQ ID NO: 1) rAAVs encapsulating a polynucleotide having a coding sequence of a native (wild-type) human ARSA (SEQ ID NO: 5) or human ARSA variant having 202V, T286L, and R291N substitutions (referred to herein as “Hyper-ARSA”) (SEQ ID NO: 6) operably linked to a UbC promoter (SEQ ID NO: 10), CAG promoter (SEQ ID NO: 12) or CMV promoter (SEQ ID NO: 13) were designed. Hyper-ARSA has been reported to have substantially increased activity compared to native human ARSA (see, Simonis et al., 2019, Human Molecular Genetics 28(11):1810-1821; WO 2018/141958).

Coding sequences for native human ARSA included a native coding sequence (SEQ ID NO: 4) and two codon-optimized coding sequences, referred to as COGS and COGA (SEQ ID NO: 2 and SEQ ID NO: 3, respectively). Coding sequences for Hyper-ARSA included two codon-optimized sequences, referred to as COGS-Hyper and COGA-Hyper (SEQ ID NO: 7 and SEQ ID NO: 8, respectfully).

Constructs further included 5′ and 3′ ITRs (SEQ ID NOs: 17-18, respectively), a woodchuck hepatitis virus post-transcriptional regulatory element (WPRE) (SEQ ID NO: 15), and SV40 late polyadenylation signal sequence (SEQ ID NO: 16).

Applicant's initial helper plasmid and gene of interest (G01) plasmid contained the L5 Ad5 fiber coding gene. The presence of the L5 Ad5 fiber coding gene was a legacy holdover from when helper plasmids were originally designed for triple transfection AAV production. To prevent possible contamination of rAAV preparations with this protein, the fiber gene was removed from both plasmids. There was no obvious consequence of removing the fiber gene on yield, genomic integrity, percent full capsids, or capsid purity (data not shown).

### 7.6. Example 5: In Vitro ARSA Expression and Activity in Cells Transfected with rAAVs of Example 4

In vitro studies were performed with HEK293 cells transfected with rAAVs of Example 4 to assess ARSA protein expression and to determine any differences in ARSA enzyme activity among the different rAAVs. ARSA protein level analysis was performed using the ProteinSimple Jess instrument.

Protein expression, enzyme activity, and normalized enzyme activity for four constructs are shown in Table 10. Normalized enzyme activity is enzyme activity (OD Units) divided by protein expression (Peak Area) to provide an estimate of the enzyme activity per protein molecule. For both the CMV and UbC promoter constructs, the Hyper version of the protein produced ˜2-fold higher enzyme activity per protein molecule than the native (COGS) version. The data indicates that the UbC promoter outperformed the CMV promoter.

TABLE 10

| | Protein | Enzyme | Normalized |
|---|---|---|---|
| | Expression | Activity | Enzyme |
| Construct | (Peak Area) | (OD Units) | Activity |
| CMV-COGS | 1277681.3 | 0.044 | 0.334 |
| (ATP0139) | | | |
| CMV-COGS- | 1514657.6 | 0.109 | 0.712 |
| Hyper (ATP0138) | | | |
| UbC-COGS | 2032804.0 | 0.131 | 0.618 |
| (ATP0123) | | | |
| UbC-COGS- | 1675256.7 | 0.170 | 1.034 |
| Hyper (ATP0137) | | | |

### 7.7. Example 6: ARSA Expression in Wild-Type Mice Administered rAAVs of Example 4

A study was performed to evaluate ARSA RNA and protein expression following intracerebroventricular (ICV) injection of rAAVs of Example 4 to wild-type mice.

### 7.7.1. Materials and Methods

The groups used in the study are shown in Table 11.

TABLE 11

| | Dose | Dose | Sample |
|---|---|---|---|
| Construct | (vg/mouse) | (vg/g brain weight) | size |
| NA (saline control) | NA | NA | 5 |
| CAG-COGA | 5e10 | 1.1e11 | 5 |
| CAG-COGA- | 5e10 | 1.1e11 | 5 |
| Hyper | | | |
| CAG-COGS | 5e10 | 1.1e11 | 5 |
| UbC-COGA | 5e10 | 1.1e11 | 5 |
| UbC-COGS | 5e10 | 1.1e11 | 5 |
| UbC-Native | 5e10 | 1.1e11 | 5 |

rAAVs were administered to the lateral ventricle (5 mL of viral suspension/injection site), using a 33G sharp needle attached to a 10-mL Hamilton syringe (Sigma-Aldrich, St. Louis, Mo., USA), at a rate of 0.2 mL/min. Stereotactic coordinates of injection sites were calculated from bregma (lateral ventricle coordinates: anteroposterior+0.25 mm, mediolateral ±0.7 mm, and dorsoventral 2 mm). ARSA RNA and protein expression was evaluated 14 days post injection.

### 7.7.2. Results

Vector genome biodistribution (VGC/DG), RNA expression, normalized RNA expression and normalized protein expression as observed in WT mouse brains are shown in Table 6. Normalized RNA expression is RNA Expression (% of reference) divided by DNA biodistribution (VGC/DG) to provide an estimate of the number of RNA molecules generated per vector genome. In the context of both CAG and UbC promoters, the COGS version of the ARSA gene produced the highest RNA expression of the three codon-optimized versions. Normalized protein expression is peak area adjusted for total protein load. The COGS version also produced more protein than other codon optimized versions for both promoters.

TABLE 12

| | DNA | RNA | | Normalized |
|---|---|---|---|---|
| | Biodis- | Expression | Normalized | Protein |
| | tribution | (% of | RNA | Expression |
| Construct | (VGC/DG) | Reference) | Expression | (Peak Area) |
| CAG-COGA | 0.03 | 9.8 | 407.2 | 308,743.3 |
| CAG-COGA- | 0.04 | 8.3 | 425.8 | 331,067.3 |
| Hyper | | | | |
| CAG-COGS | 0.08 | 54.1 | 872.6 | 552,228.2 |
| UbC-COGA | 0.03 | 7.1 | 215.1 | 256,695.0 |
| UbC-COGS | 0.04 | 11.4 | 692.7 | 267,879.4 |
| UbC-Native | 0.03 | 0.8 | 37.0 | 150,680.6 |

### 7.8. Example 7: Sulfatide Levels in ARSA Knock-Out Mice

ARSA knock-out (KO) mice exhibit abnormal sulfatide storage patterns in the nervous system, similar to MLD, making ARSA knock-out mice a useful model for studying MLD (Hess et al., 1996, PNAS 93(25):14821-14826). A longitudinal study was performed to characterize sulfatide and lysosulfatide levels in ARSA−/− mice and ARSA+/− mice 4 to 14 months of age.

### 7.8.1. Materials and Methods

ARSA−/− mice and ARSA+/− littermates were evaluated every two months from 4 to 14 months of age for lysosulfatide and sulfatide levels.

TABLE 13

| | Age | ARSA −/− | ARSA +/− | |
|---|---|---|---|---|
| Group | (months) | N-Value | N-Value | Total |
| 1 | 4 | 2 | 2 | 4 |
| 2 | 6 | 2 | 2 | 4 |
| 3 | 8 | 2 | 2 | 4 |
| 4 | 10 | 2 | 2 | 4 |
| 5 | 12 | 2 | 2 | 4 |
| 6 | 14 | 2 | 2 | 4 |

Lysosulfatide and short chain (C16:0, C18:0) and long chain (C24:0, C24:1) sulfatide levels were evaluated in brain and spinal cord from each animal by high-performance liquid chromatography—mass spectrometry (HPLC-MS/MS).

### 7.8.2. Results

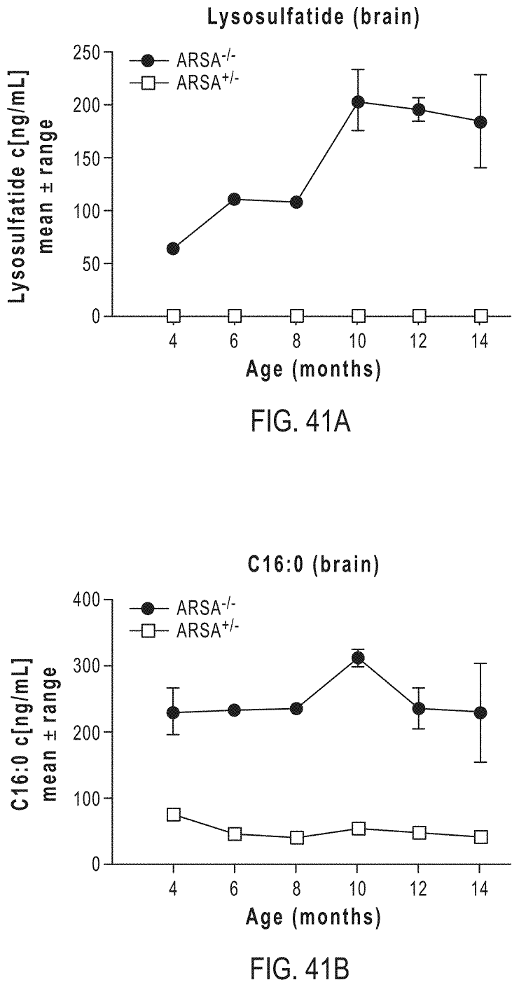

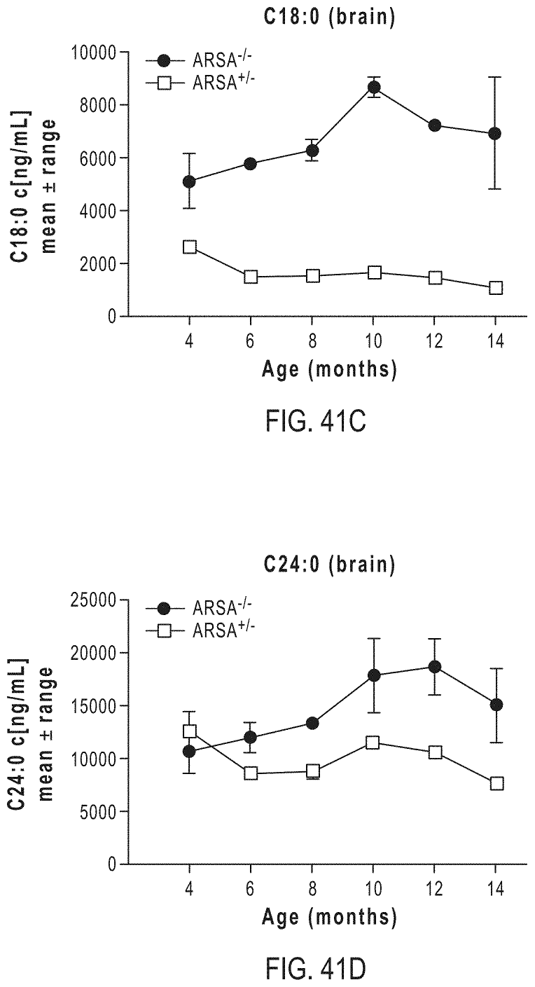

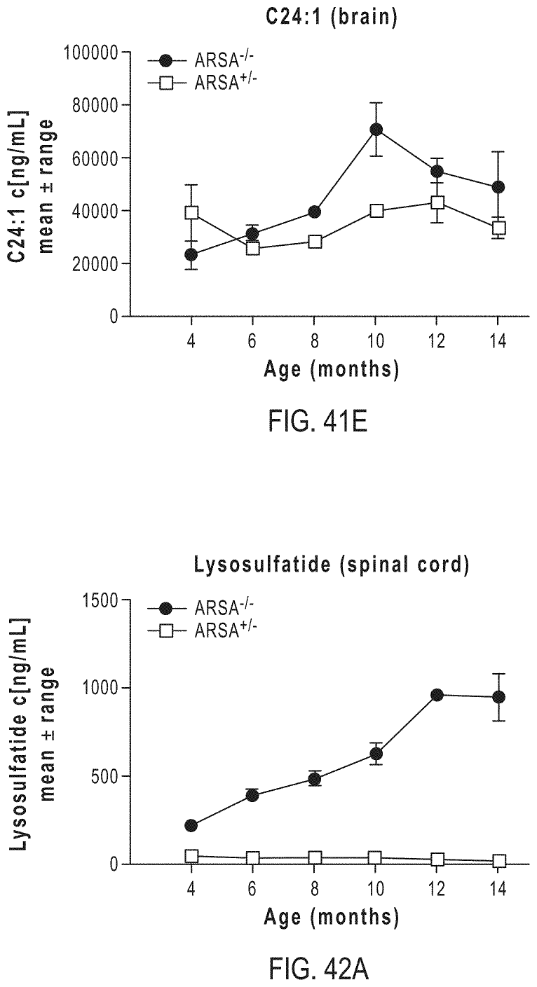

Lysosulfatide and sulfatide levels in brain are shown in FIGS. 41A-41E. Lysosulfatide (FIG. 41A) and short-chain sulfatide species C16:0 (FIG. 41B) and C18:0 (FIG. 41C) were more abundant in ARSA−/− mice than ARSA+/− mice as early as 4 months of age. C24:0 sulfatide (FIG. 41D) was more abundant in ARSA−/− mice at 8 to 10 months of age compared to ARSA+/− mice, while C24:1 sulfatide (FIG. 41E) appeared similar in ARSA−/− and ARSA+/− mice at the ages studied.

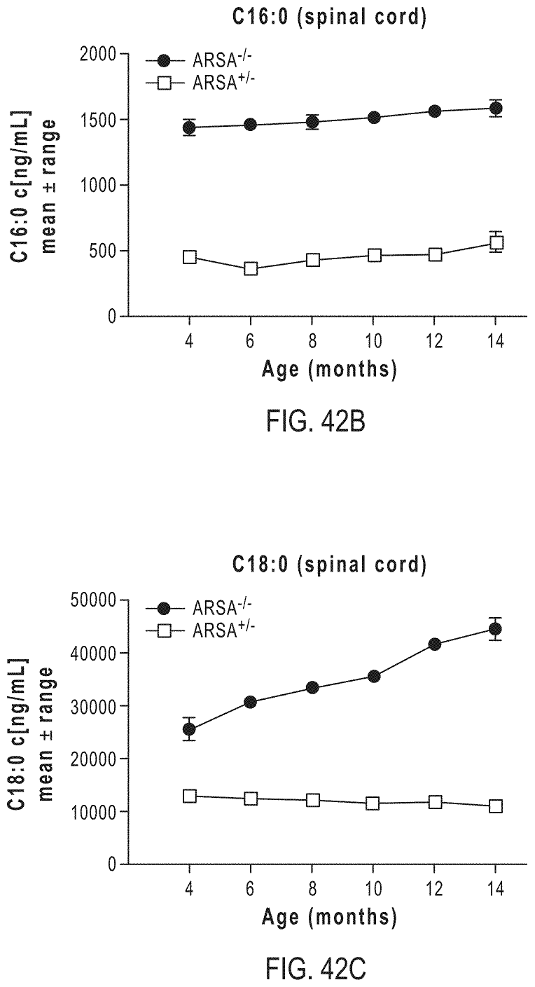

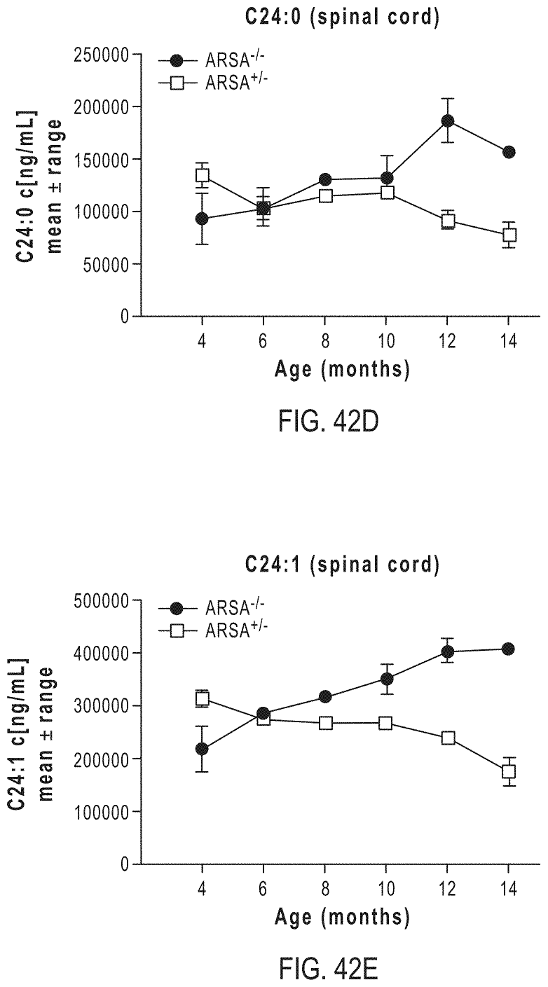

Lysosulfatide and sulfatide levels in spinal cord are shown in FIGS. 42A-42E. Lysosulfatide (FIG. 42A) and short-chain sulfatide species C16:0 (FIG. 42B) and C18:0 (FIG. 42C) were more abundant in ARSA−/− mice than ARSA+/− mice as early as 4 months of age, and continued to accumulate throughout follow-up. C24:0 sulfatide (FIG. 42D) increased by 12 months of age, and C24:1 sulfatide (FIG. 42E) increased by 8 months in ARSA−/− mice relative to ARSA+/− mice.

In summary, lysosulfatide and short-chain sulfatides were observed to accumulate as early as four months of age in brain and spinal cord of ARSA−/− mice. Long-chain sulfatides showed delayed accumulation in ARSA−/− mice, with increases generally beginning sometime between 8 and 10 months of age. In total, the data supports the use of ARSA−/− mice to evaluate sulfatide lowering strategies for the treatment of MLD.

### 7.9. Example 8: ARSA Enzyme Activity and Sulfatide Levels in ARSA Knock-Out Mice Administered rAAVs of Example 4

Sulfatides are a major component of the myelin sheath in the nervous system, and sulfatide accumulation in oligodendrocytes leads to severe demyelination. Lysosulfatide is a cytotoxic compound in cell culture and suggested to be involved in MLD pathology. A study was performed to evaluate ARSA expression and sulfatide-reducing activity following intracerebroventricular (ICV) injection of rAAVs of Example 4 (specifically, UbC-COGS, UbC-COGS-Hyper and CMV-COGS-Hyper) to ARSA knock-out (KO) mice.

### 7.9.1. Materials and Methods

Adult ARSO KO mice 8 months of age at study start were used in this study. The groups used in the study as shown in Table 14.

TABLE 14

| | Dose | Dose | Sample |
|---|---|---|---|
| Construct | (vg/mouse) | (vg/g brain weight) | size |
| NA (formulation | NA | NA | 4 |
| buffer) | | | |
| UbC-COGS | 5e10 | 1.1e11 | 6 |
| UbC-COGS-Hyper | 5e10 | 1.1e11 | 6 |
| CMV-COGS-Hyper | 5e10 | 1.1e11 | 6 |

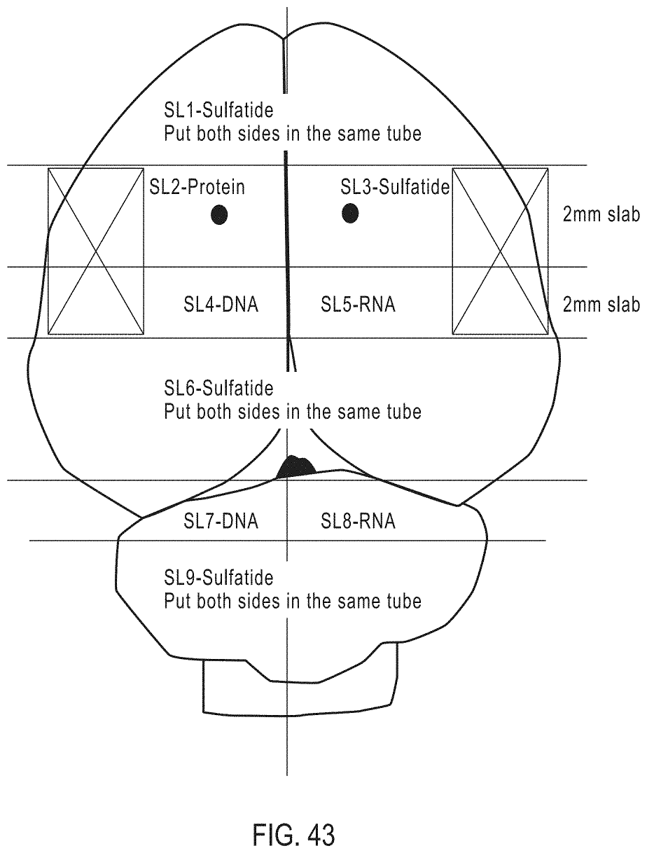

rAAVs were administered as in Example 6. ARSA expression and distribution was evaluated 28 days post injection. Brain and spinal cord samples were collected for analysis (see, FIG. 43). Total protein concentration was determined by BCA assay. Samples were normalized to 500 μg/mL prior to analysis. ARSA protein level analysis was performed using the ProteinSimple Jess instrument, with untreated wild-type controls run on each cartridge.

Sulfatides and lysosulfatides were measured by LC/MS.

### 7.9.2. Results

### 7.9.2.1 Lysosulfatide and Sulfatide Reduction

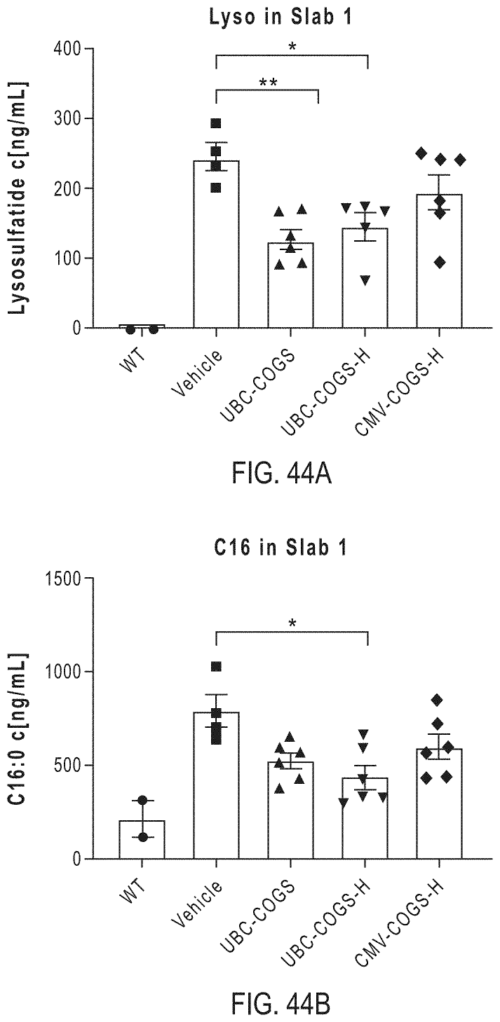

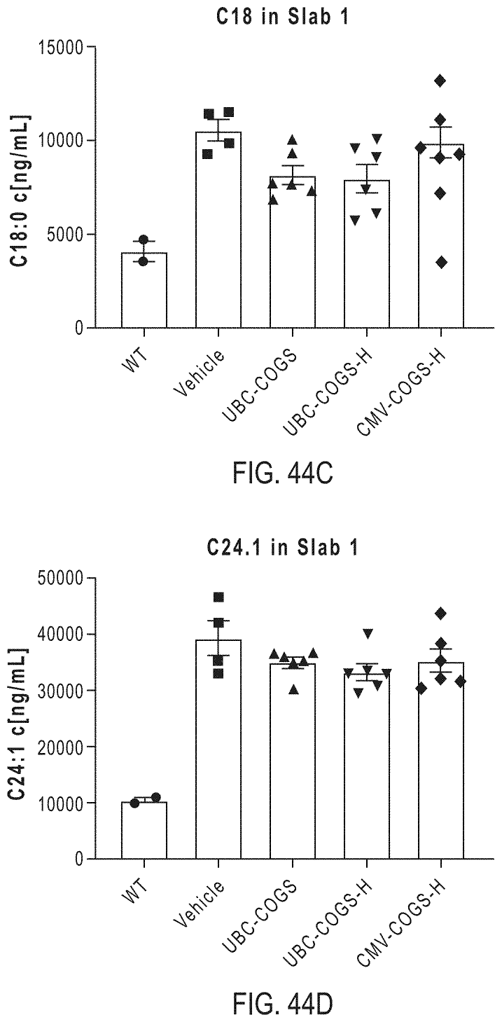

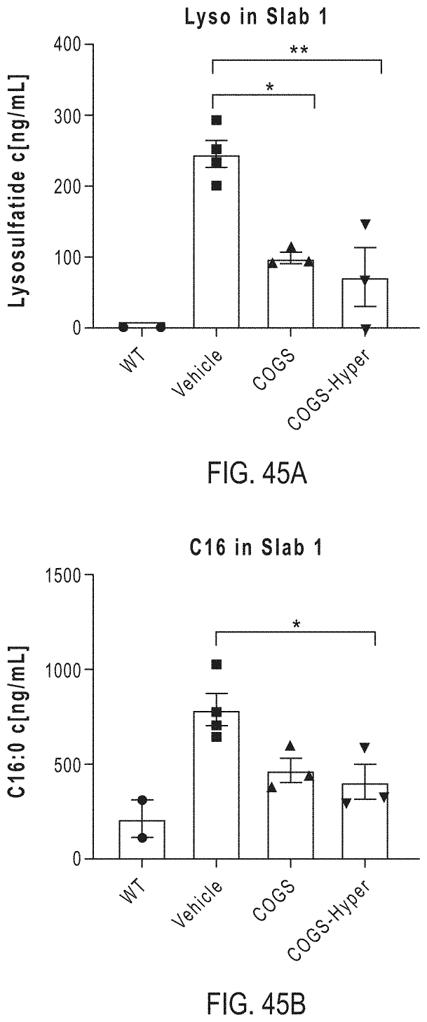

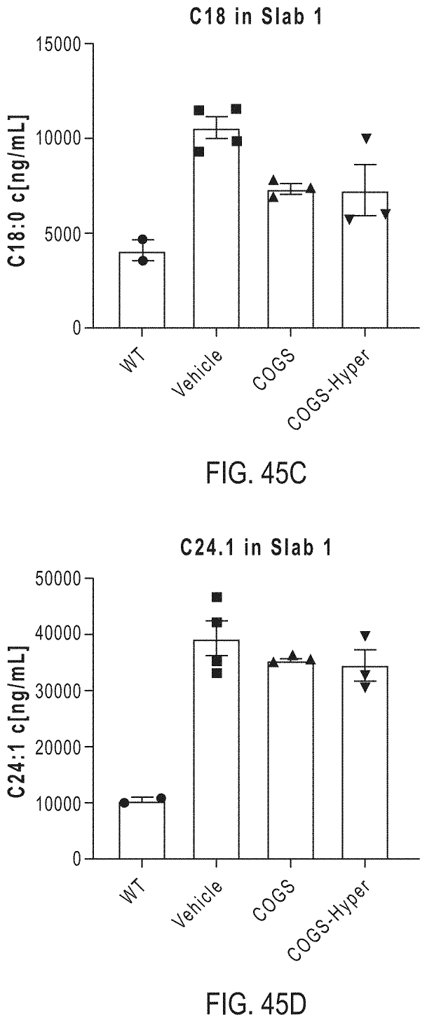

rAAV treatment groups showed reductions in lysosulfatide and sulfatides in brain slab 1 (FIGS. 44A-44D), with significant reduction in lysosulfatide for UbC-COGS and UbC-COGS-Hyper (FIG. 44A) and C16 sulfatide for UbC-COGS-Hyper (FIG. 44B) compared to vehicle. When comparing data for UbC constructs from high ARSA expressing mice, the Hyper-ARSA construct provided a greater reduction in lysofulfatide and C16 sulfatide compared to COGS-ARSA (FIGS. 45A-45B).

Statistically significant differences in lysosulfatide and sulfatide levels in brain slabs 3, 6, and 9 between vehicle and rAAV treatment groups were not observed (data not shown).

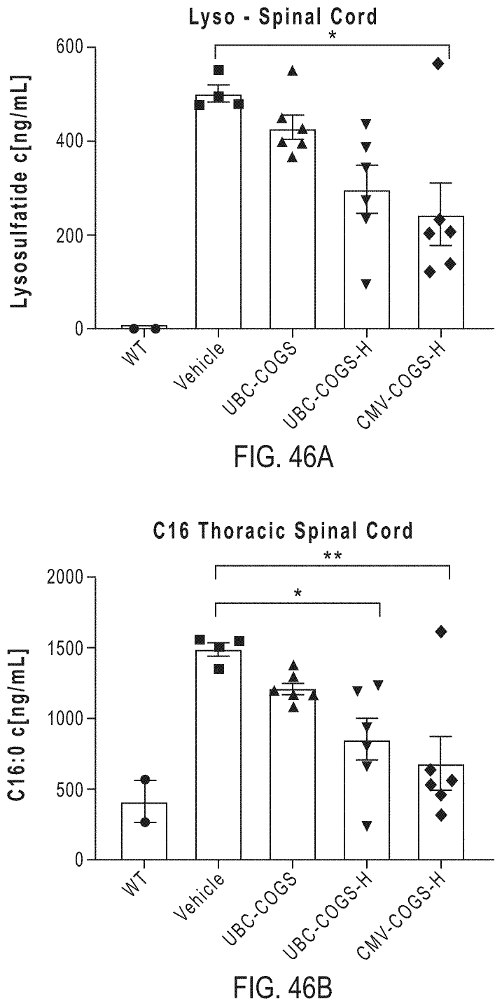

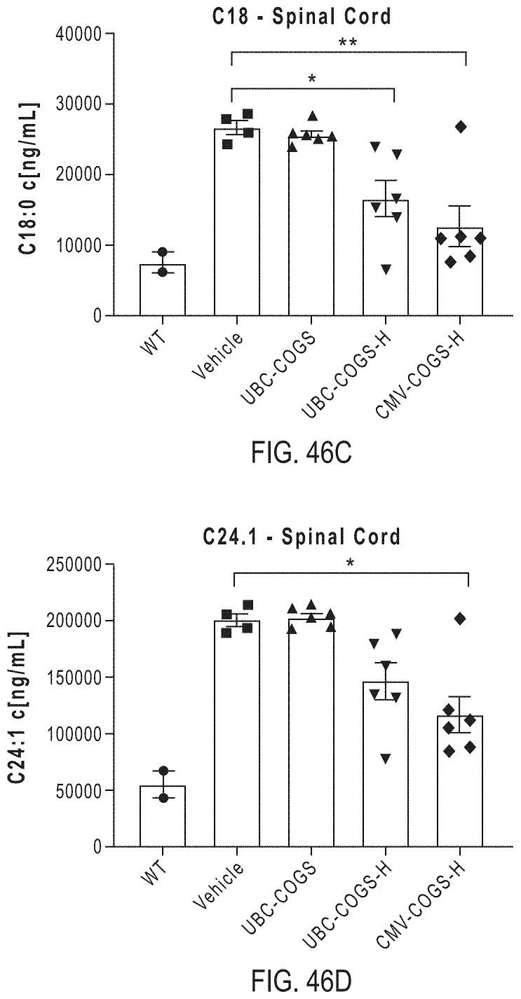

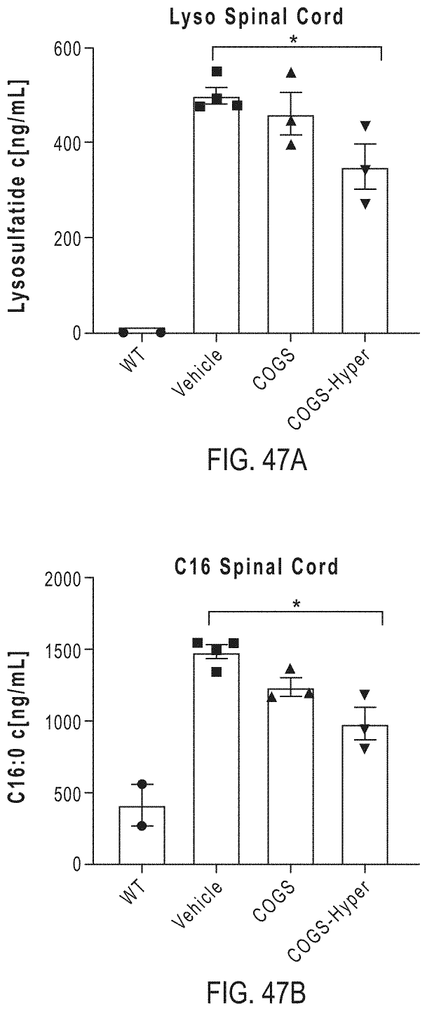

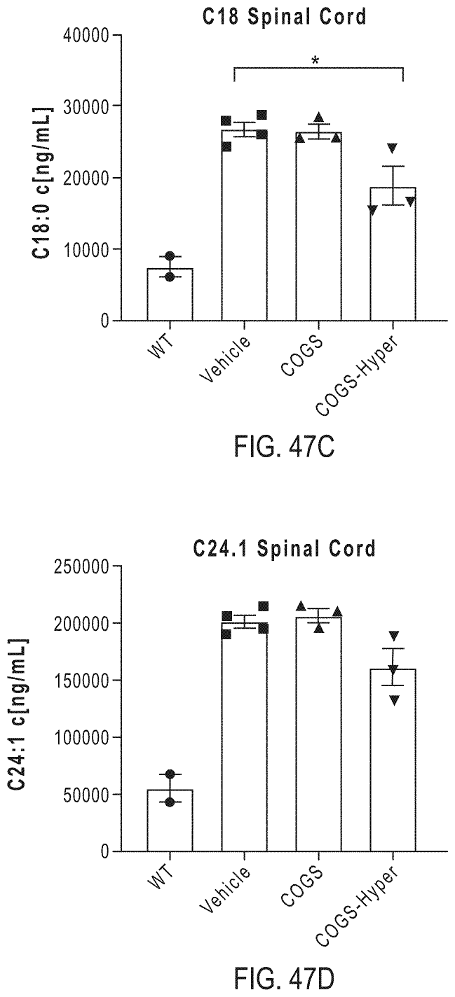

All Hyper-ARSA constructs, regardless of promoter, showed reductions in lysosulfatide (FIG. 46A) and sulfatides (FIGS. 46B-46D) in the thoracic spinal cord. Between the UbC constructs, the Hyper-ARSA construct showed greater reductions in lysosulfatide (FIG. 47A) and sulfatides (FIGS. 47B-47D) in the thoracic spinal cord when comparing data from high ARSA expressing mice.

In summary, significant changes in lysosulfatide and C16 sulfatide in Slab 1 (the area of highest transduction) were observed for UbC-COGS and UbC-COGS-Hyper constructs. Statistically significant changes in sulfatide levels were not observed in brain slabs 3, 6, and 9, possibly due to low AAV transduction in these regions (greater distance from injection site). Significant changes in lysosulfatide and sulfatide levels were observed for UbC-COGS-Hyper and CMV-COGS-Hyper constructs in thoracic spinal cord. Without being bound by theory, reducing lysosulfatide and sulfatide levels is believed to be therapeutic, therefore supporting the use of the rAAVs described herein for treating MLD.

### 7.9.2.2 DNA Distribution and ARSA RNA Expression

DNA biodistribution in slab 7 (VGC/DG) and RNA expression in slab 8 (% of reference) as observed in treated brains from ARSA knockout mice are shown in Table 15. UbC-COGS-Hyper showed higher levels of vector genome biodistribution and RNA expression than the other constructs evaluated.

TABLE 15

| | DNA Biodistribution | RNA Expression |
|---|---|---|
| Construct | (VGC/DG) | (% of Reference) |
| UbC-COGS | 0.05 | 47.6 |
| UbC-COGS-Hyper | 1.58 | 209.1 |
| CMV-COGS-Hyper | 0.02 | 12.5 |

### 7.9.2.3 ARSA Protein Expression

Normalized protein expression levels and protein expression as a percentage of wild-type expression are shown in Table 16. Normalized protein expression is the peak area adjusted for total protein load. Percentage of WT is the peak area of treated samples divided by the average of the peak area from untreated wild-type mice, presented as a percentage. With both measures, the UbC-COGS-Hyper construct produced the highest levels of protein.

TABLE 16

| | Normalized Protein | Protein |
|---|---|---|
| | Expression | Expression |
| Construct | (Peak Area) | (% of WT) |
| UbC-COGS | 872121.4 | 777.9 |
| UbC-COGS-Hyper | 1646020.8 | 927.5 |
| CMV-COGS-Hyper | 487543.4 | 259.1 |

### 7.10. Example 9: rAAV Manufacturability

rAAVs of Example 4 were assessed for manufacturability. In particular, genomic integrity, harvest yield, capsid purity, and polydispersity were assessed for selected constructs of Example 4.

### 7.10.1. Genomic Integrity

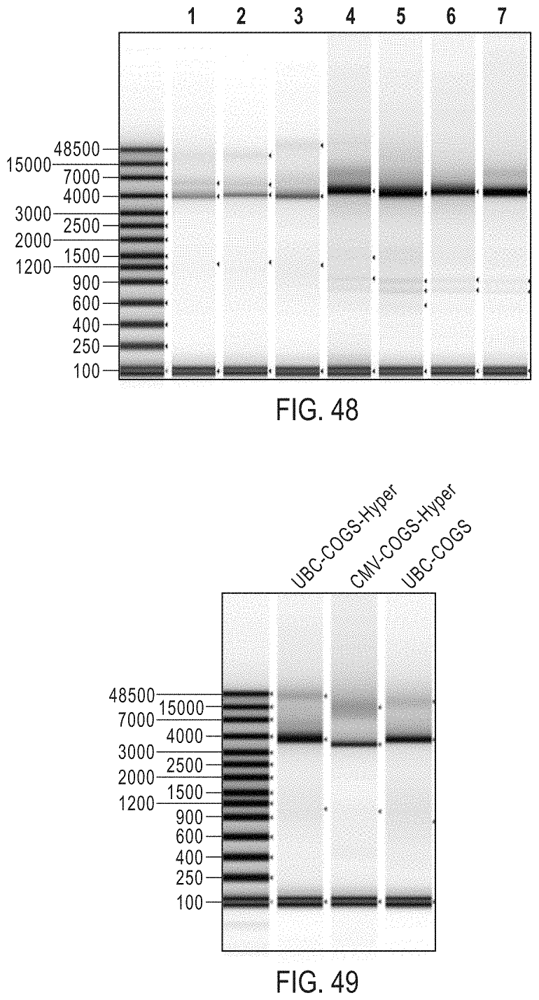

In initial studies with UbC and CAG constructs, CAG constructs were observed to have multiple lower sized bands when analyzed by the Agilent TapeStation system (FIG. 48, lanes labeled 4-7). In contrast, when UbC-COGS, UbC-COS-Hyper and CMV-COGS-Hyper vectors were analyzed, the smaller bands were not observed, indicating comparable genomic integrity of the UbC-COGS, UbC-COS-Hyper and CMV-COGS-Hyper vectors (FIG. 49).

### 7.10.2. Harvest Yield

Two different lots of the UbC-COGS, UbC-COS-Hyper, and CMV-COGS-Hyper vectors were produced along with a small scale run to assess vector yield.

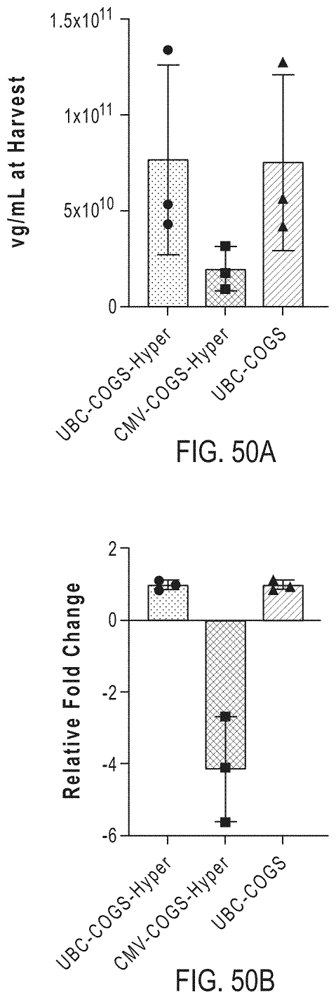

For each vector, some run-to-run variation was observed, but yield of the CMV construct was consistently lower than the UbC constructs (FIGS. 50A-50B).

### 7.10.3. Capsid Purity

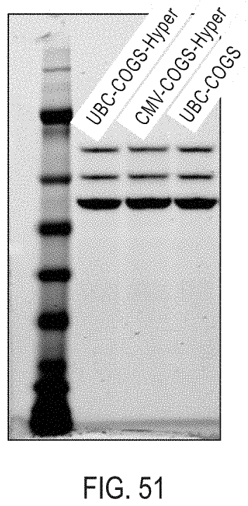

Capsid purity for UbC-COGS, UbC-COS-Hyper, and CMV-COGS-Hyper vectors was assessed by SGS-PAGE. VP1:VP2:VP3 ratios were as expected for each vector, with no other bands observed (FIG. 51).

### 7.10.4. Polydispersity

Polydispersity of the UbC-COGS, UbC-COGS-Hyper, and CMV-COGS-Hyper vectors were assessed by analytical ultracentrifugation (AUC). AUC data was comparable for all vectors (Table 17).

TABLE 17

| rAAV | Full/Partial/Empty |
|---|---|
| UbC-COGS-Hyper | 83.1%/11.4%/5.5% |
| CMV-COGS-Hyper | 80.6%/11.8%/7.6% |
| UbC-COGS | 84.6%/11.1%/4.3% |

### 7.11. Example 10: Study of ARSA-Encoding rAAV in Aged ARSA KO Mouse

A study to evaluate the therapeutic efficacy of the UbC-COGS-Hyper construct (Example 4) in aged ARSA KO mice was performed.

### 7.11.1. Materials and Methods

### 7.11.1.1 Study Design

Eight-month-old ARSA knockout (KO) (see Example 7) or ARSA+/− (Het) mice were assigned to the following four treatment groups.

TABLE 18

| | | | | | vg/g | | |
|---|---|---|---|---|---|---|---|
| | | | Dose | Dose | brain | Timepoint | |
| Group | Genotype | Treatment | level | (vg/mouse) | weight | (months) | N-value |
| 1 | Het | Formulation | NA | NA | | 3 | 6 |
| | | buffer | | | | | |
| 2 | KO | Formulation | NA | Na | | 3 | 4 |
| | | buffer | | | | | |
| 3 | KO | UbC-COGS- | low | 2e10 | 5e10 | 3 | 7 |
| | | Hyper | | | | | |
| 4 | KO | UbC-COGS- | high | 2e11 | 5e11 | 3 | 8 |
| | | Hyper | | | | | |

Formulation buffer = 1 × PBS with 172 mM NaCl (total) and 0.001% poloxamer 188

Treatments were administered via ICV injection at 9 months of age. Behavioral assessments were performed at 8 months of age before dosing and at 12 months of age prior to sacrifice. Biochemical assessments (brain weight, body weight, sulfatide levels, lysosulfatide levels, vector genome distribution (VGC/DG), ARSA enzyme activity, and RNA expression) were performed at necropsy (12 months of age).

### 7.11.1.2 Behavioral Assessments

### 7.11.1.2.1 Rotarod

Coordination and balance were measured by the rotarod test (RotaRod; Ugo Basile). A decrease in the latency to fall indicates coordination impairment. Briefly, testing consisted of an acclimation phase (Day 1), conditioning phase (Day 2), and testing phase (Day 3). For each phase, mice were gently lifted by the tail and placed on their lane (up to 4 mice per trial) facing away from the tester. During the acclimation phase, three acclimation trials were performed with the rod rotating for 2 minute (120 seconds) at a constant speed of 5 revolutions per minute (RPM). Mice that fell off will immediately were placed back on the rotarod. In the conditioning phase, mice were placed on the rod starting at 5 RPM and then the speed was accelerated from 5 RPM to 40 RPM over 5 minutes (300 seconds). If animals fell off the rod, they were not placed back on the rotarod and were returned to their cages. For the test phase, the procedure was the same as the conditioning phase except that the fall latency (defined as the time between the initiation of rod acceleration and trial termination) for each animal was recorded. For each animal, the testing trial was considered terminated when the mouse fell off the rod, completed two passive revolutions, or 5 minutes had elapsed. A total of three sequential replicates was performed for the mice in each trial, with a 1-3 minute pause between runs to allow the animals to rest. All testing was performed by personnel blinded to the treatment group.

### 7.11.1.2.2 Splay

Motor dysfunction was measured by assessing hindlimb clasping (splay) behavior. Briefly, mice were suspended by the base of the tail for no more than 15 seconds and the position of their hindlimbs recorded; the behavior was scored from 0 to 3 according to the following criteria:

| Observation | Score |
|---|---|
| Hindlimbs splayed outward and away from the abdomen (Normal | 0 |
| Behavior) | |
| One hindlimb retracted inwards towards the abdomen for at | 1 |
| least 50% of the observation period | |
| Both hindlimbs partially retracted inwards towards the abdomen | 2 |
| Both hindlimbs completely retracted inwards towards the abdomen | 3 |

All assessment were performed by personnel blinded to treatment group.

### 7.11.1.2.3 Pole Test

Motor dysfunction was assessed using the pole test. Briefly, a 50-60 cm pole with 1 cm diameter attached to a stable metal base was wrapped with bandage wrap to provide a surface for mice to grip. Mice were placed at the top of the vertical pole with their head facing the top of the pole for 3 trials. Real-time assessment of the time for the mouse to turn downward (T-turn) and reach the bottom of the pole (T total) was recorded. Assessments were captured on video. Following the timed assessment, a blinded observer noted the number of descent attempts (animal faces downward and moves a full body length down the pole), and whether the animal reached the bottom of the pole for each trial. The blinded observer also noted how the animal descended the pole (straight or corkscrew) and other observations, including animal location on the pole and animal falling from the pole. Average Total time (average of three trials), number of descent attempts, number of successful trials, and the descriptive events were used to define the pole test phenotype for each animal. Significance of differences between mean scores on each trial was assessed with one-way analysis variance (ANOVA) for repeated measures or two-tailed Student's t-test for comparison between pairs of means. Poisson regression was used to test significant success rate difference among each treatment group adjusting for baseline characteristics (gender, body weight, etc.).

### 7.11.1.3 Biochemical Assessments

### 7.11.1.3.1 Vector Genome Biodistribution (ddPCR)

Distribution of vector genomes in mouse tissues were analyzed by Bio-Rad droplet digital PCR analysis. Briefly, droplet digital PCR uses TaqMan technology to generate a fluorescent signal when PCR occurs across a specific target amplicon. PCR reactions are divided into thousands of nano-droplets prior to thermal cycling. The presence of fluorescent signal is used to sort droplets into positive and negative groups. Positive droplets are counted to determine the number of template molecules in the original sample.

Vector genome copies were detected by a primer/probe set targeted within the coding region of the ARSA transgene and specific to the COGS codon-optimized sequence. RPP30 was quantified in a duplexed reaction and vector genome copies per diploid genome were reported to assess biodistribution.

### 7.11.1.3.2 ARSA Transgene Expression (RT-ddPCR)

Expression of the therapeutic transgene in mouse tissue was analyzed by Bio-Rad One-Step reverse transcription droplet digital PCR. Copies of the therapeutic gene transcript were detected by a primer/probe set targeted within the coding region of the ARSA transgene and specific to the COGS codon-optimized sequence. RPP30 transcripts were quantified in a duplex reaction and transgene expression was reported as a percentage of RPP30 expression.

### 7.11.1.3.3 Sulfatide Levels

Quantitative analysis of selected sulfatides (C16:0, C18:0, C24:0, C24:1) and lysosulfatide in mouse brain and spinal cord was performed by HPLC-MS/MS assays. The calibration curves were linear over the concentration range of 5-1000 ng/mL (R2≥0.99). Tissue samples were homogenized, followed by liquid:liquid extraction of the homogenates. Extraction steps were optimized to accommodate significant variance in levels of different sulfatides present within each sample, resulting in two final preparations per sample. Due to the presence of high levels of sulfatides in wild type mouse brain, a surrogate matrix was used for preparation of calibration standards and QC samples, and justification was performed during assay development to confirm a suitable matrix was chosen. Test samples for each tissue were analyzed by HPLC hyphenated with a tandem triple-quadrupole mass-spectrometric detection (MS), with short LC gradients comprising ACN and MeOH as organic modifiers and ammonium formate as an additive. Chromatographic separation of the analytes was followed by MRM (multiple reaction monitoring) data acquisition by MS with electrospray ionization in negative ion mode. Up to 6 precursor-product transitions were monitored within one run for the selected sulfatides, lysosulfatide, and corresponding internal standards.

### 7.11.1.3.4 ARSA Enzyme Activity

ARSA-specific sulfatase activity was evaluated in various regions of the mouse brain. Tissue was dissected and flash-frozen in liquid nitrogen at the time of sacrifice. Tissues were homogenized in a bead-beater instrument (30 Hz for 2 minutes) in a mild Tris-HCL buffer with no detergent (10 mM Tris/HCl pH7.5+ protease inhibitor) and then further processed in a Covaris ultrasonicator to completely lyse cells. Lysates were clarified by spinning at 17,000×g for 20 minutes at 4° C. The total protein concentration of the lysate was determined by BCA assay (Pierce 23225). A DEAE sepharose (Cytiva 17070910) column was prepared by equilibrating with 10CV of nuclease free water followed by 10CV of equilibration buffer (25 mM Tris/HCl pH 7.5). A volume of lysate containing ˜650 μg of total protein was added to the column and incubated for 1.5 hours at 4° C. on a rotating mixer. The column was centrifuged at 1000×g for 1 minute at 4° C. (all subsequent elutions used the same settings) and washed with 10CV of Wash Buffer (25 mM Tris/HCl+50 mM NaCl pH 7.5). A series of four elutions of 100 μL each were performed with Elution Buffer (25 mM Tris/HCl+250 mM NaCl pH 7.0). The four-step elution recovered approximately 80-90% of the ARSA enzyme activity from the samples based on spike-recovery experiments. Enzyme activity was measured using a sulfatase activity kit (Abcam 204731). The internally developed assay method used a seven-point standard curve using a 2-fold dilution series from 20 nmol to 0.3125 nmol of 4-Nitrochatechol. The LOD for this method is ˜0.3 nmol of 4-nitrochatechol. Samples were run in triplicate on the plate. Samples were incubated in reaction mix at 37° C. for 30 minutes. The OD of each well was read at 515 nm absorbance and results were averaged across the 3 technical replicates. Results are reported in units of nmol of 4-nitrochatechol/mg total protein× minute.

### 7.11.2. Results

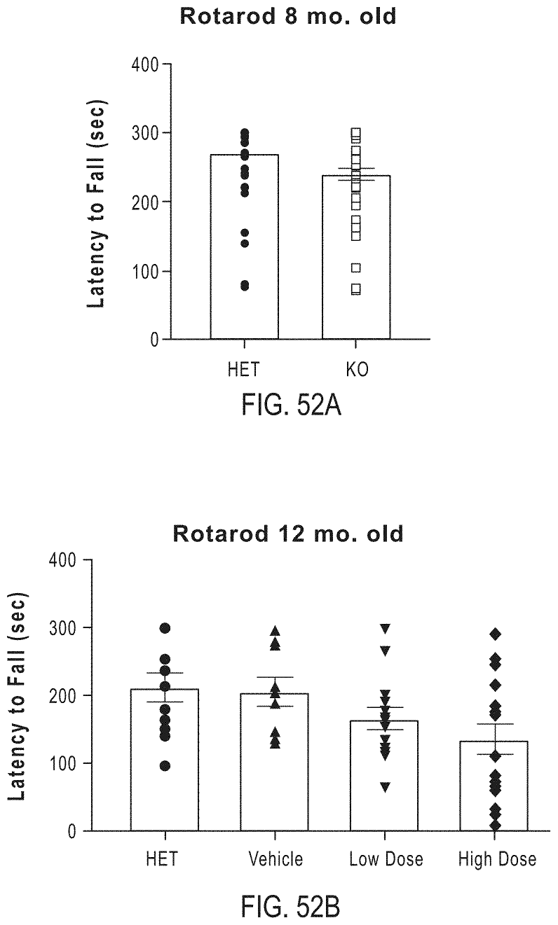

Rotarod results are shown in FIGS. 52A-52B. No statistically significant differences were observed between groups.

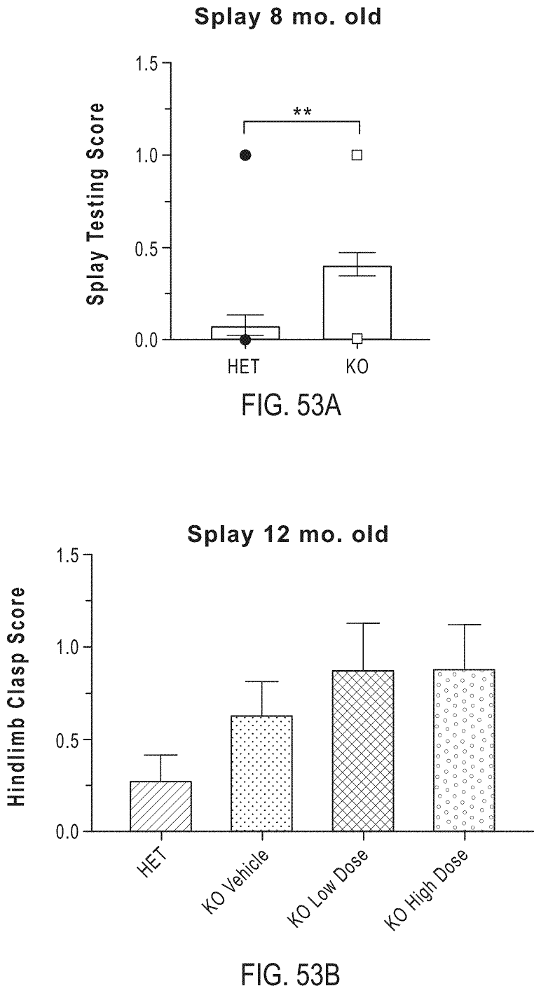

Hindlimb clasping results (splay) are shown in FIGS. 53A-53B. No statistically significant differences were observed between groups.

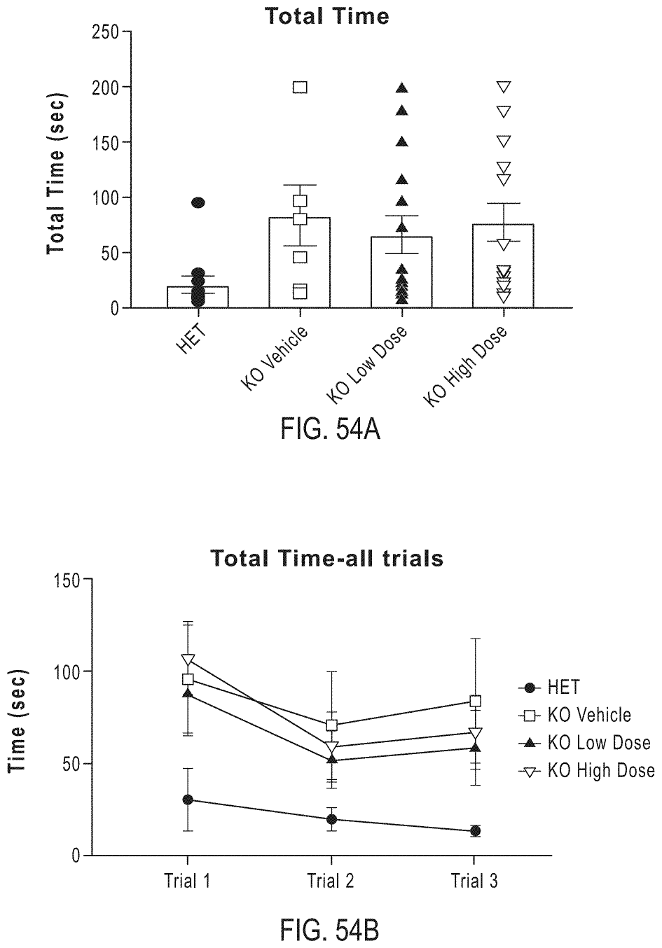

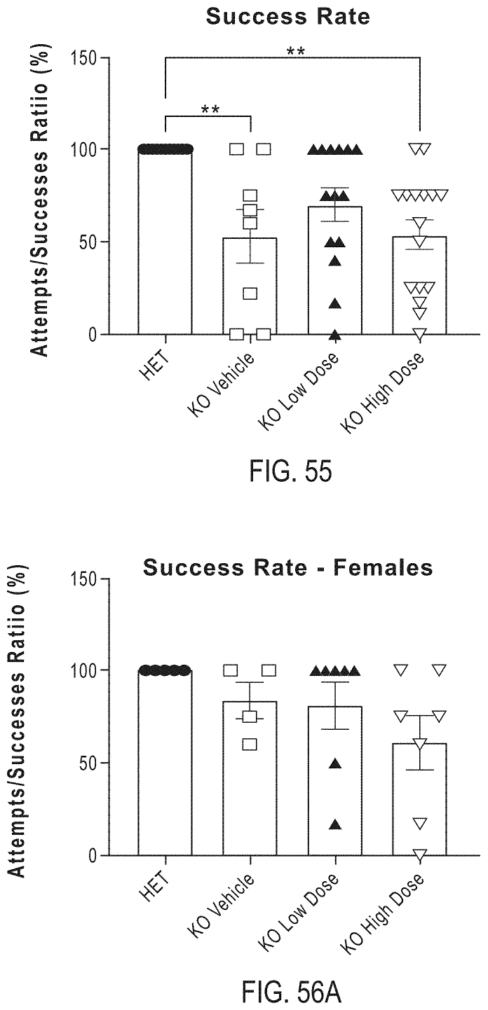

Pole test total time results are shown in FIGS. 54A-54B. A trend toward a performance deficit (not reaching statistical significance) was observed in ARSA KO mice compared to Het controls. ARSA KO mice were observed to have a reduced success rate on the pole test compared to Het mice, with low dose treated animals showing improved performance three months post-injection (FIG. 55 and Tables 19A-19B).

TABLE 19A
ANOVA summary

| | |
|---|---|
| F | 5.782 |
| P value | 0.0020 |
| P value summary | ** |
| Significant diff. among means | Yes |
| (P < 0.05)? | |
| R squared | 0.2782 |

TABLE 19B

| Tukey's multiple | Mean | 95.00% CI | Below | | Adjusted P |
|---|---|---|---|---|---|
| comparisons test | Diff. | of diff. | threshold? | Summary | Value |
| HET vs. KO Vehicle | 47.00 | 9.042 to 84.96 | Yes | ** | 0.0098 |
| HET vs. KO Low Dose | 29.86 | −3.057 to 62.77 | No | ns | 0.0877 |
| HET vs. KO High Dose | 46.06 | 14.07 to 78.06 | Yes | ** | 0.0021 |
| KO Vehicle vs. KO Low | −17.14 | −53.35 to 19.06 | No | ns | 0.5907 |
| Dose | | | | | |
| KO Vehicle vs. KO High | −0.9375 | −36.31 to 34.44 | No | ns | 0.9999 |
| Dose | | | | | |
| KO Low Dose vs. KO | 16.21 | −13.69 to 46.10 | No | ns | 0.4780 |
| High Dose | | | | | |

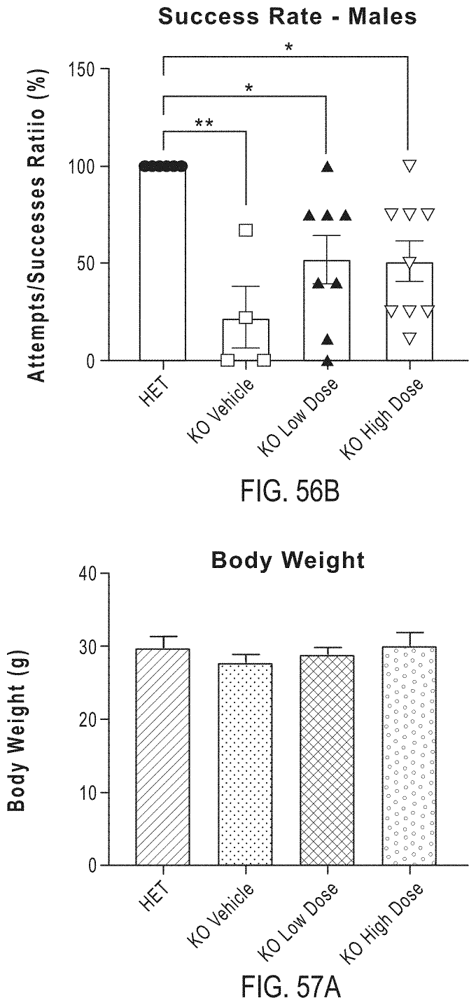

Male ARSA KO mice were observed to have a reduced success rate on the pole test compared to male HET mice, with low dose and high dose treated animals showing improved performance three months post-injection (FIGS. 56A-56B).

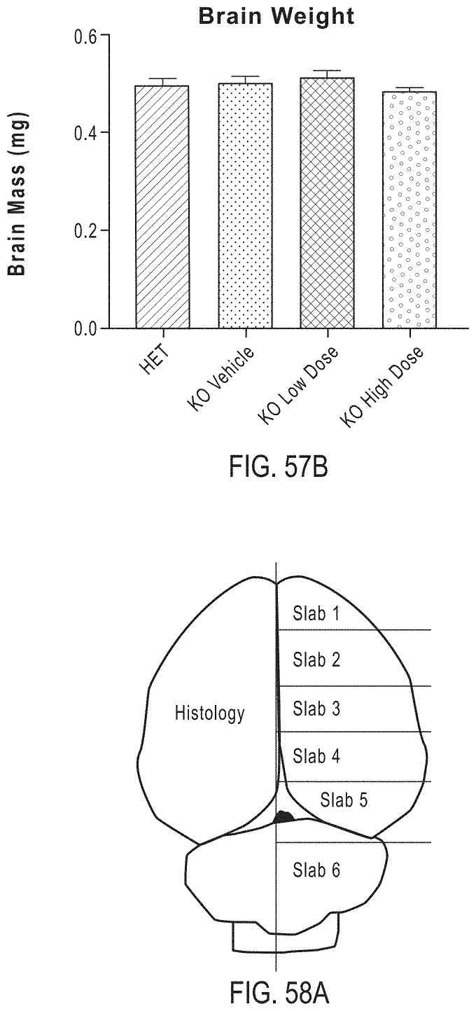

No significant differences were observed in body weight and brain weight at necropsy among the groups (FIGS. 57A-57B).

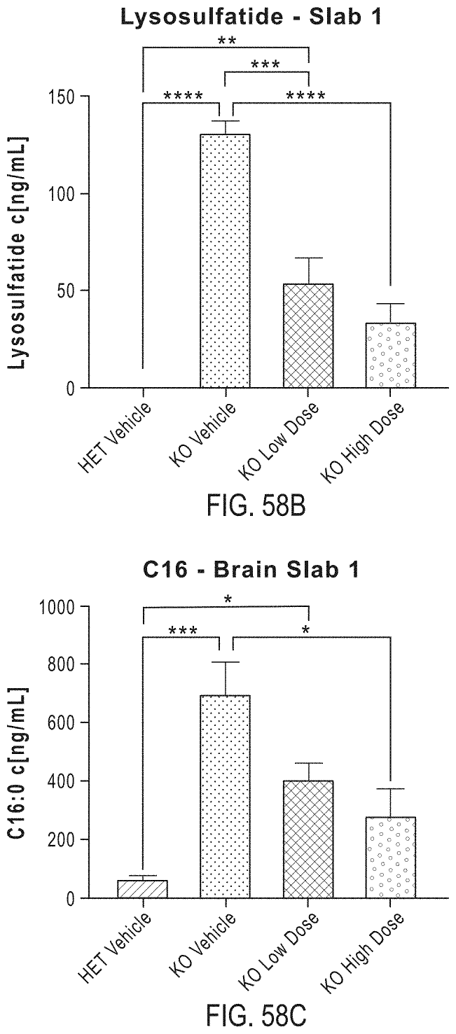

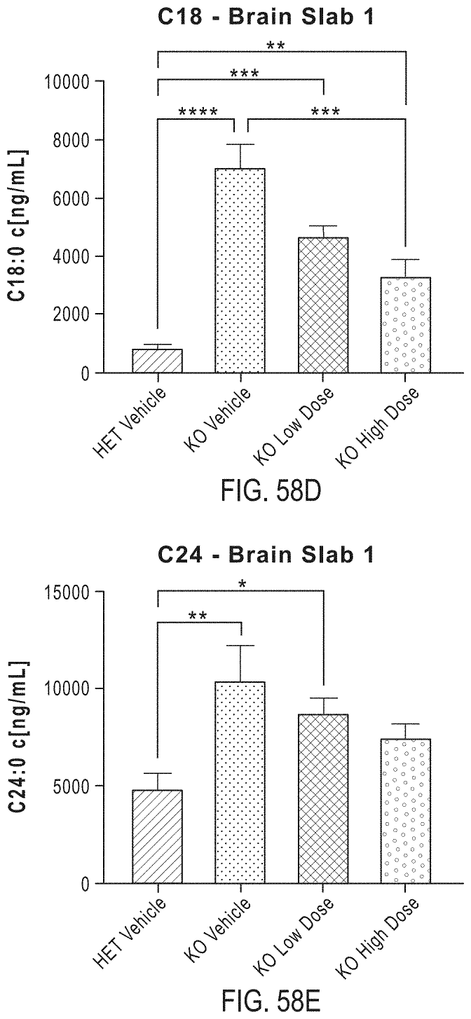

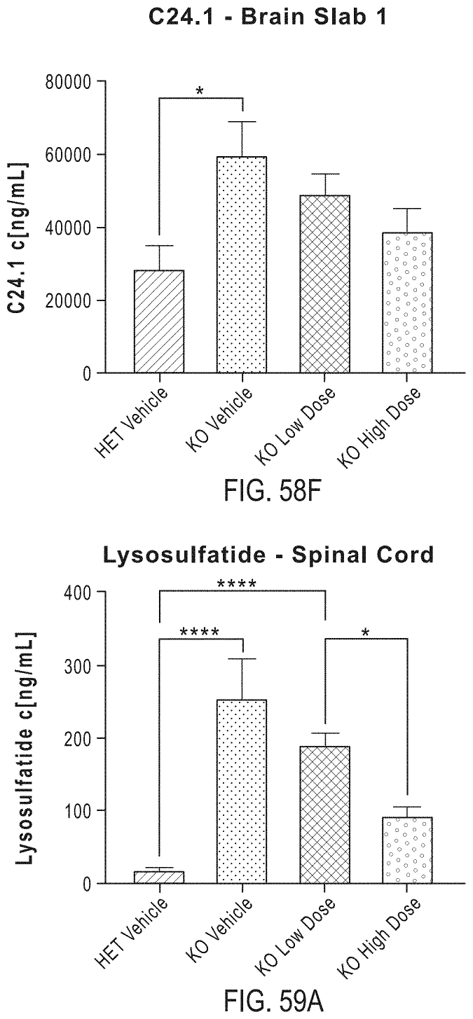

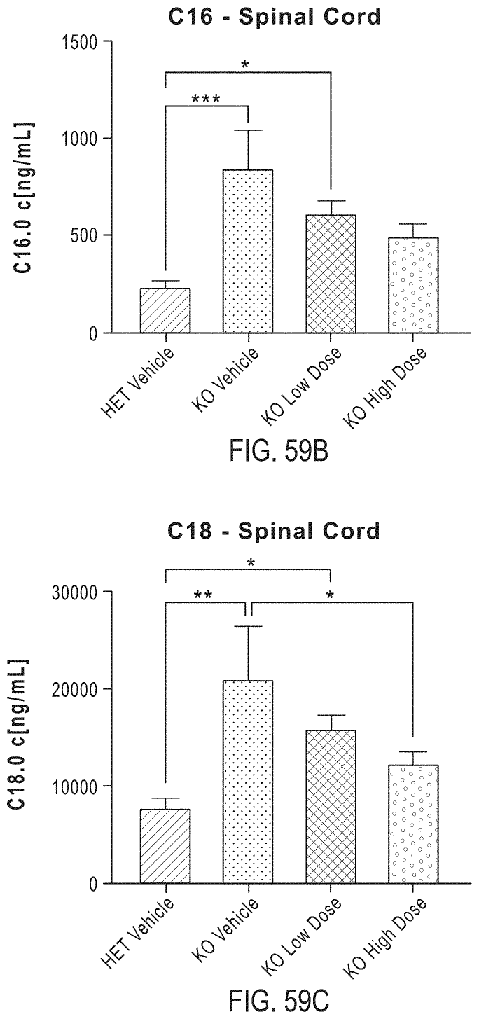

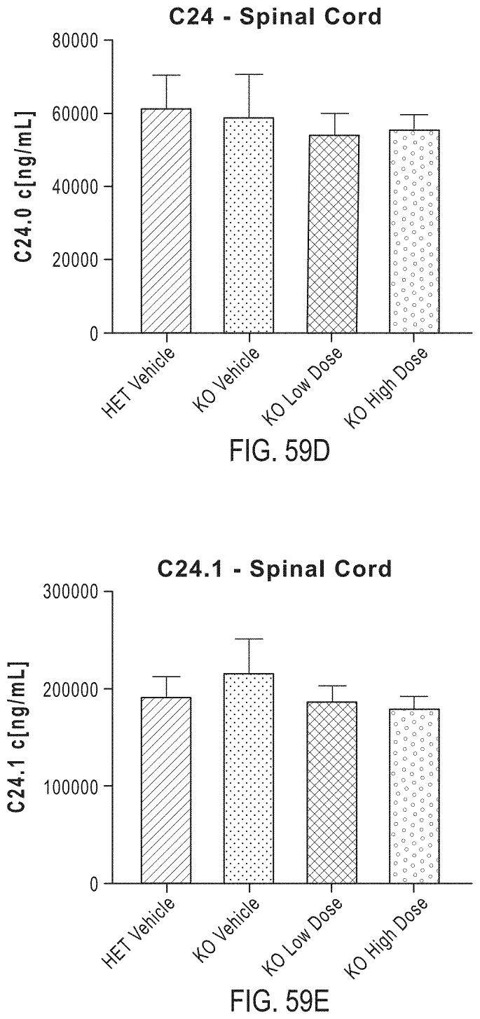

Reductions in sulfatide and lysosulfatide levels in brain slab 1 (FIG. 58A) and thoracic spinal cord were observed at the low and high dose (brain: FIGS. 58B-58F and Table 20; thoracic spinal cord: FIGS. 59A-59E).

TABLE 20
Lysosulfatide and sulfatides in brain slab 1 (ng/ml)

| | Het Vehicle | | KO Vehicle | | KO low | | KO High | |
|---|---|---|---|---|---|---|---|---|
| | Mean | St. dev. | Mean | St. dev. | Mean | St. dev. | Mean | St. dev. |
| Lyso- | 0 | 0 | 130.8625 | 12.59943 | 63.37743 | 37.97095 | 33.98075 | 26.94383 |
| sulfatide | | | | | | | | |
| C16 | 61.39783 | 38.63736 | 696 | 221.5797 | 513.6729 | 315.6153 | 279.16125 | 268.3219 |
| C18 | 815.77 | 367.4205 | 7097.75 | 1572.32 | 5175.257 | 1407.847 | 3311.8 | 1638.421 |
| C24 | 4819.8 | 2027.41 | 10405.2 | 3582.288 | 9266.743 | 2270.613 | 7431.975 | 2131.572 |
| C24.1 | 28344.6 | 15741.13 | 59899.5 | 18166.9 | 49217.14 | 13790.5 | 38867.25 | 17258.97 |

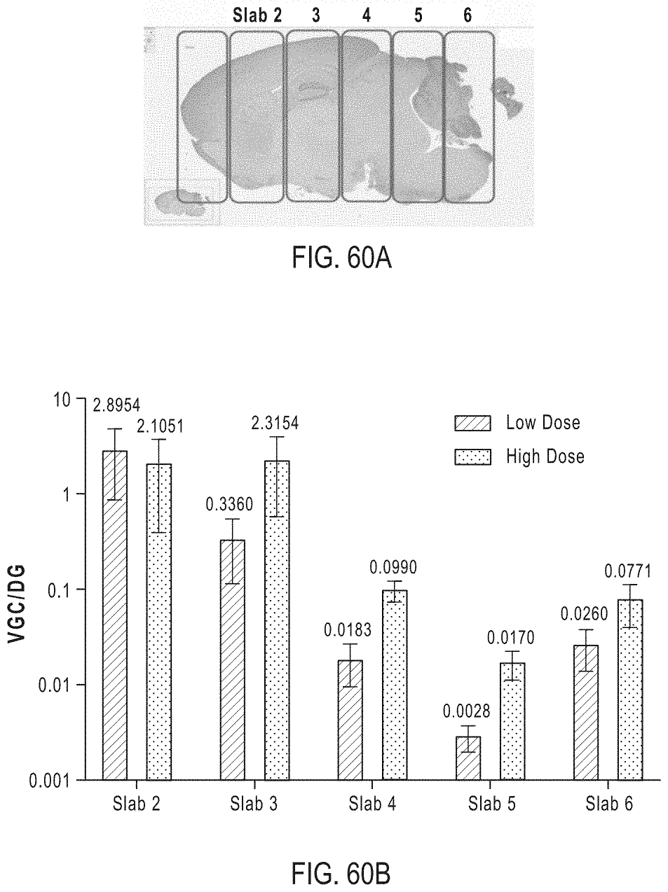

Vector genome biodistribution in different brain slabs (FIG. 60A) is shown in FIG. 60B and Table 21.

TABLE 21
Vector genome biodistribution (VGC/DG)

| | | | | | | | Average |
|---|---|---|---|---|---|---|---|
| | | Brain | Brain | Brain | Brain | Brain | across all |
| Group | N-value | slab 2 | slab 3 | slab 4 | slab 5 | slab 6 | brain slabs |
| HET vehicle | 6 | 0 | 0 | 0 | 0 | 0 | 0 |
| KO vehicle | 4 | 0 | 0 | 0 | 0 | 0 | 0 |
| KO low dose | 7 | 2.9 | 0.34 | 0.02 | 0.003 | 0.03 | 0.659 |
| KO high dose | 8 | 2.11 | 2.3 | 0.10 | 0.02 | 0.10 | 0.926 |

ARSA mRNA expression is shown in Table 22.

TABLE 22
RNA expression (% of reference)

| | | | | | | | Average |
|---|---|---|---|---|---|---|---|
| | | Brain | Brain | Brain | Brain | Brain | across all |
| Group | N-value | slab 2 | slab 3 | slab 4 | slab 5 | slab 6 | brain slabs |
| HET vehicle | 6 | 0.35 | 1.05 | 0.26 | 0.2 | 0.85 | 0.54 |
| KO vehicle | 4 | 0.04 | 0.5 | 0.20 | 0.1 | 0.65 | 0.298 |
| KO low dose | 7 | 1314.4 | 2496.4 | 701.7 | 9.03 | 21.4 | 908.586 |
| KO high dose | 8 | 6332.7 | 5413.0 | 1882.9 | 123.4 | 142.5 | 27778.9 |

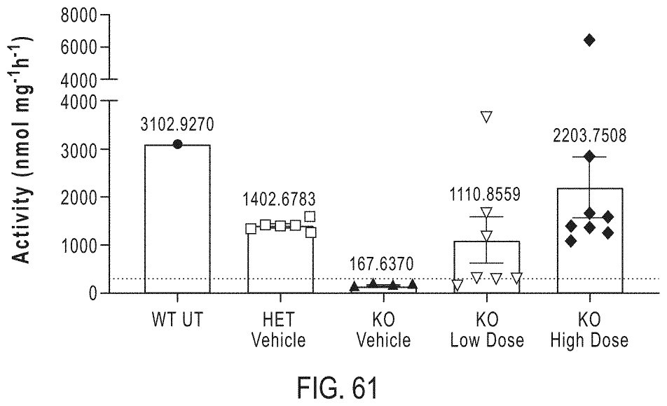

ARSA enzyme activity in brain slabs 2-6 (combined) is shown in FIG. 61.

### 8. SPECIFIC EMBODIMENTS

The present disclosure is exemplified by the specific embodiments below.

1. A method of transferring a polynucleotide to the central nervous system (CNS) of a subject, the method comprising:
administering to the subject an effective dose of:
   a recombinant adeno-associated virus (rAAV) comprising:
      a capsid comprising: a capsid protein having the amino acid
      sequence of SEQ ID NO: 1 or a variant thereof, and
      the polynucleotide encapsulated by the capsid;
thereby transferring the polynucleotide to the CNS.

2. The method of embodiment 1, wherein the polynucleotide comprises a coding sequence of a therapeutic protein.

3. The method of embodiment 2, wherein the subject has a CNS disease.

4. The method of embodiment 3, wherein the CNS disease is a lysosomal storage disease (LSD).

5. The method of embodiment 3, wherein the CNS disease is a leukodystrophy.

6. The method of embodiment 5, wherein the CNS disease is metachromatic leukodystrophy (MLD).

7. The method of embodiment 6, wherein the polynucleotide comprises a coding sequence encoding Arylsulfatase A (ARSA) or a functional variant thereof.

8. The method of embodiment 7, wherein the polynucleotide comprises a coding sequence selected from SEQ ID NO: 2-4.

9. The method of embodiment 7, wherein the polynucleotide comprises a coding sequence of SEQ ID NO: 7.

10. The method of embodiment 7, wherein the polynucleotide comprises a coding sequence of SEQ ID NO: 8.

11. The method of embodiment 5, wherein the CNS disease is Krabbe's leukodystrophy.

12. The method of embodiment 11, wherein the polynucleotide comprises a coding sequence of galactocerebroside beta-galactosidase or a functional variant thereof.

13. The method of embodiment 3, wherein the CNS disease is GM1 gangliosidosis.

14. The method of embodiment 13, wherein the polynucleotide comprises a coding sequence of galactosidase beta 1 (GLB-1) or a functional variant thereof.

15. The method of embodiment 3, wherein the CNS disease is a cancer.

16. The method of embodiment 15, wherein the CNS disease is metastatic breast cancer.

17. The method of embodiment 16, wherein the therapeutic protein is an antigen binding protein against human epidermal growth factor receptor 2 (HER2).

18. The method of embodiment 17, wherein the polynucleotide comprises a sequence of SEQ ID NO: 23.

19. The method of embodiment 1, wherein the polynucleotide comprises a coding sequence of an antigen.

20. The method of embodiment 19, wherein the antigen is a viral or bacterial antigen.

21. The method of embodiment 19, wherein the effective dose is sufficient to immunize the subject.

22. The method of embodiment 19, wherein the effective dose is sufficient to induce an immune response to the antigen.

23. The method of any one of embodiments 2-22, wherein the polynucleotide further comprises a regulatory sequence operably linked to the coding sequence.

24. The method of embodiment 23, wherein the regulatory sequence comprises a CMV promoter or a UbC promoter.

25. The method of embodiment 24, wherein the regulatory sequence comprises a UbC promoter.

26. The method of embodiment 24, wherein the regulatory sequence comprises a CMV promoter.

27. The method of embodiment 24, wherein the regulatory sequence comprises a UbC promoter and wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 9.

28. The method of embodiment 24, wherein the regulatory sequence comprises a UbC promoter having the sequence of SEQ ID NO: 9

29. The method of embodiment 24, wherein the regulatory sequence comprises a UbC promoter and wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 10.

30. The method of embodiment 24, wherein the regulatory sequence comprises a UbC promoter having the sequence of SEQ ID NO: 10.

31. The method of embodiment 24, wherein the regulatory sequence comprises a UbC promoter and wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 11.

32. The method of embodiment 24, wherein the regulatory sequence comprises a UbC promoter having the sequence of SEQ ID NO: 11.

33. The method of any one of embodiments 1-32, wherein the administration induces protein expression from the polynucleotide in the Substantia nigra of the subject.

34. The method of any one of embodiments 1-32, wherein the administration induces protein expression from the polynucleotide in the caudate nuclei of the subject.

35. The method of any one of embodiments 1-32, wherein the administration induces protein expression from the polynucleotide in the ependyma of the subject.

36. The method of any one of embodiments 1-32, wherein the administration induces protein expression from the polynucleotide in the cortex of the subject.

37. The method of any of embodiments 1-36, wherein the administration is to the cerebrospinal fluid (CSF) of the subject.

38. The method of embodiment 37, wherein the administration is selected from intrathecal administration, intracranial administration, intracerebroventricular (ICV) administration and administration to the lateral ventricles of the brain of the subject.

39. The method of embodiment 38, wherein the intrathecal administration is by lumbar puncture (LP) and/or intra cisterna magna (ICM) injection.

40. The method of embodiment 39, wherein the step of administering is performed by ICM injection.

41. The method of embodiment 39, wherein the step of administering is performed by lumbar puncture (LP).

42. The method of any one of embodiments 1-41, wherein the effective dose is between 1E10 to 1E16 genome copy numbers (GC) of the rAAV.

43. The method of any one of embodiments 1-41, wherein the effective dose is 1E9 GC to 1E14 GC per gram brain mass.

44. The method of any one of embodiments 1-41, wherein the effective dose is administered at a concentration of 1E12 GC/ml to 1E17 GC/ml.

45. The method of any one of embodiments 1-44, wherein the effective dose is administered systemically.

46. The method of embodiment 45, wherein the step of administration is performed intravenously.

47. The method of any one of embodiments 1-45, wherein the effective dose is between 1E10-1E16 genome copy numbers (GC) of the rAAV.

48. The method of any one of embodiments 1-45, wherein the effective dose is between 1E9-1E15 genome copy numbers (GC) of the rAAV per kg body weight.

49. The method of any one of embodiments 2-48, wherein the effective dose is an amount sufficient to induce detectable expression of the therapeutic protein in the CNS.

50. The method of any one of embodiments 2-48, wherein the effective dose is an amount sufficient to induce detectable expression of the therapeutic protein in the Substantia nigra.

51. The method of any one of embodiments 2-48, wherein the effective dose is an amount sufficient to induce detectable expression of the therapeutic protein in the caudate nuclei.

52. The method of any one of embodiments 2-48, wherein the effective dose is an amount sufficient to induce detectable expression of the therapeutic protein in the ependyma.

53. The method of any one of embodiments 2-48, wherein the effective dose is an amount sufficient to induce detectable expression of the therapeutic protein in the cortex.

54. A method of treating a disease of the central nervous system (CNS), the method comprising:
administering to the CNS of a subject an effective dose of:
   a recombinant adeno-associated virus (rAAV), the rAAV comprising:
      a capsid polypeptide having the amino acid sequence of SEQ ID NO: 1
      or a variant thereof, and
      a polynucleotide encoding a therapeutic protein.

55. A method of vaccination with a transgene, the method comprising:
administering to the central nervous system (CNS) of a subject an effective dose of:
   a recombinant adeno-associated virus (rAAV), the rAAV comprising:
      a capsid polypeptide having the amino acid sequence of SEQ ID NO: 1
      or a variant thereof, and
      a polynucleotide encoding an antigen.

56. A recombinant adeno-associated virus (rAAV) comprising:
a capsid comprising: a capsid protein having the amino acid sequence of SEQ ID NO: 1 or a variant thereof, and a polynucleotide encapsulated by the capsid,
wherein the polynucleotide comprises a coding sequence of a therapeutic protein associated with a CNS disease.

57. The rAAV of embodiment 56, wherein the CNS disease is metachromatic leukodystrophy (MLD).

58. The rAAV of embodiment 57, wherein the therapeutic protein is Arylsulfatase A (ARSA) or a functional variant thereof.

59. The rAAV of embodiment 58, wherein the polynucleotide comprises a coding sequence selected from SEQ ID NO: 2-4.

60. The rAAV of embodiment 58, wherein the polynucleotide comprises a coding sequence of SEQ ID NO: 7.

61. The rAAV of embodiment 58, wherein the polynucleotide comprises a coding sequence of SEQ ID NO: 8.

62. The method of embodiment 56, wherein the CNS disease is Krabbe's leukodystrophy.

63. The method of embodiment 62, wherein the polynucleotide encodes galactocerebrosidase or a functional variant thereof.

64. The rAAV of embodiment 56, wherein the CNS disease is GM1 gangliosidosis.

65. The rAAV of embodiment 64, wherein the therapeutic protein is galactosidase, beta 1 (GLB-1) or a functional variant thereof.

66. The rAAV of embodiment 56, wherein the CNS disease is cancer.

67. The rAAV of embodiment 66, wherein the CNS disease is metastatic breast cancer.

68. The rAAV of embodiment 67, wherein the therapeutic protein is an antigen binding protein (ABP) against human epidermal growth factor receptor 2 (HER2).

69. The rAAV of embodiment 68, wherein the ABP against HER2 is trastuzumab.

70. The rAAV of embodiment 68 or embodiment 69, wherein the coding sequence comprises from 5′ to 3′, a coding sequence of a heavy chain of the ABP against HER2 and a coding sequence of a light chain of the ABP against HER2.

71. The rAAV of embodiment 68 or embodiment 69, wherein the coding sequence comprises from 5′ to 3′, a coding sequence of a light chain of the ABP against HER2 and a coding sequence of a heavy chain of the ABP against HER2.

72. The rAAV of embodiment 70 or embodiment 71, wherein the coding sequence of a heavy chain comprises a sequence of SEQ ID NO: 29, 31 or 33.

73. The rAAV of any one of embodiments 70-72, wherein the coding sequence of a light chain comprises a sequence of SEQ ID NO: 30, 32 or 34.

74. The rAAV of any one of embodiments 68-73, wherein the coding sequence comprises:
a. a heavy chain coding sequence of SEQ ID NO: 29 and a light chain coding sequence of SEQ ID NO: 30;
b. a heavy chain coding sequence of SEQ ID NO: 31 and a light chain coding sequence of SEQ ID NO: 32; or
c. a heavy chain coding sequence of SEQ ID NO: 33 and a light chain coding sequence of SEQ ID NO: 34.

75. The rAAV of any one of embodiments 70-74, further comprising a self-cleaving peptide between the coding sequence of the heavy chain and the coding sequence of the light chain.

76. The rAAV of embodiment 75, wherein the self-cleaving peptide is selected from the group consisting of F2A, P2A, T2A and E2A.

77. The rAAV of embodiment 76, wherein the self-cleaving peptide has the sequence of SEQ ID NO: 37.

78. The rAAV of any one of embodiments 70-77, further comprising one or more coding sequence of interleukin 2 signal sequence (IL255).

79. The rAAV of embodiment 78, wherein one coding sequence of IL2SS is located at 5′ end of the heavy chain coding sequence.

80. The rAAV of embodiment 78, wherein one coding sequence of IL2SS is located at 5′ end of the light chain coding sequence.

81. The rAAV of embodiment 78, wherein a first coding sequence of IL2SS is located at 5′ end of the heavy chain coding sequence and a second coding sequence of IL2SS is located at 5′ end of the light chain coding sequence.

82. The rAAV of embodiment 68, wherein the polynucleotide comprises a coding sequence of SEQ ID NO: 23.

83. The rAAV of embodiment 68, wherein the polynucleotide comprises a coding sequence having at least 80%, 90%, 95%, 96%, 97%, 98%, or 99% sequence identity to SEQ ID NO: 23.

84. The rAAV of embodiment 68, wherein the polynucleotide comprises the sequence of SEQ ID NO: 24-34, or a fragment thereof.

85. The rAAV of embodiment 84, wherein the polynucleotide comprises the sequence of SEQ ID NO: 24.

86. The rAAV of embodiment 84, wherein the polynucleotide comprises the sequence of SEQ ID NO: 25.

87. The rAAV of any one of embodiments 56-86, wherein the capsid comprises a capsid protein whose amino acid sequence comprises an amino acid sequence that is at least 95% identical to SEQ ID NO: 1.

88. The rAAV of embodiment 87, wherein the capsid comprises a capsid protein whose amino acid sequence comprises an amino acid sequence that is at least 96% identical to SEQ ID NO: 1.

89. The rAAV of embodiment 87, wherein the capsid comprises a capsid protein whose amino acid sequence comprises an amino acid sequence that is at least 97% identical to SEQ ID NO: 1.

90. The rAAV of embodiment 87, wherein the capsid comprises a capsid protein whose amino acid sequence comprises an amino acid sequence that is at least 98% identical to SEQ ID NO: 1.

91. The rAAV of embodiment 87, wherein the capsid comprises a capsid protein whose amino acid sequence comprises an amino acid sequence that is at least 99% identical to SEQ ID NO: 1.

92. The rAAV of embodiment 87, wherein the capsid comprises a capsid protein whose amino acid sequence comprises an amino acid sequence that is 100% identical to SEQ ID NO: 1.

93. The rAAV of any one of embodiments 56-92, wherein the polynucleotide further comprises a regulatory sequence operably linked to the coding sequence.

94. The rAAV of embodiment 93, wherein the regulatory sequence comprises a CMV promoter or a UbC promoter.

95. The rAAV of embodiment 93, wherein the regulatory sequence comprises a UbC promoter.

96. The rAAV of embodiment 94, wherein the regulatory sequence comprises a UbC promoter and wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 9.

97. The rAAV of embodiment 94, wherein the regulatory sequence comprises a UbC promoter having the sequence of SEQ ID NO: 9.

98. The rAAV of embodiment 94, wherein the regulatory sequence comprises a UbC promoter and wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 10.

99. The rAAV of embodiment 94, wherein the regulatory sequence comprises a UbC promoter having the sequence of SEQ ID NO: 10.

100. The rAAV of embodiment 94, wherein the regulatory sequence comprises a UbC promoter and wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 11.

101. The rAAV of embodiment 94, wherein the regulatory sequence comprises a UbC promoter having the sequence of SEQ ID NO: 11.

102. A recombinant adeno-associated virus (rAAV) comprising:
a. a capsid comprising a capsid protein whose amino acid sequence comprises the amino acid sequence of SEQ ID NO: 1 or a variant thereof; and
b. a polynucleotide encapsulated by the capsid, wherein the polynucleotide comprises, in the 5′ to 3′ direction, (i) a 5′ inverted terminal repeat (ITR), (ii) a promoter which is a UbC promoter, a CAG promoter, or a CMV promoter, (iii) a coding sequence of Arylsulfatase A (ARSA) or a functional variant thereof, and (iv) a 3′ ITR.

103. The rAAV of embodiment 102, wherein the capsid comprises a capsid protein whose amino acid sequence comprises an amino acid sequence that is at least 95% identical to SEQ ID NO: 1.

104. The rAAV of embodiment 102, wherein the capsid comprises a capsid protein whose amino acid sequence comprises an amino acid sequence that is at least 96% identical to SEQ ID NO: 1.

105. The rAAV of embodiment 102, wherein the capsid comprises a capsid protein whose amino acid sequence comprises an amino acid sequence that is at least 97% identical to SEQ ID NO: 1.

106. The rAAV of embodiment 102, wherein the capsid comprises a capsid protein whose amino acid sequence comprises an amino acid sequence that is at least 98% identical to SEQ ID NO: 1.

107. The rAAV of embodiment 102, wherein the capsid comprises a capsid protein whose amino acid sequence comprises an amino acid sequence that is at least 99% identical to SEQ ID NO: 1.

108. The rAAV of embodiment 102, wherein the capsid comprises a capsid protein whose amino acid sequence comprises an amino acid sequence that is 100% identical to SEQ ID NO: 1.

109. The rAAV of any one of embodiments 102-108, wherein the coding sequence is codon optimized for human cells.

110. The rAAV of any one of embodiments 102-109, wherein the coding sequence encodes ARSA or a functional variant thereof whose amino acid sequence is at least 95%, at least 96%, at least 97%, or at least 98%, at least 99%, or 100% identical to SEQ ID NO: 5.

111. The rAAV of any one of embodiments 102-110, wherein the coding sequence encodes an ARSA functional variant having one or more amino acid substitutions relative to the amino acid sequence of SEQ ID NO: 5.

112. The rAAV of embodiment 111, wherein the coding sequence encodes an ARSA functional variant comprising M202V and/or T286L and/or R291N substitutions, wherein the position(s) of the substitution(s) is/are identified by reference to the amino acid numbering in SEQ ID NO: 5.

113. The rAAV of embodiment 112, wherein the coding sequence encodes an ARSA functional variant comprising M202V, T286L, and R291N substitutions.

114. The rAAV of embodiment 113, wherein the coding sequence encodes an ARSA functional variant whose amino acid sequence comprises the amino acid sequence of SEQ ID NO: 6.

115. The rAAV of embodiment 114, wherein the coding sequence comprises a nucleotide sequence having at least 80%, at least 85%, at least 90%, at least 95%, at least 96%, at least 97%, or at least 98%, at least 99%, or 100% sequence identity to SEQ ID NO: 7.

116. The rAAV of embodiment 115, wherein the coding sequence comprises the nucleotide sequence of SEQ ID NO: 7.

117. The rAAV of embodiment 114, wherein the coding sequence comprises a nucleotide sequence having at least 80%, at least 85%, at least 90%, at least 95%, at least 96%, at least 97%, or at least 98%, at least 99%, or 100% sequence identity to SEQ ID NO: 8.

118. The rAAV of embodiment 117, wherein the coding sequence comprises the nucleotide sequence of SEQ ID NO: 8.

119. The rAAV of any one of embodiments 102-110, wherein the coding sequence encodes ARSA or a functional variant thereof whose amino acid sequence comprises the amino acid sequence of SEQ ID NO: 5

120. The rAAV of embodiment 119, wherein the coding sequence comprises a nucleotide sequence having at least 80%, at least 85%, at least 90%, at least 95%, at least 96%, at least 97%, or at least 98%, at least 99%, or 100% sequence identity to SEQ ID NO: 2.

121. The rAAV of embodiment 120, wherein the coding sequence comprises the nucleotide sequence of SEQ ID NO: 2.

122. The rAAV of embodiment 119, wherein the coding sequence comprises a nucleotide sequence having at least 80%, at least 85%, at least 90%, at least 95%, at least 96%, at least 97%, or at least 98%, at least 99%, or 100% sequence identity to SEQ ID NO: 3.

123. The rAAV of embodiment 122, wherein the coding sequence comprises the nucleotide sequence of SEQ ID NO: 3.

124. The rAAV of embodiment 119, wherein the coding sequences comprises a nucleotide sequence having at least 80%, at least 85%, at least 90%, at least 95%, at least 96%, at least 97%, or at least 98%, at least 99%, or 100% sequence identity to SEQ ID NO: 4.

125. The rAAV of any one of embodiments 102-124, wherein the promoter is a UbC promoter.

126. The rAAV of embodiment 125, wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 9.

127. The rAAV of embodiment 126, wherein the nucleotide sequence of the UbC promoter comprises the nucleotide sequence of SEQ ID NO: 9.

128. The rAAV of any one of embodiments 125-127, wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 10.

129. The rAAV of embodiment 128, wherein the nucleotide sequence of the UbC promoter comprises the nucleotide sequence of SEQ ID NO: 10.

130. The rAAV of any one of embodiments 125-127, wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 11.

131. The rAAV of embodiment 130, wherein the nucleotide sequence of the UbC promoter comprises the nucleotide sequence of SEQ ID NO: 11.

132. The rAAV of any one of embodiments 102-124, wherein the promoter is a CAG promoter.

133. The rAAV of embodiment 132, wherein the nucleotide sequence of the CAG promoter comprises a nucleotide sequence having at least 90%, at least 95%, at least 96%, at least 97%, or at least 98%, at least 99%, or 100% sequence identity to SEQ ID NO: 12.

134. The rAAV of embodiment 133, wherein the nucleotide sequence of the CAG promoter comprises the nucleotide sequence of SEQ ID NO: 12.

135. The rAAV of any one of embodiments 102-124, wherein the promoter is a CMV promoter.

136. The rAAV of embodiment 135, wherein the nucleotide sequence of the CMV promoter comprises a nucleotide sequence having at least 90%, at least 95%, at least 96%, at least 97%, or at least 98%, at least 99%, or 100% sequence identity to SEQ ID NO: 13.

137. The rAAV of embodiment 136, wherein the nucleotide sequence of the CMV promoter comprises the nucleotide sequence of SEQ ID NO: 13.

138. The rAAV of any one of embodiments 135-137, which comprises a CMV enhancer-promoter.

139. The rAAV of embodiment 138 wherein the nucleotide sequence of the CMV enhancer-promoter comprises a nucleotide sequence having at least 90%, at least 95%, at least 96%, at least 97%, or at least 98%, at least 99%, or 100% sequence identity to SEQ ID NO: 14.

140. The rAAV of embodiment 139, wherein the nucleotide sequence of the CMV promoter enhancer comprises the nucleotide sequence of SEQ ID NO: 14.

141. The rAAV of any one of embodiments 102-140, wherein the polynucleotide further comprises a post-transcriptional regulatory element 3′ to the polynucleotide encoding the ARSA or a functional variant thereof.

142. The rAAV of embodiment 141, wherein the post transcriptional regulatory element comprises a woodchuck hepatitis virus post-transcriptional regulatory element (WPRE).

143. The rAAV of embodiment 142, wherein the nucleotide sequence of the WPRE comprises a nucleotide sequence having at least 90%, at least 95%, at least 96%, at least 97%, or at least 98%, at least 99%, or 100% sequence identity to SEQ ID NO: 15.

144. The rAAV of embodiment 143, wherein nucleotide sequence of the WPRE comprises the nucleotide sequence of SEQ ID NO: 15.

145. The rAAV of any one of embodiments 102-144, wherein the polynucleotide further comprises a polyadenylation signal sequence 3′ to the polynucleotide encoding the ARSA or a functional variant thereof.

146. The rAAV of embodiment 145, wherein the polyadenylation signal sequence comprises a SV40 late polyadenylation signal sequence.

147. The rAAV of embodiment 146, wherein the nucleotide sequence of the SV40 late polyadenylation signal sequence comprises the nucleotide sequence of SEQ ID NO: 16.

148. The rAAV of any one of embodiments 102-147, wherein the nucleotide sequence of the 5′ ITR comprises the nucleotide sequence of SEQ ID NO: 17.

149. The rAAV of any one of embodiments 102-148, wherein the nucleotide sequence of the 3′ ITR comprises the nucleotide sequence of SEQ ID NO: 18.

150. The rAAV of any one of embodiments 102-149, wherein the polynucleotide comprises, in the 5′ to 3′ direction, the 5′ ITR, the promoter, the coding sequence, a post-transcriptional regulatory element, a polyadenylation signal sequence, and the 3′ ITR.

151. The rAAV of embodiment 102, wherein the polynucleotide comprises the nucleotide sequence of SEQ ID NO: 19.

152. The rAAV of embodiment 102, wherein the polynucleotide comprises the nucleotide sequence of SEQ ID NO: 20.

153. The rAAV of embodiment 102, wherein the polynucleotide comprises the nucleotide sequence of SEQ ID NO: 21.

154. The rAAV of embodiment 102, wherein the polynucleotide comprises the nucleotide sequence of SEQ ID NO: 22.

155. The rAAV of any one of embodiments 56-154, wherein the capsid comprises a VP2 capsid protein.

156. The rAAV of embodiment 155, wherein the capsid comprises a VP2 capsid protein whose amino acid sequence comprises an amino acid sequence that is at least 95% identical to amino acids 138 to 736 of SEQ ID NO: 1.

157. The rAAV of embodiment 155, wherein the capsid comprises a VP2 capsid protein whose amino acid sequence comprises an amino acid sequence that is at least 96% identical to amino acids 138 to 736 of SEQ ID NO: 1.

158. The rAAV of embodiment 155, wherein the capsid comprises a VP2 capsid protein whose amino acid sequence comprises an amino acid sequence that is at least 97% identical to amino acids 138 to 736 of SEQ ID NO: 1.

159. The rAAV of embodiment 155, wherein the capsid comprises a VP2 capsid protein whose amino acid sequence comprises an amino acid sequence that is at least 98% identical to amino acids 138 to 736 of SEQ ID NO: 1.

160. The rAAV of embodiment 155, wherein the capsid comprises a VP2 capsid protein whose amino acid sequence comprises an amino acid sequence that is at least 99% identical to amino acids 138 to 736 of SEQ ID NO: 1.

161. The rAAV of embodiment 155, wherein the capsid comprises a VP2 capsid protein whose amino acid sequence comprises an amino acid sequence that is 100% identical to amino acids 138 to 736 of SEQ ID NO: 1.

162. The rAAV of any one of embodiments 56-162, wherein the capsid comprises a VP3 capsid protein.

163. The rAAV of embodiment 162, wherein the capsid comprises a VP3 capsid protein whose amino acid sequence comprises an amino acid sequence that is at least 95% identical to amino acids 203 to 736 of SEQ ID NO: 1.

164. The rAAV of embodiment 162, wherein the capsid comprises a VP3 capsid protein whose amino acid sequence comprises an amino acid sequence that is at least 96% identical to amino acids 203 to 736 of SEQ ID NO: 1.

165. The rAAV of embodiment 162, wherein the capsid comprises a VP3 capsid protein whose amino acid sequence comprises an amino acid sequence that is at least 97% identical to amino acids 203 to 736 of SEQ ID NO: 1.

166. The rAAV of embodiment 162, wherein the capsid comprises a VP3 capsid protein whose amino acid sequence comprises an amino acid sequence that is at least 98% identical to amino acids 203 to 736 of SEQ ID NO: 1.

167. The rAAV of embodiment 162, wherein the capsid comprises a VP3 capsid protein whose amino acid sequence comprises an amino acid sequence that is at least 99% identical to amino acids 203 to 736 of SEQ ID NO: 1.

168. The rAAV of embodiment 162, wherein the capsid comprises a VP3 capsid protein whose amino acid sequence comprises an amino acid sequence that 100% identical to amino acids 203 to 736 of SEQ ID NO: 1.

169. A pharmaceutical composition comprising the rAAV of any one of embodiments 56-168.

170. A unit dose comprising the pharmaceutical composition of embodiment 169.

171. A method of transferring a polynucleotide to the central nervous system (CNS) of a subject, the method comprising administering to the subject an effective dose of the recombinant adeno-associated virus (rAAV) of any one of embodiments 56-168, the pharmaceutical composition of embodiment 169 or the unit dose of embodiment 170.

172. The method of embodiment 171, wherein the subject has a mutation in the subject's ARSA gene.

173. The method of embodiment 171 or embodiment 172, wherein the subject has an ARSA protein deficiency.

174. The method of any one of embodiments 171-173, wherein the subject has metachromatic leukodystrophy (MLD).

175. The method of embodiment 174, wherein the polynucleotide comprises a coding sequence of ARSA or a functional variant thereof and wherein the effective dose is an amount effective to ameliorate a symptom of the MLD and/or slow or delay disease progression.

176. The method of any one of embodiments 171-175, wherein the polynucleotide comprises a coding sequence of ARSA or a functional variant thereof and wherein the administration induces expression of ARSA or functional variant thereof from the polynucleotide in the central nervous system of the subject.

177. The method of embodiment 176, wherein the administration induces expression of ARSA or functional variant thereof from the polynucleotide in the brain of the subject.

178. The method of any embodiment 176 or embodiment 177, wherein the administration induces expression of ARSA or functional variant thereof from the polynucleotide in the spinal cord of the subject.

179. The method of embodiment 176, wherein the administration induces expression of ARSA or functional variant thereof from the polynucleotide in the Substantia nigra of the subject.

180. The method of embodiment 176, wherein the administration induces expression of ARSA or functional variant thereof from the polynucleotide in the caudate nuclei of the subject.

181. The method of embodiment 176, wherein the administration induces expression of ARSA or functional variant thereof from the polynucleotide in the ependyma of the subject.

182. The method of embodiment 176, wherein the administration induces expression of ARSA or functional variant thereof from the polynucleotide in the cortex of the subject.

183. The method of any one of embodiments 171-182, wherein the administration is to the cerebrospinal fluid (CSF) of the subject.

184. The method of embodiment 183, wherein the administration is selected from intrathecal administration, intracranial administration, intracerebroventricular (ICV) administration and administration to the lateral ventricles of the brain of the subject.

185. The method of embodiment 184, wherein the intrathecal administration is by lumbar puncture (LP) and/or intra cisterna magna (ICM) injection.

186. The method of embodiment 185, wherein the step of administering is performed by ICM injection.

187. The method of embodiment 185, wherein the step of administering is performed by lumbar puncture (LP).

188. The method of any one of embodiments 171-187, wherein the polynucleotide comprises a coding sequence of ARSA or a functional variant thereof and wherein the effective dose is an amount effective to reduce sulfatide and/or lysosulfatide levels in the brain and/or spinal cord.

189. The method of any one of embodiments 171-188, wherein the effective dose is between 1E10 to 1E16 genome copy numbers (GC) of the rAAV.

190. The method of any one of embodiments 171-189, wherein the polynucleotide comprises a coding sequence of ARSA or a functional variant thereof and wherein the effective dose is less than 4E13 genome copy numbers (GC) of the rAAV.

191. The method of any one of embodiments 171-190, wherein the effective dose is 1E9 GC to 1E14 GC per gram brain mass.

192. The method of any one of embodiments 171-191, wherein the effective dose is administered at a concentration of 1E12 GC/ml to 1E17 GC/ml.

193. A method of transferring a polynucleotide to the central nervous system (CNS) of a subject, the method comprising:
administering to the CNS an effective dose of:
   a recombinant adeno-associated virus (rAAV) comprising:
      a capsid having the amino acid sequence of SEQ ID NO: 1 or a variant thereof, and
      a polynucleotide having the nucleic acid sequence of SEQ ID NO: 19 or 20, wherein the polynucleotide is encapsulated by the capsid,

wherein the subject has MLD.

194. A recombinant adeno-associated virus (rAAV) comprising:
a capsid having the amino acid sequence of SEQ ID NO: 1, and
a polynucleotide encapsulated by the capsid having the nucleic acid sequence of SEQ ID NO: 19 or 20.

195. A method of transferring a polynucleotide to the central nervous system (CNS) of a subject, the method comprising:
administering to the CNS an effective dose of:
   a recombinant adeno-associated virus (rAAV) comprising:
      a capsid having the amino acid sequence of SEQ ID NO: 1 or a variant thereof, and
      a polynucleotide having the nucleic acid sequence of SEQ ID NO: 24 or 25, wherein the polynucleotide is encapsulated by the capsid,
wherein the subject has metastatic breast cancer.

196. A recombinant adeno-associated virus (rAAV) comprising:
a capsid having the amino acid sequence of SEQ ID NO: 1 or a variant thereof, and
a polynucleotide encapsulated by the capsid having the nucleic acid sequence of SEQ ID NO: 24 or 25.

### 9. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### 10. SEQUENCE LISTING

| | | SEQ |
|---|---|---|
| Description | Sequence | ID NO |
| Anc80L65 vp1 | MAADGYLPDWLEDNLSEGTREWWDLKPGAPKPKANQQKQDDGRGLVLP | 1 |
| capsid protein | GYKYLGPFNGLDKGEPVNAADAAALEHDKAYDQQLKAGDNPYLRYNHAD | |
| | AEFQERLQEDTSFGGNLGRAVFQAKKRVLEPLGLVEEGAKTAPGKKRPVE | |
| | QSPQEPDSSSGTGKKGQQPARKRLNFGQTGDSESVPDPQPLGEPPAAPS | |
| | GVGSNTMAAGGGAPMADNNEGADGVGNASGNWHCDSTWLGDRVITTST | |
| | RTWALPTYNNHLYKQISSQSGGSTNDNTYFGYSTPWGYFDFNRFHCHFS | |
| | PRDWQRLINNNWGFRPKKLNFKLFNIQVKEVTTNDGTTTIANNLTSTVQVF | |
| | TDSEYQLPYVLGSAHQGCLPPFPADVFMIPQYGYLTLNNGSQAVGRSSFY | |
| | CLEYFPSQMLRTGNNFQFSYTFEDVPFHSSYAHSQSLDRLMNPLIDQYLY | |
| | YLSRTQTTSGTAGNRTLQFSQAGPSSMANQAKNWLPGPCYRQQRVSKTT | |
| | NQNNNSNFAWTGATKYHLNGRDSLVNPGPAMATHKDDEDKFFPMSGVLI | |
| | FGKQGAGNSNVDLDNVMITNEEEIKTTNPVATEEYGTVATNLQSANTAPAT | |
| | GTVNSQGALPGMVWQDRDVYLQGPIWAKIPHTDGHFHPSPLMGGFGLKH | |
| | PPPQILIKNTPVPANPPTTFSPAKFASFITQYSTGQVSVEIEWELQKENSKR | |
| | WNPEIQYTSNYNKSTNVDFAVDTNGVYSEPRPIGTRYLTRNL | |
| ARSA codon | ATGAGCATGGGCGCTCCTAGAAGCCTGCTGCTGGCCCTGGCTGCCGG | 2 |
| optimized | CCTGGCCGTGGCTAGACCTCCAAACATCGTGCTGATCTTCGCCGACGA | |
| sequence(S) | CCTGGGCTATGGTGACCTGGGCTGCTACGGCCACCCCTCTTCTACAAC | |
| | ACCCAATCTGGACCAGCTGGCCGCTGGCGGCCTGAGATTCACAGACTT | |
| | CTACGTGCCAGTGTCCCTGTGCACCCCTTCTAGAGCCGCTCTCCTGAC | |
| | CGGCAGACTGCCTGTGCGGATGGGCATGTACCCCGGAGTGCTGGTGC | |
| | CCAGCAGTAGAGGAGGACTGCCTCTGGAAGAGGTGACCGTGGCCGAG | |
| | GTGCTGGCCGCCAGAGGCTACCTGACAGGAATGGCCGGAAAATGGCA | |
| | CCTGGGAGTGGGCCCAGAAGGCGCCTTCCTGCCACCACACCAGGGCT | |
| | TTCACCGGTTCCTGGGGATCCCTTACAGCCACGACCAAGGCCCTTGTC | |
| | AGAACCTGACATGCTTCCCCCCCGCCACACCTTGCGACGGCGGCTGT | |
| | GACCAGGGCCTTGTGCCTATCCCCCTGCTGGCCAACCTGAGCGTGGA | |
| | AGCCCAGCCTCCATGGCTGCCTGGCCTCGAGGCCAGATACATGGCCTT | |
| | CGCTCATGATCTGATGGCCGATGCCCAGAGACAGGACAGACCTTTTTT | |
| | CCTGTATTACGCCAGCCACCACACCCACTACCCTCAGTTCAGCGGACA | |
| | GAGCTTCGCCGAGCGGAGCGGCAGAGGCCCCTTCGGCGACAGCCTGA | |
| | TGGAACTGGACGCCGCTGTTGGAACCCTGATGACCGCCATTGGCGATC | |
| | TGGGCCTGCTCGAGGAAACCCTGGTGATCTTCACCGCCGATAACGGCC | |
| | CTGAGACAATGCGGATGTCTAGAGGCGGCTGCAGCGGCCTGCTGCGG | |
| | TGCGGCAAGGGCACCACCTACGAGGGCGGCGTGCGGGAACCCGCCC | |
| | TGGCTTTTTGGCCTGGCCACATCGCCCCTGGCGTTACCCACGAGCTGG | |
| | CTTCTAGCCTGGACCTGCTGCCCACCCTGGCCGCACTGGCCGGAGCT | |
| | CCACTGCCTAATGTGACCCTGGATGGCTTCGACCTGTCCCCTCTGCTG | |
| | CTCGGCACCGGCAAGAGCCCTAGACAGAGCCTGTTCTTCTACCCCTCC | |
| | TACCCTGATGAGGTGCGGGGCGTCTTTGCCGTCAGGACCGGCAAATAC | |
| | AAGGCCCATTTCTTTACACAGGGCAGCGCCCACTCTGATACCACAGCC | |
| | GACCCTGCCTGCCACGCCAGCTCCAGCCTGACCGCCCACGAGCCTCC | |
| | TCTGCTATACGACCTGAGCAAGGACCCTGGCGAGAACTACAACCTGCT | |
| | GGGTGGCGTGGCCGGCGCTACACCTGAGGTGCTGCAGGCCCTGAAGC | |
| | AGCTGCAGCTGCTTAAGGCCCAACTGGACGCCGCTGTGACCTTCGGC | |
| | CCTAGCCAGGTGGCCAGAGGAGAAGATCCCGCCCTGCAAATCTGCTG | |
| | CCACCCTGGATGTACCCCTCGGCCCGCTTGTTGTCACTGCCCCGACCC | |
| | TCACGCCTGA | |
| ARSA codon | ATGTCTATGGGAGCCCCTAGATCTCTGCTGCTGGCTCTGGCTGCTGGA | 3 |
| optimized | CTGGCAGTTGCCAGACCTCCTAACATCGTGCTGATCTTCGCCGACGAT | |
| sequence (A) | CTCGGCTATGGCGATCTGGGCTGTTACGGACACCCCAGCAGCACCACA | |
| | CCTAACCTGGATCAACTTGCCGCTGGCGGCCTGAGATTCACCGATTTC | |
| | TACGTGCCCGTGTCTCTGTGCACCCCTTCTAGAGCTGCTCTGCTGACA | |
| | GGCAGACTCCCTGTGCGGATGGGAATGTATCCTGGCGTGCTGGTGCC | |
| | TAGCTCTAGAGGCGGACTGCCTCTGGAAGAAGTGACAGTTGCCGAAGT | |
| | GCTGGCCGCCAGAGGATATCTGACTGGCATGGCCGGAAAGTGGCACC | |
| | TCGGAGTTGGACCTGAAGGCGCTTTTCTGCCTCCTCACCAGGGCTTCC | |
| | ACCGGTTTCTGGGCATCCCTTACTCTCACGATCAGGGCCCCTGCCAGA | |
| | ACCTGACCTGTTTTCCTCCTGCCACACCTTGCGACGGCGGCTGTGATC | |
| | AAGGACTGGTGCCAATTCCTCTGCTGGCCAACCTGAGCGTGGAAGCTC | |
| | AACCTCCTTGGCTGCCAGGACTGGAAGCCCGGTATATGGCCTTCGCTC | |
| | ACGACCTGATGGCCGACGCTCAGAGACAGGACAGACCATTCTTCCTGT | |
| | ACTACGCCAGCCACCACACACACTACCCTCAGTTTAGCGGCCAGAGCT | |
| | TCGCCGAGAGATCTGGCAGAGGACCTTTCGGCGACAGCCTGATGGAA | |
| | CTGGATGCCGCTGTGGGCACACTGATGACAGCCATCGGAGATCTGGG | |
| | ACTGCTGGAAGAGACACTGGTCATCTTCACCGCCGACAACGGCCCCGA | |
| | GACAATGAGAATGAGCAGAGGCGGCTGTAGCGGCCTGCTGAGATGTG | |
| | GCAAGGGCACCACATATGAAGGCGGCGTCAGAGAACCTGCTCTGGCC | |
| | TTTTGGCCTGGCCATATTGCTCCAGGCGTGACACACGAGCTGGCCTCT | |
| | TCTCTGGATCTGCTGCCTACACTGGCAGCTCTTGCTGGTGCTCCCCTG | |
| | CCTAATGTGACCCTGGATGGCTTCGATCTGAGCCCACTGCTGCTCGGC | |
| | ACAGGCAAGTCTCCAAGACAGAGCCTGTTCTTCTACCCTAGCTACCCC | |
| | GATGAAGTGCGGGGAGTGTTTGCCGTGCGGACCGGAAAGTATAAGGC | |
| | CCACTTCTTCACCCAAGGCAGCGCCCACTCTGACACCACAGCTGATCC | |
| | TGCTTGTCACGCCAGCTCTAGCCTGACAGCCCATGAACCTCCACTGCT | |
| | GTACGACCTGAGCAAGGACCCCGGCGAGAACTACAATCTGCTTGGCG | |
| | GAGTTGCCGGCGCTACACCTGAAGTTCTGCAGGCCCTGAAACAGCTCC | |
| | AGCTGCTGAAAGCCCAGCTGGACGCTGCCGTGACATTTGGACCTAGTC | |
| | AGGTGGCCAGAGGCGAGGATCCTGCTCTGCAGATCTGTTGTCACCCTG | |
| | GCTGCACACCCAGACCTGCCTGCTGTCATTGTCCTGATCCTCACGCCT | |
| | GA | |
| ARSA coding | ATGTCCATGGGGGCACCGCGGTCCCTCCTCCTGGCCCTGGCTGCTGG | 4 |
| sequence | CCTGGCCGTTGCCCGTCCGCCCAACATCGTGCTGATCTTTGCCGACGA | |
| (native) | CCTCGGCTATGGGGACCTGGGCTGCTATGGGCACCCCAGCTCTACCA | |
| | CTCCCAACCTGGACCAGCTGGCGGCGGGAGGGCTGCGGTTCACAGAC | |
| | TTCTACGTGCCTGTGTCTCTGTGCACACCCTCTAGGGCCGCCCTCCTG | |
| | ACCGGCCGGCTCCCGGTTCGGATGGGCATGTACCCTGGCGTCCTGGT | |
| | GCCCAGCTCCCGGGGGGGCCTGCCCCTGGAGGAGGTGACCGTGGCC | |
| | GAAGTCCTGGCTGCCCGAGGCTACCTCACAGGAATGGCCGGCAAGTG | |
| | GCACCTTGGGGTGGGGCCTGAGGGGGCCTTCCTGCCCCCCCATCAGG | |
| | GCTTCCATCGATTTCTAGGCATCCCGTACTCCCACGACCAGGGCCCCT | |
| | GCCAGAACCTGACCTGCTTCCCGCCGGCCACTCCTTGCGACGGTGGC | |
| | TGTGACCAGGGCCTGGTCCCCATCCCACTGTTGGCCAACCTGTCCGTG | |
| | GAGGCGCAGCCCCCCTGGCTGCCCGGACTAGAGGCCCGCTACATGGC | |
| | TTTCGCCCATGACCTCATGGCCGACGCCCAGCGCCAGGATCGCCCCTT | |
| | CTTCCTGTACTATGCCTCTCACCACACCCACTACCCTCAGTTCAGTGGG | |
| | CAGAGCTTTGCAGAGCGTTCAGGCCGCGGGCCATTTGGGGACTCCCT | |
| | GATGGAGCTGGATGCAGCTGTGGGGACCCTGATGACAGCCATAGGGG | |
| | ACCTGGGGCTGCTTGAAGAGACGCTGGTCATCTTCACTGCAGACAATG | |
| | GACCTGAGACCATGCGTATGTCCCGAGGCGGCTGCTCCGGTCTCTTGC | |
| | GGTGTGGAAAGGGAACGACCTACGAGGGCGGTGTCCGAGAGCCTGCC | |
| | TTGGCCTTCTGGCCAGGTCATATCGCTCCCGGCGTGACCCACGAGCTG | |
| | GCCAGCTCCCTGGACCTGCTGCCTACCCTGGCAGCCCTGGCTGGGGC | |
| | CCCACTGCCCAATGTCACCTTGGATGGCTTTGACCTCAGCCCCCTGCT | |
| | GCTGGGCACAGGCAAGAGCCCTCGGCAGTCTCTCTTCTTCTACCCGTC | |
| | CTACCCAGACGAGGTCCGTGGGGTTTTTGCTGTGCGGACTGGAAAGTA | |
| | CAAGGCTCACTTCTTCACCCAGGGCTCTGCCCACAGTGATACCACTGC | |
| | AGACCCTGCCTGCCACGCCTCCAGCTCTCTGACTGCTCATGAGCCCCC | |
| | GCTGCTCTATGACCTGTCCAAGGACCCTGGTGAGAACTACAACCTGCT | |
| | GGGGGGTGTGGCCGGGGCCACCCCAGAGGTGCTGCAAGCCCTGAAA | |
| | CAGCTTCAGCTGCTCAAGGCCCAGTTAGACGCAGCTGTGACCTTCGGC | |
| | CCCAGCCAGGTGGCCCGGGGCGAGGACCCCGCCCTGCAGATCTGCT | |
| | GTCATCCTGGCTGCACCCCCCGCCCAGCTTGCTGCCATTGCCCAGATC | |
| | CCCATGCCTGA | |
| ARSA (native) | MGAPRSLLLALAAGLAVARPPNIVLIFADDLGYGDLGCYGHPSSTTPNLDQ | 5 |
| (amino acid | LAAGGLRFTDFYVPVSLCTPSRAALLTGRLPVRMGMYPGVLVPSSRGGLP | |
| sequence) | LEEVTVAEVLAARGYLTGMAGKWHLGVGPEGAFLPPHQGFHRFLGTPYSH | |
| | DQGPCQNLTCFPPATPCDGGCDQGLVPIPLLANLSVEAQPPWLPGLEARY | |
| | MAFAHDLMADAQRQDRPFFLYYASHHTHYPQFSGQSFAERSGRGPFGDS | |
| | LMELDAAVGTLMTAIGDLGLLEETLVIFTADNGPETMRMSRGGCSGLLRCG | |
| | KGTTYEGGVREPALAFWPGHIAPGVTHELASSLDLLPTLAALAGAPLPNVT | |
| | LDGFDLSPLLLGTGKSPRQSLFFYPSYPDEVRGVFAVRTGKYKAHFFTQG | |
| | SAHSDTTADPACHASSSLTAHEPPLLYDLSKDPGENYNLLGGVAGATPEV | |
| | LQALKQLQLLKAQLDAAVTFGPSQVARGEDPALQICCHPGCTPRPACCHC | |
| | PDPHA | |
| Hyper-ARSA | MGAPRSLLLALAAGLAVARPPNIVLIFADDLGYGDLGCYGHPSSTTPNLDQ | 6 |
| (amino acid | LAAGGLRFTDFYVPVSLCTPSRAALLTGRLPVRMGMYPGVLVPSSRGGLP | |
| sequence) | LEEVTVAEVLAARGYLTGMAGKWHLGVGPEGAFLPPHQGFHRFLGTPYSH | |
| | DQGPCQNLTCFPPATPCDGGCDQGLVPIPLLANLSVEAQPPWLPGLEARY | |
| | VAFAHDLMADAQRQDRPFFLYYASHHTHYPQFSGQSFAERSGRGPFGDS | |
| | LMELDAAVGTLMTAIGDLGLLEETLVIFTADNGPELMRMSNGGCSGLLRCG | |
| | KGTTYEGGVREPALAFWPGHIAPGVTHELASSLDLLPTLAALAGAPLPNVT | |
| | LDGFDLSPLLLGTGKSPRQSLFFYPSYPDEVRGVFAVRTGKYKAHFFTQG | |
| | SAHSDTTADPACHASSSLTAHEPPLLYDLSKDPGENYNLLGGVAGATPEV | |
| | LQALKQLQLLKAQLDAAVTFGPSQVARGEDPALQICCHPGCTPRPACCHC | |
| | PDPHA | |
| Hyper-ARSA | ATGAGCATGGGCGCTCCTAGAAGCCTGCTGCTGGCCCTGGCTGCCGG | 7 |
| codon | CCTGGCCGTGGCTAGACCTCCAAACATCGTGCTGATCTTCGCCGACGA | |
| optimized | CCTGGGCTATGGTGACCTGGGCTGCTACGGCCACCCCTCTTCTACAAC | |
| sequence(S) | ACCCAATCTGGACCAGCTGGCCGCTGGCGGCCTGAGATTCACAGACTT | |
| | CTACGTGCCAGTGTCCCTGTGCACCCCTTCTAGAGCCGCTCTCCTGAC | |
| | CGGCAGACTGCCTGTGCGGATGGGCATGTACCCCGGAGTGCTGGTGC | |
| | CCAGCAGTAGAGGAGGACTGCCTCTGGAAGAGGTGACCGTGGCCGAG | |
| | GTGCTGGCCGCCAGAGGCTACCTGACAGGAATGGCCGGAAAATGGCA | |
| | CCTGGGAGTGGGCCCAGAAGGCGCCTTCCTGCCACCACACCAGGGCT | |
| | TTCACCGGTTCCTGGGGATCCCTTACAGCCACGACCAAGGCCCTTGTC | |
| | AGAACCTGACATGCTTCCCCCCCGCCACACCTTGCGACGGCGGCTGT | |
| | GACCAGGGCCTTGTGCCTATCCCCCTGCTGGCCAACCTGAGCGTGGA | |
| | AGCCCAGCCTCCATGGCTGCCTGGCCTCGAGGCCAGATACGTGGCCT | |
| | TCGCTCATGATCTGATGGCCGATGCCCAGAGACAGGACAGACCTTTTT | |
| | TCCTGTATTACGCCAGCCACCACACCCACTACCCTCAGTTCAGCGGAC | |
| | AGAGCTTCGCCGAGCGGAGCGGCAGAGGCCCCTTCGGCGACAGCCTG | |
| | ATGGAACTGGACGCCGCTGTTGGAACCCTGATGACCGCCATTGGCGAT | |
| | CTGGGCCTGCTCGAGGAAACCCTGGTGATCTTCACCGCCGATAACGGC | |
| | CCTGAGCTGATGCGGATGTCTAACGGCGGCTGCAGCGGCCTGCTGCG | |
| | GTGCGGCAAGGGCACCACCTACGAGGGCGGCGTGCGGGAACCCGCC | |
| | CTGGCTTTTTGGCCTGGCCACATCGCCCCTGGCGTTACCCACGAGCTG | |
| | GCTTCTAGCCTGGACCTGCTGCCCACCCTGGCCGCACTGGCCGGAGC | |
| | TCCACTGCCTAATGTGACCCTGGATGGCTTCGACCTGTCCCCTCTGCT | |
| | GCTCGGCACCGGCAAGAGCCCTAGACAGAGCCTGTTCTTCTACCCCTC | |
| | CTACCCTGATGAGGTGCGGGGCGTCTTTGCCGTCAGGACCGGCAAATA | |
| | CAAGGCCCATTTCTTTACACAGGGCAGCGCCCACTCTGATACCACAGC | |
| | CGACCCTGCCTGCCACGCCAGCTCCAGCCTGACCGCCCACGAGCCTC | |
| | CTCTGCTATACGACCTGAGCAAGGACCCTGGCGAGAACTACAACCTGC | |
| | TGGGTGGCGTGGCCGGCGCTACACCTGAGGTGCTGCAGGCCCTGAAG | |
| | CAGCTGCAGCTGCTTAAGGCCCAACTGGACGCCGCTGTGACCTTCGG | |
| | CCCTAGCCAGGTGGCCAGAGGAGAAGATCCCGCCCTGCAAATCTGCT | |
| | GCCACCCTGGATGTACCCCTCGGCCCGCTTGTTGTCACTGCCCCGACC | |
| | CTCACGCCTGA | |
| Hyper-ARSA | ATGTCTATGGGAGCCCCTAGATCTCTGCTGCTGGCTCTGGCTGCTGGA | 8 |
| codon- codon | CTGGCAGTTGCCAGACCTCCTAACATCGTGCTGATCTTCGCCGACGAT | |
| optimized | CTCGGCTATGGCGATCTGGGCTGTTACGGACACCCCAGCAGCACCACA | |
| sequence (A) | CCTAACCTGGATCAACTTGCCGCTGGCGGCCTGAGATTCACCGATTTC | |
| | TACGTGCCCGTGTCTCTGTGCACCCCTTCTAGAGCTGCTCTGCTGACA | |
| | GGCAGACTCCCTGTGCGGATGGGAATGTATCCTGGCGTGCTGGTGCC | |
| | TAGCTCTAGAGGCGGACTGCCTCTGGAAGAAGTGACAGTTGCCGAAGT | |
| | GCTGGCCGCCAGAGGATATCTGACTGGCATGGCCGGAAAGTGGCACC | |
| | TCGGAGTTGGACCTGAAGGCGCTTTTCTGCCTCCTCACCAGGGCTTCC | |
| | ACCGGTTTCTGGGCATCCCTTACTCTCACGATCAGGGCCCCTGCCAGA | |
| | ACCTGACCTGTTTTCCTCCTGCCACACCTTGCGACGGCGGCTGTGATC | |
| | AAGGACTGGTGCCAATTCCTCTGCTGGCCAACCTGAGCGTGGAAGCTC | |
| | AACCTCCTTGGCTGCCAGGACTGGAAGCCCGGTATGTGGCCTTCGCTC | |
| | ACGACCTGATGGCCGACGCTCAGAGACAGGACAGACCATTCTTCCTGT | |
| | ACTACGCCAGCCACCACACACACTACCCTCAGTTTAGCGGCCAGAGCT | |
| | TCGCCGAGAGATCTGGCAGAGGACCTTTCGGCGACAGCCTGATGGAA | |
| | CTGGATGCCGCTGTGGGCACACTGATGACAGCCATCGGAGATCTGGG | |
| | ACTGCTGGAAGAGACACTGGTCATCTTCACCGCCGACAACGGCCCCGA | |
| | GCTGATGAGAATGAGCAACGGCGGCTGTAGCGGCCTGCTGAGATGTG | |
| | GCAAGGGCACCACATATGAAGGCGGCGTCAGAGAACCTGCTCTGGCC | |
| | TTTTGGCCTGGCCATATTGCTCCAGGCGTGACACACGAGCTGGCCTCT | |
| | TCTCTGGATCTGCTGCCTACACTGGCAGCTCTTGCTGGTGCTCCCCTG | |
| | CCTAATGTGACCCTGGATGGCTTCGATCTGAGCCCACTGCTGCTCGGC | |
| | ACAGGCAAGTCTCCAAGACAGAGCCTGTTCTTCTACCCTAGCTACCCC | |
| | GATGAAGTGCGGGGAGTGTTTGCCGTGCGGACCGGAAAGTATAAGGC | |
| | CCACTTCTTCACCCAAGGCAGCGCCCACTCTGACACCACAGCTGATCC | |
| | TGCTTGTCACGCCAGCTCTAGCCTGACAGCCCATGAACCTCCACTGCT | |
| | GTACGACCTGAGCAAGGACCCCGGCGAGAACTACAATCTGCTTGGCG | |
| | GAGTTGCCGGCGCTACACCTGAAGTTCTGCAGGCCCTGAAACAGCTCC | |
| | AGCTGCTGAAAGCCCAGCTGGACGCTGCCGTGACATTTGGACCTAGTC | |
| | AGGTGGCCAGAGGCGAGGATCCTGCTCTGCAGATCTGTTGTCACCCTG | |
| | GCTGCACACCCAGACCTGCCTGCTGTCATTGTCCTGATCCTCACGCCT | |
| | GA | |
| UbC promoter | GGCCTCCGCGCCGGGTTTTGGCGCCTCCCGCGGGCGCCCCCCTCCTC | 9 |
| minimal | ACGGCGAGCGCTGCCACGTCAGACGAAGGGCGCAGCGAGCGTCCTGA | |
| (nucleotide | TCCTTCCGCCCGGACGCTCAGGACAGCGGCCCGCTGCTCATAAGACT | |
| sequence) | CGGCCTTAGAACCCCAGTATCAGCAGAAGGACATTTTAGGACGGGACT | |
| | TGGGTGACTCTAGGGCACTGGTTTTCTTTCCAGAGAGCGGAACAGGCG | |
| | AGGAAAAGTAGTCCCTTCTCGGCGATTCTGCGGAGGGATCTCCGTGGG | |
| | GCGGTGAACGCCGATGATTATATAAGGACGCGCCGGGTGTGGCACAG | |
| | CT | |
| UbC promoter | GGCCTCCGCGCCGGGTTTTGGCGCCTCCCGCGGGCGCCCCCCTCCTC | 10 |
| full (nucleotide | ACGGCGAGCGCTGCCACGTCAGACGAAGGGCGCAGCGAGCGTCCTGA | |
| sequence) | TCCTTCCGCCCGGACGCTCAGGACAGCGGCCCGCTGCTCATAAGACT | |
| | CGGCCTTAGAACCCCAGTATCAGCAGAAGGACATTTTAGGACGGGACT | |
| | TGGGTGACTCTAGGGCACTGGTTTTCTTTCCAGAGAGCGGAACAGGCG | |
| | AGGAAAAGTAGTCCCTTCTCGGCGATTCTGCGGAGGGATCTCCGTGGG | |
| | GCGGTGAACGCCGATGATTATATAAGGACGCGCCGGGTGTGGCACAG | |
| | CTAGTTCCGTCGCAGCCGGGATTTGGGTCGCAGTTCTTGTTTGTGGAT | |
| | CGCTGTGATCGTCACTTGGTGAGTAGCGGGCTGCTGGGCTGGCCGGG | |
| | GCTTTCGTGGCCGCCGGGCCGCTCGGTGGGACGGAGGCGTGTGGAG | |
| | AGACCGCCAAGGGCTGTAGTCTGGGTCCGCGAGCAAGGTTGCCCTGA | |
| | ACTGGGGGTTGGGGGGAGCGCAGCAAAATGGCGGCTGTTCCCGAGTC | |
| | TTGAATGGAAGACGCTTGTGAGGCGGGCTGTGAGGTCGTTGAAACAAG | |
| | GTGGGGGGCATGGTGGGCGGCAAGAACCCAAGGTCTTGAGGCCTTCG | |
| | CTAATGCGGGAAAGCTCTTATTCGGGTGAGATGGGCTGGGGCACCATC | |
| | TGGGGACCCTGACGTGAAGTTTGTCACTGACTGGAGAACTCGGTTTGT | |
| | CGTCTGTTGCGGGGGCGGCAGTTATGGCGGTGCCGTTGGGCAGTGCA | |
| | CCCGTACCTTTGGGAGCGCGCGCCCTCGTCGTGTCGTGACGTCACCC | |
| | GTTCTGTTGGCTTATAATGCAGGGTGGGGCCACCTGCCGGTAGGTGTG | |
| | CGGTAGGCTTTTCTCCGTCGCAGGACGCAGGGTTCGGGCCAAGGGTA | |
| | GGCTCTCCTGAATCGACAGGCGCCGGACCTCTGGTGAGGGGAGGGAT | |
| | AAGTGAGGCGTCAGTTTCTCTGGTCGGTTTTATGTACCTATCTTCTTAA | |
| | GTAGCTGAAGCTCCGGTTTTGAACTATGCGCTCGGGGTTGGCGAGTGT | |
| | GTTTTGTGAAGTTTTTTAGGCACCTTTTGAAATGTAATCATTTGGGTCAA | |
| | TATGTAATTTTCAGTGTTAGACTAGTAAATTGTCCGCTAAATTCTGGCCG | |
| | TTTTTGGCTTTTTTGTTAGAC | |
| UbC promoter | GGCCTCCGCGCCGGGTTTTGGCGCCTCCCGCGGGCGCCCCCCTCCTC | 11 |
| full - variant | ACGGCGAGCGCTGCCACGTCAGACGAAGGGCGCAGCGAGCGTCCTGA | |
| sequence | TCCTTCCGCCCGGACGCTCAGGACAGCGGCCCGCTGCTCATAAGACT | |
| (nucleotide | CGGCCTTAGAACCCCAGTATCAGCAGAAGGACATTTTAGGACGGGACT | |
| sequence) | TGGGTGACTCTAGGGCACTGGTTTTCTTTCCAGAGAGCGGAACAGGCG | |
| | AGGAAAAGTAGTCCCTTCTCGGCGATTCTGCGGAGGGATCTCCGTGGG | |
| | GCGGTGAACGCCGATGATTATATAAGGACGCGCCGGGTGTGGCACAG | |
| | CTAGTTCCGTCGCAGCCGGGATTTGGGTCGCAGTTCTTGTTTGTGGAT | |
| | CGCTGTGATCGTCACTTGGTGAGTAGCGGGCTGCTGGGCTGGCCGGG | |
| | GCTTTCGTGGCCGCCGGGCCGCTCGGTGGGACGGAGGCGTGTGGAG | |
| | AGCCCGCCAAGGGCTGTAGTCTGGGTCCGCGAGCAAGGTTGCCCTGA | |
| | ACTGGGGGTTGGGGGGAGCGCAGCAAAATGGCGGCTGTTCCCGAGTC | |
| | TTGAATGGAAGACGCTTGTGAGGCGGGCTGTGAGGTCGTTGAAACAAG | |
| | GTGGGGGGCATGGTGGGCGGCAAGAACCCAAGGTCTTGAGGCCTTCG | |
| | CTAATGCGGGAAAGCTCTTATTCGGGTGAGATGGGCTGGGGCACCATC | |
| | TGGGGACCCTGACGTGAAGTTTGTCACTGACTGGAGAACTCGGTTTGT | |
| | CGTCTGTTGCGGGGGCGGCAGTTATGGCGGTGCCGTTGGGCAGTGCA | |
| | CCCGTACCTTTGGGAGCGCGCGCCCTCGTCGTGTCGTGACGTCACCC | |
| | GTTCTGTTGGCTTATAATGCAGGGTGGGGCCACCTGCCGGTAGGTGTG | |
| | CGGTAGGCTTTTCTCCGTCGCAGGACGCAGGGTTCGGGCCAAGGGTA | |
| | GGCTCTCCTGAATCGACAGGCGCCGGACCTCTGGTGAGGGGAGGGAT | |
| | AAGTGAGGCGTCAGTTTCTCTGGTCGGTTTTATGTACCTATCTTCTTAA | |
| | GTAGCTGAAGCTCCGGTTTTGAACTATGCGCTCGGGGTTGGCGAGTGT | |
| | GTTTTGTGAAGTTTTTTAGGCACCTTTTGAAATGTAATCATTTGGGTCAA | |
| | TATGTAATTTTCAGTGTTAGACTAGTAAATTGTCCGCTAAATTCTGGCCG | |
| | TTTTTGGCTTTTTTGTTAGAC | |
| CAG promoter | GCGTTACATAACTTACGGTAAATGGCCCGCCTGGCTGACCGCCCAACG | 12 |
| (nucleotide | ACCCCCGCCCATTGACGTCAATAATGACGTATGTTCCCATAGTAACGCC | |
| sequence) | AATAGGGACTTTCCATTGACGTCAATGGGTGGAGTATTTACGGTAAACT | |
| | GCCCACTTGGCAGTACATCAAGTGTATCATATGCCAAGTACGCCCCCTA | |
| | TTGACGTCAATGACGGTAAATGGCCCGCCTGGCATTATGCCCAGTACA | |
| | TGACCTTATGGGACTTTCCTACTTGGCAGTACATCTACGTATTAGTCAT | |
| | CGCTATTACCATGGTCGAGGTGAGCCCCACGTTCTGCTTCACTCTCCC | |
| | CATCTCCCCCCCCTCCCCACCCCCAATTTTGTATTTATTTATTTTTTAATT | |
| | ATTTTGTGCAGCGATGGGGGCGGGGGGGGGGGGGGGGCGCGCGCCA | |
| | GGCGGGGCGGGGCGGGGCGAGGGGCGGGGCGGGGCGAGGCGGAG | |
| | AGGTGCGGCGGCAGCCAATCAGAGCGGCGCGCTCCGAAAGTTTCCTT | |
| | TTATGGCGAGGCGGCGGCGGCGGCGGCCCTATAAAAAGCGAAGCGCG | |
| | CGGCGGGCG | |
| CMV promoter | GTGATGCGGTTTTGGCAGTACACCAATGGGCGTGGATAGCGGTTTGAC | 13 |
| (nucleotide | TCACGGGGATTTCCAAGTCTCCACCCCATTGACGTCAATGGGAGTTTGT | |
| sequence) | TTTGGCACCAAAATCAACGGGACTTTCCAAAATGTCGTAATAACCCCGC | |
| | CCCGTTGACGCAAATGGGCGGTAGGCGTGTACGGTGGGAGGTCTATA | |
| | TAAGCAGAGCT | |
| CMV enhancer- | GCATTGATTATTGACTAGTTATTAATAGTAATCAATTACGGGGTCATTAG | 14 |
| promoter | TTCATAGCCCATATATGGAGTTCCGCGTTACATAACTTACGGTAAATGG | |
| (nucleotide | CCCGCCTGGCTGACCGCCCAACGACCCCCGCCCATTGACGTCAATAAT | |
| sequence) | GACGTATGTTCCCATAGTAACGCCAATAGGGACTTTCCATTGACGTCAA | |
| | TGGGTGGAGTATTTACGGTAAACTGCCCACTTGGCAGTACATCAAGTGT | |
| | ATCATATGCCAAGTCCGCCCCCTATTGACGTCAATGACGGTAAATGGC | |
| | CCGCCTGGCATTATGCCCAGTACATGACCTTACGGGACTTTCCTACTTG | |
| | GCAGTACATCTACGTATTAGTCATCGCTATTACCATGGTGATGCGGTTT | |
| | TGGCAGTACACCAATGGGCGTGGATAGCGGTTTGACTCACGGGGATTT | |
| | CCAAGTCTCCACCCCATTGACGTCAATGGGAGTTTGTTTTGGCACCAAA | |
| | ATCAACGGGACTTTCCAAAATGTCGTAATAACCCCGCCCCGTTGACGC | |
| | AAATGGGCGGTAGGCGTGTACGGTGGGAGGTCTATATAAGCAGAGCTC | |
| | GTTTAGTGAACCGTCAGA | |
| WPRE | AATCAACCTCTGGATTACAAAATTTGTGAAAGATTGACTGGTATTCTTAA | 15 |
| (nucleotide | CTATGTTGCTCCTTTTACGCTATGTGGATACGCTGCTTTAATGCCTTTGT | |
| sequence) | ATCATGCTATTGCTTCCCGTATGGCTTTCATTTTCTCCTCCTTGTATAAA | |
| | TCCTGGTTGCTGTCTCTTTATGAGGAGTTGTGGCCCGTTGTCAGGCAA | |
| | CGTGGCGTGGTGTGCACTGTGTTTGCTGACGCAACCCCCACTGGTTGG | |
| | GGCATTGCCACCACCTGTCAGCTCCTTTCCGGGACTTTCGCTTTCCCC | |
| | CTCCCTATTGCCACGGCGGAACTCATCGCCGCCTGCCTTGCCCGCTGC | |
| | TGGACAGGGGCTCGGCTGTTGGGCACTGACAATTCCGTGGTGTTGTCG | |
| | GGGAAATCATCGTCCTTTCCTTGGCTGCTCGCCTGTGTTGCCACCTGG | |
| | ATTCTGCGCGGGACGTCCTTCTGCTACGTCCCTTCGGCCCTCAATCCA | |
| | GCGGACCTTCCTTCCCGCGGCCTGCTGCCGGCTCTGCGGCCTCTTCC | |
| | GCGTCTTCGCCTTCGCCCTCAGACGAGTCGGATCTCCCTTTGGGCCGC | |
| | CTCCCCGC | |
| SV40 LPA | ATTTGTGAAATTTGTGATGCTATTGCTTTATTTGTAACCATTATAAGCTG | 16 |
| (nucleotide | CAATAAACAAGTTAACAACAACAATTGCATTCATTTTATGTTTCAGGTTC | |
| sequence) | AGGGGGAGGTGTGGGAGGTTTTTT | |
| 5′ ITR | GCGCGCTCGCTCGCTCACTGAGGCCGCCCGGGCAAAGCCCGGGCGT | 17 |
| (nucleotide | CGGGCGACCTTTGGTCGCCCGGCCTCAGTGAGCGAGCGAGCGCGC | |
| sequence) | | |
| 3′ ITR | AGGAACCCCTAGTGATGGAGTTGGCCACTCCCTCTCTGCGCGCTCGCT | 18 |
| (nucleotide | CGCTCACTGAGGCCGGGCGACCAAAGGTCGCCCGACGCCCGGGCTTT | |
| sequence) | GCCCGGGCGGCCTCAGTGAGCGAGCGAGCGCGCAGAGAGGGAGTGG | |
| | CCAA | |
| UbC-COGS | GCGCGCTCGCTCGCTCACTGAGGCCGCCCGGGCAAAGCCCGGGCGT | 19 |
| (ATP0123) | CGGGCGACCTTTGGTCGCCCGGCCTCAGTGAGCGAGCGAGCGCGCA | |
| | GAGAGGGAGTGGCCAACTCCATCACTAGGGGTTCCTTTGTCGACTCGG | |
| | CCTCCGCGCCGGGTTTTGGCGCCTCCCGCGGGCGCCCCCCTCCTCAC | |
| | GGCGAGCGCTGCCACGTCAGACGAAGGGCGCAGCGAGCGTCCTGATC | |
| | CTTCCGCCCGGACGCTCAGGACAGCGGCCCGCTGCTCATAAGACTCG | |
| | GCCTTAGAACCCCAGTATCAGCAGAAGGACATTTTAGGACGGGACTTG | |
| | GGTGACTCTAGGGCACTGGTTTTCTTTCCAGAGAGCGGAACAGGCGAG | |
| | GAAAAGTAGTCCCTTCTCGGCGATTCTGCGGAGGGATCTCCGTGGGGC | |
| | GGTGAACGCCGATGATTATATAAGGACGCGCCGGGTGTGGCACAGCTA | |
| | GTTCCGTCGCAGCCGGGATTTGGGTCGCAGTTCTTGTTTGTGGATCGC | |
| | TGTGATCGTCACTTGGTGAGTAGCGGGCTGCTGGGCTGGCCGGGGCT | |
| | TTCGTGGCCGCCGGGCCGCTCGGTGGGACGGAGGCGTGTGGAGAGA | |
| | CCGCCAAGGGCTGTAGTCTGGGTCCGCGAGCAAGGTTGCCCTGAACT | |
| | GGGGGTTGGGGGGAGCGCAGCAAAATGGCGGCTGTTCCCGAGTCTTG | |
| | AATGGAAGACGCTTGTGAGGCGGGCTGTGAGGTCGTTGAAACAAGGT | |
| | GGGGGGCATGGTGGGCGGCAAGAACCCAAGGTCTTGAGGCCTTCGCT | |
| | AATGCGGGAAAGCTCTTATTCGGGTGAGATGGGCTGGGGCACCATCTG | |
| | GGGACCCTGACGTGAAGTTTGTCACTGACTGGAGAACTCGGTTTGTCG | |
| | TCTGTTGCGGGGGCGGCAGTTATGGCGGTGCCGTTGGGCAGTGCACC | |
| | CGTACCTTTGGGAGCGCGCGCCCTCGTCGTGTCGTGACGTCACCCGTT | |
| | CTGTTGGCTTATAATGCAGGGTGGGGCCACCTGCCGGTAGGTGTGCG | |
| | GTAGGCTTTTCTCCGTCGCAGGACGCAGGGTTCGGGCCAAGGGTAGG | |
| | CTCTCCTGAATCGACAGGCGCCGGACCTCTGGTGAGGGGAGGGATAA | |
| | GTGAGGCGTCAGTTTCTCTGGTCGGTTTTATGTACCTATCTTCTTAAGT | |
| | AGCTGAAGCTCCGGTTTTGAACTATGCGCTCGGGGTTGGCGAGTGTGT | |
| | TTTGTGAAGTTTTTTAGGCACCTTTTGAAATGTAATCATTTGGGTCAATA | |
| | TGTAATTTTCAGTGTTAGACTAGTAAATTGTCCGCTAAATTCTGGCCGTT | |
| | TTTGGCTTTTTTGTTAGACAGATCTATGAGCATGGGCGCTCCTAGAAGC | |
| | CTGCTGCTGGCCCTGGCTGCCGGCCTGGCCGTGGCTAGACCTCCAAA | |
| | CATCGTGCTGATCTTCGCCGACGACCTGGGCTATGGTGACCTGGGCTG | |
| | CTACGGCCACCCCTCTTCTACAACACCCAATCTGGACCAGCTGGCCGC | |
| | TGGCGGCCTGAGATTCACAGACTTCTACGTGCCAGTGTCCCTGTGCAC | |
| | CCCTTCTAGAGCCGCTCTCCTGACCGGCAGACTGCCTGTGCGGATGG | |
| | GCATGTACCCCGGAGTGCTGGTGCCCAGCAGTAGAGGAGGACTGCCT | |
| | CTGGAAGAGGTGACCGTGGCCGAGGTGCTGGCCGCCAGAGGCTACCT | |
| | GACAGGAATGGCCGGAAAATGGCACCTGGGAGTGGGCCCAGAAGGCG | |
| | CCTTCCTGCCACCACACCAGGGCTTTCACCGGTTCCTGGGGATCCCTT | |
| | ACAGCCACGACCAAGGCCCTTGTCAGAACCTGACATGCTTCCCCCCCG | |
| | CCACACCTTGCGACGGCGGCTGTGACCAGGGCCTTGTGCCTATCCCC | |
| | CTGCTGGCCAACCTGAGCGTGGAAGCCCAGCCTCCATGGCTGCCTGG | |
| | CCTCGAGGCCAGATACATGGCCTTCGCTCATGATCTGATGGCCGATGC | |
| | CCAGAGACAGGACAGACCTTTTTTCCTGTATTACGCCAGCCACCACAC | |
| | CCACTACCCTCAGTTCAGCGGACAGAGCTTCGCCGAGCGGAGCGGCA | |
| | GAGGCCCCTTCGGCGACAGCCTGATGGAACTGGACGCCGCTGTTGGA | |
| | ACCCTGATGACCGCCATTGGCGATCTGGGCCTGCTCGAGGAAACCCTG | |
| | GTGATCTTCACCGCCGATAACGGCCCTGAGACAATGCGGATGTCTAGA | |
| | GGCGGCTGCAGCGGCCTGCTGCGGTGCGGCAAGGGCACCACCTACG | |
| | AGGGCGGCGTGCGGGAACCCGCCCTGGCTTTTTGGCCTGGCCACATC | |
| | GCCCCTGGCGTTACCCACGAGCTGGCTTCTAGCCTGGACCTGCTGCC | |
| | CACCCTGGCCGCACTGGCCGGAGCTCCACTGCCTAATGTGACCCTGG | |
| | ATGGCTTCGACCTGTCCCCTCTGCTGCTCGGCACCGGCAAGAGCCCTA | |
| | GACAGAGCCTGTTCTTCTACCCCTCCTACCCTGATGAGGTGCGGGGCG | |
| | TCTTTGCCGTCAGGACCGGCAAATACAAGGCCCATTTCTTTACACAGG | |
| | GCAGCGCCCACTCTGATACCACAGCCGACCCTGCCTGCCACGCCAGC | |
| | TCCAGCCTGACCGCCCACGAGCCTCCTCTGCTATACGACCTGAGCAAG | |
| | GACCCTGGCGAGAACTACAACCTGCTGGGTGGCGTGGCCGGCGCTAC | |
| | ACCTGAGGTGCTGCAGGCCCTGAAGCAGCTGCAGCTGCTTAAGGCCC | |
| | AACTGGACGCCGCTGTGACCTTCGGCCCTAGCCAGGTGGCCAGAGGA | |
| | GAAGATCCCGCCCTGCAAATCTGCTGCCACCCTGGATGTACCCCTCGG | |
| | CCCGCTTGTTGTCACTGCCCCGACCCTCACGCCTGAGGTACCAATCAA | |
| | CCTCTGGATTACAAAATTTGTGAAAGATTGACTGGTATTCTTAACTATGT | |
| | TGCTCCTTTTACGCTATGTGGATACGCTGCTTTAATGCCTTTGTATCATG | |
| | CTATTGCTTCCCGTATGGCTTTCATTTTCTCCTCCTTGTATAAATCCTGG | |
| | TTGCTGTCTCTTTATGAGGAGTTGTGGCCCGTTGTCAGGCAACGTGGC | |
| | GTGGTGTGCACTGTGTTTGCTGACGCAACCCCCACTGGTTGGGGCATT | |
| | GCCACCACCTGTCAGCTCCTTTCCGGGACTTTCGCTTTCCCCCTCCCTA | |
| | TTGCCACGGCGGAACTCATCGCCGCCTGCCTTGCCCGCTGCTGGACA | |
| | GGGGCTCGGCTGTTGGGCACTGACAATTCCGTGGTGTTGTCGGGGAA | |
| | ATCATCGTCCTTTCCTTGGCTGCTCGCCTGTGTTGCCACCTGGATTCTG | |
| | CGCGGGACGTCCTTCTGCTACGTCCCTTCGGCCCTCAATCCAGCGGAC | |
| | CTTCCTTCCCGCGGCCTGCTGCCGGCTCTGCGGCCTCTTCCGCGTCTT | |
| | CGCCTTCGCCCTCAGACGAGTCGGATCTCCCTTTGGGCCGCCTCCCC | |
| | GCGAGCTCATTTGTGAAATTTGTGATGCTATTGCTTTATTTGTAACCATT | |
| | ATAAGCTGCAATAAACAAGTTAACAACAACAATTGCATTCATTTTATGTT | |
| | TCAGGTTCAGGGGGAGGTGTGGGAGGTTTTTTGAGTCCTAGGAGGAAC | |
| | CCCTAGTGATGGAGTTGGCCACTCCCTCTCTGCGCGCTCGCTCGCTCA | |
| | CTGAGGCCGGGCGACCAAAGGTCGCCCGACGCCCGGGCTTTGCCCG | |
| | GGCGGCCTCAGTGAGCGAGCGAGCGCGCAGAGAGGGAGTGGCCAA | |
| UbC-COGS- | GCGCGCTCGCTCGCTCACTGAGGCCGCCCGGGCAAAGCCCGGGCGT | 20 |
| Hyper | CGGGCGACCTTTGGTCGCCCGGCCTCAGTGAGCGAGCGAGCGCGCA | |
| (ATP0137) | GAGAGGGAGTGGCCAACTCCATCACTAGGGGTTCCTTTGTCGACTCGG | |
| | CCTCCGCGCCGGGTTTTGGCGCCTCCCGCGGGCGCCCCCCTCCTCAC | |
| | GGCGAGCGCTGCCACGTCAGACGAAGGGCGCAGCGAGCGTCCTGATC | |
| | CTTCCGCCCGGACGCTCAGGACAGCGGCCCGCTGCTCATAAGACTCG | |
| | GCCTTAGAACCCCAGTATCAGCAGAAGGACATTTTAGGACGGGACTTG | |
| | GGTGACTCTAGGGCACTGGTTTTCTTTCCAGAGAGCGGAACAGGCGAG | |
| | GAAAAGTAGTCCCTTCTCGGCGATTCTGCGGAGGGATCTCCGTGGGGC | |
| | GGTGAACGCCGATGATTATATAAGGACGCGCCGGGTGTGGCACAGCTA | |
| | GTTCCGTCGCAGCCGGGATTTGGGTCGCAGTTCTTGTTTGTGGATCGC | |
| | TGTGATCGTCACTTGGTGAGTAGCGGGCTGCTGGGCTGGCCGGGGCT | |
| | TTCGTGGCCGCCGGGCCGCTCGGTGGGACGGAGGCGTGTGGAGAGA | |
| | CCGCCAAGGGCTGTAGTCTGGGTCCGCGAGCAAGGTTGCCCTGAACT | |
| | GGGGGTTGGGGGGAGCGCAGCAAAATGGCGGCTGTTCCCGAGTCTTG | |
| | AATGGAAGACGCTTGTGAGGCGGGCTGTGAGGTCGTTGAAACAAGGT | |
| | GGGGGGCATGGTGGGCGGCAAGAACCCAAGGTCTTGAGGCCTTCGCT | |
| | AATGCGGGAAAGCTCTTATTCGGGTGAGATGGGCTGGGGCACCATCTG | |
| | GGGACCCTGACGTGAAGTTTGTCACTGACTGGAGAACTCGGTTTGTCG | |
| | TCTGTTGCGGGGGCGGCAGTTATGGCGGTGCCGTTGGGCAGTGCACC | |
| | CGTACCTTTGGGAGCGCGCGCCCTCGTCGTGTCGTGACGTCACCCGTT | |
| | CTGTTGGCTTATAATGCAGGGTGGGGCCACCTGCCGGTAGGTGTGCG | |
| | GTAGGCTTTTCTCCGTCGCAGGACGCAGGGTTCGGGCCAAGGGTAGG | |
| | CTCTCCTGAATCGACAGGCGCCGGACCTCTGGTGAGGGGAGGGATAA | |
| | GTGAGGCGTCAGTTTCTCTGGTCGGTTTTATGTACCTATCTTCTTAAGT | |
| | AGCTGAAGCTCCGGTTTTGAACTATGCGCTCGGGGTTGGCGAGTGTGT | |
| | TTTGTGAAGTTTTTTAGGCACCTTTTGAAATGTAATCATTTGGGTCAATA | |
| | TGTAATTTTCAGTGTTAGACTAGTAAATTGTCCGCTAAATTCTGGCCGTT | |
| | TTTGGCTTTTTTGTTAGACAGATCTATGAGCATGGGCGCTCCTAGAAGC | |
| | CTGCTGCTGGCCCTGGCTGCCGGCCTGGCCGTGGCTAGACCTCCAAA | |
| | CATCGTGCTGATCTTCGCCGACGACCTGGGCTATGGTGACCTGGGCTG | |
| | CTACGGCCACCCCTCTTCTACAACACCCAATCTGGACCAGCTGGCCGC | |
| | TGGCGGCCTGAGATTCACAGACTTCTACGTGCCAGTGTCCCTGTGCAC | |
| | CCCTTCTAGAGCCGCTCTCCTGACCGGCAGACTGCCTGTGCGGATGG | |
| | GCATGTACCCCGGAGTGCTGGTGCCCAGCAGTAGAGGAGGACTGCCT | |
| | CTGGAAGAGGTGACCGTGGCCGAGGTGCTGGCCGCCAGAGGCTACCT | |
| | GACAGGAATGGCCGGAAAATGGCACCTGGGAGTGGGCCCAGAAGGCG | |
| | CCTTCCTGCCACCACACCAGGGCTTTCACCGGTTCCTGGGGATCCCTT | |
| | ACAGCCACGACCAAGGCCCTTGTCAGAACCTGACATGCTTCCCCCCCG | |
| | CCACACCTTGCGACGGCGGCTGTGACCAGGGCCTTGTGCCTATCCCC | |
| | CTGCTGGCCAACCTGAGCGTGGAAGCCCAGCCTCCATGGCTGCCTGG | |
| | CCTCGAGGCCAGATACGTGGCCTTCGCTCATGATCTGATGGCCGATGC | |
| | CCAGAGACAGGACAGACCTTTTTTCCTGTATTACGCCAGCCACCACAC | |
| | CCACTACCCTCAGTTCAGCGGACAGAGCTTCGCCGAGCGGAGCGGCA | |
| | GAGGCCCCTTCGGCGACAGCCTGATGGAACTGGACGCCGCTGTTGGA | |
| | ACCCTGATGACCGCCATTGGCGATCTGGGCCTGCTCGAGGAAACCCTG | |
| | GTGATCTTCACCGCCGATAACGGCCCTGAGCTGATGCGGATGTCTAAC | |
| | GGCGGCTGCAGCGGCCTGCTGCGGTGCGGCAAGGGCACCACCTACG | |
| | AGGGCGGCGTGCGGGAACCCGCCCTGGCTTTTTGGCCTGGCCACATC | |
| | GCCCCTGGCGTTACCCACGAGCTGGCTTCTAGCCTGGACCTGCTGCC | |
| | CACCCTGGCCGCACTGGCCGGAGCTCCACTGCCTAATGTGACCCTGG | |
| | ATGGCTTCGACCTGTCCCCTCTGCTGCTCGGCACCGGCAAGAGCCCTA | |
| | GACAGAGCCTGTTCTTCTACCCCTCCTACCCTGATGAGGTGCGGGGCG | |
| | TCTTTGCCGTCAGGACCGGCAAATACAAGGCCCATTTCTTTACACAGG | |
| | GCAGCGCCCACTCTGATACCACAGCCGACCCTGCCTGCCACGCCAGC | |
| | TCCAGCCTGACCGCCCACGAGCCTCCTCTGCTATACGACCTGAGCAAG | |
| | GACCCTGGCGAGAACTACAACCTGCTGGGTGGCGTGGCCGGCGCTAC | |
| | ACCTGAGGTGCTGCAGGCCCTGAAGCAGCTGCAGCTGCTTAAGGCCC | |
| | AACTGGACGCCGCTGTGACCTTCGGCCCTAGCCAGGTGGCCAGAGGA | |
| | GAAGATCCCGCCCTGCAAATCTGCTGCCACCCTGGATGTACCCCTCGG | |
| | CCCGCTTGTTGTCACTGCCCCGACCCTCACGCCTGAGGTACCAATCAA | |
| | CCTCTGGATTACAAAATTTGTGAAAGATTGACTGGTATTCTTAACTATGT | |
| | TGCTCCTTTTACGCTATGTGGATACGCTGCTTTAATGCCTTTGTATCATG | |
| | CTATTGCTTCCCGTATGGCTTTCATTTTCTCCTCCTTGTATAAATCCTGG | |
| | TTGCTGTCTCTTTATGAGGAGTTGTGGCCCGTTGTCAGGCAACGTGGC | |
| | GTGGTGTGCACTGTGTTTGCTGACGCAACCCCCACTGGTTGGGGCATT | |
| | GCCACCACCTGTCAGCTCCTTTCCGGGACTTTCGCTTTCCCCCTCCCTA | |
| | TTGCCACGGCGGAACTCATCGCCGCCTGCCTTGCCCGCTGCTGGACA | |
| | GGGGCTCGGCTGTTGGGCACTGACAATTCCGTGGTGTTGTCGGGGAA | |
| | ATCATCGTCCTTTCCTTGGCTGCTCGCCTGTGTTGCCACCTGGATTCTG | |
| | CGCGGGACGTCCTTCTGCTACGTCCCTTCGGCCCTCAATCCAGCGGAC | |
| | CTTCCTTCCCGCGGCCTGCTGCCGGCTCTGCGGCCTCTTCCGCGTCTT | |
| | CGCCTTCGCCCTCAGACGAGTCGGATCTCCCTTTGGGCCGCCTCCCC | |
| | GCGAGCTCATTTGTGAAATTTGTGATGCTATTGCTTTATTTGTAACCATT | |
| | ATAAGCTGCAATAAACAAGTTAACAACAACAATTGCATTCATTTTATGTT | |
| | TCAGGTTCAGGGGGAGGTGTGGGAGGTTTTTTGAGTCCTAGGAGGAAC | |
| | CCCTAGTGATGGAGTTGGCCACTCCCTCTCTGCGCGCTCGCTCGCTCA | |
| | CTGAGGCCGGGCGACCAAAGGTCGCCCGACGCCCGGGCTTTGCCCG | |
| | GGCGGCCTCAGTGAGCGAGCGAGCGCGCAGAGAGGGAGTGGCCAA | |
| CMV-COGS | GCGCGCTCGCTCGCTCACTGAGGCCGCCCGGGCAAAGCCCGGGCGT | 21 |
| (ATP0139) | CGGGCGACCTTTGGTCGCCCGGCCTCAGTGAGCGAGCGAGCGCGCA | |
| | GAGAGGGAGTGGCCAACTCCATCACTAGGGGTTCCTTTGTCGACCTCT | |
| | AGGAAGACCTTCAATATTGGCCATTAGCCATATTATTCATTGGTTATATA | |
| | GCATAAATCAATATTGGCTATTGGCCATTGCATACGTTGTATCTATATCA | |
| | TAATATGTACATTTATATTGGCTCATGTCCAATATGACCGCCATGTTGCA | |
| | TTGATTATTGACTAGTTATTAATAGTAATCAATTACGGGGTCATTAGTTC | |
| | ATAGCCCATATATGGAGTTCCGCGTTACATAACTTACGGTAAATGGCCC | |
| | GCCTGGCTGACCGCCCAACGACCCCCGCCCATTGACGTCAATAATGAC | |
| | GTATGTTCCCATAGTAACGCCAATAGGGACTTTCCATTGACGTCAATGG | |
| | GTGGAGTATTTACGGTAAACTGCCCACTTGGCAGTACATCAAGTGTATC | |
| | ATATGCCAAGTCCGCCCCCTATTGACGTCAATGACGGTAAATGGCCCG | |
| | CCTGGCATTATGCCCAGTACATGACCTTACGGGACTTTCCTACTTGGCA | |
| | GTACATCTACGTATTAGTCATCGCTATTACCATGGTGATGCGGTTTTGG | |
| | CAGTACACCAATGGGCGTGGATAGCGGTTTGACTCACGGGGATTTCCA | |
| | AGTCTCCACCCCATTGACGTCAATGGGAGTTTGTTTTGGCACCAAAATC | |
| | AACGGGACTTTCCAAAATGTCGTAATAACCCCGCCCCGTTGACGCAAAT | |
| | GGGCGGTAGGCGTGTACGGTGGGAGGTCTATATAAGCAGAGCTCGTTT | |
| | AGTGAACCGTCAGATCACTAGACACTTTGTGGCGGTAGTTTATCACAGT | |
| | TAAATTGCTAACGCAGTCAGTGCTTCTGACACAACAGTCTCGAACTTAA | |
| | GCTGCAGAAGTTGGTCGTGAGGCACTGGGCAGGTAAGTATCAAGGTTA | |
| | CAAGACAGGTTTAAGGCGACCAATAGAAACTGGGCTTGTCGAGACAGA | |
| | GAAGACTCTTGCGTTTCTGATAGGCACCTATTGGTCTTACTGACATCCA | |
| | CTTTGCCTTTCTCTCCACAGGTGTCCACTCCCAGTTCAATTACAGCTCT | |
| | TAAGGCTAGAGTACTTAATACGACTCACTATAGGAGATCTATGAGCATG | |
| | GGCGCTCCTAGAAGCCTGCTGCTGGCCCTGGCTGCCGGCCTGGCCGT | |
| | GGCTAGACCTCCAAACATCGTGCTGATCTTCGCCGACGACCTGGGCTA | |
| | TGGTGACCTGGGCTGCTACGGCCACCCCTCTTCTACAACACCCAATCT | |
| | GGACCAGCTGGCCGCTGGCGGCCTGAGATTCACAGACTTCTACGTGC | |
| | CAGTGTCCCTGTGCACCCCTTCTAGAGCCGCTCTCCTGACCGGCAGAC | |
| | TGCCTGTGCGGATGGGCATGTACCCCGGAGTGCTGGTGCCCAGCAGT | |
| | AGAGGAGGACTGCCTCTGGAAGAGGTGACCGTGGCCGAGGTGCTGGC | |
| | CGCCAGAGGCTACCTGACAGGAATGGCCGGAAAATGGCACCTGGGAG | |
| | TGGGCCCAGAAGGCGCCTTCCTGCCACCACACCAGGGCTTTCACCGG | |
| | TTCCTGGGGATCCCTTACAGCCACGACCAAGGCCCTTGTCAGAACCTG | |
| | ACATGCTTCCCCCCCGCCACACCTTGCGACGGCGGCTGTGACCAGGG | |
| | CCTTGTGCCTATCCCCCTGCTGGCCAACCTGAGCGTGGAAGCCCAGCC | |
| | TCCATGGCTGCCTGGCCTCGAGGCCAGATACATGGCCTTCGCTCATGA | |
| | TCTGATGGCCGATGCCCAGAGACAGGACAGACCTTTTTTCCTGTATTAC | |
| | GCCAGCCACCACACCCACTACCCTCAGTTCAGCGGACAGAGCTTCGCC | |
| | GAGCGGAGCGGCAGAGGCCCCTTCGGCGACAGCCTGATGGAACTGGA | |
| | CGCCGCTGTTGGAACCCTGATGACCGCCATTGGCGATCTGGGCCTGCT | |
| | CGAGGAAACCCTGGTGATCTTCACCGCCGATAACGGCCCTGAGACAAT | |
| | GCGGATGTCTAGAGGCGGCTGCAGCGGCCTGCTGCGGTGCGGCAAG | |
| | GGCACCACCTACGAGGGCGGCGTGCGGGAACCCGCCCTGGCTTTTTG | |
| | GCCTGGCCACATCGCCCCTGGCGTTACCCACGAGCTGGCTTCTAGCCT | |
| | GGACCTGCTGCCCACCCTGGCCGCACTGGCCGGAGCTCCACTGCCTA | |
| | ATGTGACCCTGGATGGCTTCGACCTGTCCCCTCTGCTGCTCGGCACCG | |
| | GCAAGAGCCCTAGACAGAGCCTGTTCTTCTACCCCTCCTACCCTGATG | |
| | AGGTGCGGGGCGTCTTTGCCGTCAGGACCGGCAAATACAAGGCCCAT | |
| | TTCTTTACACAGGGCAGCGCCCACTCTGATACCACAGCCGACCCTGCC | |
| | TGCCACGCCAGCTCCAGCCTGACCGCCCACGAGCCTCCTCTGCTATAC | |
| | GACCTGAGCAAGGACCCTGGCGAGAACTACAACCTGCTGGGTGGCGT | |
| | GGCCGGCGCTACACCTGAGGTGCTGCAGGCCCTGAAGCAGCTGCAGC | |
| | TGCTTAAGGCCCAACTGGACGCCGCTGTGACCTTCGGCCCTAGCCAG | |
| | GTGGCCAGAGGAGAAGATCCCGCCCTGCAAATCTGCTGCCACCCTGG | |
| | ATGTACCCCTCGGCCCGCTTGTTGTCACTGCCCCGACCCTCACGCCTG | |
| | AGGTACCAATCAACCTCTGGATTACAAAATTTGTGAAAGATTGACTGGT | |
| | ATTCTTAACTATGTTGCTCCTTTTACGCTATGTGGATACGCTGCTTTAAT | |
| | GCCTTTGTATCATGCTATTGCTTCCCGTATGGCTTTCATTTTCTCCTCCT | |
| | TGTATAAATCCTGGTTGCTGTCTCTTTATGAGGAGTTGTGGCCCGTTGT | |
| | CAGGCAACGTGGCGTGGTGTGCACTGTGTTTGCTGACGCAACCCCCAC | |
| | TGGTTGGGGCATTGCCACCACCTGTCAGCTCCTTTCCGGGACTTTCGC | |
| | TTTCCCCCTCCCTATTGCCACGGCGGAACTCATCGCCGCCTGCCTTGC | |
| | CCGCTGCTGGACAGGGGCTCGGCTGTTGGGCACTGACAATTCCGTGG | |
| | TGTTGTCGGGGAAATCATCGTCCTTTCCTTGGCTGCTCGCCTGTGTTGC | |
| | CACCTGGATTCTGCGCGGGACGTCCTTCTGCTACGTCCCTTCGGCCCT | |
| | CAATCCAGCGGACCTTCCTTCCCGCGGCCTGCTGCCGGCTCTGCGGC | |
| | CTCTTCCGCGTCTTCGCCTTCGCCCTCAGACGAGTCGGATCTCCCTTT | |
| | GGGCCGCCTCCCCGCGAGCTCATTTGTGAAATTTGTGATGCTATTGCTT | |
| | TATTTGTAACCATTATAAGCTGCAATAAACAAGTTAACAACAACAATTGC | |
| | ATTCATTTTATGTTTCAGGTTCAGGGGGAGGTGTGGGAGGTTTTTTGAG | |
| | TCCTAGGAGGAACCCCTAGTGATGGAGTTGGCCACTCCCTCTCTGCGC | |
| | GCTCGCTCGCTCACTGAGGCCGGGCGACCAAAGGTCGCCCGACGCCC | |
| | GGGCTTTGCCCGGGCGGCCTCAGTGAGCGAGCGAGCGCGCAGAGAG | |
| | GGAGTGGCCAA | |
| CMV-COGS- | GCGCGCTCGCTCGCTCACTGAGGCCGCCCGGGCAAAGCCCGGGCGT | 22 |
| Hyper | CGGGCGACCTTTGGTCGCCCGGCCTCAGTGAGCGAGCGAGCGCGCA | |
| (ATP0138) | GAGAGGGAGTGGCCAACTCCATCACTAGGGGTTCCTTTGTCGACCTCT | |
| | AGGAAGACCTTCAATATTGGCCATTAGCCATATTATTCATTGGTTATATA | |
| | GCATAAATCAATATTGGCTATTGGCCATTGCATACGTTGTATCTATATCA | |
| | TAATATGTACATTTATATTGGCTCATGTCCAATATGACCGCCATGTTGCA | |
| | TTGATTATTGACTAGTTATTAATAGTAATCAATTACGGGGTCATTAGTTC | |
| | ATAGCCCATATATGGAGTTCCGCGTTACATAACTTACGGTAAATGGCCC | |
| | GCCTGGCTGACCGCCCAACGACCCCCGCCCATTGACGTCAATAATGAC | |
| | GTATGTTCCCATAGTAACGCCAATAGGGACTTTCCATTGACGTCAATGG | |
| | GTGGAGTATTTACGGTAAACTGCCCACTTGGCAGTACATCAAGTGTATC | |
| | ATATGCCAAGTCCGCCCCCTATTGACGTCAATGACGGTAAATGGCCCG | |
| | CCTGGCATTATGCCCAGTACATGACCTTACGGGACTTTCCTACTTGGCA | |
| | GTACATCTACGTATTAGTCATCGCTATTACCATGGTGATGCGGTTTTGG | |
| | CAGTACACCAATGGGCGTGGATAGCGGTTTGACTCACGGGGATTTCCA | |
| | AGTCTCCACCCCATTGACGTCAATGGGAGTTTGTTTTGGCACCAAAATC | |
| | AACGGGACTTTCCAAAATGTCGTAATAACCCCGCCCCGTTGACGCAAAT | |
| | GGGCGGTAGGCGTGTACGGTGGGAGGTCTATATAAGCAGAGCTCGTTT | |
| | AGTGAACCGTCAGATCACTAGACACTTTGTGGCGGTAGTTTATCACAGT | |
| | TAAATTGCTAACGCAGTCAGTGCTTCTGACACAACAGTCTCGAACTTAA | |
| | GCTGCAGAAGTTGGTCGTGAGGCACTGGGCAGGTAAGTATCAAGGTTA | |
| | CAAGACAGGTTTAAGGCGACCAATAGAAACTGGGCTTGTCGAGACAGA | |
| | GAAGACTCTTGCGTTTCTGATAGGCACCTATTGGTCTTACTGACATCCA | |
| | CTTTGCCTTTCTCTCCACAGGTGTCCACTCCCAGTTCAATTACAGCTCT | |
| | TAAGGCTAGAGTACTTAATACGACTCACTATAGGAGATCTATGAGCATG | |
| | GGCGCTCCTAGAAGCCTGCTGCTGGCCCTGGCTGCCGGCCTGGCCGT | |
| | GGCTAGACCTCCAAACATCGTGCTGATCTTCGCCGACGACCTGGGCTA | |
| | TGGTGACCTGGGCTGCTACGGCCACCCCTCTTCTACAACACCCAATCT | |
| | GGACCAGCTGGCCGCTGGCGGCCTGAGATTCACAGACTTCTACGTGC | |
| | CAGTGTCCCTGTGCACCCCTTCTAGAGCCGCTCTCCTGACCGGCAGAC | |
| | TGCCTGTGCGGATGGGCATGTACCCCGGAGTGCTGGTGCCCAGCAGT | |
| | AGAGGAGGACTGCCTCTGGAAGAGGTGACCGTGGCCGAGGTGCTGGC | |
| | CGCCAGAGGCTACCTGACAGGAATGGCCGGAAAATGGCACCTGGGAG | |
| | TGGGCCCAGAAGGCGCCTTCCTGCCACCACACCAGGGCTTTCACCGG | |
| | TTCCTGGGGATCCCTTACAGCCACGACCAAGGCCCTTGTCAGAACCTG | |
| | ACATGCTTCCCCCCCGCCACACCTTGCGACGGCGGCTGTGACCAGGG | |
| | CCTTGTGCCTATCCCCCTGCTGGCCAACCTGAGCGTGGAAGCCCAGCC | |
| | TCCATGGCTGCCTGGCCTCGAGGCCAGATACGTGGCCTTCGCTCATGA | |
| | TCTGATGGCCGATGCCCAGAGACAGGACAGACCTTTTTTCCTGTATTAC | |
| | GCCAGCCACCACACCCACTACCCTCAGTTCAGCGGACAGAGCTTCGCC | |
| | GAGCGGAGCGGCAGAGGCCCCTTCGGCGACAGCCTGATGGAACTGGA | |
| | CGCCGCTGTTGGAACCCTGATGACCGCCATTGGCGATCTGGGCCTGCT | |
| | CGAGGAAACCCTGGTGATCTTCACCGCCGATAACGGCCCTGAGCTGAT | |
| | GCGGATGTCTAACGGCGGCTGCAGCGGCCTGCTGCGGTGCGGCAAG | |
| | GGCACCACCTACGAGGGCGGCGTGCGGGAACCCGCCCTGGCTTTTTG | |
| | GCCTGGCCACATCGCCCCTGGCGTTACCCACGAGCTGGCTTCTAGCCT | |
| | GGACCTGCTGCCCACCCTGGCCGCACTGGCCGGAGCTCCACTGCCTA | |
| | ATGTGACCCTGGATGGCTTCGACCTGTCCCCTCTGCTGCTCGGCACCG | |
| | GCAAGAGCCCTAGACAGAGCCTGTTCTTCTACCCCTCCTACCCTGATG | |
| | AGGTGCGGGGCGTCTTTGCCGTCAGGACCGGCAAATACAAGGCCCAT | |
| | TTCTTTACACAGGGCAGCGCCCACTCTGATACCACAGCCGACCCTGCC | |
| | TGCCACGCCAGCTCCAGCCTGACCGCCCACGAGCCTCCTCTGCTATAC | |
| | GACCTGAGCAAGGACCCTGGCGAGAACTACAACCTGCTGGGTGGCGT | |
| | GGCCGGCGCTACACCTGAGGTGCTGCAGGCCCTGAAGCAGCTGCAGC | |
| | TGCTTAAGGCCCAACTGGACGCCGCTGTGACCTTCGGCCCTAGCCAG | |
| | GTGGCCAGAGGAGAAGATCCCGCCCTGCAAATCTGCTGCCACCCTGG | |
| | ATGTACCCCTCGGCCCGCTTGTTGTCACTGCCCCGACCCTCACGCCTG | |
| | AGGTACCAATCAACCTCTGGATTACAAAATTTGTGAAAGATTGACTGGT | |
| | ATTCTTAACTATGTTGCTCCTTTTACGCTATGTGGATACGCTGCTTTAAT | |
| | GCCTTTGTATCATGCTATTGCTTCCCGTATGGCTTTCATTTTCTCCTCCT | |
| | TGTATAAATCCTGGTTGCTGTCTCTTTATGAGGAGTTGTGGCCCGTTGT | |
| | CAGGCAACGTGGCGTGGTGTGCACTGTGTTTGCTGACGCAACCCCCAC | |
| | TGGTTGGGGCATTGCCACCACCTGTCAGCTCCTTTCCGGGACTTTCGC | |
| | TTTCCCCCTCCCTATTGCCACGGCGGAACTCATCGCCGCCTGCCTTGC | |
| | CCGCTGCTGGACAGGGGCTCGGCTGTTGGGCACTGACAATTCCGTGG | |
| | TGTTGTCGGGGAAATCATCGTCCTTTCCTTGGCTGCTCGCCTGTGTTGC | |
| | CACCTGGATTCTGCGCGGGACGTCCTTCTGCTACGTCCCTTCGGCCCT | |
| | CAATCCAGCGGACCTTCCTTCCCGCGGCCTGCTGCCGGCTCTGCGGC | |
| | CTCTTCCGCGTCTTCGCCTTCGCCCTCAGACGAGTCGGATCTCCCTTT | |
| | GGGCCGCCTCCCCGCGAGCTCATTTGTGAAATTTGTGATGCTATTGCTT | |
| | TATTTGTAACCATTATAAGCTGCAATAAACAAGTTAACAACAACAATTGC | |
| | ATTCATTTTATGTTTCAGGTTCAGGGGGAGGTGTGGGAGGTTTTTTGAG | |
| | TCCTAGGAGGAACCCCTAGTGATGGAGTTGGCCACTCCCTCTCTGCGC | |
| | GCTCGCTCGCTCACTGAGGCCGGGCGACCAAAGGTCGCCCGACGCCC | |
| | GGGCTTTGCCCGGGCGGCCTCAGTGAGCGAGCGAGCGCGCAGAGAG | |
| | GGAGTGGCCAA | |
| IL2SS- | ATGTATCGTATGCAACTCCTTTCTTGCATCGCCCTCTCTCTGGCTCTCG | 23 |
| HerW2.HC - | TGACCAATTCCGAGGTGCAGCTGGTGGAGAGCGGCGGCGGCCTGGTG | |
| P2A - IL2SS- | CAGCCTGGCGGCAGCCTGAGGCTGAGCTGCGCCGCCTCCGGCTTCAA | |
| HERW2.LC - | CATCAAGGACACCTACATCCACTGGGTCCGGCAGGCCCCCGGCAAGG | |
| coding | GCCTGGAGTGGGTGGCCAGGATCTACCCCACCAACGGCTACACCAGG | |
| sequence/ | TACGCCGACAGCGTGAAGGGCAGGTTCACCATCAGCGCCGACACCAG | |
| includes D356E | CAAGAACACCGCCTACCTGCAGATGAACTCCCTGAGGGCCGAGGACA | |
| and L358M | CCGCCGTGTACTACTGCAGCAGATGGGGCGGCGACGGCTTATACGCC | |
| modifications | ATGGACTACTGGGGCCAGGGCACCCTGGTGACCACCTCCAGCGCCAG | |
| | CACCAAGGGGCCCTCGGTCTTCCCCCTGGCACCCTCCTCCAAGAGCA | |
| | CCTCTGGGGGCACAGCGGCCCTGGGCTGCCTGGTCAAGGACTACTTC | |
| | CCCGAACCGGTGACGGTGTCGTGGAACTCAGGCGCCCTGACCAGCGG | |
| | CGTGCACACCTTCCCGGCTGTCCTACAGTCCTCAGGACTCTACTCCCT | |
| | CAGCAGCGTGGTGACCGTGCCCTCCAGCAGCTTGGGCACCCAGACCT | |
| | ACATCTGCAACGTGAATCACAAGCCCAGCAACACCAAGGTGGACAAGA | |
| | AAGTTGAGCCCAAATCTTGTGACAAAACTCACACATGCCCACCGTGCC | |
| | CAGCACCTGAACTCCTGGGGGGACCGTCAGTCTTCCTCTTCCCCCCAA | |
| | AACCCAAGGACACCCTCATGATCTCCCGGACCCCTGAGGTCACATGCG | |
| | TGGTGGTGGACGTGAGCCACGAAGACCCTGAGGTCAAGTTCAACTGGT | |
| | ACGTGGACGGCGTGGAGGTGCATAATGCCAAGACAAAGCCGCGGGAG | |
| | GAGCAGTACAACAGCACGTACCGTGTGGTCAGCGTCCTCACCGTCCTG | |
| | CACCAGGACTGGCTGAATGGCAAGGAGTACAAGTGCAAGGTGTCCAAC | |
| | AAAGCCCTCCCAGCCCCCATCGAGAAAACCATCTCCAAAGCCAAAGGG | |
| | CAGCCCCGAGAACCACAGGTGTACACCCTGCCCCCATCCCGGGAGGA | |
| | GATGACCAAGAACCAGGTCAGCCTGACCTGCCTGGTCAAAGGCTTCTA | |
| | TCCCAGCGACATCGCCGTGGAGTGGGAGAGCAATGGGCAGCCGGAGA | |
| | ACAACTACAAGACCACGCCTCCCGTGCTGGACTCCGACGGCTCCTTCT | |
| | TCCTATACAGCAAGCTCACCGTGGACAAGAGCAGGTGGCAGCAGGGG | |
| | AACGTCTTCTCATGCTCCGTGATGCATGAGGCTCTGCACAACCACTACA | |
| | CGCAGAAGAGCCTCTCCCTGTCTCCGGGTAAACGTGCCAAGCGTGGCT | |
| | CTGGGGCTACAAACTTTAGCCTCCTGAAGCAGGCTGGCGACGTGGAG | |
| | GAAAATCCCGGCCCAATGTATAGGATGCAGTTGCTGTCCTGTATCGCA | |
| | CTCAGTCTTGCACTCGTTACAAACAGTGACATCCAGATGACCCAGAGC | |
| | CCCTCCAGCCTGTCCGCCAGCGTGGGCGACAGGGTGACCATCACCTG | |
| | CCGGGCCTCCCAGGACGTGAACACCGCCGTGGCCTGGTACCAGCAGA | |
| | AGCCCGGCAAGGCCCCCAAGCTGCTGATCTACAGCGCCAGCTTCCTGT | |
| | ACAGCGGCGTGCCCAGCAGGTTCTCCGGCAGCAGGAGCGGCACCGAC | |
| | TTCACCCTGACCATCAGCAGCCTGCAGCCCGAGGACTTCGCCACCTAC | |
| | TACTGCCAGCAGCACTACACCACCCCCCCCACCTTCGGCCAGGGCAC | |
| | CAAGGTGGAGATCAAGCGTACGGTGGCTGCACCATCTGTCTTCATCTT | |
| | CCCGCCATCTGATGAGCAGTTGAAATCTGGAACTGCCTCTGTTGTGTG | |
| | CCTGCTGAATAACTTCTATCCCAGAGAGGCCAAAGTACAGTGGAAGGT | |
| | GGATAACGCCCTCCAATCGGGTAACTCCCAGGAGAGTGTCACAGAGCA | |
| | GGACAGCAAGGACAGCACCTACAGCCTCAGCAGCACCCTGACGCTGA | |
| | GCAAAGCAGACTACGAGAAACACAAAGTCTACGCCTGCGAAGTCACCC | |
| | ATCAGGGCCTGAGCTCGCCCGTCACAAAGAGCTTCAACAGGGGAGAG | |
| | TGT | |
| ATP0142 | GCATTGATTATTGACTAGTTATTAATAGTAATCAATTACGGGGTCATTAG | 24 |
| (CMV.HER.W2. | TTCATAGCCCATATATGGAGTTCCGCGTTACATAACTTACGGTAAATGG | |
| HC.LC.DELM. | CCCGCCTGGCTGACCGCCCAACGACCCCCGCCCATTGACGTCAATAAT | |
| SV40pA) | GACGTATGTTCCCATAGTAACGCCAATAGGGACTTTCCATTGACGTCAA | |
| | TGGGTGGAGTATTTACGGTAAACTGCCCACTTGGCAGTACATCAAGTGT | |
| | ATCATATGCCAAGTCCGCCCCCTATTGACGTCAATGACGGTAAATGGC | |
| | CCGCCTGGCATTATGCCCAGTACATGACCTTACGGGACTTTCCTACTTG | |
| | GCAGTACATCTACGTATTAGTCATCGCTATTACCATGGTGATGCGGTTT | |
| | TGGCAGTACACCAATGGGCGTGGATAGCGGTTTGACTCACGGGGATTT | |
| | CCAAGTCTCCACCCCATTGACGTCAATGGGAGTTTGTTTTGGCACCAAA | |
| | ATCAACGGGACTTTCCAAAATGTCGTAATAACCCCGCCCCGTTGACGC | |
| | AAATGGGCGGTAGGCGTGTACGGTGGGAGGTCTATATAAGCAGAGCTC | |
| | GTTTAGTGAACCGTCAGATCACTAGACACTTTGTGGCGGTAGTTTATCA | |
| | CAGTTAAATTGCTAACGCAGTCAGTGCTTCTGACACAACAGTCTCGAAC | |
| | TTAAGCTGCAGAAGTTGGTCGTGAGGCACTGGGCAGGTAAGTATCAAG | |
| | GTTACAAGACAGGTTTAAGGAGACCAATAGAAACTGGGCTTGTCGAGA | |
| | CAGAGAAGACTCTTGCGTTTCTGATAGGCACCTATTGGTCTTACTGACA | |
| | TCCACTTTGCCTTTCTCTCCACAGGTGTCCACTCCCAGTTCAATTACAG | |
| | CTCTTAAGGCTAGAGTACTTAATACGACTCACTATAGGCTGCCGCCACC | |
| | ATGTATCGTATGCAACTCCTTTCTTGCATCGCCCTCTCTCTGGCTCTCG | |
| | TGACCAATTCCGAGGTGCAGCTGGTGGAGAGCGGCGGCGGCCTGGTG | |
| | CAGCCTGGCGGCAGCCTGAGGCTGAGCTGCGCCGCCTCCGGCTTCAA | |
| | CATCAAGGACACCTACATCCACTGGGTCCGGCAGGCCCCCGGCAAGG | |
| | GCCTGGAGTGGGTGGCCAGGATCTACCCCACCAACGGCTACACCAGG | |
| | TACGCCGACAGCGTGAAGGGCAGGTTCACCATCAGCGCCGACACCAG | |
| | CAAGAACACCGCCTACCTGCAGATGAACTCCCTGAGGGCCGAGGACA | |
| | CCGCCGTGTACTACTGCAGCAGATGGGGCGGCGACGGCTTATACGCC | |
| | ATGGACTACTGGGGCCAGGGCACCCTGGTGACCACCTCCAGCGCCAG | |
| | CACCAAGGGGCCCTCGGTCTTCCCCCTGGCACCCTCCTCCAAGAGCA | |
| | CCTCTGGGGGCACAGCGGCCCTGGGCTGCCTGGTCAAGGACTACTTC | |
| | CCCGAACCGGTGACGGTGTCGTGGAACTCAGGCGCCCTGACCAGCGG | |
| | CGTGCACACCTTCCCGGCTGTCCTACAGTCCTCAGGACTCTACTCCCT | |
| | CAGCAGCGTGGTGACCGTGCCCTCCAGCAGCTTGGGCACCCAGACCT | |
| | ACATCTGCAACGTGAATCACAAGCCCAGCAACACCAAGGTGGACAAGA | |
| | AAGTTGAGCCCAAATCTTGTGACAAAACTCACACATGCCCACCGTGCC | |
| | CAGCACCTGAACTCCTGGGGGGACCGTCAGTCTTCCTCTTCCCCCCAA | |
| | AACCCAAGGACACCCTCATGATCTCCCGGACCCCTGAGGTCACATGCG | |
| | TGGTGGTGGACGTGAGCCACGAAGACCCTGAGGTCAAGTTCAACTGGT | |
| | ACGTGGACGGCGTGGAGGTGCATAATGCCAAGACAAAGCCGCGGGAG | |
| | GAGCAGTACAACAGCACGTACCGTGTGGTCAGCGTCCTCACCGTCCTG | |
| | CACCAGGACTGGCTGAATGGCAAGGAGTACAAGTGCAAGGTGTCCAAC | |
| | AAAGCCCTCCCAGCCCCCATCGAGAAAACCATCTCCAAAGCCAAAGGG | |
| | CAGCCCCGAGAACCACAGGTGTACACCCTGCCCCCATCCCGGGAGGA | |
| | GATGACCAAGAACCAGGTCAGCCTGACCTGCCTGGTCAAAGGCTTCTA | |
| | TCCCAGCGACATCGCCGTGGAGTGGGAGAGCAATGGGCAGCCGGAGA | |
| | ACAACTACAAGACCACGCCTCCCGTGCTGGACTCCGACGGCTCCTTCT | |
| | TCCTATACAGCAAGCTCACCGTGGACAAGAGCAGGTGGCAGCAGGGG | |
| | AACGTCTTCTCATGCTCCGTGATGCATGAGGCTCTGCACAACCACTACA | |
| | CGCAGAAGAGCCTCTCCCTGTCTCCGGGTAAACGTGCCAAGCGTGGCT | |
| | CTGGGGCTACAAACTTTAGCCTCCTGAAGCAGGCTGGCGACGTGGAG | |
| | GAAAATCCCGGCCCAATGTATAGGATGCAGTTGCTGTCCTGTATCGCA | |
| | CTCAGTCTTGCACTCGTTACAAACAGTGACATCCAGATGACCCAGAGC | |
| | CCCTCCAGCCTGTCCGCCAGCGTGGGCGACAGGGTGACCATCACCTG | |
| | CCGGGCCTCCCAGGACGTGAACACCGCCGTGGCCTGGTACCAGCAGA | |
| | AGCCCGGCAAGGCCCCCAAGCTGCTGATCTACAGCGCCAGCTTCCTGT | |
| | ACAGCGGCGTGCCCAGCAGGTTCTCCGGCAGCAGGAGCGGCACCGAC | |
| | TTCACCCTGACCATCAGCAGCCTGCAGCCCGAGGACTTCGCCACCTAC | |
| | TACTGCCAGCAGCACTACACCACCCCCCCCACCTTCGGCCAGGGCAC | |
| | CAAGGTGGAGATCAAGCGTACGGTGGCTGCACCATCTGTCTTCATCTT | |
| | CCCGCCATCTGATGAGCAGTTGAAATCTGGAACTGCCTCTGTTGTGTG | |
| | CCTGCTGAATAACTTCTATCCCAGAGAGGCCAAAGTACAGTGGAAGGT | |
| | GGATAACGCCCTCCAATCGGGTAACTCCCAGGAGAGTGTCACAGAGCA | |
| | GGACAGCAAGGACAGCACCTACAGCCTCAGCAGCACCCTGACGCTGA | |
| | GCAAAGCAGACTACGAGAAACACAAAGTCTACGCCTGCGAAGTCACCC | |
| | ATCAGGGCCTGAGCTCGCCCGTCACAAAGAGCTTCAACAGGGGAGAG | |
| | TGTTAATAGAGCGGCCGCTTCGAGCAGACATGATAAGATACATTGATGA | |
| | GTTTGGACAAACCACAACTAGAATGCAGTGAAAAAAATGCTTTATTTGT | |
| | GAAATTTGTGATGCTATTGCTTTATTTGTAACCATTATAAGCTGCAATAA | |
| | ACAAGTTAACAACAACAATTGCATTCATTTTATGTTTCAGGTTCAGGGG | |
| | GAGATGTGGGAGGTTTTTTAAAGCAAGTAAAACCTCTACAAATGTGGTA | |
| | AA | |
| ATPO146 | GGCCTCCGCGCCGGGTTTTGGCGCCTCCCGCGGGCGCCCCCCTCCTC | 25 |
| (UBC.HER.W2. | ACGGCGAGCGCTGCCACGTCAGACGAAGGGCGCAGCGAGCGTCCTGA | |
| HC.LC.DELM. | TCCTTCCGCCCGGACGCTCAGGACAGCGGCCCGCTGCTCATAAGACT | |
| SV40pA) | CGGCCTTAGAACCCCAGTATCAGCAGAAGGACATTTTAGGACGGGACT | |
| | TGGGTGACTCTAGGGCACTGGTTTTCTTTCCAGAGAGCGGAACAGGCG | |
| | AGGAAAAGTAGTCCCTTCTCGGCGATTCTGCGGAGGGATCTCCGTGGG | |
| | GCGGTGAACGCCGATGATTATATAAGGACGCGCCGGGTGTGGCACAG | |
| | CTAGTTCCGTCGCAGCCGGGATTTGGGTCGCAGTTCTTGTTTGTGGAT | |
| | CGCTGTGATCGTCACTTGGTGAGTAGCGGGCTGCTGGGCTGGCCGGG | |
| | GCTTTCGTGGCCGCCGGGCCGCTCGGTGGGACGGAGGCGTGTGGAG | |
| | AGCCCGCCAAGGGCTGTAGTCTGGGTCCGCGAGCAAGGTTGCCCTGA | |
| | ACTGGGGGTTGGGGGGAGCGCAGCAAAATGGCGGCTGTTCCCGAGTC | |
| | TTGAATGGAAGACGCTTGTGAGGCGGGCTGTGAGGTCGTTGAAACAAG | |
| | GTGGGGGGCATGGTGGGCGGCAAGAACCCAAGGTCTTGAGGCCTTCG | |
| | CTAATGCGGGAAAGCTCTTATTCGGGTGAGATGGGCTGGGGCACCATC | |
| | TGGGGACCCTGACGTGAAGTTTGTCACTGACTGGAGAACTCGGTTTGT | |
| | CGTCTGTTGCGGGGGCGGCAGTTATGGCGGTGCCGTTGGGCAGTGCA | |
| | CCCGTACCTTTGGGAGCGCGCGCCCTCGTCGTGTCGTGACGTCACCC | |
| | GTTCTGTTGGCTTATAATGCAGGGTGGGGCCACCTGCCGGTAGGTGTG | |
| | CGGTAGGCTTTTCTCCGTCGCAGGACGCAGGGTTCGGGCCAAGGGTA | |
| | GGCTCTCCTGAATCGACAGGCGCCGGACCTCTGGTGAGGGGAGGGAT | |
| | AAGTGAGGCGTCAGTTTCTCTGGTCGGTTTTATGTACCTATCTTCTTAA | |
| | GTAGCTGAAGCTCCGGTTTTGAACTATGCGCTCGGGGTTGGCGAGTGT | |
| | GTTTTGTGAAGTTTTTTAGGCACCTTTTGAAATGTAATCATTTGGGTCAA | |
| | TATGTAATTTTCAGTGTTAGACTAGTAAATTGTCCGCTAAATTCTGGCCG | |
| | TTTTTGGCTTTTTTGTTAGACCACTTTGTGGCGGTAGTTTATCACAGTTA | |
| | AATTGCTAACGCAGTCAGTGCTTCTGACACAACAGTCTCGAACTTAAGC | |
| | TGCAGAAGTTGGTCGTGAGGCACTGGGCAGGTAAGTATCAAGGTTACA | |
| | AGACAGGTTTAAGGCGACCAATAGAAACTGGGCTTGTCGAGACAGAGA | |
| | AGACTCTTGCGTTTCTGATAGGCACCTATTGGTCTTACTGACATCCACT | |
| | TTGCCTTTCTCTCCACAGGTGTCCACTCCCAGTTCAATTACAGCTCTTA | |
| | AGGCTAGAGTACTTAATACGACTCACTATAGGCTGCCGCCACCATGTAT | |
| | CGTATGCAACTCCTTTCTTGCATCGCCCTCTCTCTGGCTCTCGTGACCA | |
| | ATTCCGAGGTGCAGCTGGTGGAGAGCGGCGGCGGCCTGGTGCAGCCT | |
| | GGCGGCAGCCTGAGGCTGAGCTGCGCCGCCTCCGGCTTCAACATCAA | |
| | GGACACCTACATCCACTGGGTCCGGCAGGCCCCCGGCAAGGGCCTGG | |
| | AGTGGGTGGCCAGGATCTACCCCACCAACGGCTACACCAGGTACGCC | |
| | GACAGCGTGAAGGGCAGGTTCACCATCAGCGCCGACACCAGCAAGAA | |
| | CACCGCCTACCTGCAGATGAACTCCCTGAGGGCCGAGGACACCGCCG | |
| | TGTACTACTGCAGCAGATGGGGCGGCGACGGCTTATACGCCATGGACT | |
| | ACTGGGGCCAGGGCACCCTGGTGACCACCTCCAGCGCCAGCACCAAG | |
| | GGGCCCTCGGTCTTCCCCCTGGCACCCTCCTCCAAGAGCACCTCTGG | |
| | GGGCACAGCGGCCCTGGGCTGCCTGGTCAAGGACTACTTCCCCGAAC | |
| | CGGTGACGGTGTCGTGGAACTCAGGCGCCCTGACCAGCGGCGTGCAC | |
| | ACCTTCCCGGCTGTCCTACAGTCCTCAGGACTCTACTCCCTCAGCAGC | |
| | GTGGTGACCGTGCCCTCCAGCAGCTTGGGCACCCAGACCTACATCTGC | |
| | AACGTGAATCACAAGCCCAGCAACACCAAGGTGGACAAGAAAGTTGAG | |
| | CCCAAATCTTGTGACAAAACTCACACATGCCCACCGTGCCCAGCACCT | |
| | GAACTCCTGGGGGGACCGTCAGTCTTCCTCTTCCCCCCAAAACCCAAG | |
| | GACACCCTCATGATCTCCCGGACCCCTGAGGTCACATGCGTGGTGGTG | |
| | GACGTGAGCCACGAAGACCCTGAGGTCAAGTTCAACTGGTACGTGGAC | |
| | GGCGTGGAGGTGCATAATGCCAAGACAAAGCCGCGGGAGGAGCAGTA | |
| | CAACAGCACGTACCGTGTGGTCAGCGTCCTCACCGTCCTGCACCAGGA | |
| | CTGGCTGAATGGCAAGGAGTACAAGTGCAAGGTGTCCAACAAAGCCCT | |
| | CCCAGCCCCCATCGAGAAAACCATCTCCAAAGCCAAAGGGCAGCCCC | |
| | GAGAACCACAGGTGTACACCCTGCCCCCATCCCGGGAGGAGATGACC | |
| | AAGAACCAGGTCAGCCTGACCTGCCTGGTCAAAGGCTTCTATCCCAGC | |
| | GACATCGCCGTGGAGTGGGAGAGCAATGGGCAGCCGGAGAACAACTA | |
| | CAAGACCACGCCTCCCGTGCTGGACTCCGACGGCTCCTTCTTCCTATA | |
| | CAGCAAGCTCACCGTGGACAAGAGCAGGTGGCAGCAGGGGAACGTCT | |
| | TCTCATGCTCCGTGATGCATGAGGCTCTGCACAACCACTACACGCAGA | |
| | AGAGCCTCTCCCTGTCTCCGGGTAAACGTGCCAAGCGTGGCTCTGGG | |
| | GCTACAAACTTTAGCCTCCTGAAGCAGGCTGGCGACGTGGAGGAAAAT | |
| | CCCGGCCCAATGTATAGGATGCAGTTGCTGTCCTGTATCGCACTCAGT | |
| | CTTGCACTCGTTACAAACAGTGACATCCAGATGACCCAGAGCCCCTCC | |
| | AGCCTGTCCGCCAGCGTGGGCGACAGGGTGACCATCACCTGCCGGGC | |
| | CTCCCAGGACGTGAACACCGCCGTGGCCTGGTACCAGCAGAAGCCCG | |
| | GCAAGGCCCCCAAGCTGCTGATCTACAGCGCCAGCTTCCTGTACAGCG | |
| | GCGTGCCCAGCAGGTTCTCCGGCAGCAGGAGCGGCACCGACTTCACC | |
| | CTGACCATCAGCAGCCTGCAGCCCGAGGACTTCGCCACCTACTACTGC | |
| | CAGCAGCACTACACCACCCCCCCCACCTTCGGCCAGGGCACCAAGGT | |
| | GGAGATCAAGCGTACGGTGGCTGCACCATCTGTCTTCATCTTCCCGCC | |
| | ATCTGATGAGCAGTTGAAATCTGGAACTGCCTCTGTTGTGTGCCTGCTG | |
| | AATAACTTCTATCCCAGAGAGGCCAAAGTACAGTGGAAGGTGGATAAC | |
| | GCCCTCCAATCGGGTAACTCCCAGGAGAGTGTCACAGAGCAGGACAG | |
| | CAAGGACAGCACCTACAGCCTCAGCAGCACCCTGACGCTGAGCAAAG | |
| | CAGACTACGAGAAACACAAAGTCTACGCCTGCGAAGTCACCCATCAGG | |
| | GCCTGAGCTCGCCCGTCACAAAGAGCTTCAACAGGGGAGAGTGTTAAT | |
| | AGAGCGGCCGCTTCGAGCAGACATGATAAGATACATTGATGAGTTTGG | |
| | ACAAACCACAACTAGAATGCAGTGAAAAAAATGCTTTATTTGTGAAATTT | |
| | GTGATGCTATTGCTTTATTTGTAACCATTATAAGCTGCAATAAACAAGTT | |
| | AACAACAACAATTGCATTCATTTTATGTTTCAGGTTCAGGGGGAGATGT | |
| | GGGAGGTTTTTTAAAGCAAGTAAAACCTCTACAAATGTGGTAAA | |
| ATP0090 | GCATTGATTATTGACTAGTTATTAATAGTAATCAATTACGGGGTCATTAG | 26 |
| (CMV.W1.HER2. | TTCATAGCCCATATATGGAGTTCCGCGTTACATAACTTACGGTAAATGG | |
| HC.LC.SV40pA) | CCCGCCTGGCTGACCGCCCAACGACCCCCGCCCATTGACGTCAATAAT | |
| | GACGTATGTTCCCATAGTAACGCCAATAGGGACTTTCCATTGACGTCAA | |
| | TGGGTGGAGTATTTACGGTAAACTGCCCACTTGGCAGTACATCAAGTGT | |
| | ATCATATGCCAAGTCCGCCCCCTATTGACGTCAATGACGGTAAATGGC | |
| | CCGCCTGGCATTATGCCCAGTACATGACCTTACGGGACTTTCCTACTTG | |
| | GCAGTACATCTACGTATTAGTCATCGCTATTACCATGGTGATGCGGTTT | |
| | TGGCAGTACACCAATGGGCGTGGATAGCGGTTTGACTCACGGGGATTT | |
| | CCAAGTCTCCACCCCATTGACGTCAATGGGAGTTTGTTTTGGCACCAAA | |
| | ATCAACGGGACTTTCCAAAATGTCGTAATAACCCCGCCCCGTTGACGC | |
| | AAATGGGCGGTAGGCGTGTACGGTGGGAGGTCTATATAAGCAGAGCTC | |
| | GTTTAGTGAACCGTCAGATCACTAGACACTTTGTGGCGGTAGTTTATCA | |
| | CAGTTAAATTGCTAACGCAGTCAGTGCTTCTGACACAACAGTCTCGAAC | |
| | TTAAGCTGCAGAAGTTGGTCGTGAGGCACTGGGCAGGTAAGTATCAAG | |
| | GTTACAAGACAGGTTTAAGGAGACCAATAGAAACTGGGCTTGTCGAGA | |
| | CAGAGAAGACTCTTGCGTTTCTGATAGGCACCTATTGGTCTTACTGACA | |
| | TCCACTTTGCCTTTCTCTCCACAGGTGTCCACTCCCAGTTCAATTACAG | |
| | CTCTTAAGGCTAGAGTACTTAATACGACTCACTATAGGCTGCCGCCACC | |
| | ATGTACCGGATGCAGCTGCTGAGCTGTATCGCCCTGTCTCTGGCCCTC | |
| | GTGACCAACAGCGAAGTGCAGCTGGTGGAAAGCGGCGGAGGACTGGT | |
| | GCAGCCTGGCGGATCTCTGAGACTGAGCTGTGCCGCCAGCGGCTTCA | |
| | ACATCAAGGACACCTACATCCACTGCGTGCGCCAGGCCCCTGGCAAG | |
| | GGACTGGAATGGGTGGCCAGAATCTACCCCACCAACGGCTACACCAGA | |
| | TACGCCGACAGCGTGAAGGGCCGGTTCACCATCAGCGCCGACACCAG | |
| | CAAGAACACCGCCTACCTGCAGATGAACAGCCTGCGGGCCGAGGACA | |
| | CCGCCGTGTACTACTGTAGTAGATGGGGAGGCGACGGCTTCTACGCCA | |
| | TGGACTATTGGGGCCAGGGCACCCTCGTGACAGTGTCTAGTGCATCGA | |
| | CCAAGGGACCTTCGGTCTTCCCCCTGGCACCCTCCTCCAAGAGCACCT | |
| | CTGGGGGCACAGCGGCCCTGGGCTGCCTGGTCAAGGACTACTTCCCC | |
| | GAACCGGTGACGGTGTCGTGGAACTCAGGCGCCCTGACCAGCGGCGT | |
| | GCACACCTTCCCGGCTGTCCTACAGTCCTCAGGACTCTACTCCCTCAG | |
| | CAGCGTGGTGACCGTGCCCTCCAGCAGCTTGGGCACCCAGACCTACA | |
| | TCTGCAACGTGAATCACAAGCCCAGCAACACCAAGGTGGACAAGAAAG | |
| | TTGAACCAAAGAGCTGCGACAAGACCCACACGTGTCCCCCCTGCCCTG | |
| | CCCCTGAACTGCTGGGAGGCCCCAGCGTGTTCCTGTTCCCCCCAAAGC | |
| | CCAAGGACACCCTGATGATCAGCCGGACCCCCGAAGTGACCTGCGTG | |
| | GTGGTGGACGTGTCCCACGAGGACCCTGAAGTGAAGTTTAATTGGTAC | |
| | GTGGACGGCGTGGAAGTGCACAACGCCAAGACCAAGCCCAGAGAGGA | |
| | ACAGTACAACAGCACCTACCGGGTGGTGTCCGTGCTGACCGTGCTGCA | |
| | CCAGGACTGGCTGAACGGCAAAGAGTACAAGTGCAAGGTGTCCAACAA | |
| | GGCCCTGCCTGCCCCCATCGAGAAAACCATCAGCAAGGCCAAGGGCC | |
| | AGCCCCGCGAGCCTCAGGTCTACACACTGCCCCCCAGCCGGGAAGAG | |
| | ATGACCAAGAACCAGGTGTCCCTGACCTGCCTGGTCAAGGGCTTCTAC | |
| | CCCAGCGACATCGCCGTGGAATGGGAGAGCAACGGCCAGCCCGAGAA | |
| | CAACTACAAGACCACCCCCCCTGTGCTGGACAGCGACGGCTCATTCTT | |
| | CCTGTATAGCAAGCTGACCGTGGACAAGAGCCGGTGGCAGCAGGGCA | |
| | ACGTGTTCAGCTGCAGCGTGATGCACGAGGCCCTGCACAACCACTACA | |
| | CCCAGAAGTCCCTGAGCCTGAGCCCCAGAAAGCGGAGAGCCCCCGTG | |
| | AAGCAGACCCTGAACTTCGACCTGCTGAAGCTGGCCGGCGACGTGGA | |
| | AAGCAACCCTGGCCCTATGTACAGAATGCAGCTGCTGCTGCTGATCGC | |
| | CCTGAGCCTGGCCCTGGTGACCAACAGCGATATCCAGATGACCCAGAG | |
| | CCCCAGCAGCCTGTCTGCCAGCGTGGGCGACAGAGTGACCATCACCT | |
| | GTAGAGCCAGCCAGGACGTGAACACCGCCGTGGCCTGGTATCAGCAG | |
| | AAGCCTGGCAAGGCCCCCAAGCTGCTGATCTACAGCGCCAGCTTCCTG | |
| | TACAGCGGCGTGCCCAGCAGATTCAGCGGCAGCAGATCCGGCACCGA | |
| | CTTCACCCTGACCATCAGCTCCCTGCAGCCCGAGGACTTCGCCACCTA | |
| | CTACTGCCAGCAGCACTACACCACCCCCCCCACATTTGGCCAGGGCAC | |
| | CAAGGTGGAAATCAAGCGTACGGTGGCCGCCCCAAGCGTGTTCATCTT | |
| | CCCACCAAGCGATGAGCAGCTGAAGAGCGGAACCGCCAGCGTGGTGT | |
| | GCCTGCTGAACAACTTCTACCCACGGGAGGCCAAGGTGCAGTGGAAG | |
| | GTGGATAACGCCCTGCAGAGCGGAAACAGCCAGGAGAGCGTGACCGA | |
| | GCAGGATAGCAAGGATAGCACCTACAGCCTGAGCAGCACCCTGACCCT | |
| | GAGCAAGGCCGATTACGAGAAGCACAAGGTATACGCCTGCGAGGTGA | |
| | CCCACCAGGGACTGAGCAGCCCAGTGACCAAGAGCTTCAACCGCGGA | |
| | GAGTGCTGATAAAGCGGCCGCTTCGAGCAGACATGATAAGATACATTG | |
| | ATGAGTTTGGACAAACCACAACTAGAATGCAGTGAAAAAAATGCTTTAT | |
| | TTGTGAAATTTGTGATGCTATTGCTTTATTTGTAACCATTATAAGCTGCA | |
| | ATAAACAAGTTAACAACAACAATTGCATTCATTTTATGTTTCAGGTTCAG | |
| | GGGGAGATGTGGGAGGTTTTTTAAAGCAAGTAAAACCTCTACAAATGTG | |
| | GTAAA | |
| ATP0089 | GCATTGATTATTGACTAGTTATTAATAGTAATCAATTACGGGGTCATTAG | 27 |
| (CMV.ATX.HC. | TTCATAGCCCATATATGGAGTTCCGCGTTACATAACTTACGGTAAATGG | |
| LC.SV40pA) | CCCGCCTGGCTGACCGCCCAACGACCCCCGCCCATTGACGTCAATAAT | |
| | GACGTATGTTCCCATAGTAACGCCAATAGGGACTTTCCATTGACGTCAA | |
| | TGGGTGGAGTATTTACGGTAAACTGCCCACTTGGCAGTACATCAAGTGT | |
| | ATCATATGCCAAGTCCGCCCCCTATTGACGTCAATGACGGTAAATGGC | |
| | CCGCCTGGCATTATGCCCAGTACATGACCTTACGGGACTTTCCTACTTG | |
| | GCAGTACATCTACGTATTAGTCATCGCTATTACCATGGTGATGCGGTTT | |
| | TGGCAGTACACCAATGGGCGTGGATAGCGGTTTGACTCACGGGGATTT | |
| | CCAAGTCTCCACCCCATTGACGTCAATGGGAGTTTGTTTTGGCACCAAA | |
| | ATCAACGGGACTTTCCAAAATGTCGTAATAACCCCGCCCCGTTGACGC | |
| | AAATGGGCGGTAGGCGTGTACGGTGGGAGGTCTATATAAGCAGAGCTC | |
| | GTTTAGTGAACCGTCAGATCACTAGACACTTTGTGGCGGTAGTTTATCA | |
| | CAGTTAAATTGCTAACGCAGTCAGTGCTTCTGACACAACAGTCTCGAAC | |
| | TTAAGCTGCAGAAGTTGGTCGTGAGGCACTGGGCAGGTAAGTATCAAG | |
| | GTTACAAGACAGGTTTAAGGCGACCAATAGAAACTGGGCTTGTCGAGA | |
| | CAGAGAAGACTCTTGCGTTTCTGATAGGCACCTATTGGTCTTACTGACA | |
| | TCCACTTTGCCTTTCTCTCCACAGGTGTCCACTCCCAGTTCAATTACAG | |
| | CTCTTAAGGCTAGAGTACTTAATACGACTCACTATAGGCTGCCGCCACC | |
| | ATGTATCGTATGCAACTCCTTTCTTGCATCGCCCTCTCTCTGGCTCTCG | |
| | TGACCAATTCCGAAGTCCAACTGGTGGAGAGCGGAGGCGGGCTGGTG | |
| | CAACCAGGCGGAAGCCTTCGGCTGTCATGTGCCGCTTCTGGCTTCAAC | |
| | ATCAAGGATACCTACATCCACTGGGTAAGACAGGCTCCAGGGAAGGGA | |
| | CTGGAATGGGTAGCCCGTATTTATCCCACAAATGGTTACACCCGTTACG | |
| | CCGATAGCGTGAAGGGGAGGTTCACAATCTCCGCCGATACAAGTAAGA | |
| | ACACCGCTTACTTGCAGATGAACAGTCTTCGTGCTGAAGATACCGCTGT | |
| | TTACTATTGTAGCCGTTGGGGAGGGGACGGGTTCTATGCTATGGACTA | |
| | CTGGGGTCAGGGCACACTTGTGACCGTGTCCTCCGCATCCACCAAGG | |
| | GACCCAGCGTGTTCCCCTTGGCACCTTCCTCTAAATCAACATCTGGTG | |
| | GAACTGCTGCCCTCGGCTGTTTGGTCAAGGACTACTTTCCTGAGCCAG | |
| | TTACCGTATCTTGGAACTCTGGAGCCCTGACCAGCGGAGTTCACACGT | |
| | TCCCCGCTGTTCTCCAGTCTTCAGGACTCTACAGCCTGTCCAGCGTCG | |
| | TGACCGTGCCGTCCTCTTCCCTCGGCACCCAAACTTATATCTGCAATGT | |
| | GAACCATAAACCCTCCAACACTAAGGTGGACAAGAAAGTAGAGCCCAA | |
| | GAGTTGCGACAAAACCCATACCTGTCCACCCTGTCCTGCCCCTGAACT | |
| | GCTCGGAGGCCCTTCTGTGTTCCTCTTTCCGCCAAAGCCCAAGGATAC | |
| | TCTTATGATTTCACGCACCCCTGAGGTGACATGTGTTGTGGTAGATGTG | |
| | TCACACGAAGACCCTGAGGTGAAGTTCAACTGGTATGTGGACGGCGTA | |
| | GAAGTCCACAATGCTAAAACCAAACCCCGCGAGGAGCAGTATAATAGC | |
| | ACCTACCGTGTCGTGAGCGTTCTGACCGTGCTGCATCAGGACTGGCTG | |
| | AACGGAAAGGAATACAAGTGTAAGGTAAGCAATAAGGCTCTCCCTGCC | |
| | CCCATTGAGAAGACCATTTCCAAGGCAAAGGGGCAGCCCCGCGAACCT | |
| | CAGGTTTACACCCTCCCGCCCAGCCGCGATGAATTGACTAAAAATCAG | |
| | GTGAGCCTTACATGTCTGGTGAAGGGCTTTTATCCTTCCGACATCGCTG | |
| | TGGAATGGGAGAGCAACGGACAACCTGAGAATAACTATAAGACCACAC | |
| | CCCCAGTGCTGGACAGCGACGGCTCCTTTTTCCTGTATTCCAAACTGA | |
| | CAGTGGACAAGTCCCGCTGGCAACAGGGCAACGTTTTCTCTTGTAGCG | |
| | TCATGCACGAGGCTCTGCACAACCATTACACCCAGAAATCCTTGTCTCT | |
| | GTCCCCTGGCAAGCGTGCCAAGCGTGGCTCTGGGGCTACAAACTTTAG | |
| | CCTCCTGAAGCAGGCTGGCGACGTGGAGGAAAATCCCGGCCCAATGT | |
| | ATAGGATGCAGTTGCTGTCCTGTATCGCACTCAGTCTTGCACTCGTTAC | |
| | AAACAGTGACATCCAGATGACACAGAGCCCTTCCAGCCTGTCAGCGTC | |
| | AGTCGGCGACCGCGTGACCATCACTTGCAGAGCCTCACAGGATGTGAA | |
| | TACTGCTGTGGCGTGGTATCAACAGAAGCCCGGCAAAGCCCCCAAACT | |
| | GCTCATCTACTCCGCCAGTTTCCTCTACAGCGGCGTCCCATCACGGTT | |
| | CTCTGGCTCTCGTAGCGGCACGGATTTCACCCTTACTATCTCTAGTCTT | |
| | CAGCCTGAGGATTTTGCCACTTACTATTGCCAACAGCACTATACTACAC | |
| | CACCTACATTTGGGCAGGGCACTAAGGTAGAAATCAAACGCACCGTGG | |
| | CTGCCCCTTCAGTTTTCATCTTCCCACCCAGCGACGAGCAACTGAAGTC | |
| | AGGAACTGCCAGCGTGGTCTGCCTGCTCAATAACTTCTACCCCCGCGA | |
| | GGCTAAAGTTCAGTGGAAAGTGGACAACGCTCTCCAAAGTGGCAATTC | |
| | CCAAGAAAGCGTGACCGAGCAGGACAGTAAGGATAGCACATACAGCCT | |
| | GTCTTCAACACTTACCCTTTCCAAAGCCGACTACGAAAAACATAAGGTT | |
| | TATGCCTGCGAAGTTACCCATCAGGGTCTGTCCTCACCTGTTACCAAGT | |
| | CTTTCAACCGCGGCGAATGTTAATAGAGCGGCCGCTTCGAGCAGACAT | |
| | GATAAGATACATTGATGAGTTTGGACAAACCACAACTAGAATGCAGTGA | |
| | AAAAAATGCTTTATTTGTGAAATTTGTGATGCTATTGCTTTATTTGTAACC | |
| | ATTATAAGCTGCAATAAACAAGTTAACAACAACAATTGCATTCATTTTAT | |
| | GTTTCAGGTTCAGGGGGAGATGTGGGAGGTTTTTTAAAGCAAGTAAAA | |
| | CCTCTACAAATGTGGTAAA | |
| ATP0092 | GCATTGATTATTGACTAGTTATTAATAGTAATCAATTACGGGGTCATTAG | 28 |
| (CMV.ATX. | TTCATAGCCCATATATGGAGTTCCGCGTTACATAACTTACGGTAAATGG | |
| HER.LC.HC. | CCCGCCTGGCTGACCGCCCAACGACCCCCGCCCATTGACGTCAATAAT | |
| SV40pA) | GACGTATGTTCCCATAGTAACGCCAATAGGGACTTTCCATTGACGTCAA | |
| | TGGGTGGAGTATTTACGGTAAACTGCCCACTTGGCAGTACATCAAGTGT | |
| | ATCATATGCCAAGTCCGCCCCCTATTGACGTCAATGACGGTAAATGGC | |
| | CCGCCTGGCATTATGCCCAGTACATGACCTTACGGGACTTTCCTACTTG | |
| | GCAGTACATCTACGTATTAGTCATCGCTATTACCATGGTGATGCGGTTT | |
| | TGGCAGTACACCAATGGGCGTGGATAGCGGTTTGACTCACGGGGATTT | |
| | CCAAGTCTCCACCCCATTGACGTCAATGGGAGTTTGTTTTGGCACCAAA | |
| | ATCAACGGGACTTTCCAAAATGTCGTAATAACCCCGCCCCGTTGACGC | |
| | AAATGGGCGGTAGGCGTGTACGGTGGGAGGTCTATATAAGCAGAGCTC | |
| | GTTTAGTGAACCGTCAGATCACTAGACACTTTGTGGCGGTAGTTTATCA | |
| | CAGTTAAATTGCTAACGCAGTCAGTGCTTCTGACACAACAGTCTCGAAC | |
| | TTAAGCTGCAGAAGTTGGTCGTGAGGCACTGGGCAGGTAAGTATCAAG | |
| | GTTACAAGACAGGTTTAAGGCGACCAATAGAAACTGGGCTTGTCGAGA | |
| | CAGAGAAGACTCTTGCGTTTCTGATAGGCACCTATTGGTCTTACTGACA | |
| | TCCACTTTGCCTTTCTCTCCACAGGTGTCCACTCCCAGTTCAATTACAG | |
| | CTCTTAAGGCTAGAGTACTTAATACGACTCACTATAGGCTGCCGCCACC | |
| | ATGTATCGTATGCAACTCCTTTCTTGCATCGCCCTCTCTCTGGCTCTCG | |
| | TGACCAATTCCGACATCCAGATGACACAGAGCCCTTCCAGCCTGTCAG | |
| | CGTCAGTCGGCGACCGCGTGACCATCACTTGCAGAGCCTCACAGGAT | |
| | GTGAATACTGCTGTGGCGTGGTATCAACAGAAGCCCGGCAAAGCCCCC | |
| | AAACTGCTCATCTACTCCGCCAGTTTCCTCTACAGCGGCGTCCCATCAC | |
| | GGTTCTCTGGCTCTCGTAGCGGCACGGATTTCACCCTTACTATCTCTAG | |
| | TCTTCAGCCTGAGGATTTTGCCACTTACTATTGCCAACAGCACTATACT | |
| | ACACCACCTACATTTGGGCAGGGCACTAAGGTAGAAATCAAACGCACC | |
| | GTGGCTGCCCCTTCAGTTTTCATCTTCCCACCCAGCGACGAGCAACTG | |
| | AAGTCAGGAACTGCCAGCGTGGTCTGCCTGCTCAATAACTTCTACCCC | |
| | CGCGAGGCTAAAGTTCAGTGGAAAGTGGACAACGCTCTCCAAAGTGGC | |
| | AATTCCCAAGAAAGCGTGACCGAGCAGGACAGTAAGGATAGCACATAC | |
| | AGCCTGTCTTCAACACTTACCCTTTCCAAAGCCGACTACGAAAAACATA | |
| | AGGTTTATGCCTGCGAAGTTACCCATCAGGGTCTGTCCTCACCTGTTAC | |
| | CAAGTCTTTCAACCGCGGCGAATGTCGTGCCAAGCGTGGCTCTGGGG | |
| | CTACAAACTTTAGCCTCCTGAAGCAGGCTGGCGACGTGGAGGAAAATC | |
| | CCGGCCCAATGTATAGGATGCAGTTGCTGTCCTGTATCGCACTCAGTC | |
| | TTGCACTCGTTACAAACAGTGAAGTCCAACTGGTGGAGAGCGGAGGCG | |
| | GGCTGGTGCAACCAGGCGGAAGCCTTCGGCTGTCATGTGCCGCTTCT | |
| | GGCTTCAACATCAAGGATACCTACATCCACTGGGTAAGACAGGCTCCA | |
| | GGGAAGGGACTGGAATGGGTAGCCCGTATTTATCCCACAAATGGTTAC | |
| | ACCCGTTACGCCGATAGCGTGAAGGGGAGGTTCACAATCTCCGCCGAT | |
| | ACAAGTAAGAACACCGCTTACTTGCAGATGAACAGTCTTCGTGCTGAAG | |
| | ATACCGCTGTTTACTATTGTAGCCGTTGGGGAGGGGACGGGTTCTATG | |
| | CTATGGACTACTGGGGTCAGGGCACACTTGTGACCGTGTCCTCCGCAT | |
| | CCACCAAGGGACCCAGCGTGTTCCCCTTGGCACCTTCCTCTAAATCAA | |
| | CATCTGGTGGAACTGCTGCCCTCGGCTGTTTGGTCAAGGACTACTTTC | |
| | CTGAGCCAGTTACCGTATCTTGGAACTCTGGAGCCCTGACCAGCGGAG | |
| | TTCACACGTTCCCCGCTGTTCTCCAGTCTTCAGGACTCTACAGCCTGTC | |
| | CAGCGTCGTGACCGTGCCGTCCTCTTCCCTCGGCACCCAAACTTATAT | |
| | CTGCAATGTGAACCATAAACCCTCCAACACTAAGGTGGACAAGAAAGTA | |
| | GAGCCCAAGAGTTGCGACAAAACCCATACCTGTCCACCCTGTCCTGCC | |
| | CCTGAACTGCTCGGAGGCCCTTCTGTGTTCCTCTTTCCGCCAAAGCCC | |
| | AAGGATACTCTTATGATTTCACGCACCCCTGAGGTGACATGTGTTGTGG | |
| | TAGATGTGTCACACGAAGACCCTGAGGTGAAGTTCAACTGGTATGTGG | |
| | ACGGCGTAGAAGTCCACAATGCTAAAACCAAACCCCGCGAGGAGCAGT | |
| | ATAATAGCACCTACCGTGTCGTGAGCGTTCTGACCGTGCTGCATCAGG | |
| | ACTGGCTGAACGGAAAGGAATACAAGTGTAAGGTAAGCAATAAGGCTC | |
| | TCCCTGCCCCCATTGAGAAGACCATTTCCAAGGCAAAGGGGCAGCCCC | |
| | GCGAACCTCAGGTTTACACCCTCCCGCCCAGCCGCGATGAATTGACTA | |
| | AAAATCAGGTGAGCCTTACATGTCTGGTGAAGGGCTTTTATCCTTCCGA | |
| | CATCGCTGTGGAATGGGAGAGCAACGGACAACCTGAGAATAACTATAA | |
| | GACCACACCCCCAGTGCTGGACAGCGACGGCTCCTTTTTCCTGTATTC | |
| | CAAACTGACAGTGGACAAGTCCCGCTGGCAACAGGGCAACGTTTTCTC | |
| | TTGTAGCGTCATGCACGAGGCTCTGCACAACCATTACACCCAGAAATC | |
| | CTTGTCTCTGTCCCCTGGCAAGTAATAGAGCGGCCGCTTCGAGCAGAC | |
| | ATGATAAGATACATTGATGAGTTTGGACAAACCACAACTAGAATGCAGT | |
| | GAAAAAAATGCTTTATTTGTGAAATTTGTGATGCTATTGCTTTATTTGTAA | |
| | CCATTATAAGCTGCAATAAACAAGTTAACAACAACAATTGCATTCATTTT | |
| | ATGTTTCAGGTTCAGGGGGAGATGTGGGAGGTTTTTTAAAGCAAGTAAA | |
| | ACCTCTACAAATGTGGTAAA | |
| Coding | GAAGTCCAACTGGTGGAGAGCGGAGGCGGGCTGGTGCAACCAGGCG | 29 |
| sequence of | GAAGCCTTCGGCTGTCATGTGCCGCTTCTGGCTTCAACATCAAGGATA | |
| ATX HC | CCTACATCCACTGGGTAAGACAGGCTCCAGGGAAGGGACTGGAATGG | |
| | GTAGCCCGTATTTATCCCACAAATGGTTACACCCGTTACGCCGATAGCG | |
| | TGAAGGGGAGGTTCACAATCTCCGCCGATACAAGTAAGAACACCGCTT | |
| | ACTTGCAGATGAACAGTCTTCGTGCTGAAGATACCGCTGTTTACTATTG | |
| | TAGCCGTTGGGGAGGGGACGGGTTCTATGCTATGGACTACTGGGGTC | |
| | AGGGCACACTTGTGACCGTGTCCTCCGCATCCACCAAGGGACCCAGC | |
| | GTGTTCCCCTTGGCACCTTCCTCTAAATCAACATCTGGTGGAACTGCTG | |
| | CCCTCGGCTGTTTGGTCAAGGACTACTTTCCTGAGCCAGTTACCGTATC | |
| | TTGGAACTCTGGAGCCCTGACCAGCGGAGTTCACACGTTCCCCGCTGT | |
| | TCTCCAGTCTTCAGGACTCTACAGCCTGTCCAGCGTCGTGACCGTGCC | |
| | GTCCTCTTCCCTCGGCACCCAAACTTATATCTGCAATGTGAACCATAAA | |
| | CCCTCCAACACTAAGGTGGACAAGAAAGTAGAGCCCAAGAGTTGCGAC | |
| | AAAACCCATACCTGTCCACCCTGTCCTGCCCCTGAACTGCTCGGAGGC | |
| | CCTTCTGTGTTCCTCTTTCCGCCAAAGCCCAAGGATACTCTTATGATTT | |
| | CACGCACCCCTGAGGTGACATGTGTTGTGGTAGATGTGTCACACGAAG | |
| | ACCCTGAGGTGAAGTTCAACTGGTATGTGGACGGCGTAGAAGTCCACA | |
| | ATGCTAAAACCAAACCCCGCGAGGAGCAGTATAATAGCACCTACCGTG | |
| | TCGTGAGCGTTCTGACCGTGCTGCATCAGGACTGGCTGAACGGAAAGG | |
| | AATACAAGTGTAAGGTAAGCAATAAGGCTCTCCCTGCCCCCATTGAGAA | |
| | GACCATTTCCAAGGCAAAGGGGCAGCCCCGCGAACCTCAGGTTTACAC | |
| | CCTCCCGCCCAGCCGCGATGAATTGACTAAAAATCAGGTGAGCCTTAC | |
| | ATGTCTGGTGAAGGGCTTTTATCCTTCCGACATCGCTGTGGAATGGGA | |
| | GAGCAACGGACAACCTGAGAATAACTATAAGACCACACCCCCAGTGCT | |
| | GGACAGCGACGGCTCCTTTTTCCTGTATTCCAAACTGACAGTGGACAA | |
| | GTCCCGCTGGCAACAGGGCAACGTTTTCTCTTGTAGCGTCATGCACGA | |
| | GGCTCTGCACAACCATTACACCCAGAAATCCTTGTCTCTGTCCCCTGGC | |
| | AAG | |
| Coding | GACATCCAGATGACACAGAGCCCTTCCAGCCTGTCAGCGTCAGTCGGC | 30 |
| sequence of | GACCGCGTGACCATCACTTGCAGAGCCTCACAGGATGTGAATACTGCT | |
| ATX LC | GTGGCGTGGTATCAACAGAAGCCCGGCAAAGCCCCCAAACTGCTCATC | |
| | TACTCCGCCAGTTTCCTCTACAGCGGCGTCCCATCACGGTTCTCTGGC | |
| | TCTCGTAGCGGCACGGATTTCACCCTTACTATCTCTAGTCTTCAGCCTG | |
| | AGGATTTTGCCACTTACTATTGCCAACAGCACTATACTACACCACCTAC | |
| | ATTTGGGCAGGGCACTAAGGTAGAAATCAAACGCACCGTGGCTGCCCC | |
| | TTCAGTTTTCATCTTCCCACCCAGCGACGAGCAACTGAAGTCAGGAACT | |
| | GCCAGCGTGGTCTGCCTGCTCAATAACTTCTACCCCCGCGAGGCTAAA | |
| | GTTCAGTGGAAAGTGGACAACGCTCTCCAAAGTGGCAATTCCCAAGAA | |
| | AGCGTGACCGAGCAGGACAGTAAGGATAGCACATACAGCCTGTCTTCA | |
| | ACACTTACCCTTTCCAAAGCCGACTACGAAAAACATAAGGTTTATGCCT | |
| | GCGAAGTTACCCATCAGGGTCTGTCCTCACCTGTTACCAAGTCTTTCAA | |
| | CCGCGGCGAATGT | |
| Coding | GAAGTGCAGCTGGTGGAAAGCGGCGGAGGACTGGTGCAGCCTGGCG | 31 |
| sequence of | GATCTCTGAGACTGAGCTGTGCCGCCAGCGGCTTCAACATCAAGGACA | |
| W1 HC | CCTACATCCACTGCGTGCGCCAGGCCCCTGGCAAGGGACTGGAATGG | |
| | GTGGCCAGAATCTACCCCACCAACGGCTACACCAGATACGCCGACAGC | |
| | GTGAAGGGCCGGTTCACCATCAGCGCCGACACCAGCAAGAACACCGC | |
| | CTACCTGCAGATGAACAGCCTGCGGGCCGAGGACACCGCCGTGTACT | |
| | ACTGTAGTAGATGGGGAGGCGACGGCTTCTACGCCATGGACTATTGGG | |
| | GCCAGGGCACCCTCGTGACAGTGTCTAGTGCAGCATCGACCAAGGGA | |
| | CCTTCGGTCTTCCCCCTGGCACCCTCCTCCAAGAGCACCTCTGGGGGC | |
| | ACAGCGGCCCTGGGCTGCCTGGTCAAGGACTACTTCCCCGAACCGGT | |
| | GACGGTGTCGTGGAACTCAGGCGCCCTGACCAGCGGCGTGCACACCT | |
| | TCCCGGCTGTCCTACAGTCCTCAGGACTCTACTCCCTCAGCAGCGTGG | |
| | TGACCGTGCCCTCCAGCAGCTTGGGCACCCAGACCTACATCTGCAACG | |
| | TGAATCACAAGCCCAGCAACACCAAGGTGGACAAGAAAGTTGAACCAA | |
| | AGAGCTGCGACAAGACCCACACGTGTCCCCCCTGCCCTGCCCCTGAA | |
| | CTGCTGGGAGGCCCCAGCGTGTTCCTGTTCCCCCCAAAGCCCAAGGA | |
| | CACCCTGATGATCAGCCGGACCCCCGAAGTGACCTGCGTGGTGGTGG | |
| | ACGTGTCCCACGAGGACCCTGAAGTGAAGTTTAATTGGTACGTGGACG | |
| | GCGTGGAAGTGCACAACGCCAAGACCAAGCCCAGAGAGGAACAGTAC | |
| | AACAGCACCTACCGGGTGGTGTCCGTGCTGACCGTGCTGCACCAGGA | |
| | CTGGCTGAACGGCAAAGAGTACAAGTGCAAGGTGTCCAACAAGGCCCT | |
| | GCCTGCCCCCATCGAGAAAACCATCAGCAAGGCCAAGGGCCAGCCCC | |
| | GCGAGCCTCAGGTCTACACACTGCCCCCCAGCCGGGAAGAGATGACC | |
| | AAGAACCAGGTGTCCCTGACCTGCCTGGTCAAGGGCTTCTACCCCAGC | |
| | GACATCGCCGTGGAATGGGAGAGCAACGGCCAGCCCGAGAACAACTA | |
| | CAAGACCACCCCCCCTGTGCTGGACAGCGACGGCTCATTCTTCCTGTA | |
| | TAGCAAGCTGACCGTGGACAAGAGCCGGTGGCAGCAGGGCAACGTGT | |
| | TCAGCTGCAGCGTGATGCACGAGGCCCTGCACAACCACTACACCCAGA | |
| | AGTCCCTGAGCCTGAGCCCCAGAAAG | |
| Coding | GATATCCAGATGACCCAGAGCCCCAGCAGCCTGTCTGCCAGCGTGGG | 32 |
| sequence of | CGACAGAGTGACCATCACCTGTAGAGCCAGCCAGGACGTGAACACCG | |
| W1 LC | CCGTGGCCTGGTATCAGCAGAAGCCTGGCAAGGCCCCCAAGCTGCTG | |
| | ATCTACAGCGCCAGCTTCCTGTACAGCGGCGTGCCCAGCAGATTCAGC | |
| | GGCAGCAGATCCGGCACCGACTTCACCCTGACCATCAGCTCCCTGCAG | |
| | CCCGAGGACTTCGCCACCTACTACTGCCAGCAGCACTACACCACCCCC | |
| | CCCACATTTGGCCAGGGCACCAAGGTGGAAATCAAGCGTACGGTGGC | |
| | CGCCCCAAGCGTGTTCATCTTCCCACCAAGCGATGAGCAGCTGAAGAG | |
| | CGGAACCGCCAGCGTGGTGTGCCTGCTGAACAACTTCTACCCACGGG | |
| | AGGCCAAGGTGCAGTGGAAGGTGGATAACGCCCTGCAGAGCGGAAAC | |
| | AGCCAGGAGAGCGTGACCGAGCAGGATAGCAAGGATAGCACCTACAG | |
| | CCTGAGCAGCACCCTGACCCTGAGCAAGGCCGATTACGAGAAGCACAA | |
| | GGTATACGCCTGCGAGGTGACCCACCAGGGACTGAGCAGCCCAGTGA | |
| | CCAAGAGCTTCAACCGCGGAGAGTGC | |
| Coding | GAGGTGCAGCTGGTGGAGAGCGGCGGCGGCCTGGTGCAGCCTGGCG | 33 |
| sequence of | GCAGCCTGAGGCTGAGCTGCGCCGCCTCCGGCTTCAACATCAAGGAC | |
| W2 HC | ACCTACATCCACTGGGTCCGGCAGGCCCCCGGCAAGGGCCTGGAGTG | |
| | GGTGGCCAGGATCTACCCCACCAACGGCTACACCAGGTACGCCGACA | |
| | GCGTGAAGGGCAGGTTCACCATCAGCGCCGACACCAGCAAGAACACC | |
| | GCCTACCTGCAGATGAACTCCCTGAGGGCCGAGGACACCGCCGTGTA | |
| | CTACTGCAGCAGATGGGGCGGCGACGGCTTATACGCCATGGACTACT | |
| | GGGGCCAGGGCACCCTGGTGACCACCTCCAGCGCCAGCACCAAGGG | |
| | GCCCTCGGTCTTCCCCCTGGCACCCTCCTCCAAGAGCACCTCTGGGG | |
| | GCACAGCGGCCCTGGGCTGCCTGGTCAAGGACTACTTCCCCGAACCG | |
| | GTGACGGTGTCGTGGAACTCAGGCGCCCTGACCAGCGGCGTGCACAC | |
| | CTTCCCGGCTGTCCTACAGTCCTCAGGACTCTACTCCCTCAGCAGCGT | |
| | GGTGACCGTGCCCTCCAGCAGCTTGGGCACCCAGACCTACATCTGCAA | |
| | CGTGAATCACAAGCCCAGCAACACCAAGGTGGACAAGAAAGTTGAGCC | |
| | CAAATCTTGTGACAAAACTCACACATGCCCACCGTGCCCAGCACCTGA | |
| | ACTCCTGGGGGGACCGTCAGTCTTCCTCTTCCCCCCAAAACCCAAGGA | |
| | CACCCTCATGATCTCCCGGACCCCTGAGGTCACATGCGTGGTGGTGGA | |
| | CGTGAGCCACGAAGACCCTGAGGTCAAGTTCAACTGGTACGTGGACG | |
| | GCGTGGAGGTGCATAATGCCAAGACAAAGCCGCGGGAGGAGCAGTAC | |
| | AACAGCACGTACCGTGTGGTCAGCGTCCTCACCGTCCTGCACCAGGAC | |
| | TGGCTGAATGGCAAGGAGTACAAGTGCAAGGTGTCCAACAAAGCCCTC | |
| | CCAGCCCCCATCGAGAAAACCATCTCCAAAGCCAAAGGGCAGCCCCGA | |
| | GAACCACAGGTGTACACCCTGCCCCCATCCCGGGAGGAGATGACCAA | |
| | GAACCAGGTCAGCCTGACCTGCCTGGTCAAAGGCTTCTATCCCAGCGA | |
| | CATCGCCGTGGAGTGGGAGAGCAATGGGCAGCCGGAGAACAACTACA | |
| | AGACCACGCCTCCCGTGCTGGACTCCGACGGCTCCTTCTTCCTATACA | |
| | GCAAGCTCACCGTGGACAAGAGCAGGTGGCAGCAGGGGAACGTCTTC | |
| | TCATGCTCCGTGATGCATGAGGCTCTGCACAACCACTACACGCAGAAG | |
| | AGCCTCTCCCTGTCTCCGGGTAAA | |
| Coding | GACATCCAGATGACCCAGAGCCCCTCCAGCCTGTCCGCCAGCGTGGG | 34 |
| sequence of | CGACAGGGTGACCATCACCTGCCGGGCCTCCCAGGACGTGAACACCG | |
| W2 LC | CCGTGGCCTGGTACCAGCAGAAGCCCGGCAAGGCCCCCAAGCTGCTG | |
| | ATCTACAGCGCCAGCTTCCTGTACAGCGGCGTGCCCAGCAGGTTCTCC | |
| | GGCAGCAGGAGCGGCACCGACTTCACCCTGACCATCAGCAGCCTGCA | |
| | GCCCGAGGACTTCGCCACCTACTACTGCCAGCAGCACTACACCACCCC | |
| | CCCCACCTTCGGCCAGGGCACCAAGGTGGAGATCAAGCGTACGGTGG | |
| | CTGCACCATCTGTCTTCATCTTCCCGCCATCTGATGAGCAGTTGAAATC | |
| | TGGAACTGCCTCTGTTGTGTGCCTGCTGAATAACTTCTATCCCAGAGAG | |
| | GCCAAAGTACAGTGGAAGGTGGATAACGCCCTCCAATCGGGTAACTCC | |
| | CAGGAGAGTGTCACAGAGCAGGACAGCAAGGACAGCACCTACAGCCT | |
| | CAGCAGCACCCTGACGCTGAGCAAAGCAGACTACGAGAAACACAAAGT | |
| | CTACGCCTGCGAAGTCACCCATCAGGGCCTGAGCTCGCCCGTCACAAA | |
| | GAGCTTCAACAGGGGAGAGTGT | |
| Trastuzumab | EVQLVESGGGLVQPGGSLRLSCAASGFNIKDTYIHWVRQAPGKGLEWVA | 35 |
| heavy chain | RIYPTNGYTRYADSVKGRFTISADTSKNTAYLQMNSLRAEDTAVYYCSRW | |
| protein | GGDGFYAMDYWGQGTLVTVSSASTKGPSVFPLAPSSKSTSGGTAALGCL | |
| sequence | VKDYFPEPVTVSWNSGALTSGVHTFPAVLQSSGLYSLSSVVTVPSSSLGT | |
| | QTYICNVNHKPSNTKVDKKVEPKSCDKTHTCPPCPAPELLGGPSVFLFPPK | |
| | PKDTLMISRTPEVTCVVVDVSHEDPEVKFNWYVDGVEVHNAKTKPREEQY | |
| | NSTYRVVSVLTVLHQDWLNGKEYKCKVSNKALPAPIEKTISKAKGQPREPQ | |
| | VYTLPPSREEMTKNQVSLTCLVKGFYPSDIAVEWESNGQPENNYKTTPPV | |
| | LDSDGSFFLYSKLTVDKSRWQQGNVFSCSVMHEALHNHYTQKSLSLSPG | |
| Trastuzumab | DIQMTQSPSSLSASVGDRVTITCRASQDVNTAVAWYQQKPGKAPKLLIYSA | 36 |
| light chain | SFLYSGVPSRFSGSRSGTDFTLTISSLQPEDFATYYCQQHYTTPPTFGQGT | |
| protein | KVEIKRTVAAPSVFIFPPSDEQLKSGTASVVCLLNNFYPREAKVQWKVDNA | |
| sequence | LQSGNSQESVTEQDSKDSTYSLSSTLTLSKADYEKHKVYACEVTHQGLSS | |
| | PVTKSFNRGEC | |
| Coding | CGTGCCAAGCGTGGCTCTGGGGCTACAAACTTTAGCCTCCTGAAGCAG | 37 |
| sequence for | GCTGGCGACGTGGAGGAAAATCCCGGCCCA | |
| Furin P2A | | |
| Coding | ATGTATCGTATGCAACTCCTTTCTTGCATCGCCCTCTCTCTGGCTCTCG | 38 |
| sequence for | TGACCAATTCC | |
| Interleukin-2 | | |
| signal | | |
| sequence | | |
| ATP0091 | GCATTGATTATTGACTAGTTATTAATAGTAATCAATTACGGGGTCATTAG | 39 |
| (CMV.W2.HCLC. | TTCATAGCCCATATATGGAGTTCCGCGTTACATAACTTACGGTAAATGG | |
| SV40pA) | CCCGCCTGGCTGACCGCCCAACGACCCCCGCCCATTGACGTCAATAAT | |
| | GACGTATGTTCCCATAGTAACGCCAATAGGGACTTTCCATTGACGTCAA | |
| | TGGGTGGAGTATTTACGGTAAACTGCCCACTTGGCAGTACATCAAGTGT | |
| | ATCATATGCCAAGTCCGCCCCCTATTGACGTCAATGACGGTAAATGGC | |
| | CCGCCTGGCATTATGCCCAGTACATGACCTTACGGGACTTTCCTACTTG | |
| | GCAGTACATCTACGTATTAGTCATCGCTATTACCATGGTGATGCGGTTT | |
| | TGGCAGTACACCAATGGGCGTGGATAGCGGTTTGACTCACGGGGATTT | |
| | CCAAGTCTCCACCCCATTGACGTCAATGGGAGTTTGTTTTGGCACCAAA | |
| | ATCAACGGGACTTTCCAAAATGTCGTAATAACCCCGCCCCGTTGACGC | |
| | AAATGGGCGGTAGGCGTGTACGGTGGGAGGTCTATATAAGCAGAGCTC | |
| | GTTTAGTGAACCGTCAGATCACTAGACACTTTGTGGCGGTAGTTTATCA | |
| | CAGTTAAATTGCTAACGCAGTCAGTGCTTCTGACACAACAGTCTCGAAC | |
| | TTAAGCTGCAGAAGTTGGTCGTGAGGCACTGGGCAGGTAAGTATCAAG | |
| | GTTACAAGACAGGTTTAAGGAGACCAATAGAAACTGGGCTTGTCGAGA | |
| | CAGAGAAGACTCTTGCGTTTCTGATAGGCACCTATTGGTCTTACTGACA | |
| | TCCACTTTGCCTTTCTCTCCACAGGTGTCCACTCCCAGTTCAATTACAG | |
| | CTCTTAAGGCTAGAGTACTTAATACGACTCACTATAGGCTGCCGCCACC | |
| | ATGTATCGTATGCAACTCCTTTCTTGCATCGCCCTCTCTCTGGCTCTCG | |
| | TGACCAATTCCGAGGTGCAGCTGGTGGAGAGCGGCGGCGGCCTGGTG | |
| | CAGCCTGGCGGCAGCCTGAGGCTGAGCTGCGCCGCCTCCGGCTTCAA | |
| | CATCAAGGACACCTACATCCACTGGGTCCGGCAGGCCCCCGGCAAGG | |
| | GCCTGGAGTGGGTGGCCAGGATCTACCCCACCAACGGCTACACCAGG | |
| | TACGCCGACAGCGTGAAGGGCAGGTTCACCATCAGCGCCGACACCAG | |
| | CAAGAACACCGCCTACCTGCAGATGAACTCCCTGAGGGCCGAGGACA | |
| | CCGCCGTGTACTACTGCAGCAGATGGGGCGGCGACGGCTTATACGCC | |
| | ATGGACTACTGGGGCCAGGGCACCCTGGTGACCACCTCCAGCGCCAG | |
| | CACCAAGGGGCCCTCGGTCTTCCCCCTGGCACCCTCCTCCAAGAGCA | |
| | CCTCTGGGGGCACAGCGGCCCTGGGCTGCCTGGTCAAGGACTACTTC | |
| | CCCGAACCGGTGACGGTGTCGTGGAACTCAGGCGCCCTGACCAGCGG | |
| | CGTGCACACCTTCCCGGCTGTCCTACAGTCCTCAGGACTCTACTCCCT | |
| | CAGCAGCGTGGTGACCGTGCCCTCCAGCAGCTTGGGCACCCAGACCT | |
| | ACATCTGCAACGTGAATCACAAGCCCAGCAACACCAAGGTGGACAAGA | |
| | AAGTTGAGCCCAAATCTTGTGACAAAACTCACACATGCCCACCGTGCC | |
| | CAGCACCTGAACTCCTGGGGGGACCGTCAGTCTTCCTCTTCCCCCCAA | |
| | AACCCAAGGACACCCTCATGATCTCCCGGACCCCTGAGGTCACATGCG | |
| | TGGTGGTGGACGTGAGCCACGAAGACCCTGAGGTCAAGTTCAACTGGT | |
| | ACGTGGACGGCGTGGAGGTGCATAATGCCAAGACAAAGCCGCGGGAG | |
| | GAGCAGTACAACAGCACGTACCGTGTGGTCAGCGTCCTCACCGTCCTG | |
| | CACCAGGACTGGCTGAATGGCAAGGAGTACAAGTGCAAGGTGTCCAAC | |
| | AAAGCCCTCCCAGCCCCCATCGAGAAAACCATCTCCAAAGCCAAAGGG | |
| | CAGCCCCGAGAACCACAGGTGTACACCCTGCCCCCATCCCGGGATGA | |
| | GCTGACCAAGAACCAGGTCAGCCTGACCTGCCTGGTCAAAGGCTTCTA | |
| | TCCCAGCGACATCGCCGTGGAGTGGGAGAGCAATGGGCAGCCGGAGA | |
| | ACAACTACAAGACCACGCCTCCCGTGCTGGACTCCGACGGCTCCTTCT | |
| | TCCTATACAGCAAGCTCACCGTGGACAAGAGCAGGTGGCAGCAGGGG | |
| | AACGTCTTCTCATGCTCCGTGATGCATGAGGCTCTGCACAACCACTACA | |
| | CGCAGAAGAGCCTCTCCCTGTCTCCGGGTAAACGTGCCAAGCGTGGCT | |
| | CTGGGGCTACAAACTTTAGCCTCCTGAAGCAGGCTGGCGACGTGGAG | |
| | GAAAATCCCGGCCCAATGTATAGGATGCAGTTGCTGTCCTGTATCGCA | |
| | CTCAGTCTTGCACTCGTTACAAACAGTGACATCCAGATGACCCAGAGC | |
| | CCCTCCAGCCTGTCCGCCAGCGTGGGCGACAGGGTGACCATCACCTG | |
| | CCGGGCCTCCCAGGACGTGAACACCGCCGTGGCCTGGTACCAGCAGA | |
| | AGCCCGGCAAGGCCCCCAAGCTGCTGATCTACAGCGCCAGCTTCCTGT | |
| | ACAGCGGCGTGCCCAGCAGGTTCTCCGGCAGCAGGAGCGGCACCGAC | |
| | TTCACCCTGACCATCAGCAGCCTGCAGCCCGAGGACTTCGCCACCTAC | |
| | TACTGCCAGCAGCACTACACCACCCCCCCCACCTTCGGCCAGGGCAC | |
| | CAAGGTGGAGATCAAGCGTACGGTGGCTGCACCATCTGTCTTCATCTT | |
| | CCCGCCATCTGATGAGCAGTTGAAATCTGGAACTGCCTCTGTTGTGTG | |
| | CCTGCTGAATAACTTCTATCCCAGAGAGGCCAAAGTACAGTGGAAGGT | |
| | GGATAACGCCCTCCAATCGGGTAACTCCCAGGAGAGTGTCACAGAGCA | |
| | GGACAGCAAGGACAGCACCTACAGCCTCAGCAGCACCCTGACGCTGA | |
| | GCAAAGCAGACTACGAGAAACACAAAGTCTACGCCTGCGAAGTCACCC | |
| | ATCAGGGCCTGAGCTCGCCCGTCACAAAGAGCTTCAACAGGGGAGAG | |
| | TGTTAATAGAGCGGCCGCTTCGAGCAGACATGATAAGATACATTGATGA | |
| | GTTTGGACAAACCACAACTAGAATGCAGTGAAAAAAATGCTTTATTTGT | |
| | GAAATTTGTGATGCTATTGCTTTATTTGTAACCATTATAAGCTGCAATAA | |
| | ACAAGTTAACAACAACAATTGCATTCATTTTATGTTTCAGGTTCAGGGG | |
| | GAGATGTGGGAGGTTTTTTAAAGCAAGTAAAACCTCTACAAATGTGGTA | |
| | AA | |
| Coding | GAGGTGCAGCTGGTGGAGAGCGGCGGCGGCCTGGTGCAGCCTGGCG | 40 |
| sequence for | GCAGCCTGAGGCTGAGCTGCGCCGCCTCCGGCTTCAACATCAAGGAC | |
| ATP0091 HC | ACCTACATCCACTGGGTCCGGCAGGCCCCCGGCAAGGGCCTGGAGTG | |
| | GGTGGCCAGGATCTACCCCACCAACGGCTACACCAGGTACGCCGACA | |
| | GCGTGAAGGGCAGGTTCACCATCAGCGCCGACACCAGCAAGAACACC | |
| | GCCTACCTGCAGATGAACTCCCTGAGGGCCGAGGACACCGCCGTGTA | |
| | CTACTGCAGCAGATGGGGCGGCGACGGCTTATACGCCATGGACTACT | |
| | GGGGCCAGGGCACCCTGGTGACCACCTCCAGCGCCAGCACCAAGGG | |
| | GCCCTCGGTCTTCCCCCTGGCACCCTCCTCCAAGAGCACCTCTGGGG | |
| | GCACAGCGGCCCTGGGCTGCCTGGTCAAGGACTACTTCCCCGAACCG | |
| | GTGACGGTGTCGTGGAACTCAGGCGCCCTGACCAGCGGCGTGCACAC | |
| | CTTCCCGGCTGTCCTACAGTCCTCAGGACTCTACTCCCTCAGCAGCGT | |
| | GGTGACCGTGCCCTCCAGCAGCTTGGGCACCCAGACCTACATCTGCAA | |
| | CGTGAATCACAAGCCCAGCAACACCAAGGTGGACAAGAAAGTTGAGCC | |
| | CAAATCTTGTGACAAAACTCACACATGCCCACCGTGCCCAGCACCTGA | |
| | ACTCCTGGGGGGACCGTCAGTCTTCCTCTTCCCCCCAAAACCCAAGGA | |
| | CACCCTCATGATCTCCCGGACCCCTGAGGTCACATGCGTGGTGGTGGA | |
| | CGTGAGCCACGAAGACCCTGAGGTCAAGTTCAACTGGTACGTGGACG | |
| | GCGTGGAGGTGCATAATGCCAAGACAAAGCCGCGGGAGGAGCAGTAC | |
| | AACAGCACGTACCGTGTGGTCAGCGTCCTCACCGTCCTGCACCAGGAC | |
| | TGGCTGAATGGCAAGGAGTACAAGTGCAAGGTGTCCAACAAAGCCCTC | |
| | CCAGCCCCCATCGAGAAAACCATCTCCAAAGCCAAAGGGCAGCCCCGA | |
| | GAACCACAGGTGTACACCCTGCCCCCATCCCGGGATGAGCTGACCAA | |
| | GAACCAGGTCAGCCTGACCTGCCTGGTCAAAGGCTTCTATCCCAGCGA | |
| | CATCGCCGTGGAGTGGGAGAGCAATGGGCAGCCGGAGAACAACTACA | |
| | AGACCACGCCTCCCGTGCTGGACTCCGACGGCTCCTTCTTCCTATACA | |
| | GCAAGCTCACCGTGGACAAGAGCAGGTGGCAGCAGGGGAACGTCTTC | |
| | TCATGCTCCGTGATGCATGAGGCTCTGCACAACCACTACACGCAGAAG | |
| | AGCCTCTCCCTGTCTCCGGGTAAA | |
| Coding | AGGACGTGAACACCGCCGTGGCCTGGTACCAGCAGAAGCCCGGCAAG | 41 |
| sequence for | GCCCCCAAGCTGCTGATCTACAGCGCCAGCTTCCTGTACAGCGGCGTG | |
| ATP0091 LC | CCCAGCAGGTTCTCCGGCAGCAGGAGCGGCACCGACTTCACCCTGAC | |
| | CATCAGCAGCCTGCAGCCCGAGGACTTCGCCACCTACTACTGCCAGCA | |
| | GCACTACACCACCCCCCCCACCTTCGGCCAGGGCACCAAGGTGGAGA | |
| | TCAAGCGTACGGTGGCTGCACCATCTGTCTTCATCTTCCCGCCATCTGA | |
| | TGAGCAGTTGAAATCTGGAACTGCCTCTGTTGTGTGCCTGCTGAATAAC | |
| | TTCTATCCCAGAGAGGCCAAAGTACAGTGGAAGGTGGATAACGCCCTC | |
| | CAATCGGGTAACTCCCAGGAGAGTGTCACAGAGCAGGACAGCAAGGA | |
| | CAGCACCTACAGCCTCAGCAGCACCCTGACGCTGAGCAAAGCAGACTA | |
| | CGAGAAACACAAAGTCTACGCCTGCGAAGTCACCCATCAGGGCCTGAG | |
| | CTCGCCCGTCACAAAGAGCTTCAACAGGGGAGAGTGT | |
| Coding | GAAGTCCAACTGGTGGAGAGCGGAGGCGGGCTGGTGCAACCAGGCG | 42 |
| sequence for | GAAGCCTTCGGCTGTCATGTGCCGCTTCTGGCTTCAACATCAAGGATA | |
| heavy chain | CCTACATCCACTGGGTAAGACAGGCTCCAGGGAAGGGACTGGAATGG | |
| variable domain | GTAGCCCGTATTTATCCCACAAATGGTTACACCCGTTACGCCGATAGCG | |
| (VH) for ATX | TGAAGGGGAGGTTCACAATCTCCGCCGATACAAGTAAGAACACCGCTT | |
| | ACTTGCAGATGAACAGTCTTCGTGCTGAAGATACCGCTGTTTACTATTG | |
| | TAGCCGTTGGGGAGGGGACGGGTTCTATGCTATGGACTACTGGGGTC | |
| | AGGGCACACTTGTGACCGTGTCCTCC | |
| Coding | GACATCCAGATGACACAGAGCCCTTCCAGCCTGTCAGCGTCAGTCGGC | 43 |
| sequence for | GACCGCGTGACCATCACTTGCAGAGCCTCACAGGATGTGAATACTGCT | |
| light chain | GTGGCGTGGTATCAACAGAAGCCCGGCAAAGCCCCCAAACTGCTCATC | |
| variable domain | TACTCCGCCAGTTTCCTCTACAGCGGCGTCCCATCACGGTTCTCTGGC | |
| (VL) for ATX | TCTCGTAGCGGCACGGATTTCACCCTTACTATCTCTAGTCTTCAGCCTG | |
| | AGGATTTTGCCACTTACTATTGCCAACAGCACTATACTACACCACCTAC | |
| | ATTTGGGCAGGGCACTAAGGTAGAAATCAAA | |
| Coding | GAAGTGCAGCTGGTGGAAAGCGGCGGAGGACTGGTGCAGCCTGGCG | 44 |
| sequence for | GATCTCTGAGACTGAGCTGTGCCGCCAGCGGCTTCAACATCAAGGACA | |
| heavy chain | CCTACATCCACTGCGTGCGCCAGGCCCCTGGCAAGGGACTGGAATGG | |
| variable domain | GTGGCCAGAATCTACCCCACCAACGGCTACACCAGATACGCCGACAGC | |
| (VH) for W1 | GTGAAGGGCCGGTTCACCATCAGCGCCGACACCAGCAAGAACACCGC | |
| | CTACCTGCAGATGAACAGCCTGCGGGCCGAGGACACCGCCGTGTACT | |
| | ACTGTAGTAGATGGGGAGGCGACGGCTTCTACGCCATGGACTATTGGG | |
| | GCCAGGGCACCCTCGTGACAGTGTCTAGTGCA | |
| Coding | GATATCCAGATGACCCAGAGCCCCAGCAGCCTGTCTGCCAGCGTGGG | 45 |
| sequence for | CGACAGAGTGACCATCACCTGTAGAGCCAGCCAGGACGTGAACACCG | |
| light chain | CCGTGGCCTGGTATCAGCAGAAGCCTGGCAAGGCCCCCAAGCTGCTG | |
| variable domain | ATCTACAGCGCCAGCTTCCTGTACAGCGGCGTGCCCAGCAGATTCAGC | |
| (VL) for W1 | GGCAGCAGATCCGGCACCGACTTCACCCTGACCATCAGCTCCCTGCAG | |
| | CCCGAGGACTTCGCCACCTACTACTGCCAGCAGCACTACACCACCCCC | |
| | CCCACATTTGGCCAGGGCACCAAGGTGGAAATCAAG | |
| Coding | GAGGTGCAGCTGGTGGAGAGCGGCGGCGGCCTGGTGCAGCCTGGCG | 46 |
| sequence for | GCAGCCTGAGGCTGAGCTGCGCCGCCTCCGGCTTCAACATCAAGGAC | |
| heavy chain | ACCTACATCCACTGGGTCCGGCAGGCCCCCGGCAAGGGCCTGGAGTG | |
| variable domain | GGTGGCCAGGATCTACCCCACCAACGGCTACACCAGGTACGCCGACA | |
| (VH) for W2 | GCGTGAAGGGCAGGTTCACCATCAGCGCCGACACCAGCAAGAACACC | |
| | GCCTACCTGCAGATGAACTCCCTGAGGGCCGAGGACACCGCCGTGTA | |
| | CTACTGCAGCAGATGGGGCGGCGACGGCTTATACGCCATGGACTACT | |
| | GGGGCCAGGGCACCCTGGTGACCACCTCCAGC | |
| Coding | GACATCCAGATGACCCAGAGCCCCTCCAGCCTGTCCGCCAGCGTGGG | 47 |
| sequence for | CGACAGGGTGACCATCACCTGCCGGGCCTCCCAGGACGTGAACACCG | |
| light chain | CCGTGGCCTGGTACCAGCAGAAGCCCGGCAAGGCCCCCAAGCTGCTG | |
| variable domain | ATCTACAGCGCCAGCTTCCTGTACAGCGGCGTGCCCAGCAGGTTCTCC | |
| (VL) for W2 | GGCAGCAGGAGCGGCACCGACTTCACCCTGACCATCAGCAGCCTGCA | |
| | GCCCGAGGACTTCGCCACCTACTACTGCCAGCAGCACTACACCACCCC | |
| | CCCCACCTTCGGCCAGGGCACCAAGGTGGAGATCAAG | |
| Coding | TGGGGCGGCGACGGCTTATACGCCATGGACTAC | 48 |
| sequence for | | |
| W2 HC CDR3 | | |
| Amino acid | WGGDGLYAMDY | 49 |
| sequence for | | |
| W2 HC CDR3 | | |
| Coding | TGGGGAGGCGACGGCTTCTACGCCATGGACTAT | 50 |
| sequence for | | |
| W1 HC CDR3 | | |
| Amino acid | WGGDGFYAMDY | 51 |
| sequence for | | |
| W1 HC CDR3 | | |

1. A method of transferring a polynucleotide to the central nervous system (CNS) of a subject, the method comprising:
administering to the subject an effective dose of:
a recombinant adeno-associated virus (rAAV) comprising:
a capsid comprising: a capsid protein having the amino acid sequence of SEQ ID NO: 1, and
the polynucleotide encapsulated by the capsid;
thereby transferring the polynucleotide to the CNS.

2. The method of claim 1, wherein the polynucleotide comprises a coding sequence of a therapeutic protein, optionally wherein the subject has a CNS disease.

3. (canceled)

4. The method of claim 2, wherein the CNS disease is:
(a) a lysosomal storage disease (LSD);
(b) a leukodystrophy, optionally wherein the CNS disease is:
(i) metachromatic leukodystrophy (MLD), optionally wherein the polynucleotide comprises a coding sequence encoding Arylsulfatase A (ARSA) or a functional variant thereof, optionally wherein:
(1) the polynucleotide comprises a coding sequence encoding Arylsulfatase A (ARSA) and wherein the polynucleotide comprises a coding sequence selected from SEQ ID NO: 2-4; or
(2) the polynucleotide comprises a coding sequence encoding Arylsulfatase A (ARSA) and wherein the polynucleotide comprises a coding sequence of SEQ ID NO: 7 or SEQ ID NO:8;
or
(ii) Krabbe's leukodystrophy, optionally wherein the polynucleotide comprises a coding sequence of galactocerebroside beta-galactosidase or a functional variant thereof;
(c) GM1 gangliosidosis, optionally wherein the polynucleotide comprises a coding sequence of galactosidase beta 1 (GLB-1) or a functional variant thereof; or
(d) a cancer, optionally wherein CNS disease is metastatic breast cancer, optionally wherein the therapeutic protein is an antigen binding protein against human epidermal growth factor receptor 2 (HER2), optionally wherein the polynucleotide comprises a sequence of SEQ ID NO: 23.

5-13. (canceled)

14. The method of claim 1, wherein the polynucleotide comprises a coding sequence of an antigen, optionally wherein:
(a) the antigen is a viral or bacterial antigen;
(b) the effective dose is sufficient to immunize the subject; or
(c) the effective dose is sufficient to induce an immune response to the antigen.

15. (canceled)

16. The method of claim 2, wherein the polynucleotide further comprises a regulatory sequence operably linked to the coding sequence, optionally wherein the regulatory sequence comprises a UbC promoter or a CMV promoter.

17. (canceled)

18. The method of claim 16, wherein the regulatory sequence comprises a UbC promoter and wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 9, SEQ ID NO:10, or SEQ ID NO:11.

19. The method of claim 1, wherein the administration:
(a) induces protein expression from the polynucleotide in the Substantia nigra, caudate nuclei, ependyma, or cortex of the subject; and/or
(b) is to the cerebrospinal fluid (CSF) of the subject, optionally wherein the administration is selected from intrathecal administration, intracranial administration, intracerebroventricular (ICV) administration and administration to the lateral ventricles of the brain of the subject, optionally wherein the intrathecal administration is by lumbar puncture (LP) and/or intra cisterna magna (ICM) injection.

20-21. (canceled)

22. The method of claim 1, wherein the effective dose is:
(a) between 1E10 to 1E16 genome copy numbers (GC) of the rAAV, 1E9 GC to 1E14 GC per gram brain mass, or administered at a concentration of 1E12 GC/ml to 1E17 GC/ml;
(b) administered systemically, optionally wherein the step of administration is performed intravenously;
(c) between 1E10-1E16 genome copy numbers (GC) of the rAAV or between 1E9-1E15 genome copy numbers (GC) of the rAAV per kg body weight; or
(d) an amount sufficient to induce detectable expression of the therapeutic protein in the CNS, Substantia nigra, caudate nuclei, ependyma, or cortex.

23-25. (canceled)

26. A method of treating a disease of the central nervous system (CNS), the method comprising:
administering to the CNS of a subject an effective dose of:
a recombinant adeno-associated virus (rAAV), the rAAV comprising:
a capsid polypeptide having the amino acid sequence of SEQ ID NO: 1 or
a variant thereof, and
a polynucleotide encoding a therapeutic protein.

27. A method of vaccination with a transgene, the method comprising:
administering to the central nervous system (CNS) of a subject an effective dose of:
a recombinant adeno-associated virus (rAAV), the rAAV comprising:
a capsid polypeptide having the amino acid sequence of SEQ ID NO: 1 or
a variant thereof, and
a polynucleotide encoding an antigen.

28. A recombinant adeno-associated virus (rAAV) comprising:
a capsid comprising: a capsid protein having the amino acid sequence of SEQ ID NO: 1 or a variant thereof, and a polynucleotide encapsulated by the capsid, wherein the polynucleotide comprises a coding sequence of a therapeutic protein associated with a CNS disease.

29. The rAAV of claim 28, wherein the CNS disease is:
(a) metachromatic leukodystrophy (MLD), optionally wherein the therapeutic protein is Arylsulfatase A (ARSA) or a functional variant thereof, optionally wherein:
(i) the therapeutic protein is Arylsulfatase A (ARSA) or a functional variant thereof and wherein the polynucleotide comprises a coding sequence selected from SEQ ID NO: 2-4; or
(ii) the therapeutic protein is Arylsulfatase A (ARSA) or a functional variant thereof and wherein the polynucleotide comprises a coding sequence of SEQ ID NO: 7 or SEQ ID NO:8;
(b) Krabbe's leukodystrophy, optionally wherein the polynucleotide encodes galactocerebrosidase or a functional variant thereof;
(c) GM1 gangliosidosis, optionally wherein the therapeutic protein is galactosidase, beta 1 (GLB-1) or a functional variant thereof; or
(d) cancer, optionally wherein the CNS disease is metastatic breast cancer, optionally wherein the therapeutic protein is an antigen binding protein (ABP) against human epidermal growth factor receptor 2 (HER2), optionally wherein the ABP against HER2 is trastuzumab, optionally wherein:
(i) the coding sequence comprises from 5′ to 3′, a coding sequence of a heavy chain of the ABP against HER2 and a coding sequence of a light chain of the ABP against HER2, optionally wherein (A) the coding sequence of a heavy chain comprises a sequence of SEQ ID NO: 29, 31 or 33 and/or (B) the coding sequence of a light chain comprises a sequence of SEQ ID NO: 30, 32 or 34; or
(ii) the coding sequence comprises from 5′ to 3′, a coding sequence of a light chain of the ABP against HER2 and a coding sequence of a heavy chain of the ABP against HER2, optionally wherein (A) the coding sequence of a heavy chain comprises a sequence of SEQ ID NO: 29, 31 or 33 and/or (B) the coding sequence of a light chain comprises a sequence of SEQ ID NO: 30, 32 or 34.

30-47. (canceled)

48. The rAAV of claim 28, wherein the polynucleotide:
(a) comprises a coding sequence of SEQ ID NO: 23;
(b) comprises a coding sequence having at least 80%, 90%, 95%, 96%, 97%, 98%, or 99% sequence identity to SEQ ID NO: 23;
(c) comprises the sequence of SEQ ID NO: 24-34, or a fragment thereof;
(d) comprises the sequence of SEQ ID NO: 24; or
(e) comprises the sequence of SEQ ID NO: 25.

49-53. (canceled)

54. The rAAV of claim 28, wherein the polynucleotide further comprises a regulatory sequence operably linked to the coding sequence, optionally wherein the regulatory sequence comprises a UbC promoter or a CMV promoter.

55. (canceled)

56. The rAAV of claim 54, wherein the regulatory sequence comprises a UbC promoter and wherein the nucleotide sequence of the UbC promoter comprises a nucleotide sequence having at least 90%, at least 95%, at least 96%, at least 97%, or at least 98%, at least 99%, or 100% sequence identity to SEQ ID NO: 9, SEQ ID NO:10, or SEQ ID NO:11.

57. A recombinant adeno-associated virus (rAAV) comprising:
a. a capsid comprising a capsid protein whose amino acid sequence comprises the amino acid sequence of SEQ ID NO: 1 or a variant thereof; and
b. a polynucleotide encapsulated by the capsid, wherein the polynucleotide comprises, in the 5′ to 3′ direction, (i) a 5′ inverted terminal repeat (ITR), (ii) a promoter which is a UbC promoter, a CAG promoter, or a CMV promoter, (iii) a coding sequence of Arylsulfatase A (ARSA) or a functional variant thereof, and (iv) a 3′ ITR.

58-66. (canceled)

67. The rAAV of claim 57, wherein the polynucleotide comprises the nucleotide sequence of SEQ ID NO: 19, SEQ ID NO:20, SEQ ID NO:21, or SEQ ID NO:22.

68. A pharmaceutical composition comprising the rAAV of claim 28.

69. A unit dose comprising the pharmaceutical composition of claim 68.

70. A method of transferring a polynucleotide to the central nervous system (CNS) of a subject, the method comprising administering to the subject an effective dose of the recombinant adeno-associated virus (rAAV) of claim 28.

71. (canceled)

72. A method of transferring a polynucleotide to the central nervous system (CNS) of a subject, the method comprising:
administering to the CNS an effective dose of:
a recombinant adeno-associated virus (rAAV) comprising:
a capsid having the amino acid sequence of SEQ ID NO: 1 or a variant thereof, and
a polynucleotide having the nucleic acid sequence of SEQ ID NO: 19 or 20, wherein the polynucleotide is encapsulated by the capsid,
wherein the subject has MLD.

73. A recombinant adeno-associated virus (rAAV) comprising:
a capsid having the amino acid sequence of SEQ ID NO: 1, and
a polynucleotide encapsulated by the capsid having the nucleic acid sequence of SEQ ID NO: 19 or 20.

74. A method of transferring a polynucleotide to the central nervous system (CNS) of a subject, the method comprising:
administering to the CNS an effective dose of:
a recombinant adeno-associated virus (rAAV) comprising:
a capsid having the amino acid sequence of SEQ ID NO: 1 or a variant thereof, and
a polynucleotide having the nucleic acid sequence of SEQ ID NO: 24 or 25, wherein the polynucleotide is encapsulated by the capsid,
wherein the subject has metastatic breast cancer.

75. A recombinant adeno-associated virus (rAAV) comprising:
a capsid having the amino acid sequence of SEQ ID NO: 1 or a variant thereof, and
a polynucleotide encapsulated by the capsid having the nucleic acid sequence of SEQ ID NO: 24 or 25.
